(19) United States
(12) Patent Application Publication
(10) Pub. No.: US 2024/0191270 A1
(43) Pub. Date: Jun. 13, 2024

(54) FERMENTATIVE PRODUCTION

(71) Applicant: INBIOSE N.V., Zwijnaarde (BE)

(72) Inventors: Joeri Beauprez, Zwijnaarde (BE); Pieter Coussement, Zwijnaarde (BE); Thomas Decoene, Zwijnaarde (BE)

(21) Appl. No.: 18/555,611

(22) PCT Filed: Apr. 15, 2022
(86) PCT No.: PCT/EP2022/060179

§ 371 Date: Oct. 16, 2023

(30) Foreign Application Priority Data
Apr. 16, 2021 (EP) 21168994.8

(51) Int. Cl. C12P 19/12; C12N 9/00; C12N 9/04; C12N 9/10; C12N 9/12; C12N 9/88; C12N 9/90; C12N 15/52; C12P 19/26

(52) U.S. Cl. CPC C12P 19/12; C12N 9/0006; C12N 9/1025; C12N 9/1048; C12N 9/1051; C12N 9/1085; C12N 9/1205; C12N 9/1241; C12N 9/88; C12N 9/90; C12N 9/93; C12N 15/52; C12P 19/26; C12Y 101/01271; C12Y 203/03009; C12Y 204/01038; C12Y 204/01069; C12Y 204/01152; C12Y 207/01006; C12Y 207/01052; C12Y 207/07023; C12Y 207/0703; C12Y 207/07043; C12Y 401/03001; C12Y 402/01047; C12Y 504/02008; C12Y 504/0201; C12Y 602/01001

(57) ABSTRACT

The present invention is in the technical field of synthetic biology and metabolic engineering. More particularly, the present invention is in the technical field of metabolically engineered cells and use of said cell in a cultivation, preferably a fermentation. The present invention describes a cell for the production of a compound. The cell comprises a pathway for the production of the compound, which can be a disaccharide, oligosaccharide and/or a Neu(n)Ac-containing bioproduct, wherein (n) is 4, 5, 7, 8 or 9 or a combination thereof. The cell is metabolically engineered for enhanced synthesis of acetyl-Coenzyme A. The invention also resides in a method of producing such compound by cultivation, preferably a fermentation, with such a cell.

### FIELD OF THE INVENTION

The present invention is in the technical field of synthetic biology and metabolic engineering. More particularly, the present invention is in the technical field of metabolically engineered cells and use of said cell in a cultivation, preferably a fermentation. The present invention describes a cell for the production of a compound. The cell comprises a pathway for the production of the compound, which can be a disaccharide, oligosaccharide and/or a Neu(n)Ac-containing bioproduct, wherein (n) is 4, 5, 7, 8 or 9 or a combination thereof. The cell is metabolically engineered for enhanced synthesis of acetyl-Coenzyme A. The invention also resides in a method of producing such compound by cultivation, preferably a fermentation, with such a cell.

### BACKGROUND

In current microbial fermentative production, compounds such as disaccharides, oligosaccharides and Neu(n)Ac-containing bioproducts are being produced by an industrial production host.

Fermentation using e.g. microorganism using inexpensive carbon sources such as glucose or other sugars to produce compounds such as oligosaccharides and Neu(n)Ac-containing bioproducts are known (see for example, WO2012/007481, WO15197082 and WO07101862). Ways are being sought to obtain higher yields in the fermentative production of the compounds by among others sufficient use of the carbon sources provided.

### DESCRIPTION

### Summary of the Invention

Acetyl-CoA is a central metabolite involved in fatty acid/lipid metabolism, polyketides synthesis, isoprenoids synthesis, amino acids synthesis, the central carbon metabolism, such as glycolysis, glyoxylate pathway, the Krebs cycle and the Calvin cycle, but is not considered to impact carbohydrate synthesis. Surprisingly it has now been found that enhancing the synthesis of acetyl-CoA can be effective for disaccharide, oligosaccharide and/or Neu(n)Ac-containing bioproducts production. The cell and method used in the present invention provide for cell and methods for production of a compound being a disaccharide, oligosaccharide and/or a Neu(n)Ac-containing bioproduct, wherein n is 4, 5, 7, 8 or 9 or a combination thereof, wherein said cell is metabolically engineered, preferably has been metabolically engineered, for enhanced synthesis of acetyl-Coenzyme A and having a positive effect on fermentative production of said compound, providing a better yield, productivity, specific productivity and/or growth speed when used to genetically engineer a cell producing said compound.

### Definitions

The words used in this specification to describe the invention and its various embodiments are to be understood not only in the sense of their commonly defined meanings, but to include by special definition in this specification structure, material or acts beyond the scope of the commonly defined meanings. Thus, if an element can be understood in the context of this specification as including more than one meaning, then its use in a claim must be understood as being generic to all possible meanings supported by the specification and by the word itself.

The various embodiments and aspects of the invention disclosed herein are to be understood not only in the order and context specifically described in this specification, but to include any order and any combination thereof. Whenever the context requires, all words used in the singular number shall be deemed to include the plural and vice versa. Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization described herein are those well-known and commonly employed in the art. Standard techniques are used for nucleic acid and peptide synthesis. Generally, enzymatic reactions and purification steps are performed according to the manufacturer's specifications.

In the specification, there have been disclosed embodiments of the invention, and although specific terms are employed, the terms are used in a descriptive sense only and not for purposes of limitation, the scope of the invention being set forth in the following claims. It must be understood that the illustrated embodiments have been set forth only for the purposes of example and that it should not be taken as limiting the invention. It will be apparent to those skilled in the art that alterations, other embodiments, improvements, details and uses can be made consistent with the letter and spirit of the disclosure herein and within the scope of this disclosure, which is limited only by the claims, construed in accordance with the patent law, including the doctrine of equivalents. In the claims which follow, reference characters used to designate claim steps are provided for convenience of description only, and are not intended to imply any particular order for performing the steps.

In this document and in its claims, the verb “to comprise” and its conjugations is used in its non-limiting sense to mean that items following the word are included, but items not specifically mentioned are not excluded. Throughout the application, the verb “to comprise” may be replaced by “to consist” or “to consist essentially of” and vice versa. In addition, the verb “to consist” may be replaced by “to consist essentially of” meaning that a composition as defined herein may comprise additional component(s) than the ones specifically identified, said additional component(s) not altering the unique characteristic of the invention. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

Throughout the application, unless explicitly stated otherwise, the articles “a” and “an” are preferably replaced by “at least two”, more preferably by “at least three”, even more preferably by “at least four”, even more preferably by “at least five”, even more preferably by “at least six”, most preferably by “at least two”.

Throughout the application, unless explicitly stated otherwise, the features “synthesize”, “synthesized” and “synthesis” are interchangeably used with the features “produce”, “produced” and “production”, respectively.

Throughout the application, unless explicitly stated otherwise, the expressions “capable of . . . <verb>” and “capable to . . . <verb>” are preferably replaced with the active voice of said verb and vice versa. For example, the expression “capable of expressing” is preferably replaced with “expresses” and vice versa, i.e. “expresses” is preferably replaced with “capable of expressing”.

Each embodiment as identified herein may be combined together unless otherwise indicated. All publications, patents, and patent applications mentioned in this specification are herein incorporated by reference to the same extent as if each individual publication, patent, or patent application was specifically and individually indicated to be incorporated by reference.

According to the present invention, the term “polynucleotide(s)” generally refers to any polyribonucleotide or polydeoxyribonucleotide, which may be unmodified RNA or DNA or modified RNA or DNA. “Polynucleotide(s)” include, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. In addition, “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term “polynucleotide(s)” also includes DNAs or RNAs as described above that contain one or more modified bases. Thus, DNAs or RNAs with backbones modified for stability or for other reasons are “polynucleotide(s)” according to the present invention. Moreover, DNAs or RNAs comprising unusual bases, such as inosine, or modified bases, such as tritylated bases, are to be understood to be covered by the term “polynucleotides”. It will be appreciated that a great variety of modifications have been made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. The term “polynucleotide(s)” also embraces short polynucleotides often referred to as oligonucleotide(s).

“Polypeptide(s)” refers to any peptide or protein comprising two or more amino acids joined to each other by peptide bonds or modified peptide bonds. “Polypeptide(s)” refers to both short chains, commonly referred to as peptides, oligopeptides and oligomers and to longer chains generally referred to as proteins. Polypeptides may contain amino acids other than the 20 gene encoded amino acids. “Polypeptide(s)” include those modified either by natural processes, such as processing and other post-translational modifications, but also by chemical modification techniques. Such modifications are well described in basic texts and in more detailed monographs, as well as in a voluminous research literature, and they are well known to the skilled person. The same type of modification may be present in the same or varying degree at several sites in a given polypeptide. Furthermore, a given polypeptide may contain many types of modifications. Modifications can occur anywhere in a polypeptide, including the peptide backbone, the amino acid sidechains, and the amino or carboxyl termini. Modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulphide bond formation, demethylation, formation of covalent cross-links, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, lipid attachment, sulfation, gamma-carboxylation of glutamic acid residues, hydroxylation and ADP-ribosylation, selenoylation, transfer-RNA mediated addition of amino acids to proteins, such as arginylation, and ubiquitination. Polypeptides may be branched or cyclic, with or without branching. Cyclic, branched and branched circular polypeptides may result from post-translational natural processes and may be made by entirely synthetic methods, as well.

“Isolated” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated”, as the term is employed herein. Similarly, a “synthetic” sequence, as the term is used herein, means any sequence that has been generated synthetically and not directly isolated from a natural source. “Synthesized”, as the term is used herein, means any synthetically generated sequence and not directly isolated from a natural source.

The terms “recombinant” or “transgenic” or “engineered” or “metabolically engineered” or “genetically engineered” or “genetically modified” as used herein with reference to a cell or host cell are used interchangeably and indicates that the cell replicates a heterologous nucleic acid, or expresses a peptide or protein encoded by a heterologous nucleic acid (i.e., a sequence “foreign to said cell” or a sequence “foreign to said location or environment in said cell”). Such cells are described to be transformed with at least one heterologous or exogenous gene, or are described to be transformed by the introduction of at least one heterologous or exogenous gene. Metabolically engineered or recombinant or transgenic or genetically engineered cells can contain genes that are not found within the native (non-recombinant) form of the cell. Recombinant cells can also contain genes found in the native form of the cell wherein the genes are re-introduced into the cell by artificial means. The native genes of the cell can also be modified before they are re-introduced into the recombinant cells.

The terms also encompass cells that contain a nucleic acid endogenous to the cell that has been modified or its expression or activity has been modified without removing the nucleic acid from the cell; such modifications include those obtained by gene replacement, replacement of a promoter; site-specific mutation; and related techniques. Accordingly, a “recombinant polypeptide” is one which has been produced by a recombinant cell.

The terms also encompass cells that have been modified by removing a nucleic acid endogenous to the cell by means of common well-known technologies for a skilled person (like e.g. knocking-out genes).

A “heterologous sequence” or a “heterologous nucleic acid”, as used herein, is one that originates from a source foreign to the particular cell (e.g. from a different species), or, if from the same source, is modified from its original form or place in the genome. Thus, a heterologous nucleic acid operably linked to a promoter is from a source different from that from which the promoter was derived, or, if from the same source, is modified from its original form or place in the genome. The heterologous sequence may be stably introduced, e.g. by transfection, transformation, conjugation or transduction, into the genome of the host microorganism cell, wherein techniques may be applied which will depend on the cell and the sequence that is to be introduced. Various techniques are known to a person skilled in the art and are, e.g., disclosed in Sambrook et al., Molecular Cloning: A Laboratory Manual, 2nd Ed., Cold Spring Harbor Laboratory Press, Cold Spring Harbor, N.Y. (1989). The term “mutant” or “engineered” cell or microorganism as used within the context of the present disclosure refers to a cell or microorganism which is genetically engineered.

The term “endogenous,” within the context of the present disclosure refers to any polynucleotide, polypeptide or protein sequence which is a natural part of a cell and is occurring at its natural location in the cell chromosome. The term “exogenous” refers to any polynucleotide, polypeptide or protein sequence which originates from outside the cell under study and not a natural part of the cell or which is not occurring at its natural location in the cell chromosome or plasmid.

The term “heterologous” when used in reference to a polynucleotide, gene, nucleic acid, polypeptide, or enzyme refers to a polynucleotide, gene, nucleic acid, polypeptide, or enzyme that is from a source or derived from a source other than the host organism. In contrast a “homologous” polynucleotide, gene, nucleic acid, polypeptide, or enzyme is used herein to denote a polynucleotide, gene, nucleic acid, polypeptide, or enzyme that is derived from the host organism. When referring to a gene regulatory sequence or to an auxiliary nucleic acid sequence used for maintaining or manipulating a gene sequence (e.g. a promoter, a 5′ untranslated region, 3′ untranslated region, poly A addition sequence, intron sequence, splice site, ribosome binding site, internal ribosome entry sequence, genome homology region, recombination site, etc.), “heterologous” means that the regulatory sequence or auxiliary sequence is not naturally associated with the gene with which the regulatory or auxiliary nucleic acid sequence is juxtaposed in a construct, genome, chromosome, or episome. Thus, a promoter operably linked to a gene to which it is not operably linked to in its natural state (i.e. in the genome of a non-genetically engineered organism) is referred to herein as a “heterologous promoter”, even though the promoter may be derived from the same species (or, in some cases, the same organism) as the gene to which it is linked.

The term “polynucleotide encoding a polypeptide” as used herein encompasses polynucleotides that include a sequence encoding a polypeptide of the invention. The term also encompasses polynucleotides that include a single continuous region or discontinuous regions encoding the polypeptide (for example, interrupted by integrated phage or an insertion sequence or editing) together with additional regions that also may contain coding and/or non-coding sequences.

The term “modified expression” of a gene relates to a change in expression compared to the wild type expression of said gene in any phase of the production process of the compound being a disaccharide, oligosaccharide and/or Neu(n)Ac-containing bioproduct. Said modified expression is either a lower or higher expression compared to the wild type, wherein the terms “higher expression” or “enhanced expression” are also defined as “overexpression” of said gene in the case of an endogenous gene or “expression” in the case of a heterologous gene that is not present in the wild type strain. Lower or reduced expression is obtained by means of common well-known technologies for a skilled person (such as the usage of siRNA, CrispR, CrispRi, riboswitches, recombineering, homologous recombination, ssDNA mutagenesis, RNAi, miRNA, asRNA, mutating genes, knocking-out genes, transposon mutagenesis, . . . ) which are used to change the genes in such a way that they are less-able (i.e. statistically significantly ‘less-able’ compared to a functional wild-type gene) or completely unable (such as knocked-out genes) to produce functional final products. The term “riboswitch” as used herein is defined to be part of the messenger RNA that folds into intricate structures that block expression by interfering with translation. Binding of an effector molecule induces conformational change(s) permitting regulated expression post-transcriptionally. Next to changing the gene of interest in such a way that lower expression is obtained as described above, lower expression can also be obtained by changing the transcription unit, the promoter, an untranslated region, the ribosome binding site, the Shine Dalgarno sequence or the transcription terminator. Lower expression or reduced expression can for instance be obtained by mutating one or more base pairs in the promoter sequence or changing the promoter sequence fully to a constitutive promoter with a lower expression strength compared to the wild type or an inducible promoter which result in regulated expression or a repressible promoter which results in regulated expression. Overexpression or expression is obtained by means of common well-known technologies for a skilled person (such as the usage of artificial transcription factors, de novo design of a promoter sequence, ribosome engineering, introduction or re-introduction of an expression module at euchromatin, usage of high-copy-number plasmids), wherein said gene is part of an “expression cassette” which relates to any sequence in which a promoter sequence, untranslated region sequence (containing either a ribosome binding sequence or Kozak sequence or Shine Dalgarno sequence), a coding sequence (for instance an acetyl-Coenzyme A ligase gene sequence) and optionally a transcription terminator is present, and leading to the expression of a functional active protein. Said expression is either constitutive or conditional or regulated or tuneable.

The term “constitutive expression” is defined as expression that is not regulated by transcription factors other than the subunits of RNA polymerase (e.g., the bacterial sigma factors like s⁷⁰, s⁵⁴, or related s-factors and the yeast mitochondrial RNA polymerase specificity factor MTF1 that co-associate with the RNA polymerase core enzyme) under certain growth conditions. Non-limiting examples of such transcription factors are CRP, LacI, ArcA, Cra, IclR in E. coli, or, Aft2p, Crz1p, Skn7 in Saccharomyces cerevisiae, or, DeoR, GntR, Fur in B. subtilis. These transcription factors bind on a specific sequence and may block or enhance expression in certain growth conditions. The RNA polymerase is the catalytic machinery for the synthesis of RNA from a DNA template. RNA polymerase binds a specific DNA sequence to initiate transcription, for instance via a sigma factor in prokaryotic hosts or via MTF1 in yeasts. Constitutive expression offers a constant level of expression with no need for induction or repression.

The term “regulated expression” is defined as a facultative or regulatory or tuneable expression of a gene that is only expressed upon a certain natural condition of the host (e.g. mating phase of budding yeast, stationary phase of bacteria), as a response to an inducer or repressor such as but not limited to glucose, allo-lactose, lactose, galactose, glycerol, arabinose, rhamnose, fucose, IPTG, methanol, ethanol, acetate, formate, aluminium, copper, zinc, nitrogen, phosphates, xylene, carbon or nitrogen depletion, or substrates or the produced product or chemical repression, as a response to an environmental change (e.g. anaerobic or aerobic growth, oxidative stress, pH shifts, temperature changes like e.g. heat-shock or cold-shock, osmolarity, light conditions, starvation) or dependent on the position of the developmental stage or the cell cycle of said host cell including but not limited to apoptosis and autophagy. Regulated expression allows for control as to when a gene is expressed.

The term “control sequences” refers to sequences recognized by the cells transcriptional and translational systems, allowing transcription and translation of a polynucleotide sequence to a polypeptide. Such DNA sequences are thus necessary for the expression of an operably linked coding sequence in a particular cell or organism. Such control sequences can be, but are not limited to, promoter sequences, ribosome binding sequences, Shine Dalgarno sequences, Kozak sequences, transcription terminator sequences. The control sequences that are suitable for prokaryotes, for example, include a promoter, optionally an operator sequence, and a ribosome binding site. Eukaryotic cells are known to utilize promoters, polyadenylation signals, and enhancers. DNA for a presequence or secretory leader may be operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it affects the transcription of the sequence; or a ribosome binding site is operably linked to a coding sequence if it is positioned so as to facilitate translation. Said control sequences can furthermore be controlled with external chemicals, such as, but not limited to, IPTG, arabinose, lactose, allo-lactose, rhamnose or fucose via an inducible promoter or via a genetic circuit that either induces or represses the transcription or translation of said polynucleotide to a polypeptide.

Generally, “operably linked” means that the DNA sequences being linked are contiguous, and, in the case of a secretory leader, contiguous and in reading phase. However, enhancers do not have to be contiguous.

The term “wild type” refers to the commonly known genetic or phenotypical situation as it occurs in nature.

The term “modified activity” of a protein relates to a non-native activity of said protein in any phase of the production process of the desired disaccharide, oligosaccharide and/or Neu(n)Ac-containing bioproduct.

The term “non-native”, as used herein with reference to a protein, indicates that the protein is i) not naturally produced or ii) when naturally produced not in the same amounts by the cell; and that the cell has been metabolically engineered to be able to produce said protein or to have a lower or a higher production of the protein or to produce a protein with an abolished, impaired, reduced, delayed, accelerated or enhanced activity compared to the wild type activity of the protein. The term “non-native”, as used herein with reference to a cell producing a disaccharide, oligosaccharide and/or Neu(n)Ac-containing bioproduct, indicates that the disaccharide, oligosaccharide and/or Neu(n)Ac-containing bioproduct is i) not naturally produced or ii) when naturally produced not in the same amounts by the cell; and that the cell has been metabolically engineered to be able to produce said disaccharide, oligosaccharide and/or Neu(n)Ac-containing bioproduct or to have a higher production of the disaccharide, oligosaccharide and/or Neu(n)Ac-containing bioproduct.

The term “enhanced expression or activity” of a protein as used herein refers to i) higher expression or overexpression of an endogenous protein, ii) expression of a heterologous protein or iii) expression and/or overexpression of a mutant protein that has a higher or an accelerated activity compared to the wild-type (i.e. native) protein. Furthermore, expression of the gene may be enhanced by, as described in WO 00/18935, WO98/04715, substituting an expression regulatory sequence such as the native promoter with a stronger promoter, whether the gene is present on the chromosome or a plasmid, amplifying a regulatory element that is able to increase expression of the gene, or deleting or attenuating a regulatory element that decreases expression of the gene. Examples of known strong promoters include the lac promoter, trp promoter, trc promoter, tac promoter, lambda phage PR promoter, PL promoter, and tet promoter.

A method to evaluate the strength of a promoter and examples of strong promoters are described in Goldstein et al. (Prokaryotic promoters in biotechnology. Biotechnol. Annu. Rev., 1995, 1, 105-128) or the like. In addition, it is known that a spacer sequence between the ribosome binding site (RBS) and the translation initiation codon, especially, several nucleotides just upstream of the initiation codon, has a great influence on translation efficiency. Therefore, this sequence may be modified.

In addition, to enhance the activity of a protein encoded by the gene, a mutation that increases the activity may be introduced into the gene. Examples of such a mutation include a mutation in the promoter sequence to increase the transcription level of the gene, and a mutation in the coding region to increase the specific activities of the protein.

“Variant(s)” as the term is used herein, is a polynucleotide or polypeptide that differs from a reference polynucleotide or polypeptide respectively but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be naturally occurring such as an allelic variant, or it may be a variant that is not known to occur naturally. Non-naturally occurring variants of polynucleotides and polypeptides may be made by mutagenesis techniques, by direct synthesis, and by other recombinant methods known to the persons skilled in the art.

In some embodiments, the present disclosure contemplates making functional variants by modifying the structure of a protein as used in the present invention. Variants can be produced by amino acid substitution, deletion, addition, or combinations thereof. For instance, it is reasonable to expect that an isolated replacement of a leucine with an isoleucine or valine, an aspartate with a glutamate, a threonine with a serine, or a similar replacement of an amino acid with a structurally related amino acid (e.g., conservative mutations) will not have a major effect on the biological activity of the resulting molecule. Conservative replacements are those that take place within a family of amino acids that are related in their side chains. Whether a change in the amino acid sequence of a polypeptide of the disclosure results in a functional homolog can be readily determined by assessing the ability of the variant polypeptide to produce a response in cells in a fashion similar to the wild-type polypeptide, and in some cases to provide better yield, productivity, and/or growth speed than a cell without the variant.

The term “functional homolog” as used herein describes those molecules that have sequence similarity and also share at least one functional characteristic such as a biochemical activity. More specifically, the term “functional homolog” as used herein describes those proteins that have sequence similarity (in other words, homology) and at the same time have at least one functional similarity such as a biochemical activity (Altenhoff et al., PLoS Comput. Biol. 8 (2012) e1002514).

Functional homologs are sometimes referred to as orthologs, where “ortholog” refers to a homologous gene or protein that is the functional equivalent of the referenced gene or protein in another species. Functional homologs will typically give rise to the same characteristics to a similar, but not necessarily the same, degree. Functionally homologous proteins give the same characteristics where the quantitative measurement produced by one homolog is at least 10 percent of the other; more typically, at least 20 percent, between about 30 percent and about 40 percent; for example, between about 50 percent and about 60 percent; between about 70 percent and about 80 percent; or between about 90 percent and about 95 percent; between about 98 percent and about 100 percent, or greater than 100 percent of that produced by the original molecule. Thus, where the molecule has enzymatic activity the functional homolog will have the above-recited percent enzymatic activities compared to the original enzyme. Where the molecule is a DNA-binding molecule (e.g., a polypeptide) the homolog will have the above-recited percentage of binding affinity as measured by weight of bound molecule compared to the original molecule.

A functional homolog and the reference polypeptide may be naturally occurring polypeptides, and the sequence similarity may be due to convergent or divergent evolutionary events.

Functional homologs can be identified by analysis of nucleotide and polypeptide sequence alignments. For example, performing a query on a database of nucleotide or polypeptide sequences can identify homologs of biomass-modulating polypeptides. Sequence analysis can involve BLAST, Reciprocal BLAST, or PSI-BLAST analysis of non-redundant databases using amino acid sequence of a biomass-modulating polypeptide as the reference sequence. Amino acid sequence is, in some instances, deduced from the nucleotide sequence. Typically, those polypeptides in the database that have greater than 40 percent sequence identity are candidates for further evaluation for suitability as a biomass-modulating polypeptide. Amino acid sequence similarity allows for conservative amino acid substitutions, such as substitution of one hydrophobic residue for another or substitution of one polar residue for another. If desired, manual inspection of such candidates can be carried out in order to narrow the number of candidates to be further evaluated. Manual inspection can be performed by selecting those candidates that appear to have domains present in productivity-modulating polypeptides, e.g., conserved functional domains.

“Fragment”, with respect to a polynucleotide, refers to a clone or any part of a polynucleotide molecule, particularly a part of a polynucleotide that retains a usable, functional characteristic of the full-length polynucleotide molecule. Useful fragments include oligonucleotides and polynucleotides that may be used in hybridization or amplification technologies or in the regulation of replication, transcription, or translation. A “polynucleotide fragment” refers to any subsequence of a polynucleotide SEQ ID NO, typically, of at least about 9, 10, 11, 12 consecutive nucleotides, for example at least about 30 nucleotides or at least about 50 nucleotides of any of the polynucleotide sequences provided herein. Exemplary fragments can additionally or alternatively include fragments that comprise, consist essentially of, or consist of a region that encodes a conserved family domain of a polypeptide. Exemplary fragments can additionally or alternatively include fragments that comprise a conserved domain of a polypeptide.

As such, a fragment of a polynucleotide SEQ ID NO preferably means a nucleotide sequence which comprises or consists of said polynucleotide SEQ ID NO wherein no more than 200, 150, 100, 50 or 25 consecutive nucleotides are missing, preferably no more than 50 consecutive nucleotides are missing, and which retains a usable, functional characteristic (e.g. activity) of the full-length polynucleotide molecule which can be assessed by the skilled person through routine experimentation. Alternatively, a fragment of a polynucleotide SEQ ID NO preferably means a nucleotide sequence which comprises or consists of an amount of consecutive nucleotides from said polynucleotide SEQ ID NO and wherein said amount of consecutive nucleotides is at least 50.0%, 60.0%, 70.0%, 80.0%, 81.0%, 82.0%, 83.0%, 84.0%, 85.0%, 86.0%, 87.0%, 88.0%, 89.0%, 90.0%, 91.0%, 92.0%, 93.0%, 94.0%, 95.0%, 95.5%, 96.0%, 96.5%, 97.0%, 97.5%, 98.0%, 98.5%, 99.0%, 99.5%, 100%, preferably at least 80.0%, more preferably at least 87.0%, even more preferably at least 90.0%, even more preferably at least 95.0%, most preferably at least 97.0%, of the full-length of said polynucleotide SEQ ID NO and retains a usable, functional characteristic (e.g. activity) of the full-length polynucleotide molecule. As such, a fragment of a polynucleotide SEQ ID NO preferably means a nucleotide sequence which comprises or consists of said polynucleotide SEQ ID NO, wherein an amount of consecutive nucleotides is missing and wherein said amount is no more than 50.0%, 40.0%, 30.0% of the full-length of said polynucleotide SEQ ID NO, preferably no more than 20.0%, 15.0%, 10.0%, 9.0%, 8.0%, 7.0%, 6.0%, 5.0%, 4.5%, 4.0%, 3.5%, 3.0%, 2.5%, 2.0%, 1.5%, 1.0%, 0.5%, more preferably no more than 15.0%, even more preferably no more than 10.0%, even more preferably no more than 5.0%, most preferably no more than 2.5%, of the full-length of said polynucleotide SEQ ID NO and wherein said fragment retains a usable, functional characteristic (e.g. activity) of the full-length polynucleotide molecule which can be routinely assessed by the skilled person. Fragments may additionally or alternatively include subsequences of polypeptides and protein molecules, or a subsequence of the polypeptide. In some cases, the fragment or domain is a subsequence of the polypeptide which performs at least one biological function of the intact polypeptide in substantially the same manner, or to a similar extent, as does the intact polypeptide. A “subsequence of the polypeptide” as defined herein refers to a sequence of contiguous amino acid residues derived from the polypeptide. For example, a polypeptide fragment can comprise a recognizable structural motif or functional domain such as a DNA-binding site or domain that binds to a DNA promoter region, an activation domain, or a domain for protein-protein interactions, and may initiate transcription. Fragments can vary in size from as few as 3 amino acid residues to the full length of the intact polypeptide, for example at least 10 amino acid residues in length, for example at least about 20 amino acid residues in length, for example at least about 30 amino acid residues in length. Preferentially a fragment is a functional fragment that has at least one property or activity of the polypeptide from which it is derived, such as, for example, the fragment can include a functional domain or conserved domain of a polypeptide. As such, a fragment of a polypeptide SEQ ID NO (or UniProt ID) preferably means a polypeptide sequence which comprises or consists of said polypeptide SEQ ID NO (or UniProt ID) wherein no more than 80, 60, 50, 40, 30, 20 or 15 consecutive amino acid residues are missing, preferably no more than 40 consecutive amino acid residues are missing, and performs at least one biological function of the intact polypeptide in substantially the same manner, preferably to a similar or greater extent, as does the intact polypeptide which can be routinely assessed by the skilled person. Alternatively, a fragment of a polypeptide SEQ ID NO (or UniProt ID) preferably means a polypeptide sequence which comprises or consists of an amount of consecutive amino acid residues from said polypeptide SEQ ID NO (or UniProt ID) and wherein said amount of consecutive amino acid residues is at least 50.0%, 60.0%, 70.0%, 80.0%, 81.0%, 82.0%, 83.0%, 84.0%, 85.0%, 86.0%, 87.0%, 88.0%, 89.0%, 90.0%, 91.0%, 92.0%, 93.0%, 94.0%, 95.0%, 95.5%, 96.0%, 96.5%, 97.0%, 97.5%, 98.0%, 98.5%, 99.0%, 99.5%, 100%, preferably at least 80.0%, more preferably at least 87.0%, even more preferably at least 90.0%, even more preferably at least 95.0%, most preferably at least 97.0% of the full-length of said polypeptide SEQ ID NO (or UniProt ID) and which performs at least one biological function of the intact polypeptide in substantially the same manner, preferably to a similar or greater extent, as does the intact polypeptide which can be routinely assessed by the skilled person. As such, a fragment of a polypeptide SEQ ID NO (or UniProt ID) preferably means a polypeptide sequence which comprises or consists of said polypeptide SEQ ID NO (or UniProt ID), wherein an amount of consecutive amino acid residues is missing and wherein said amount is no more than 50.0%, 40.0%, 30.0% of the full-length of said polypeptide SEQ ID NO (or UniProt ID), preferably no more than 20.0%, 15.0%, 10.0%, 9.0%, 8.0%, 7.0%, 6.0%, 5.0%, 4.5%, 4.0%, 3.5%, 3.0%, 2.5%, 2.0%, 1.5%, 1.0%, 0.5%, more preferably no more than 15.0%, even more preferably no more than 10.0%, even more preferably no more than 5.0%, most preferably no more than 2.5%, of the full-length of said polypeptide SEQ ID NO (or UniProt ID) and which performs at least one biological function of the intact polypeptide in substantially the same manner, preferably to a similar or greater extent, as does the intact polypeptide which can be routinely assessed by the skilled person.

Throughout the application, the sequence of a polypeptide can be represented by a SEQ ID NO or alternatively by an UniProt ID. Therefore, the terms “polypeptide SEQ ID NO” and “polypeptide UniProt ID” can be interchangeably used, unless explicitly stated otherwise.

A “functional fragment” of a polypeptide has at least one property or activity of the polypeptide from which it is derived, preferably to a similar or greater extent. A functional fragment can, for example, include a functional domain or conserved domain of a polypeptide. It is understood that a polypeptide or a fragment thereof may have conservative amino acid substitutions which have substantially no effect on the polypeptide's activity. By conservative substitutions is intended substitutions of one hydrophobic amino acid for another or substitution of one polar amino acid for another or substitution of one acidic amino acid for another or substitution of one basic amino acid for another etc. Preferably, by conservative substitutions is intended combinations such as glycine by alanine and vice versa; valine, isoleucine and leucine by methionine and vice versa; aspartate by glutamate and vice versa; asparagine by glutamine and vice versa; serine by threonine and vice versa; lysine by arginine and vice versa; cysteine by methionine and vice versa; and phenylalanine and tyrosine by tryptophan and vice versa.

Homologous sequences as used herein describes those nucleotide sequences that have sequence similarity and encode polypeptides that share at least one functional characteristic such as a biochemical activity. More specifically, the term “functional homolog” as used herein describes those polypeptides that have sequence similarity (in other words, homology) and at the same time have at least one functional similarity such as a biochemical activity (Altenhoff et al., PLoS Comput. Biol. 8 (2012) e1002514).

Homologs can be identified by analysis of nucleotide and polypeptide sequence alignments. For example, performing a query on a database of nucleotide or polypeptide sequences can identify homologs of the nucleotide or polypeptide of interest. Sequence analysis can involve BLAST, Reciprocal BLAST, or PSI-BLAST analysis of non-redundant databases using the nucleotide or the amino acid sequence of a reference nucleotide or polypeptide sequence. The amino acid sequence is, in some instances, deduced from the nucleotide sequence. Typically, those polypeptides in the database that have greater than 40 percent sequence identity to a polypeptide of interest are candidates for further evaluation for suitability as a homologous polypeptide. Amino acid sequence similarity allows for conservative amino acid substitutions, such as substitution of one hydrophobic residue for another or substitution of one polar residue for another or substitution of one acidic amino acid for another or substitution of one basic amino acid for another etc. Preferably, by conservative substitutions is intended combinations such as glycine by alanine and vice versa; valine, isoleucine and leucine by methionine and vice versa; aspartate by glutamate and vice versa; asparagine by glutamine and vice versa; serine by threonine and vice versa; lysine by arginine and vice versa; cysteine by methionine and vice versa; and phenylalanine and tyrosine by tryptophan and vice versa. If desired, manual inspection of such candidates can be carried out to narrow the number of candidates to be further evaluated.

A domain can be characterized, for example, by a Pfam (El-Gebali et al., Nucleic Acids Res. 47 (2019) D427-D432), an IPR (InterPro domain) (http://ebi.ac.uk/interpro) (Mitchell et al., Nucleic Acids Res. 47 (2019) D351-D360), a protein fingerprint domain (PRINTS) (Attwood et al., Nucleic Acids Res. 31 (2003) 400-402), a SUBFAM domain (Gough et al., J. Mol. Biol. 313 (2001) 903-919), a TIGRFAM domain (Selengut et al., Nucleic Acids Res. 35 (2007) D260-D264), a Conserved Domain Database (CDD) designation (https://www.ncbi.nlm.nih.gov/cdd) (Lu et al., Nucleic Acids Res. 48 (2020) D265-D268), or a PTHR domain (http://www.pantherdb.org) (Mi et al., Nucleic Acids. Res. 41 (2013) D377-D386; Thomas et al., Genome Research 13 (2003) 2129-2141). It should be understood for those skilled in the art that for the databases used herein, comprising Pfam 32.0 (released September 2018), CDD v3.17 (released 3^{rd} Apr. 2019), eggnogdb 4.5.1 (released September 2016) and InterPro 75.0 (released 4^{th} Jul. 2019), the content of each database is fixed at each release and is not to be changed. When the content of a specific database is changed, this specific database receives a new release version with a new release date. All release versions for each database with their corresponding release dates and specific content as annotated at these specific release dates are available and known to those skilled in the art.

Protein or polypeptide sequence information and functional information can be provided by a comprehensive resource for protein sequence and annotation data like e.g., the Universal Protein Resource (UniProt) (www.uniprot.org) (Nucleic Acids Res. 2021, 49(D1), D480-D489). UniProt comprises the expertly and richly curated protein database called the UniProt Knowledgebase (UniProtKB), together with the UniProt Reference Clusters (UniRef) and the UniProt Archive (UniParc). The UniProt identifiers are unique for each protein present in the database and are defined herein as “UniProt ID” or “UniProtKB ID” or “UniProtKB” or “UniProt KB”. The UniProt identifiers as used herein are the the UniProt identifiers in the UniProt database version release 2021_02 of 7 Apr. 2021. Proteins that do not have an UniProt ID are referred herein using the respective GenBank Accession number (GenBank No.) as present in the NIH genetic sequence database (https://www.ncbi.nlm.nih.gov/genbank/) (Nucleic Acids Res. 2013, 41(D1), D36-D42) under GenBank Release 236.0 of 15 Feb. 2020.

The terms “identical” or “percent identity” or “% identity” in the context of two or more nucleic acid or polypeptide sequences, refer to two or more sequences or subsequences that are the same or have a specified percentage of nucleotides or amino acid residues that are the same, when compared and aligned for maximum correspondence, as measured using sequence comparison algorithms or by visual inspection. For sequence comparison, one sequence acts as a reference sequence, to which test sequences are compared. When using a sequence comparison algorithm, test and reference sequences are inputted into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. The percentage of sequence identity can be, preferably is, determined by alignment of the two sequences and identification of the number of positions with identical residues divided by the number of residues in the shorter of the sequences×100. Percent identity may be calculated globally over the full-length sequence of a given SEQ ID NO, i.e., the reference sequence, resulting in a global percent identity score. Alternatively, percent identity may be calculated over a partial sequence of the reference sequence, resulting in a local percent identity score. A partial sequence preferably means at least about 50%, 60%, 70%, 80%, 90% or 95% of the full-length reference sequence. In another preferred embodiment, a partial sequence of a reference polypeptide sequence means a stretch of at least 200 amino acid residues up to the total number of amino acid residues of a reference polypeptide sequence. Using the full-length of the reference sequence in a local sequence alignment results in a global percent identity score between the test and the reference sequence. Percent identity can be determined using different algorithms like for example BLAST and PSI-BLAST (Altschul et al., 1990, J Mol Biol 215:3, 403-410; Altschul et al., 1997, Nucleic Acids Res 25: 17, 3389-402), the Clustal Omega method (Sievers et al., 2011, Mol. Syst. Biol. 7:539), the MatGAT method (Campanella et al., 2003, BMC Bioinformatics, 4:29) or EMBOSS Needle (https://galaxy-iuc.github.io/emboss-5.0-docs/needle.html).

### Calculation of Percentage Identity Between Polypeptide Sequences

Methods for the alignment of sequences for comparison are well known in the art, such methods include GAP, BESTFIT, BLAST, FASTA and TFASTA. GAP uses the algorithm of Needleman and Wunsch (J. Mol. Biol. (1970) 48: 443-453) to find the global (i.e., spanning the full-length sequences) alignment of two sequences that maximizes the number of matches and minimizes the number of gaps. The BLAST algorithm (Altschul et al., J. Mol. Biol. (1990) 215: 403-10) calculates the global percentage sequence identity (i.e., over the full-length sequence) and performs a statistical analysis of the similarity between the two sequences. The software for performing BLAST analysis is publicly available through the National Centre for Biotechnology Information (NCBI). Homologs may readily be identified using, for example, the ClustalW multiple sequence alignment algorithm (version 1.83), with the default pairwise alignment parameters, and a scoring method in percentage. Global percentages of similarity and identity (i.e., spanning the full-length sequences) may also be determined using one of the methods available in the MatGAT software package (Campanella et al., BMC Bioinformatics (2003) 4:29). Minor manual editing may be performed to optimize alignment between conserved motifs, as would be apparent to a person skilled in the art. Furthermore, instead of using full-length sequences for the identification of homologs, specific domains may also be used, to determine the so-called local sequence identity. The sequence identity values may be determined over the entire nucleic acid or amino acid sequence (=local sequence identity search over the full-length sequence resulting in a global sequence identity score) or over selected domains or conserved motif(s) (=local sequence identity search over a partial sequence resulting in a local sequence identity score), using the programs mentioned above using the default parameters. For local alignments, the Smith-Waterman algorithm is particularly useful (Smith T F, Waterman M S (1981) J. Mol. Biol 147(1); 195-7).

For the purposes of this invention, percent identity is determined using MatGAT2.01 (Campanella et al., 2003, BMC Bioinformatics 4:29). The following default parameters for protein are employed: (1) Gap cost Existence: 12 and Extension: 2; (2) The Matrix employed was BLOSUM50.

As used herein, the term “mammary cell(s)” generally refers to mammary epithelial cell(s), mammary-epithelial luminal cell(s), or mammalian epithelial alveolar cell(s), or any combination thereof. As used herein, the term “mammary-like cell(s)” generally refers to cell(s) having a phenotype/genotype similar (or substantially similar) to natural mammary cell(s) but is/are derived from non-mammary cell source(s). Such mammary-like cell(s) may be engineered to remove at least one undesired genetic component and/or to include at least one predetermined genetic construct that is typical of a mammary cell. Non-limiting examples of mammary-like cell(s) may include mammary epithelial-like cell(s), mammary epithelial luminal-like cell(s), non-mammary cell(s) that exhibits one or more characteristics of a cell of a mammary cell lineage, or any combination thereof. Further non-limiting examples of mammary-like cell(s) may include cell(s) having a phenotype similar (or substantially similar) to natural mammary cell(s), or more particularly a phenotype similar (or substantially similar) to natural mammary epithelial cell(s). A cell with a phenotype or that exhibits at least one characteristic similar to (or substantially similar to) a natural mammary cell or a mammary epithelial cell may comprise a cell (e.g., derived from a mammary cell lineage or a non-mammary cell lineage) that exhibits either naturally, or has been engineered to, be capable of expressing at least one milk component.

As used herein, the term “non-mammary cell(s)” may generally include any cell of non-mammary lineage. In the context of the invention, a non-mammary cell can be any mammalian cell capable of being engineered to express at least one milk component. Non-limiting examples of such non-mammary cell(s) include hepatocyte(s), blood cell(s), kidney cell(s), cord blood cell(s), epithelial cell(s), epidermal cell(s), myocyte(s), fibroblast(s), mesenchymal cell(s), or any combination thereof. In some instances, molecular biology and genome editing techniques can be engineered to eliminate, silence, or attenuate myriad genes simultaneously.

The term “Neu(n)Ac-containing bioproduct” as used herein refers to Neu(n)Ac as defined herein as well as to a compound comprising a disaccharide, an oligosaccharide, a glycolipid and a glycoprotein that comprises one or more Neu(n)Ac molecules. Further, the term “Neu(n)Ac-containing bioproduct wherein (n) is 4, 5, 7, 8 or 9 or a combination thereof” as used herein refers to a Neu(n)Ac-containing bioproduct as defined herein wherein the (n) can be any one or more of 4, 5, 7, 8 or 9, as further explained herein and wherein the Neu(n)Ac-molecule can be chosen from the list comprising Neu4Ac; Neu5Ac; Neu4,5Ac2; Neu5,7Ac2; Neu5,8Ac2; Neu5,9Ac2; Neu4,5,9Ac3; Neu5,7,9Ac3; Neu5,8,9Ac3; Neu4,5,7,9Ac4; Neu5,7,8,9Ac4 and Neu4,5,7,8,9Ac5 and Neu5Gc.

The terms “sialic acid”, “Neu(n)Ac (wherein (n) is 4, 5, 7, 8 or 9 or a combination thereof)”, “N-acetylneuraminate”, “N-acylneuraminate”, “N-acetylneuraminic acid”, “Neu(n)Ac molecule” and “Neu(n)Ac” are used interchangeably and refer to an acidic sugar with a nine-carbon backbone comprising but not limited to Neu4Ac; Neu5Ac; Neu4,5Ac2; Neu5,7Ac2; Neu5,8Ac2; Neu5,9Ac2; Neu4,5,9Ac3; Neu5,7,9Ac3; Neu5,8,9Ac3; Neu4,5,7,9Ac4; Neu5,7,8,9Ac4 and Neu4,5,7,8,9Ac5 and Neu5Gc.

Neu4Ac is also known as 4-O-acetyl-5-amino-3,5-dideoxy-D-glycero-D-galacto-non-2-ulopyranosonic acid or 4-O-acetyl neuraminic acid and has C11H19NO9 as molecular formula. Neu5Ac is also known as 5-acetamido-3,5-dideoxy-D-glycero-D-galacto-non-2-ulopyranosonic acid, D-glycero-5-acetamido-3,5-dideoxy-D-galacto-non-2-ulo-pyranosonic acid, 5-(acetylamino)-3,5-dideoxy-D-glycero-D-galacto-2-nonulopyranosonic acid, 5-(acetylamino)-3,5-dideoxy-D-glycero-D-galacto-2-nonulosonic acid, 5-(acetylamino)-3,5-dideoxy-D-glycero-D-galacto-non-2-nonulosonic acid or 5-(acetylamino)-3,5-dideoxy-D-glycero-D-galacto-non-2-ulopyranosonic acid and has C11H19NO9 as molecular formula. Neu4,5Ac2 is also known as N-acetyl-4-O-acetylneuraminic acid, 4-O-acetyl-N-acetylneuraminic acid, 4-O-acetyl-N-acetylneuraminate, 4-acetate 5-acetamido-3,5-dideoxy-D-glycero-D-galacto-nonulosonate, 4-acetate 5-(acetylamino)-3,5-dideoxy-D-glycero-D-galacto-2-nonulosonate, 4-acetate 5-acetamido-3,5-dideoxy-D-glycero-D-galacto-nonulosonic acid or 4-acetate 5-(acetylamino)-3,5-dideoxy-D-glycero-D-galacto-2-nonulosonic acid and has C13H21NO10 as molecular formula. Neu5,7Ac2 is also known as 7-O-acetyl-N-acetylneuraminic acid, N-acetyl-7-O-acetylneuraminic acid, 7-O-acetyl-N-acetylneuraminate, 7-acetate 5-acetamido-3,5-dideoxy-D-glycero-D-galacto-nonulosonate, 7-acetate 5-(acetylamino)-3,5-dideoxy-D-glycero-D-galacto-2-nonulosonate, 7-acetate 5-acetamido-3,5-dideoxy-D-glycero-D-galacto-nonulosonic acid or 7-acetate 5-(acetylamino)-3,5-dideoxy-D-glycero-D-galacto-2-nonulosonic acid and has C13H21NO10 as molecular formula. Neu5,8Ac2 is also known as 5-n-acetyl-8-o-acetyl neuraminic acid and has C13H21NO10 as molecular formula. Neu5,9Ac2 is also known as N-acetyl-9-O-acetylneuraminic acid, 9-anana, 9-O-acetylsialic acid, 9-O-acetyl-N-acetylneuraminic acid, 5-n-acetyl-9-O-acetyl neuraminic acid, N,9-O-diacetylneuraminate or N,9-O-diacetylneuraminate and has C13H21NO10 as molecular formula. Neu4,5,9Ac3 is also known as 5-N-acetyl-4,9-di-O-acetylneuraminic acid. Neu5,7,9Ac3 is also known as 5-N-acetyl-7,9-di-O-acetylneuraminic acid. Neu5,8,9Ac3 is also known as 5-N-acetyl-8,9-di-O-acetylneuraminic acid. Neu4,5,7,9Ac4 is also known as 5-N-acetyl-4,7,9-tri-O-acetylneuraminic acid. Neu5,7,8,9Ac4 is also known as 5-N-acetyl-7,8,9-tri-O-acetylneuraminic acid. Neu4,5,7,8,9Ac5 is also known as 5-N-acetyl-4,7,8,9-tetra-O-acetylneuraminic acid. Neu5Gc is also known as N-glycolylneuraminic acid, N-glycolylneuraminic acid, N-glycolylneuraminate, N-glycoloyl-neuraminate, N-glycoloylneuraminic acid, N-glycoloylneuraminic acid, 3,5-dideoxy-5-((hydroxyacetyl)amino)-D-glycero-D-galacto-2-nonulosonic acid, 3,5-dideoxy-5-(glycoloylamino)-D-glycero-D-galacto-2-nonulopyranosonic acid, 3,5-dideoxy-5-(glycoloylamino)-D-glycero-D-galacto-non-2-ulopyranosonic acid, 3,5-dideoxy-5-[(hydroxyacetyl)amino]-D-glycero-D-galacto-non-2-ulopyranosonic acid, D-glycero-5-glycolylamido-3,5-dideoxy-D-galacto-non-2-ulo-pyranosonic acid and has C11H19NO10 as molecular formula.

The terms “Neu(n)Ac synthase”, “N-acetylneuraminic acid synthase”, “N-acetylneuraminate synthase”, “sialic acid synthase”, “NeuAc synthase”, “NeuB”, “NeuB1”, “NANA condensing enzyme”, “N-acetylneuraminate lyase synthase”, “N-acetylneuraminic acid condensing enzyme” as used herein are used interchangeably and refer to an enzyme capable to synthesize sialic acid (Neu(n)Ac) from N-acetylmannosamine (ManNAc) in a reaction using phosphoenolpyruvate (PEP).

The terms “CMP-sialic acid synthase”, “N-acylneuraminate cytidylyltransferase”, “CMP-sialate synthase”, “CMP-Neu(n)Ac synthase”, “NeuA” and “CMP-N-acetylneuraminic acid synthase” as used herein are used interchangeably and refer to an enzyme capable to synthesize CMP-N-acetylneuraminate from N-acetylneuraminate using CTP in the reaction.

The terms “L-glutamine-D-fructose-6-phosphate aminotransferase”, “glutamine-fructose-6-phosphate transaminase (isomerizing)”, “hexosephosphate aminotransferase”, “glucosamine-6-phosphate isomerase (glutamine-forming)”, “glutamine-fructose-6-phosphate transaminase (isomerizing)”, “D-fructose-6-phosphate amidotransferase”, “fructose-6-phosphate aminotransferase”, “glucosaminephosphate isomerase”, “glucosamine 6-phosphate synthase”, “GlcN6P synthase”, “GFA”, “glms”, “glmS” and “glmS*54” are used interchangeably and refer to an enzyme that catalyses the conversion of D-fructose-6-phosphate into D-glucosamine-6-phosphate using L-glutamine.

The terms “glucosamine-6-P deaminase”, “glucosamine-6-phosphate deaminase”, “GlcN6P deaminase”, “glucosamine-6-phosphate isomerase”, “glmD” and “nagB” are used interchangeably and refer to an enzyme that catalyses the reversible isomerization-deamination of glucosamine-6-phosphate (GlcN6P) to form fructose-6-phosphate and an ammonium ion.

The terms “phosphoglucosamine mutase” and “glmM” are used interchangeably and refer to an enzyme that catalyses the conversion of glucosamine-6-phosphate to glucosamine-1-phosphate. Phosphoglucosamine mutase can also catalyse the formation of glucose-6-P from glucose-1-P, although at a 1400-fold lower rate.

The terms “N-acetylglucosamine-6-P deacetylase”, “N-acetylglucosamine-6-phosphate deacetylase” and “nagA” are used interchangeably and refer to an enzyme that catalyses the hydrolysis of the N-acetyl group of N-acetylglucosamine-6-phosphate (GlcNAc-6-P) to yield glucosamine-6-phosphate (GlcN6P) and acetate.

An N-acylglucosamine 2-epimerase is an enzyme that catalyses the reaction N-acyl-D-glucosamine=N-acyl-D-mannosamine. Alternative names for this enzyme comprise N-acetylglucosamine 2-epimerase, N-acetyl-D-glucosamine 2-epimerase, GlcNAc 2-epimerase, N-acyl-D-glucosamine 2-epimerase and N-acetylglucosamine epimerase.

A UDP-N-acetylglucosamine 2-epimerase is an enzyme that catalyses the reaction N-acetyl-D-glucosamine=N-acetylmannosamine. Alternative names for this enzyme comprise UDP-N-acylglucosamine 2-epimerase, UDP-GlcNAc-2-epimerase, “neuC” and UDP-N-acetyl-D-glucosamine 2-epimerase.

A bifunctional UDP-GlcNAc 2-epimerase/kinase is a bifunctional enzyme that catalyses the reaction UDP-N-acetyl-D-glucosamine=N-acetyl-D-mannosamine and the reaction N-acetyl-D-mannosamine+ATP=ADP+N-acetyl-D-mannosamine 6-phosphate.

A glucosamine 6-phosphate N-acetyltransferase is an enzyme that catalyses the transfer of an acetyl group from acetyl-CoA to D-glucosamine-6-phosphate thereby generating a free CoA and N-acetyl-D-glucosamine 6-phosphate. Alternative names comprise aminodeoxyglucosephosphate acetyltransferase, D-glucosamine-6-P N-acetyltransferase, glucosamine 6-phosphate acetylase, glucosamine 6-phosphate N-acetyltransferase, glucosamine-phosphate N-acetyltransferase, glucosamine-6-phosphate acetylase, N-acetylglucosamine-6-phosphate synthase, phosphoglucosamine acetylase, phosphoglucosamine N-acetylase phosphoglucosamine N-acetylase, phosphoglucosamine transacetylase, GNA and GNA1.

The term “N-acetylglucosamine-6-phosphate phosphatase” refers to an enzyme that dephosphorylates N-acetylglucosamine-6-phosphate (GlcNAc-6-P) hereby synthesizing N-acetylglucosamine (GlcNAc).

The term “N-acetylmannosamine-6-phosphate phosphatase” refers to an enzyme that dephosphorylates N-acetylmannosamine-6-phosphate (ManNAc-6P) to N-acetylmannosamine (ManNAc).

The terms “N-acetylneuraminate kinase”, “ManNAc kinase”, “N-acetyl-D-mannosamine kinase” and “nanK” are used interchangeably and refer to an enzyme that phosphorylates ManNAc to synthesize N-acetylmannosamine-phosphate (ManNAc-6-P).

The terms “N-acetylmannosamine-6-phosphate 2-epimerase”, “ManNAc-6-P isomerase”, “ManNAc-6-P 2-epimerase”, N-acetylglucosamine-6P 2-epimerase and “nanE” are used interchangeably and refer to an enzyme that catalyzes the reaction ManNAc-6-P═N-acetylglucosamine-6-phosphate (GlcNAc-6-P).

The terms “phosphoacetylglucosamine mutase”, “acetylglucosamine phosphomutase”, “acetylaminodeoxyglucose phosphomutase”, “phospho-N-acetylglucosamine mutase” and “N-acetyl-D-glucosamine 1,6-phosphomutase” are used interchangeably and refer to an enzyme that catalyses the conversion of N-acetyl-glucosamine 1-phosphate into N-acetylglucosamine 6-phosphate.

The terms “N-acetylglucosamine 1-phosphate uridylyltransferase”, “N-acetylglucosamine-1-phosphate uridyltransferase”, “UDP-N-acetylglucosamine diphosphorylase”, “UDP-N-acetylglucosamine pyrophosphorylase”, “uridine diphosphoacetylglucosamine pyrophosphorylase”, “UTP:2-acetamido-2-deoxy-alpha-D-glucose-1-phosphate uridylyltransferase”, “UDP-GlcNAc pyrophosphorylase”, “GlmU uridylyltransferase”, “Acetylglucosamine 1-phosphate uridylyltransferase”, “UDP-acetylglucosamine pyrophosphorylase”, “uridine diphosphate-N-acetylglucosamine pyrophosphorylase”, “uridine diphosphoacetylglucosamine phosphorylase”, and “acetylglucosamine 1-phosphate uridylyltransferase” are used interchangeably and refer to an enzyme that catalyses the conversion of N-acetylglucosamine 1-phosphate (GlcNAc-1-P) into UDP-N-acetylglucosamine (UDP-GlcNAc) by the transfer of uridine 5-monophosphate (from uridine 5-triphosphate (UTP)).

The term glucosamine-1-phosphate acetyltransferase refers to an enzyme that catalyses the transfer of the acetyl group from acetyl coenzyme A to glucosamine-1-phosphate (GlcN-1-P) to produce N-acetylglucosamine-1-phosphate (GlcNAc-1-P).

The term “glmU” refers to a bifunctional enzyme that has both N-acetylglucosamine-1-phosphate uridyltransferase and glucosamine-1-phosphate acetyltransferase activity and that catalyses two sequential reactions in the de novo biosynthetic pathway for UDP-GlcNAc. The C-terminal domain catalyses the transfer of acetyl group from acetyl coenzyme A to GlcN-1-P to produce GlcNAc-1-P, which is converted into UDP-GlcNAc by the transfer of uridine 5-monophosphate, a reaction catalysed by the N-terminal domain.

The terms “N-acetylneuraminate lyase”, “Neu5Ac lyase”, “N-acetylneuraminate pyruvate-lyase”, “N-acetylneuraminic acid aldolase”, “NALase”, “sialate lyase”, “sialic acid aldolase”, “sialic acid lyase” and “nanA” are used interchangeably and refer to an enzyme that degrades N-acetylneuraminate into N-acetylmannosamine (ManNAc) and pyruvate.

The terms “N-acylneuraminate-9-phosphate synthase”, “N-acylneuraminate-9-phosphate synthetase”, “NANA synthase”, “NANAS”, “NANS”, “NmeNANAS”, “N-acetylneuraminate pyruvate-lyase (pyruvate-phosphorylating)” as used herein are used interchangeably and refer to an enzyme capable to synthesize N-acylneuraminate-9-phosphate from N-acetylmannosamine-6-phosphate (ManNAc-6-phosphate) in a reaction using phosphoenolpyruvate (PEP).

The term “N-acylneuraminate-9-phosphatase” refers to an enzyme capable to dephosphorylate N-acylneuraminate-9-phosphate to synthesise N-acylneuraminate.

The term “glycosyltransferase” as used herein refers to an enzyme capable to catalyse the transfer of sugar moieties from activated donor molecules to specific acceptor molecules, forming glycosidic bonds. Said activated donor molecule can be a precursor as defined herein The as such synthesized oligosaccharides can be of the linear type or of the branched type and can contain multiple monosaccharide building blocks. A classification of glycosyltransferases using nucleotide diphospho-sugar, nucleotide monophospho-sugar and sugar phosphates and related proteins into distinct sequence-based families has been described (Campbell et al., Biochem. J. 326, 929-939 (1997)) and is available on the CAZy (CArbohydrate-Active EnZymes) website (www.cazy.org).

Examples of glycosyltransferase are fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases and fucosaminyltransferases.

Fucosyltransferases are glycosyltransferases that transfer a fucose residue (Fuc) from a GDP-fucose (GDP-Fuc) donor onto an acceptor. Fucosyltransferases comprise alpha-1,2-fucosyltransferases, alpha-1,3-fucosyltransferases, alpha-1,4-fucosyltransferases, alpha-1,3/4-fucosyltransferases and alpha-1,6-fucosyltransferases that catalyse the transfer of a Fuc residue from GDP-Fuc onto an acceptor via alpha-glycosidic bonds. Fucosyltransferases can be found but are not limited to the GT10, GT11, GT23, GT65 and GT68 CAZy families. Sialyltransferases are glycosyltransferases that transfer a sialyl group (like Neu5Ac or Neu5Gc) from a donor (like CMP-Neu5Ac or CMP-Neu5Gc) onto an acceptor. Sialyltransferases comprise alpha-2,3-sialyltransferases, alpha-2,6-sialyltransferases and alpha-2,8-sialyltransferases that catalyse the transfer of a sialyl group onto an acceptor via alpha-glycosidic bonds. Sialyltransferases can be found but are not limited to the GT29, GT42, GT80 and GT97 CAZy families. Galactosyltransferases are glycosyltransferases that transfer a galactosyl group (Gal) from a UDP-galactose (UDP-Gal) donor onto an acceptor. Galactosyltransferases comprise beta-1,3-galactosyltransferases, N-acetylglucosamine beta-1,3-galactosyltransferases, beta-1,4-galactosyltransferases, N-acetylglucosamine beta-1,4-galactosyltransferases, alpha-1,3-galactosyltransferases and alpha-1,4-galactosyltransferases that transfer a Gal residue from UDP-Gal onto a acceptor via alpha- or beta-glycosidic bonds. Galactosyltransferases can be found but are not limited to the GT2, GT6, GT8, GT25 and GT92 CAZy families. Glucosyltransferases are glycosyltransferases that transfer a glucosyl group (Glc) from a UDP-glucose (UDP-Glc) donor onto an acceptor. Glucosyltransferases comprise alpha-glucosyltransferases, beta-1,2-glucosyltransferases, beta-1,3-glucosyltransferases and beta-1,4-glucosyltransferases that transfer a Glc residue from UDP-Glc onto an acceptor via alpha- or beta-glycosidic bonds. Glucosyltransferases can be found but are not limited to the GT1, GT4 and GT25 CAZy families. Mannosyltransferases are glycosyltransferases that transfer a mannose group (Man) from a GDP-mannose (GDP-Man) donor onto an acceptor. Mannosyltransferases comprise alpha-1,2-mannosyltransferases, alpha-1,3-mannosyltransferases and alpha-1,6-mannosyltransferases that transfer a Man residue from GDP-Man onto an acceptor via alpha-glycosidic bonds. Mannosyltransferases can be found but are not limited to the GT22, GT39, GT62 and GT69 CAZy families. N-acetylglucosaminyltransferases are glycosyltransferases that transfer an N-acetylglucosamine group (GlcNAc) from a UDP-N-acetylglucosamine (UDP-GlcNAc) donor onto an acceptor. N-acetylglucosaminyltransferases can be found but are not limited to GT2 and GT4 CAZy families. Galactoside beta-1,3-N-acetylglucosaminyltransferases are part of N-acetylglucosaminyltransferases and transfer GlcNAc from a UDP-GlcNAc donor onto a terminal galactose unit present in an acceptor via a beta-1,3-linkage. Beta-1,6-N-acetylglucosaminyltransferases are N-acetylglucosaminyltransferases that transfer GlcNAc from a UDP-GlcNAc donor onto an acceptor via a beta-1,6-linkage. N-acetylgalactosaminyltransferases are glycosyltransferases that transfer an N-acetylgalactosamine group (GalNAc) from a UDP-N-acetylgalactosamine (UDP-GalNAc) donor onto an acceptor. N-acetylgalactosaminyltransferases can be found but are not limited to GT7, GT12 and GT27 CAZy families. N-acetylmannosaminyltransferases are glycosyltransferases that transfer an N-acetylmannosamine group (ManNAc) from a UDP-N-acetylmannosamine (UDP-ManNAc) donor onto a acceptor. Xylosyltransferases are glycosyltransferases that transfer a xylose residue (Xyl) from a UDP-xylose (UDP-Xyl) donor onto a acceptor. Xylosyltransferases can be found but are not limited to GT14, GT61 and GT77 CAZy families. Glucuronyltransferases are glycosyltransferases that transfer a glucuronate from a UDP-glucuronate donor onto an acceptor via alpha- or beta-glycosidic bonds. Glucuronyltransferases can be found but are not limited to GT4, GT43 and GT93 CAZy families. Galacturonyltransferases are glycosyltransferases that transfer a galacturonate from a UDP-galacturonate donor onto an acceptor. N-glycolylneuraminyltransferases are glycosyltransferases that transfer an N-glycolylneuraminic acid group (Neu5Gc) from a CMP-Neu5Gc donor onto an acceptor. Rhamnosyltransferases are glycosyltransferases that transfer a rhamnose residue from a GDP-rhamnose donor onto an acceptor. Rhamnosyltransferases can be found but are not limited to the GT1, GT2 and GT102 CAZy families. N-acetylrhamnosyltransferases are glycosyltransferases that transfer an N-acetylrhamnosamine residue from a UDP-N-acetyl-L-rhamnosamine donor onto an acceptor. UDP-4-amino-4,6-dideoxy-N-acetyl-beta-L-altrosamine transaminases are glycosyltransferases that use a UDP-2-acetamido-2,6-dideoxy-L-arabino-4-hexulose in the biosynthesis of pseudaminic acid, which is a sialic acid-like sugar that is used to modify flagellin. Fucosaminyltransferases are glycosyltransferases that transfer an N-acetylfucosamine residue from a dTDP-N-acetylfucosamine or a UDP-N-acetylfucosamine donor onto an acceptor.

The term “monosaccharide” as used herein refers to a sugar that is not decomposable into simpler sugars by hydrolysis, is classed either an aldose or ketose, and contains one or more hydroxyl groups per molecule. Monosaccharides are saccharides containing only one simple sugar. Examples of monosaccharides comprise Hexose, D-Glucopyranose, D-Galactofuranose, D-Galactopyranose, L-Galactopyranose, D-Mannopyranose, D-Allopyranose, L-Altropyranose, D-Gulopyranose, L-Idopyranose, D-Talopyranose, D-Ribofuranose, D-Ribopyranose, D-Arabinofuranose, D-Arabinopyranose, L-Arabinofuranose, L-Arabinopyranose, D-Xylopyranose, D-Lyxopyranose, D-Erythrofuranose, D-Threofuranose, Heptose, L-glycero-D-manno-Heptopyranose (LDmanHep), D-glycero-D-manno-Heptopyranose (DDmanHep), 6-Deoxy-L-altropyranose, 6-Deoxy-D-gulopyranose, 6-Deoxy-D-talopyranose, 6-Deoxy-D-galactopyranose, 6-Deoxy-L-galactopyranose, 6-Deoxy-D-mannopyranose, 6-Deoxy-L-mannopyranose, 6-Deoxy-D-glucopyranose, 2-Deoxy-D-arabino-hexose, 2-Deoxy-D-erythro-pentose, 2,6-Dideoxy-D-arabino-hexopyranose, 3,6-Dideoxy-D-arabino-hexopyranose, 3,6-Dideoxy-L-arabino-hexopyranose, 3,6-Dideoxy-D-xylo-hexopyranose, 3,6-Dideoxy-D-ribo-hexopyranose, 2,6-Dideoxy-D-ribo-hexopyranose, 3,6-Dideoxy-L-xylo-hexopyranose, 2-Amino-2-deoxy-D-glucopyranose, 2-Amino-2-deoxy-D-galactopyranose, 2-Amino-2-deoxy-D-mannopyranose, 2-Amino-2-deoxy-D-allopyranose, 2-Amino-2-deoxy-L-altropyranose, 2-Amino-2-deoxy-D-gulopyranose, 2-Amino-2-deoxy-L-idopyranose, 2-Amino-2-deoxy-D-talopyranose, 2-Acetamido-2-deoxy-D-glucopyranose, 2-Acetamido-2-deoxy-D-galactopyranose, 2-Acetamido-2-deoxy-D-mannopyranose, 2-Acetamido-2-deoxy-D-allopyranose, 2-Acetamido-2-deoxy-L-altropyranose, 2-Acetamido-2-deoxy-D-gulopyranose, 2-Acetamido-2-deoxy-L-idopyranose, 2-Acetamido-2-deoxy-D-talopyranose, 2-Acetamido-2,6-dideoxy-D-galactopyranose, 2-Acetamido-2,6-dideoxy-L-galactopyranose, 2-Acetamido-2,6-dideoxy-L-mannopyranose, 2-Acetamido-2,6-dideoxy-D-glucopyranose, 2-Acetamido-2,6-dideoxy-L-altropyranose, 2-Acetamido-2,6-dideoxy-D-talopyranose, D-Glucopyranuronic acid, D-Galactopyranuronic acid, D-Mannopyranuronic acid, D-Allopyranuronic acid, L-Altropyranuronic acid, D-Gulopyranuronic acid, L-Gulopyranuronic acid, L-Idopyranuronic acid, D-Talopyranuronic acid, sialic acid, 5-Amino-3,5-dideoxy-D-glycero-D-galacto-non-2-ulosonic acid, 5-Acetamido-3,5-dideoxy-D-glycero-D-galacto-non-2-ulosonic acid, 5-Glycolylamido-3,5-dideoxy-D-glycero-D-galacto-non-2-ulosonic acid, Erythritol, Arabinitol, Xylitol, Ribitol, Glucitol, Galactitol, Mannitol, D-ribo-Hex-2-ulopyranose, D-arabino-Hex-2-ulofuranose (D-fructofuranose), D-arabino-Hex-2-ulopyranose, L-xylo-Hex-2-ulopyranose, D-lyxo-Hex-2-ulopyranose, D-threo-Pent-2-ulopyranose, D-altro-Hept-2-ulopyranose, 3-C-(Hydroxymethyl)-D-erythrofuranose, 2,4,6-Trideoxy-2,4-diamino-D-glucopyranose, 6-Deoxy-3-O-methyl-D-glucose, 3-O-Methyl-D-rhamnose, 2,6-Dideoxy-3-methyl-D-ribo-hexose, 2-Amino-3-O—[(R)-1-carboxyethyl]-2-deoxy-D-glucopyranose, 2-Acetamido-3-O—[(R)-carboxyethyl]-2-deoxy-D-glucopyranose, 2-Glycolylamido-3-O—[(R)-1-carboxyethyl]-2-deoxy-D-glucopyranose, 3-Deoxy-D-lyxo-hept-2-ulopyranosaric acid, 3-Deoxy-D-manno-oct-2-ulopyranosonic acid, 3-Deoxy-D-glycero-D-galacto-non-2-ulopyranosonic acid, 5,7-Diamino-3,5,7,9-tetradeoxy-L-glycero-L-manno-non-2-ulopyranosonic acid, 5,7-Diamino-3,5,7,9-tetradeoxy-L-glycero-L-altro-non-2-ulopyranosonic acid, 5,7-Diamino-3,5,7,9-tetradeoxy-D-glycero-D-galacto-non-2-ulopyranosonic acid, 5,7-Diamino-3,5,7,9-tetradeoxy-D-glycero-D-talo-non-2-ulopyranosonic acid, 2-acetamido-2,6-dideoxy-L-arabino-4-hexulose, 2-acetamido-2,6-dideoxy-L-lyxo-4-hexulose, N-acetyl-L-rhamnosamine, N-acetyl-D-fucosamine, N-acetyl-L-pneumosamine, N-acetylmuramic acid, N-acetyl-L-quinovosamine, glucose (Glc), galactose (Gal), N-acetylglucosamine (GlcNAc), glucosamine (Glcn), mannose (Man), xylose (Xyl), N-acetylmannosamine (ManNAc), N-glycolylneuraminic acid, N-acetylgalactosamine (GalNAc), galactosamine (Galn), fucose (Fuc), rhamnose (Rha), glucuronic acid, gluconic acid, fructose (Fru) and polyols. With the term polyol is meant an alcohol containing multiple hydroxyl groups. For example, glycerol, sorbitol, or mannitol.

The term “phosphorylated monosaccharide” as used herein refers to one of the above listed monosaccharides which is phosphorylated. Examples of phosphorylated monosaccharides include but are not limited to glucose-1-phosphate, glucose-6-phosphate, glucose-1,6-bisophosphate, galactose-1-phosphate, fructose-6-phosphate, fructose-1,6-bisphosphate, fructose-1-phosphate, glucosamine-1-phosphate, glucosamine-6-phosphate, N-acetylglucosamine-1-phosphate, mannose-1-phosphate, mannose-6-phosphate or fucose-1-phosphate. Some, but not all, of these phosphorylated monosaccharides are precursors or intermediates for the production of activated monosaccharide.

The terms “activated monosaccharide”, “nucleotide-activated sugar”, “nucleotide-sugar”, “activated sugar”, “nucleoside” or “nucleotide donor” are used herein interchangeably and refer to activated forms of monosaccharides. Examples of activated monosaccharides include but are not limited to UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), UDP-glucuronate, UDP-galacturonate, UDP-2-acetamido-2,6-dideoxy-L-arabino-4-hexulose, UDP-2-acetamido-2,6-dideoxy-L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), GDP-L-quinovose, CMP-sialic acid (CMP-Neu5Ac or CMP-N-acetylneuraminic acid), CMP-N-glycolylneuraminic acid (CMP-Neu5Gc), CMP-Neu4Ac, CMP-Neu5Ac9N₃, CMP-Neu4,5Ac₂, CMP-Neu5,7Ac₂, CMP-Neu5,9Ac₂, CMP-Neu5,7(8,9)Ac₂, GDP-fucose (GDP-Fuc), GDP-rhamnose and UDP-xylose. Nucleotide-sugars act as glycosyl donors in glycosylation reactions. Glycosylation reactions are reactions that are catalysed by glycosyltransferases.

The term “disaccharide” as used herein refers to a saccharide polymer containing two simple sugars, i.e. monosaccharides. Such disaccharides contain monosaccharides preferably selected from the list of monosaccharides as used herein above. Examples of disaccharides comprise lactose (Gal-b1,4-Glc), lacto-N-biose (Gal-b1,3-GlcNAc), N-acetyllactosamine (Gal-b1,4-GlcNAc), LacDiNAc (GalNAc-b1,4-GlcNAc), N-acetylgalactosaminylglucose (GalNAc-b1,4-Glc), Neu5Ac-a2,3-Gal, Neu5Ac-a2,6-Gal and fucopyranosyl-(1-4)-N-glycolylneuraminic acid (Fuc-(1-4)-Neu5Gc).

“Oligosaccharide” as the term is used herein and as generally understood in the state of the art, refers to a saccharide polymer containing a small number, typically three to ten, but as used herein three to twenty, of simple sugars, i.e. monosaccharides. The oligosaccharide as used in the present invention can be a linear structure or can include branches. The linkage (e.g., glycosidic linkage, galactosidic linkage, glucosidic linkage, etc.) between two sugar units can be expressed, for example, as 1,4, 1->4, or (1-4), used interchangeably herein. For example, the terms “Gal-b1,4-Glc”, “Gal-β1,4-Glc”, “b-Gal-(1->4)-Glc”, “β-Gal-(1->4)-Glc”, “Galbeta1-4-Glc”, “Gal-b(1-4)-Glc” and “Gal-β(1-4)-Glc” have the same meaning, i.e. a beta-glycosidic bond links carbon-1 of galactose (Gal) with the carbon-4 of glucose (Glc). Each monosaccharide can be in the cyclic form (e.g., pyranose or furanose form). Linkages between the individual monosaccharide units may include alpha 1->2, alpha 1->3, alpha 1->4, alpha 1->6, alpha 2->1, alpha 2->3, alpha 2->4, alpha 2->6, beta 1->2, beta 1->3, beta 1->4, beta 1->6, beta 2->1, beta 2->3, beta 2->4, and beta 2->6. An oligosaccharide can contain both alpha- and beta-glycosidic bonds or can contain only beta-glycosidic bonds. The term “polysaccharide” refers to a saccharide consisting of a large number, read more than twenty, of monosaccharides linked glycosidically.

Examples of oligosaccharides include but are not limited to Lewis-type antigen oligosaccharides, mammalian (including human) milk oligosaccharides, O-antigen, enterobacterial common antigen (ECA), the glycan chain present in lipopolysaccharides (LPS), the oligosaccharide repeats present in capsular polysaccharides, peptidoglycan (PG), amino-sugars and antigens of the human ABO blood group system. As used herein, “mammalian milk oligosaccharide” refers to oligosaccharides such as but not limited to 3-fucosyllactose, 2′-fucosyllactose, 6-fucosyllactose, 2′,3-difucosyllactose, 2′,2-difucosyllactose, 3,4-difucosyllactose, 6′-sialyllactose, 3′-sialyllactose, 3,6-disialyllactose, 6,6′-disialyllactose, 8,3-disialyllactose, 3,6-disialyllacto-N-tetraose, lactodifucotetraose, lacto-N-tetraose, lacto-N-neotetraose, lacto-N-fucopentaose II, lacto-N-fucopentaose I, lacto-N-fucopentaose III, lacto-N-fucopentaose V, lacto-N-fucopentaose VI, sialyllacto-N-tetraose c, sialyllacto-N-tetraose b, sialyllacto-N-tetraose a, lacto-N-difucohexaose I, lacto-N-difucohexaose II, lacto-N-hexaose, lacto-N-neohexaose, para-lacto-N-hexaose, monofucosylmonosialyllacto-N-tetraose c, monofucosyl para-lacto-N-hexaose, monofucosyllacto-N-hexaose III, isomeric fucosylated lacto-N-hexaose III, isomeric fucosylated lacto-N-hexaose I, sialyllacto-N-hexaose, sialyllacto-N-neohexaose II, difucosyl-para-lacto-N-hexaose, difucosyllacto-N-hexaose, difucosyllacto-N-hexaose a, difucosyllacto-N-hexaose c, galactosylated chitosan, fucosylated oligosaccharides, neutral oligosaccharide and/or sialylated oligosaccharides.

A ‘fucosylated oligosaccharide’ as used herein and as generally understood in the state of the art is an oligosaccharide that is carrying a fucose-residue. Examples comprise 2′-fucosyllactose (2′FL), 3-fucosyllactose (3FL), 4-fucosyllactose (4FL), 6-fucosyllactose (6FL), difucosyllactose (diFL), lactodifucotetraose (LDFT), Lacto-N-fucopentaose I (LNF I), Lacto-N-fucopentaose II (LNF II), Lacto-N-fucopentaose III (LNF III), lacto-N-fucopentaose V (LNF V), lacto-N-fucopentaose VI (LNF VI), lacto-N-neofucopentaose I, lacto-N-difucohexaose I (LDFH I), lacto-N-difucohexaose II (LDFH II), Monofucosyllacto-N-hexaose III (MFLNH III), Difucosyllacto-N-hexaose (DFLNHa), difucosyl-lacto-N-neohexaose.

As used herein, a ‘sialylated oligosaccharide’ is to be understood as a negatively charged sialic acid containing oligosaccharide, i.e. an oligosaccharide having a sialic acid residue. It has an acidic nature. Some examples are 3-SL (3′-sialyllactose or 3′SL or Neu5Ac-a2,3-Gal-b1,4-Glc), 3′-sialyllactosamine, 6-SL (6′-sialyllactose or 6′SL or Neu5Ac-a2,6-Gal-b1,4-Glc), 3,6-disialyllactose (Neu5Ac-a2,3-(Neu5Ac-a2,6)-Gal-b1,4-Glc), 6,6′-disialyllactose (Neu5Ac-a2,6-Gal-b1,4-(Neu5Ac-a2,6)-Glc), 8,3-disialyllactose (Neu5Ac-a2,8-Neu5Ac-a2,3-Gal-b1,4-Glc), 6′-sialyllactosamine, oligosaccharides comprising 6′-sialyllactose, SGG hexasaccharide (Neu5Acα-2,3Galβ-1,3GalNacβ-1,3Galα-1,4Galβ-1,4Gal), sialylated tetrasaccharide (Neu5Acα-2,3Galβ-1,4GlcNacβ-14GlcNAc), pentasaccharide LSTD (Neu5Acα-2,3Galβ-1,4GlcNacβ-1,3Galβ-1,4Glc), sialylated lacto-N-triose, sialylated lacto-N-tetraose, sialyllacto-N-neotetraose, monosialyllacto-N-hexaose, disialyllacto-N-hexaose I, monosialyllacto-N-neohexaose I, monosialyllacto-N-neohexaose II, disialyllacto-N-neohexaose, disialyllacto-N-tetraose, disialyllacto-N-hexaose II, sialyllacto-N-tetraose a, disialyllacto-N-hexaose I, sialyllacto-N-tetraose b, 3′-sialyl-3-fucosyllactose, disialomonofucosyllacto-N-neohexaose, monofucosylmonosialyllacto-N-octaose (sialyl Lea), sialyllacto-N-fucohexaose II, disialyllacto-N-fucopentaose II, monofucosyldisialyllacto-N-tetraose and oligosaccharides bearing one or several sialic acid residue(s), including but not limited to: oligosaccharide moieties of the gangliosides selected from GM3 (3′sialyllactose, Neu5Acα-2,3Galβ-4Glc) and oligosaccharides comprising the GM3 motif, GD3 Neu5Acα-2,8Neu5Acα-2,3Galβ-1,4Glc GT3 (Neu5Acα-2,8Neu5Acα-2,8Neu5Acα-2,3Galβ-1,4Glc); GM2 GalNAcβ-1,4(Neu5Acα-2,3)Galβ-1,4Glc, GM1 Galβ-1,3GalNAcβ-1,4(Neu5Acα-2,3)Galβ-1,4Glc, GD1a Neu5Acα-2,3Galβ-1,3GalNAcβ-1,4(Neu5Acα-2,3)Galβ-1,4Glc, GT1a Neu5Acα-2,8Neu5Acα-2,3Galβ-1,3GalNAcβ-1,4(Neu5Acα-2,3)Galβ-1,4Glc, GD2 GalNAcβ-1,4(Neu5Acα-2,8Neu5Acα2,3)Galβ-1,4Glc, GT2 GalNAcβ-1,4(Neu5Acα-2,8Neu5Acα-2,8Neu5Acα2,3)Galβ-1,4Glc, GD1b, Galβ-1,3GalNAcβ-1,4(Neu5Acα-2,8Neu5Acα2,3)Galβ-1,4Glc, GT1b Neu5Acα-2,3Galβ-1,3GalNAcβ-1,4(Neu5Acα-2,8Neu5Acα2,3)Galβ-1,4Glc, GQ1b Neu5Acα-2,8Neu5Acα-2,3Galβ-1,3GalNAc β-1,4(Neu5Acα-2,8Neu5Acα2,3)Galβ-1,4Glc, GT1c Galβ-1,3GalNAcβ-1,4(Neu5Acα-2,8Neu5Acα-2,8Neu5Acα2,3)Galβ-1,4Glc, GQ1c Neu5Acα-2,3Galβ-1,3GalNAc β-1,4(Neu5Acα-2,8Neu5Acα-2,8Neu5Acα2,3)Galβ-1,4Glc, GP1c Neu5Acα-2,8Neu5Acα-2,3Galβ-1,3GalNAc β-1,4(Neu5Acα-2,8Neu5Acα-2,8Neu5Acα2,3)Galβ-1,4Glc, GD1a Neu5Acα-2,3Galβ-1,3(Neu5Acα-2,6)GalNAcβ-1,4Galβ-1,4Glc, Fucosyl-GM1 Fuca-1,2Galβ-1,3GalNAcβ-1,4(Neu5Acα-2,3)Gal β-1,4Glc; all of which may be extended to the production of the corresponding gangliosides by reacting the above oligosaccharide moieties with ceramide or synthetizing the above oligosaccharides on a ceramide.

A ‘neutral oligosaccharide’ or a ‘non-charged oligosaccharide’ as used herein and as generally understood in the state of the art is an oligosaccharide that has no negative charge originating from a carboxylic acid group. Examples of such neutral oligosaccharide are 2′-fucosyllactose (2′FL), 3-fucosyllactose (3FL), 2′, 3-difucosyllactose (diFL), lacto-N-triose II, lacto-N-tetraose, lacto-N-neotetraose, lacto-N-fucopentaose I, lacto-N-neofucopentaose I, lacto-N-fucopentaose II, lacto-N-fucopentaose III, lacto-N-fucopentaose V, lacto-N-fucopentaose VI, lacto-N-neofucopentaose V, lacto-N-difucohexaose I, lacto-N-difucohexaose II, 6′-galactosyllactose, 3′-galactosyllactose, lacto-N-hexaose, lacto-N-neohexaose, para-lacto-N-hexaose, para-lacto-N-neohexaose, difucosyl-lacto-N-hexaose and difucosyl-lacto-N-neohexaose.

Mammalian milk oligosaccharides comprise oligosaccharides present in milk found in any phase during lactation including colostrum milk from humans and mammals including but not limited to cows (Bos taurus), sheep (Ovis aries), goats (Capra aegagrus hircus), bactrian camels (Camelus bactrianus), horses (Equus ferus caballus), pigs (Sus scropha), dogs (Canis lupus familiaris), ezo brown bears (Ursus arctos yesoensis), polar bear (Ursus maritimus), Japanese black bears (Ursus thibetanus japonicus), striped skunks (Mephitis mephitis), hooded seals (Cystophora cristata), Asian elephants (Elephas maximus), African elephant (Loxodonta africana), giant anteater (Myrmecophaga tridactyla), common bottlenose dolphins (Tursiops truncates), northern minke whales (Balaenoptera acutorostrata), tammar wallabies (Macropus eugenii), red kangaroos (Macropus rufus), common brushtail possum (Trichosurus vulpecula), koalas (Phascolarctos cinereus), eastern quolls (Dasyurus viverrinus), platypus (Ornithorhynchus anatinus).

As used herein the term “Lewis-type antigens” comprise the following oligosaccharides: H1 antigen, which is Fucα1-2Galβ1-3GlcNAc, or in short 2′FLNB; Lewis a, which is the trisaccharide Galβ1-3[Fucα1-4]GlcNAc, or in short 4-FLNB; Lewis b, which is the tetrasaccharide Fucα1-2Galβ1-3[Fucα1-4]GlcNAc, or in short DiF-LNB; sialyl Lewis a which is 5-acetylneuraminyl-(2-3)-galactosyl-(1-3)-(fucopyranosyl-(1-4))-N-acetylglucosamine, or written in short Neu5Acα2-3Galβ1-3[Fucα1-4]GlcNAc; H2 antigen, which is Fucα1-2Galβ1-4GlcNAc, or otherwise stated 2′fucosyl-N-acetyl-lactosamine, in short 2′FLacNAc; Lewis x, which is the trisaccharide Galβ1-4[Fucα1-3]GlcNAc, or otherwise known as 3-Fucosyl-N-acetyl-lactosamine, in short 3-FLacNAc, Lewis y, which is the tetrasaccharide Fucα1-2Galβ1-4[Fucα1-3]GlcNAc and sialyl Lewis x which is 5-acetylneuraminyl-(2-3)-galactosyl-(1-4)-(fucopyranosyl-(1-3))-N-acetylglucosamine, or written in short Neu5Acα2-3Galβ1-4[Fucα1-3]GlcNAc.

As used herein, the term “O-antigen” refers to the repetitive glycan component of the surface lipopolysaccharide (LPS) of Gram-negative bacteria. The term “lipopolysaccharide” or “LPS” refers to glycolipids found in the outer membrane of Gram-negative bacteria which are composed of a lipid A, a core oligosaccharide and the O-antigen. The term “capsular polysaccharides” refers to long-chain polysaccharides with oligosaccharide repeat structures that are present in bacterial capsules, the latter being a polysaccharide layer that lies outside the cell envelope. The terms “peptidoglycan” or “murein” refers to an essential structural element in the cell wall of most bacteria, being composed of sugars and amino acids, wherein the sugar components consist of alternating residues of beta-1,4 linked GlcNAc and N-acetylmuramic acid. The term “amino-sugar” as used herein refers to a sugar molecule in which a hydroxyl group has been replaced with an amine group. As used herein, an antigen of the human ABO blood group system is an oligosaccharide. Such antigens of the human ABO blood group system are not restricted to human structures. Said structures involve the A determinant GalNAc-alpha1,3(Fuc-alpha1,2)-Gal-, the B determinant Gal-alpha1,3(Fuc-alpha1,2)-Gal- and the H determinant Fuc-alpha1,2-Gal- that are present on disaccharide core structures comprising Gal-beta1,3-GlcNAc, Gal-beta1,4-GlcNAc, Gal-beta1,3-GalNAc and Gal-beta1,4-Glc.

The terms “LNT II”, “LNT-II”, “LN3”, “lacto-N-triose II”, “lacto-N-triose II”, “lacto-N-triose”, “lacto-N-triose” or “GlcNAcβ1-3Galβ1-4Glc” as used in the present invention, are used interchangeably.

The terms “LNT”, “lacto-N-tetraose”, “lacto-N-tetraose” or “Galβ1-3GlcNAcβ1-3Galβ1-4Glc” as used in the present invention, are used interchangeably.

The terms “LNnT”, “lacto-N-neotetraose”, “lacto-N-neotetraose”, “neo-LNT” or “Galβ1-4GlcNAcβ1-3Galβ1-4Glc” as used in the present invention, are used interchangeably.

The terms “LSTa”, “LS-Tetrasaccharide a”, “Sialyl-lacto-N-tetraose a”, “sialyllacto-N-tetraose a” or “Neu5Ac-a2,3-Gal-b1,3-GlcNAc-b1,3-Gal-b1,4-Glc” as used in the present invention, are used interchangeably.

The terms “LSTb”, “LS-Tetrasaccharide b”, “Sialyl-lacto-N-tetraose b”, “sialyllacto-N-tetraose b” or “Gal-b1,3-(Neu5Ac-a2,6)-GlcNAc-b1,3-Gal-b1,4-Glc” as used in the present invention, are used interchangeably.

The terms “LSTc”, “LS-Tetrasaccharide c”, “Sialyl-lacto-N-tetraose c”, “sialyllacto-N-tetraose c”, “sialyllacto-N-neotetraose c” or “Neu5Ac-a2,6-Gal-b1,4-GlcNAc-b1,3-Gal-b1,4-Glc” as used in the present invention, are used interchangeably.

The terms “LSTd”, “LS-Tetrasaccharide d”, “Sialyl-lacto-N-tetraose d”, “sialyllacto-N-tetraose d”, “sialyllacto-N-neotetraose d” or “Neu5Ac-a2,3-Gal-b1,4-GlcNAc-b1,3-Gal-b1,4-Glc”.

As used herein, the term “glycolipid” refers to any of the glycolipids which are generally known in the art. Glycolipids (GLs) can be subclassified into Simple (SGLs) and Complex (CGLs) glycolipids. Simple GLs, sometimes called saccharolipids, are two-component (glycosyl and lipid moieties) GLs in which the glycosyl and lipid moieties are directly linked to each other. Examples of SGLs include glycosylated fatty acids, fatty alcohols, carotenoids, hopanoids, sterols or paraconic acids. Bacterially produced SGLs can be classified into rhamnolipids, glucolipids, trehalolipids, other glycosylated (non-trehalose containing) mycolates, trehalose-containing oligosaccharide lipids, glycosylated fatty alcohols, glycosylated macro-lactones and macro-lactams, glycomacrodiolides (glycosylated macrocyclic dilactones), glyco-carotenoids and glyco-terpenoids, and glycosylated hopanoids/sterols. Complex glycolipids (CGLs) are, however, structurally more heterogeneous, as they contain, in addition to the glycosyl and lipid moieties, other residues like for example glycerol (glycoglycerolipids), peptide (glycopeptidolipids), acylated-sphingosine (glycosphingolipids), or other residues (lipopolysaccharides, phenolic glycolipids, nucleoside lipids). Examples of Neu(n)Ac-containing glycolipids comprise octyl-beta-sialyllactoside, sialoglycosphingolipids and gangliosides.

The term “glycoprotein” refers to any of the glycoproteins which are generally known in the art. Glycoproteins can be subclassified based on the type of glycosylation present on the amino acid residues of the glycoprotein into N-glycosylated, O-glycosylated, P-glycosylated, C-glycosylated and S-glycosylated proteins. Examples of Neu(n)Ac-containing glycoproteins comprise but are not limited to sialyl-Tn-MUC1 and sialyl-T-MUC1 glycopeptides containing Neu5Gc, sialoglycopolypeptides, sialoglycoproteins, glycophorins like e.g. glycophorin A and glycophorin C, podocalyxin, gonadotropin receptors, podoplanin, CD43 (leukosialin, sialophorin) and the prion protein PrP.

The term “pathway for the production of a compound” as used herein is a biochemical pathway consisting of the enzymes and their respective genes involved in the synthesis of a disaccharide, oligosaccharide or Neu(n)Ac-containing bioproduct as defined herein. Said pathway for production of a disaccharide, oligosaccharide or Neu(n)Ac-containing bioproduct comprises but is not limited to pathways involved in the synthesis of a nucleotide-activated sugar and the transfer of said nucleotide-activated sugar to an acceptor to create a compound as defined herein. Examples of such pathway comprise but are not limited to a fucosylation, sialylation, galactosylation, N-acetylglucosaminylation, N-acetylgalactosylation, mannosylation, N-acetylmannosaminylation pathway.

A ‘fucosylation pathway’ as used herein is a biochemical pathway comprising at least one, preferably two of the enzymes and their respective genes chosen from the list comprising mannose-6-phosphate isomerase, phosphomannomutase, mannose-1-phosphate guanylyltransferase, GDP-mannose 4,6-dehydratase, GDP-L-fucose synthase, fucose permease, fucose kinase, fucose-1-phosphate guanylyltransferase and/or the salvage pathway L-fucokinase/GDP-fucose pyrophosphorylase, combined with a fucosyltransferase leading to α 1,2; α 1,3 α 1,4 and/or α 1,6 fucosylated oligosaccharides.

A ‘sialylation pathway’ is a biochemical pathway comprising at least one, preferably two, of the enzymes and their respective genes chosen from the list comprising an L-glutamine-D-fructose-6-phosphate aminotransferase, a phosphoglucosamine mutase, an N-acetylglucosamine-6-P deacetylase, an N-acylglucosamine 2-epimerase, a UDP-N-acetylglucosamine 2-epimerase, an N-acetylmannosamine-6-phosphate 2-epimerase, a UDP-GlcNAc 2-epimerase/kinase, a glucosamine 6-phosphate N-acetyltransferase, an N-acetylglucosamine-6-phosphate phosphatase, a phosphoacetylglucosamine mutase, an N-acetylglucosamine 1-phosphate uridylyltransferase, a glucosamine-1-phosphate acetyltransferase, an Neu(n)Ac synthase, an N-acetylneuraminate lyase, an N-acylneuraminate-9-phosphate synthase, an N-acylneuraminate-9-phosphatase, a sialic acid transporter and a CMP-sialic acid synthase, combined with a sialyltransferase leading to a 2,3; a 2,6 and/or a 2,8 sialylated oligosaccharides.

A ‘galactosylation pathway’ as used herein is a biochemical pathway comprising at least one, preferably two, of the enzymes and their respective genes chosen from the list comprising galactose-1-epimerase, galactokinase, glucokinase, galactose-1-phosphate uridylyltransferase, UDP-glucose 4-epimerase, glucose-1-phosphate uridylyltransferase, and phosphoglucomutase, combined with a galactosyltransferase leading to an alpha or beta bound galactose on any one or more of the 2, 3, 4 and 6 hydroxyl group of a mono-, di-, or oligosaccharide.

An ‘N-acetylglucosaminylation pathway’ as used herein is a biochemical pathway comprising at least one, preferably two, of the enzymes and their respective genes chosen from the list comprising L-glutamine-D-fructose-6-phosphate aminotransferase, N-acetylglucosamine-6-phosphate deacetylase, phosphoglucosamine mutase, N-acetylglucosamine-1-phosphate uridylyltransferas and glucosamine-1-phosphate acetyltransferase, combined with a glycosyltransferase leading to an alpha or beta bound N-acetylglucosamine on any one or more of the 3, 4 and 6 hydroxyl group of a mono-, di- or oligosaccharide.

An ‘N-acetylgalactosaminylation pathway’ as used herein is a biochemical pathway comprising at least one, preferably two, of the enzymes and their respective genes chosen from the list comprising L-glutamine-D-fructose-6-phosphate aminotransferase, phosphoglucosamine mutase, N-acetylglucosamine 1-phosphate uridylyltransferase, glucosamine-1-phosphate acetyltransferase, UDP-N-acetylglucosamine 4-epimerase, UDP-glucose 4-epimerase, N-acetylgalactosamine kinase and UDP-N-acetylgalactosamine pyrophosphorylase, combined with a glycosyltransferase leading to an alpha or beta bound N-acetylgalactosamine on a mono-, di- or oligosaccharide.

A ‘mannosylation pathway’ as used herein is a biochemical pathway comprising at least one, preferably two, of the enzymes and their respective genes chosen from the list comprising mannose-6-phosphate isomerase, phosphomannomutase and mannose-1-phosphate guanylyltransferase combined with a glycosyltransferase leading to an alpha or beta bound mannose on a mono-, di- or oligosaccharide.

An ‘N-acetylmannosaminylation pathway’ as used herein is a biochemical pathway comprising at least one, preferably two, of the enzymes and their respective genes chosen from the list comprising L-glutamine-D-fructose-6-phosphate aminotransferase, glucosamine-6-phosphate deaminase, phosphoglucosamine mutase, N-acetylglucosamine-6-phosphate deacetylase, glucosamine 6-phosphate N-acetyltransferase, N-acetylglucosamine-1-phosphate uridylyltransferase, glucosamine-1-phosphate acetyltransferase, glucosamine-1-phosphate acetyltransferase, UDP-GlcNAc 2-epimerase and ManNAc kinase combined with a glycosyltransferase leading to an alpha or beta bound N-acetylmannosamine on a mono-, di- or oligosaccharide.

The term “membrane proteins” as used herein refers to proteins that are part of or interact with the cells membrane and control the flow of molecules and information across the cell. The membrane proteins are thus involved in transport, be it import into or export out of the cell.

The term “enabled efflux” means to introduce the activity of transport of a solute over the cytoplasm membrane and/or the cell wall. Said transport may be enabled by introducing and/or increasing the expression of a transporter protein as described in the present invention. The term “enhanced efflux” means to improve the activity of transport of a solute over the cytoplasm membrane and/or the cell wall. Said transport may be enhanced by introducing and/or increasing the expression of a transporter protein as described in the present invention. “Expression” of a transporter protein is defined as “overexpression” of the gene encoding said transporter protein in the case said gene is an endogenous gene or “expression” in the case the gene encoding said transporter protein is a heterologous gene that is not present in the wild type strain.

As used herein, the term “cell productivity index (CPI)” refers to the mass of the product produced by the recombinant cells divided by the mass of the recombinant cells produced in the culture or cultivation.

The term “purified” refers to material that is substantially or essentially free from components which interfere with the activity of the biological molecule. For cells, saccharides, nucleic acids, polypeptides, peptides, glycoproteins, glycopeptides, lipids and glycolipids the term “purified” refers to material that is substantially or essentially free from components which normally accompany the material as found in its native state. Typically, purified saccharides, oligosaccharides, peptides, glycopeptides, proteins, glycoproteins, lipids, glycolipids or nucleic acids of the invention are at least about 50%, 55%, 60%, 65%, 70%, 75%, 80% or 85% pure, usually at least about 90%, 91%, 92%, 93%, 94%, 95%, 96%, 97%, 98%, or 99% pure as measured by band intensity on a silver-stained gel or other method for determining purity. Purity or homogeneity can be indicated by a number of means well known in the art, such as polyacrylamide gel electrophoresis of a protein or nucleic acid sample, followed by visualization upon staining. For certain purposes high resolution will be needed and HPLC or a similar means for purification utilized. For oligosaccharides, purity can be determined using methods such as but not limited to thin layer chromatography, gas chromatography, NMR, HPLC, capillary electrophoresis or mass spectroscopy.

The terms “cultivation”, “incubation” and “fermentation” are used interchangeably and refer to the culture medium wherein the cell is cultivated or fermented, the cell itself, and the disaccharide, oligosaccharide and/or Neu(n)Ac-containing bioproduct that is produced by the cell in whole broth, i.e. inside (intracellularly) as well as outside (extracellularly) of the cell.

The terms “reactor” and “incubator” refer to the recipient filled with the cultivation. Examples of reactors and incubators comprise but are not limited to microfluidic devices, well plates, tubes, shake flasks, fermenters, bioreactors, process vessels, cell culture incubators, CO2 incubators.

The term “precursor” as used herein refers to substances which are taken up or synthetized by the cell for the specific production of a disaccharide, oligosaccharide and/or Neu(n)Ac-containing bioproduct according to the present invention. In this sense a precursor can be an acceptor as defined herein, but can also be another substance, metabolite, which is first modified within the cell as part of the biochemical synthesis route of the disaccharide, oligosaccharide and/or Neu(n)Ac-containing bioproduct. The term “precursor” as used herein is also to be understood as a donor that is used by a glycosyltransferase to modify an acceptor as defined herein with a sugar moiety in a glycosidic bond, as part in the metabolic pathway of a disaccharide, oligosaccharide and/or Neu(n)Ac-containing bioproduct according to the present invention.

Examples of such precursors comprise the acceptors as defined herein, and/or dihydroxyacetone, glucosamine, N-acetylglucosamine, N-acetylmannosamine, galactosamine, N-acetylgalactosamine, galactosyllactose, phosphorylated sugars like e.g. but not limited to glucose-1-phosphate, galactose-1-phosphate, glucose-6-phosphate, fructose-6-phosphate, fructose-1,6-bisphosphate, mannose-6-phosphate, mannose-1-phosphate, glycerol-3-phosphate, glyceraldehyde-3-phosphate, dihydroxyacetone-phosphate, glucosamine-6-phosphate, N-acetylglucosamine-6-phosphate, N-acetylmannosamine-6-phosphate, N-acetylglucosamine-1-phosphate, N-acetylneuraminic acid-9-phosphate and nucleotide-activated sugars as defined herein like e.g. UDP-glucose, UDP-galactose, UDP-N-acetylglucosamine, CMP-sialic acid, GDP-mannose, GDP-4-dehydro-6-deoxy-α-D-mannose, GDP-fucose.

Optionally, the cell is transformed to comprise at least one nucleic acid sequence encoding a protein selected from the group consisting of lactose transporter, N-acetylneuraminic acid transporter, fucose transporter, transporter for a nucleotide-activated sugar wherein said transporter internalizes a to the medium added precursor for the synthesis of the disaccharide, oligosaccharide and/or Neu(n)Ac-containing bioproduct of present invention.

The term “acceptor” as used herein refers to a mono-, di- or oligosaccharide which can be modified by a glycosyltransferase. Examples of such acceptors comprise glucose, galactose, fructose, glycerol, sialic acid, fucose, mannose, maltose, sucrose, lactose, lacto-N-triose, lacto-N-tetraose (LNT), lacto-N-neotetraose (LNnT), lacto-N-pentaose (LNP), lacto-N-neopentaose, para lacto-N-pentaose, para lacto-N-neopentaose, lacto-N-novopentaose I, lacto-N-hexaose (LNH), lacto-N-neohexaose (LNnH), para lacto-N-neohexaose (pLNnH), para lacto-N-hexaose (pLNH), lacto-N-heptaose, lacto-N-neoheptaose, para lacto-N-neoheptaose, para lacto-N-heptaose, lacto-N-octaose (LNO), lacto-N-neooctaose, iso lacto-N-octaose, para lacto-N-octaose, iso lacto-N-neooctaose, novo lacto-N-neooctaose, para lacto-N-neooctaose, iso lacto-N-nonaose, novo lacto-N-nonaose, lacto-N-nonaose, lacto-N-decaose, iso lacto-N-decaose, novo lacto-N-decaose, lacto-N-neodecaose, and oligosaccharide containing 1 or more N-acetyllactosamine units and/or 1 or more lacto-N-biose units or an intermediate into oligosaccharide, fucosylated and sialylated versions thereof, ceramide, N-acylated sphingoid, glucosylceramide, lactosylceramide, sphingosine, phytosphingosine, sphingosine synthons, peptide backbones with beta-GlcNAc-Asn residues, glycoproteins with terminal GlcNAc and Gal residues, immunoglobulins.

Generally, enzymes can be classified by the Enzyme Commission Number (EC Number) which is a numerical classification scheme for enzymes, based on the chemical reactions they catalyze. The chemical reaction catalyzed is the specific property that distinguishes one enzyme from another. EC numbers specify enzyme-catalysed reactions. The EC numbers are assigned by the Nomenclature Committee of the International Union of Biochemistry and Molecular Biology. The EC numbers used herein are the EC numbers in the database version of 8 Apr. 2021.

Acetyl-Coenzyme A ligase classified as EC 6.2.1.1 or 6.2.1.13, catalyzes a reaction to produce acetyl-CoA, pyrophosphate and AMP from acetic acid, coenzyme A and ATP, and is encoded by an acs gene (J Bacteriol. 1995 May; 177(10):2878-86). The terms “acetyl-Coenzyme A ligase”, “acetyl-coenzyme A synthetase”, “acs”, “AcCoA synthetase”, “acetyl-CoA synthase”, “acetate thiokinase”, “acetate-CoA ligase”, “acetyl-CoA ligase”, “acetyl-activating enzyme”, “acyl-activating enzyme” and “yfaC” are used interchangeably and refer to an enzyme that catalyses the conversion of acetate into acetyl-coenzyme A (AcCoA) in an ATP-dependent reaction.

The terms “pyruvate dehydrogenase”, “pyruvate oxidase”, “POX”, “poxB” and “pyruvate:ubiquinone-8 oxidoreductase” are used interchangeably and refer to an enzyme that catalyses the oxidative decarboxylation of pyruvate to produce acetate and CO2 (and is classified as EC 1.2.5.1).

The term “phosphate acetyltransferase” as used herein refers to an enzyme classified as EC 2.3.1.8.

The term “acetate kinase” as used herein refers to an enzyme classified as EC 2.7.2.1.

The term “acetyl phosphate-producing pyruvate oxidase” as used herein refers to an enzyme classified as EC 1.2.3.3.

The term “pyruvate decarboxylase” as used herein refers to an enzyme classified as EC 4.1.1.1.

The term “acetaldehyde dehydrogenase” as used herein refers to any one of the enzymes classified as EC 1.2.1.3, EC 1.2.1.4, or EC 1.2.1.5.

The term “Pyruvate formate lyase” as used herein refers to an enzyme classified as EC 2.3.1.54 is an enzyme directly forming acetyl-CoA from pyruvate.

The term “CoA-acetylating pyruvate oxidase” as used herein refers to an enzyme classified as EC 1.2.3.6.

The term “pyruvate dehydrogenase enzyme complex” as used herein refers to the enzyme complex comprising the enzymes pyruvate dehydrogenase E1 component classified as EC 1.2.4.1, pyruvate dehydrogenase, E2 subunit classified as EC 2.3.1.12 and, lipoamide dehydrogenase, E3 subunit classified as EC 1.8.1.4.

The term “pyruvate synthase” as used herein refers to an enzyme classified as EC 1.2.7.1.

The term “Pantothenate kinase (PanK; also named as CoaA and classified as EC 2.7.1.33) is an enzyme facilitating the reaction ATP+(R)-pantothenate=>ADP+(R)-4′-phosphopantothenate, improving CoA synthesis, the precursor of acetyl-CoA. Other terms used in the art for pantothenate kinase are “pantothenate kinase (phosphorylating”), “pantothenic acid kinase”, “ATP:pantothenate 4′-phosphotransferase” and “D-pantothenate kinase”.

The terms “lactate dehydrogenase”, “D-lactate dehydrogenase”, “IdhA”, “hslI”, “htpH”, “D-LDH”, “fermentative lactate dehydrogenase” and “D-specific 2-hydroxyacid dehydrogenase” are used interchangeably and refer to an enzyme that catalyses the conversion of lactate into pyruvate hereby generating NADH as used herein refers to any one of the enzymes classified as EC 1.1.2.3, EC 1.1.2.4, EC 1.1.2.5, EC 1.1.1.27 or EC 1.1.1.28.

The term “pyruvate carboxylase” as used herein refers to an enzyme classified as EC 6.4.1.1.

The term “isocitrate lyase” as used herein refers to an enzyme classified as EC 4.1.3.1 and is involved in the glyoxylate pathway.

The term “malate synthase” as used herein refers to an enzyme classified as EC 2.3.3.9 and is involved in the glyoxylate pathway.

The enzymes as used herein with reference to their EC classification should be understood to comprise naturally occurring enzymes as well as naturally occurring, mutant versions or synthetically constructed functional homologs, variants and functional fragments thereof which have the same enzymatic activity as the reference naturally occurring enzyme sequence. Preferably such functional homolog, variant or functional fragment has 80% or more overall sequence identity to the reference naturally occurring enzyme sequence. At least 80% overall sequence identity to the full length of any one of said naturally occurring polypeptides as used herein should be understood as at least 80%, 81%, 82%, 83%, 84%, 85%, 86%, 87%, 88%, 89%, 90%, 91%, 92%, 93%, 94%, 95%, 96%, 97%, 98%, or 99% overall sequence identity to any one of said polypeptides as used herein.

### DETAILED DESCRIPTION OF THE INVENTION

In a first embodiment, the present invention provides a cell for the production of a compound, wherein said cell comprises a pathway for the production of said compound and wherein said compound is a disaccharide, oligosaccharide and/or a Neu(n)Ac-containing bioproduct wherein (n) is 4, 5, 7, 8 or 9 or a combination thereof, and wherein said cell is further metabolically engineered for enhanced synthesis of acetyl-Coenzyme A. Examples of such pathways comprise but are not limited to pathways involved in the synthesis of monosaccharide, phosphorylated monosaccharide, nucleotide-activated sugar, lipid and/or protein and/or glycosylation pathways like e.g. a fucosylation, sialylation, galactosylation, N-acetylglucosaminylation, N-acetylgalactosylation, mannosylation and/or N-acetylmannosaminylation pathway. Said pathway for the production of a compound, comprising a disaccharide, oligosaccharide, Neu(n)Ac-containing glycolipid or Neu(n)Ac-containing glycoprotein, preferably comprises at least one glycosyltransferase that is involved in the production of said compound.

According to a second embodiment, the present invention provides a method for the production of a compound, wherein the compound is a disaccharide, oligosaccharide and/or an N-acetylneuraminic acid (Neu(n)Ac)-containing bioproduct wherein (n) is 4, 5, 7, 8 or 9 or a combination thereof by a metabolically engineered cell. The method comprises the steps of:
1) providing a cell as described herein, and
2) cultivating said cell under conditions permissive to produce said compound.

Preferably, the compound is separated from the cultivation, preferably as explained herein.

In the scope of the present invention, permissive conditions are understood to be conditions relating to physical or chemical parameters including but not limited to temperature, pH, pressure, osmotic pressure and product/precursor/acceptor concentration.

In a particular embodiment, the permissive conditions may include a temperature-range of 30+/−20 degrees centigrade, a pH-range of 2.0-10.0, preferably 7+/−3.

In a preferred embodiment of the cell and/or the method of the invention, the enhanced acetyl-Coenzyme A synthesis is obtained by enhanced expression or activity of any one or more of the enzymes: i) acetyl-Coenzyme A ligase (EC 6.2.1.1 or 6.2.1.13); ii) pyruvate dehydrogenase (EC 1.2.5.1); iii) pantothenate kinase (EC 2.7.1.33); iv) acetyl phosphate-producing pyruvate oxidase (EC 1.2.3.3); v) acetate kinase (EC 2.7.2.1); vi) phosphate acetyltransferase (EC 2.3.1.8); vii) pyruvate decarboxylase (EC 4.1.1.1); viii) acetaldehyde dehydrogenase (EC 1.2.1.3, EC 1.2.1.4, or EC 1.2.1.5); ix) pyruvate formate lyase (EC 2.3.1.54); x) CoA-acetylating pyruvate oxidase (EC 1.2.3.6), xi) pyruvate synthase (EC 1.2.7.1), xii) pyruvate dehydrogenase enzyme complex as defined herein.

In one preferred embodiment of the cell and/or the method of the invention, the enhanced acetyl-CoA synthesis is obtained by a method selected from the group consisting of: a) increasing the copy number of any one or more of the genes encoding enzyme i) acetyl-Coenzyme A ligase (EC 6.2.1.1 or 6.2.1.13); ii) pyruvate dehydrogenase (EC 1.2.5.1); iii) pantothenate kinase (EC 2.7.1.33); iv) acetyl phosphate-producing pyruvate oxidase (EC 1.2.3.3); v) acetate kinase (EC 2.7.2.1); vi) phosphate acetyltransferase (EC 2.3.1.8); vii) pyruvate decarboxylase (EC 4.1.1.1); viii) acetaldehyde dehydrogenase (EC 1.2.1.3, EC 1.2.1.4, or EC 1.2.1.5); ix) pyruvate formate lyase (EC 2.3.1.54); x) CoA-acetylating pyruvate oxidase (EC 1.2.3.6), xi) pyruvate synthase (EC 1.2.7.1), or xii) the pyruvate dehydrogenase enzyme complex as defined herein; b) modifying an expression regulatory sequence of any one or more of said genes, and c) combinations thereof.

The copy number of any gene encoding any one of the above enzymes can be increased by introducing multiple copies of the gene into the chromosomal DNA of the host, preferably the host bacterium. Introducing multiple copies of the gene into the chromosomal DNA of the host, preferably the host bacterium can be attained by homologous recombination using a target sequence present on the chromosomal DNA in multiple copies. This may be a repetitive DNA or an inverted repeat present on the end of a transposing element. Alternatively, as disclosed in JP 2-109985 A, multiple copies of the gene can be introduced into the chromosomal DNA by inserting the gene into a transposon, and transferring it so that multiple copies of the gene are integrated into the chromosomal DNA. Integration of the gene into the chromosome can be confirmed by Southern hybridization using a portion of the gene as a probe.

In an alternative preferred embodiment, enhanced synthesis is obtained by overexpressing any one or more of the genes encoding an endogenous enzyme i) acetyl-Coenzyme A ligase (EC 6.2.1.1 or 6.2.1.13); ii) pyruvate dehydrogenase (EC 1.2.5.1); iii) pantothenate kinase (EC 2.7.1.33); iv) acetyl phosphate-producing pyruvate oxidase (EC 1.2.3.3); v) acetate kinase (EC 2.7.2.1); vi) phosphate acetyltransferase (EC 2.3.1.8); vii) pyruvate decarboxylase (EC 4.1.1.1); viii) acetaldehyde dehydrogenase (EC 1.2.1.3, EC 1.2.1.4, or EC 1.2.1.5); ix) pyruvate formate lyase (EC 2.3.1.54); x) CoA-acetylating pyruvate oxidase (EC 1.2.3.6), xi) pyruvate synthase (EC 1.2.7.1), or overexpression of the enzyme complex of xii) pyruvate dehydrogenase enzyme complex as defined herein; and/or introducing and expressing any one or more of a homologous or heterologous gene encoding an enzyme i) to xi) or the enzyme complex of xii). Further preferably, any one or more of said enzyme i) to xi) or the enzyme complex of xii) is presented to the cell in one or more gene expression modules wherein expression is regulated by one or more regulatory sequences. More preferably, said expression modules are integrated in the cell's genome and/or presented to the cell on a vector comprising plasmid, cosmid, phage, liposome or virus, which is to be stably transformed into said cell.

In a preferred aspect of the method and/or cell of the invention, the metabolically engineered cell is modified with gene expression modules wherein the expression from any one of said expression modules is constitutive or is tuneable. Said expression modules are also known as transcriptional units and comprise polynucleotides for expression of recombinant genes including coding gene sequences and appropriate transcriptional and/or translational control signals that are operably linked to the coding genes. Said control signals comprise promoter sequences, untranslated regions, ribosome binding sites, terminator sequences. Said expression modules can contain elements for expression of one single recombinant gene but can also contain elements for expression of more recombinant genes or can be organized in an operon structure for integrated expression of two or more recombinant genes. Said polynucleotides may be produced by recombinant DNA technology using techniques well-known in the art. Methods which are well known to those skilled in the art to construct expression modules include, for example, in vitro recombinant DNA techniques, synthetic techniques, and in vivo genetic recombination. See, for example, the techniques described in Sambrook et al. (2001) Molecular Cloning: a laboratory manual, 3rd Edition, Cold Spring Harbor Laboratory Press, CSH, New York or to Current Protocols in Molecular Biology, John Wiley and Sons, N.Y. (1989 and yearly updates).

According to a preferred aspect of the present invention, the cell is modified with one or more expression modules. The expression modules can be integrated in the genome of said cell or can be presented to said cell on a vector. Said vector can be present in the form of a plasmid, cosmid, phage, liposome, or virus, which is to be stably transformed/transfected into said metabolically engineered cell. Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses, and vectors derived from combinations thereof, such as those derived from plasmid and bacteriophage genetic elements, such as cosmids and phagemids. These vectors may contain selection markers such as but not limited to antibiotic markers, auxotrophic markers, toxin-antitoxin markers, RNA sense/antisense markers. The expression system constructs may contain control regions that regulate as well as engender expression. Generally, any system or vector suitable to maintain, propagate or express polynucleotides and/or to express a polypeptide in a host may be used for expression in this regard. The appropriate DNA sequence may be inserted into the expression system by any of a variety of well-known and routine techniques, such as, for example, those set forth in Sambrook et al., see above. For recombinant production, cells can be genetically engineered to incorporate expression systems or portions thereof or polynucleotides of the invention. Introduction of a polynucleotide into the cell can be effected by methods described in many standard laboratory manuals, such as Davis et al., Basic Methods in Molecular Biology, (1986), and Sambrook et al., 1989, supra.

As used herein an expression module comprises polynucleotides for expression of at least one recombinant gene. Said recombinant gene is involved in the expression of a polypeptide acting in the synthesis of said compound; or said recombinant gene is linked to other pathways in said cell that are not involved in the synthesis of said compound. Said recombinant genes encode endogenous proteins with a modified expression or activity, preferably said endogenous proteins are overexpressed; or said recombinant genes encode heterologous proteins that are heterogeneously introduced and expressed in said modified cell, preferably overexpressed. The endogenous proteins can have a modified expression in the cell which also expresses a heterologous protein.

According to a preferred aspect of the present invention, the expression of each of said expression modules is constitutive or tuneable as defined herein.

In one exemplary embodiment of the present invention, the cell comprises enhanced expression or activity of acetyl-Coenzyme A ligase (EC 6.2.1.1 or 6.2.1.13). In another exemplary embodiment of the present invention, the cell comprises an overexpression of acetyl-Coenzyme A ligase (EC 6.2.1.1 or 6.2.1.13). In a further exemplary embodiment, this overexpression of acetyl-Coenzyme A ligase (EC 6.2.1.1 or 6.2.1.13) can be combined with the active expression or overexpression of pyruvate oxidase (also named pyruvate dehydrogenase EC 1.2.5.1) and/or phosphate acetyltransferase (EC 2.3.1.8) and/or acetate kinase (EC 2.7.2.1) and/or acetyl phosphate-producing pyruvate oxidase (EC 1.2.3.3) and/or pyruvate decarboxylase (EC 4.1.1.1) and/or acetaldehyde dehydrogenase (EC 1.2.1.3, EC 1.2.1.4, EC 1.2.1.5).

In a further exemplary embodiment of the invention, the cell is modified in the expression or activity of at least acetyl-coenzyme A ligase like e.g., acs from E. coli, S. cerevisiae, H. sapiens, M. musculus.

In a preferred embodiment, said acetyl-Coenzyme A ligase is originating from Escherichia coli species comprising but not limited to E. coli B, E. coli BL21, E. coli BL21(DE3), E. coli C, E. coli DH5alpha, E. coli K-12, E. coli Nissle, E. coli Top10, E. coli W, or said acetyl-Coenzyme A synthetase is originating from Salmonella typhi, Vibrio cholera, Saccharomyces cerevisiae, Bacillus subtilis, Mycobacterium tuberculosis, Campylobacter jejuni, Yersinia pestis, Corynebacterium.

In a preferred embodiment, said acetyl-coenzyme A ligase is an endogenous protein of the cell with a modified expression or activity, preferably said endogenous acetyl-coenzyme A synthetase is overexpressed; alternatively, said acetyl-coenzyme A ligase is a heterologous protein that is heterogeneously introduced and expressed in said cell, preferably overexpressed. Said endogenous acetyl-coenzyme A ligase can have a modified expression in the cell which also expresses a heterologous can have a modified expression in the cell which also expresses a heterologous. In a specific exemplary embodiment, the cell is modified in the expression or activity of the acetyl-coenzyme A ligase acs from E. coli identified in the UniProt Knowledgebase (release 2021_02) as P27550 enzyme, or from S. cerevisiae identified in UniProt Knowledgebase (release 2021_02) as Q01574 or P52910, or from B. subtilis identified in UniProt Knowledgebase (release 2021_02) as P39062, or from H. sapiens identified in UniProt Knowledgebase (release 2021_02) as Q9NR19. In another and/or additional preferred embodiment, the cell is modified in the expression or activity of a functional homolog, variant or derivative of any one or more of the acetylcoenzyme A ligase with UniProt number P27550, Q01574, P39062, P52910 or Q9NR19 having at least 80% overall sequence identity to the full-length of said polypeptide and having acetyl-coenzyme A ligase activity.

Other examples of acs gene of Escherichia coli include the acs gene which is a complementary strand of nucleotide numbers 4283436 . . . 4285394 of GenBank Accession No. NC-000913. Examples of acs genes from other sources include the acs gene of a complementary strand of nucleotide numbers 4283436 . . . 4285394 of GenBank Accession No. NC-000913 Yersinia pestis (a complementary strand of nucleotide numbers 577565 . . . 579529 of GenBank Accession No. NC-004088), the acs gene of Salmonella typhi (a complementary strand of nucleotide numbers 120832 . . . 122790 of GenBank Accession No. AL627282), the acs gene of Vibrio cholerae (a complementary strand of nucleotide numbers 305121 . . . 307121 of GenBank Accession No. NC-002505) and the acs gene of Salmonella typhimurium (a complementary strand of nucleotide numbers 4513714 . . . 4515672 of GenBank Accession No. NC-003197). Other sources include the acs gene of S. cerevisiae, the acs gene of B. subtilis, the acs gene of Mycobacterium tuberculosis, the acs gene of Campylobacter jejuni or an acs gene of any one of the Corynebacteria.

In an alternative and/or additional further aspect of the method and/or cell of the invention, the cell is modified for increased activity or expression of at least one pyruvate dehydrogenase like e.g., from E. coli, S. cerevisiae, H. sapiens and R. norvegicus. In a preferred embodiment, the cell has been modified to have at least one overexpressed pyruvate dehydrogenase encoding gene by means generally known by the person skilled in the art resulting in at least one protein with increased pyruvate dehydrogenase activity. In an alternative exemplary embodiment, Acetyl-CoA synthesis is improved by enhanced expression or activity of any one or more of the enzymes pyruvate formate lyase (EC 2.3.1.54) directly forming acetyl-CoA from pyruvate, CoA-acetylating pyruvate oxidase (EC 1.2.3.6), pyruvate synthase (EC 1.2.7.1), and the pyruvate dehydrogenase enzyme complex as defined herein. Further examples of such routes can be found in Metabolites 2020, 10, 166; doi:10.3390/metabo10040166.

Alternatively or additionally, the acetyl-CoA synthesis is improved by enhanced expression or activity of Pantothenate kinase (PanK; also named as CoaA, EC 2.7.1.33), improving CoA synthesis, the precursor of acetyl-CoA.

Further exemplary embodiments of the method or cell of the present invention provide for a cell which is modified to overexpress the acetate-CoA ligase enzyme (EC 6.2.1.1 or 6.2.1.13) combined with overexpression of pyruvate oxidase (EC 1.2.5.1); or is modified to overexpress the acetate-CoA ligase enzyme (EC 6.2.1.1 or 6.2.1.13) combined with overexpression of phosphate acetyltransferase (EC 2.3.1.8); or is modified to overexpress the acetate-CoA ligase enzyme (EC 6.2.1.1 or 6.2.1.13) combined with overexpression of acetate kinase (EC 2.7.2.1); or is modified to overexpress the acetate-CoA ligase enzyme (EC 6.2.1.1 or 6.2.1.13) combined with overexpression of acetyl phosphate-producing pyruvate oxidase (EC 1.2.3.3); or is modified to overexpress the acetate-CoA ligase enzyme (EC 6.2.1.1 or 6.2.1.13) combined with overexpression of pyruvate decarboxylase (EC 4.1.1.1); or is modified to overexpress the acetate-CoA ligase enzyme (EC 6.2.1.1 or 6.2.1.13) combined with overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpression of pyruvate oxidase (EC 1.2.5.1) and overexpression of phosphate acetyltransferase (EC 2.3.1.8); or is modified to overexpress the acetate-CoA ligase enzyme (EC 6.2.1.1 or 6.2.1.13) combined with overexpression of pyruvate oxidase (EC 1.2.5.1) and overexpression of acetate kinase (EC 2.7.2.1); or is modified to overexpress the acetate-CoA ligase enzyme (EC 6.2.1.1 or 6.2.1.13) combined with overexpression of pyruvate oxidase (EC 1.2.5.1) and overexpression of acetyl phosphate-producing pyruvate oxidase (EC 1.2.3.3); or is modified to overexpress the acetate-CoA ligase enzyme (EC 6.2.1.1 or 6.2.1.13) combined with overexpression of pyruvate oxidase (EC 1.2.5.1) and overexpression of pyruvate decarboxylase (EC 4.1.1.1); or is modified to overexpress the acetate-CoA ligase enzyme (EC 6.2.1.1 or 6.2.1.13) combined with overexpression of pyruvate oxidase (EC 1.2.5.1) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpression of pyruvate oxidase (EC 1.2.5.1) and overexpression of phosphate acetyltransferase (EC 2.3.1.8) and overexpression of acetate kinase (EC 2.7.2.1); or is modified to overexpress the acetate-CoA ligase enzyme (EC 6.2.1.1 or 6.2.1.13) combined with overexpression of pyruvate oxidase (EC 1.2.5.1) and overexpression of phosphate acetyltransferase (EC 2.3.1.8) and overexpression of acetyl phosphate-producing pyruvate oxidase (EC 1.2.3.3); or is modified to overexpress the acetate-CoA ligase enzyme (EC 6.2.1.1 or 6.2.1.13) combined with overexpression of pyruvate oxidase (EC 1.2.5.1) and overexpression of phosphate acetyltransferase (EC 2.3.1.8) and overexpression of pyruvate decarboxylase (EC 4.1.1.1); or is modified to overexpress the acetate-CoA ligase enzyme (EC 6.2.1.1 or 6.2.1.13) combined with overexpression of pyruvate oxidase (EC 1.2.5.1) and overexpression of phosphate acetyltransferase (EC 2.3.1.8) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpression of pyruvate oxidase (EC 1.2.5.1) and overexpression of phosphate acetyltransferase (EC 2.3.1.8) and overexpression of acetate kinase (EC 2.7.2.1) and overexpression of acetyl phosphate-producing pyruvate oxidase (EC 1.2.3.3); or is modified to overexpress the acetate-CoA ligase enzyme (EC 6.2.1.1 or 6.2.1.13) combined with overexpression of pyruvate oxidase (EC 1.2.5.1) and overexpression of phosphate acetyltransferase (EC 2.3.1.8) and overexpression of acetate kinase (EC 2.7.2.1) and overexpression of pyruvate decarboxylase (EC 4.1.1.1); or is modified to overexpress the acetate-CoA ligase enzyme (EC 6.2.1.1 or 6.2.1.13) combined with overexpression of pyruvate oxidase (EC 1.2.5.1) and overexpression of phosphate acetyltransferase (EC 2.3.1.8) and overexpression of acetate kinase (EC 2.7.2.1) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpression of pyruvate oxidase (EC 1.2.5.1) and overexpression of phosphate acetyltransferase (EC 2.3.1.8) and overexpression of acetate kinase (EC 2.7.2.1) and overexpression of acetyl phosphate-producing pyruvate oxidase (EC 1.2.3.3) and overexpression of pyruvate decarboxylase (EC 4.1.1.1); or is modified to overexpress the acetate-CoA ligase enzyme (EC 6.2.1.1 or 6.2.1.13) combined with overexpression of pyruvate oxidase (EC 1.2.5.1) and overexpression of phosphate acetyltransferase (EC 2.3.1.8) and overexpression of acetate kinase (EC 2.7.2.1) and overexpression of acetyl phosphate-producing pyruvate oxidase (EC 1.2.3.3) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpression of pyruvate oxidase (EC 1.2.5.1) and overexpression of phosphate acetyltransferase (EC 2.3.1.8) and overexpression of acetate kinase (EC 2.7.2.1) and overexpression of acetyl phosphate-producing pyruvate oxidase (EC 1.2.3.3) and overexpression of pyruvate decarboxylase (EC 4.1.1.1) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpression of pyruvate oxidase (EC 1.2.5.1) and overexpression of phosphate acetyltransferase (EC 2.3.1.8) and overexpression of acetate kinase (EC 2.7.2.1) and overexpression of pyruvate decarboxylase (EC 4.1.1.1) and overexpression of acetaldehyde dehydrogenase (EC 1.2.1.3, EC 1.2.1.4, or EC 1.2.1.5; or is modified to overexpress the acetate-CoA ligase enzyme (EC 6.2.1.1 or 6.2.1.13) combined with overexpression of pyruvate oxidase (EC 1.2.5.1) and overexpression of acetate kinase (EC 2.7.2.1) and overexpression of acetyl phosphate-producing pyruvate oxidase (EC 1.2.3.3); or is modified to overexpress the acetate-CoA ligase enzyme (EC 6.2.1.1 or 6.2.1.13) combined with overexpression of pyruvate oxidase (EC 1.2.5.1) and overexpression of acetate kinase (EC 2.7.2.1) and overexpression of pyruvate decarboxylase (EC 4.1.1.1); or is modified to overexpress the acetate-CoA ligase enzyme (EC 6.2.1.1 or 6.2.1.13) combined with overexpression of pyruvate oxidase (EC 1.2.5.1) and overexpression of acetate kinase (EC 2.7.2.1) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpression of pyruvate oxidase (EC 1.2.5.1) and overexpression of acetate kinase (EC 2.7.2.1) and overexpression of acetyl phosphate-producing pyruvate oxidase (EC 1.2.3.3) and overexpression of pyruvate decarboxylase (EC 4.1.1.1); or is modified to overexpress the acetate-CoA ligase enzyme (EC 6.2.1.1 or 6.2.1.13) combined with overexpression of pyruvate oxidase (EC 1.2.5.1) and overexpression of acetate kinase (EC 2.7.2.1) and overexpression of acetyl phosphate-producing pyruvate oxidase (EC 1.2.3.3) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpression of pyruvate oxidase (EC 1.2.5.1) and overexpression of acetate kinase (EC 2.7.2.1) and overexpression of acetyl phosphate-producing pyruvate oxidase (EC 1.2.3.3) and overexpression of pyruvate decarboxylase (EC 4.1.1.1) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpression of pyruvate oxidase (EC 1.2.5.1) and overexpression of phosphate acetyltransferase (EC 2.3.1.8) and overexpression of acetate kinase (EC 2.7.2.1) and overexpression of pyruvate decarboxylase (EC 4.1.1.1) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpression of pyruvate oxidase (EC 1.2.5.1) and overexpression of phosphate acetyltransferase (EC 2.3.1.8) and overexpression of acetate kinase (EC 2.7.2.1) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpression of pyruvate oxidase (EC 1.2.5.1) and overexpression of acetyl phosphate-producing pyruvate oxidase (EC 1.2.3.3) and overexpression of pyruvate decarboxylase (EC 4.1.1.1); or is modified to overexpress the acetate-CoA ligase enzyme (EC 6.2.1.1 or 6.2.1.13) combined with overexpression of pyruvate oxidase (EC 1.2.5.1) and overexpression of acetyl phosphate-producing pyruvate oxidase (EC 1.2.3.3) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpression of pyruvate oxidase (EC 1.2.5.1) and overexpression of acetyl phosphate-producing pyruvate oxidase (EC 1.2.3.3) and overexpression of pyruvate decarboxylase (EC 4.1.1.1) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpression of pyruvate oxidase (EC 1.2.5.1) overexpression of pyruvate decarboxylase (EC 4.1.1.1) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpression of pyruvate oxidase (EC 1.2.5.1) and overexpression of phosphate acetyltransferase (EC 2.3.1.8) and overexpression of acetyl phosphate-producing pyruvate oxidase (EC 1.2.3.3) and overexpression of pyruvate decarboxylase (EC 4.1.1.1); or is modified to overexpress the acetate-CoA ligase enzyme (EC 6.2.1.1 or 6.2.1.13) combined with overexpression of pyruvate oxidase (EC 1.2.5.1) and overexpression of phosphate acetyltransferase (EC 2.3.1.8) and overexpression of acetyl phosphate-producing pyruvate oxidase (EC 1.2.3.3) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpression of pyruvate oxidase (EC 1.2.5.1) and overexpression of phosphate acetyltransferase (EC 2.3.1.8) and overexpression of acetyl phosphate-producing pyruvate oxidase (EC 1.2.3.3) and overexpression of pyruvate decarboxylase (EC 4.1.1.1) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpression of pyruvate oxidase (EC 1.2.5.1) and overexpression of phosphate acetyltransferase (EC 2.3.1.8) and overexpression of pyruvate decarboxylase (EC 4.1.1.1) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpression of phosphate acetyltransferase (EC 2.3.1.8) and overexpression of acetate kinase (EC 2.7.2.1); or is modified to overexpress the acetate-CoA ligase enzyme (EC 6.2.1.1 or 6.2.1.13) combined with overexpression of phosphate acetyltransferase (EC 2.3.1.8) and overexpression of acetyl phosphate-producing pyruvate oxidase (EC 1.2.3.3); or is modified to overexpress the acetate-CoA ligase enzyme (EC 6.2.1.1 or 6.2.1.13) combined with overexpression of phosphate acetyltransferase (EC 2.3.1.8) and overexpression of pyruvate decarboxylase (EC 4.1.1.1); or is modified to overexpress the acetate-CoA ligase enzyme (EC 6.2.1.1 or 6.2.1.13) combined with overexpression of phosphate acetyltransferase (EC 2.3.1.8) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpression of phosphate acetyltransferase (EC 2.3.1.8) and overexpression of acetate kinase (EC 2.7.2.1) and overexpression of acetyl phosphate-producing pyruvate oxidase (EC 1.2.3.3); or is modified to overexpress the acetate-CoA ligase enzyme (EC 6.2.1.1 or 6.2.1.13) combined with overexpression of phosphate acetyltransferase (EC 2.3.1.8) and overexpression of acetate kinase (EC 2.7.2.1) and overexpression of pyruvate decarboxylase (EC 4.1.1.1); or is modified to overexpress the acetate-CoA ligase enzyme (EC 6.2.1.1 or 6.2.1.13) combined with overexpression of phosphate acetyltransferase (EC 2.3.1.8) and overexpression of acetate kinase (EC 2.7.2.1) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpression of phosphate acetyltransferase (EC 2.3.1.8) and overexpression of acetate kinase (EC 2.7.2.1) and overexpression of acetyl phosphate-producing pyruvate oxidase (EC 1.2.3.3) and overexpression of pyruvate decarboxylase (EC 4.1.1.1); or is modified to overexpress the acetate-CoA ligase enzyme (EC 6.2.1.1 or 6.2.1.13) combined with overexpression of phosphate acetyltransferase (EC 2.3.1.8) and overexpression of acetate kinase (EC 2.7.2.1) and overexpression of acetyl phosphate-producing pyruvate oxidase (EC 1.2.3.3) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpression of phosphate acetyltransferase (EC 2.3.1.8) and overexpression of acetate kinase (EC 2.7.2.1) and overexpression of acetyl phosphate-producing pyruvate oxidase (EC 1.2.3.3) and overexpression of pyruvate decarboxylase (EC 4.1.1.1) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpression of phosphate acetyltransferase (EC 2.3.1.8) and overexpression of acetate kinase (EC 2.7.2.1) and overexpression of pyruvate decarboxylase (EC 4.1.1.1) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpression of phosphate acetyltransferase (EC 2.3.1.8) and overexpression of acetate kinase (EC 2.7.2.1) and overexpression of pyruvate decarboxylase (EC 4.1.1.1) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpression of phosphate acetyltransferase (EC 2.3.1.8) and overexpression of acetate kinase (EC 2.7.2.1) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpression of phosphate acetyltransferase (EC 2.3.1.8) and overexpression of acetyl phosphate-producing pyruvate oxidase (EC 1.2.3.3) and overexpression of pyruvate decarboxylase (EC 4.1.1.1); or is modified to overexpress the acetate-CoA ligase enzyme (EC 6.2.1.1 or 6.2.1.13) combined with overexpression of phosphate acetyltransferase (EC 2.3.1.8) and overexpression of acetyl phosphate-producing pyruvate oxidase (EC 1.2.3.3) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpression of phosphate acetyltransferase (EC 2.3.1.8) and overexpression of acetyl phosphate-producing pyruvate oxidase (EC 1.2.3.3) and overexpression of pyruvate decarboxylase (EC 4.1.1.1) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpression of phosphate acetyltransferase (EC 2.3.1.8) and overexpression of pyruvate decarboxylase (EC 4.1.1.1) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpression of acetate kinase (EC 2.7.2.1) and overexpression of acetyl phosphate-producing pyruvate oxidase (EC 1.2.3.3); or is modified to overexpress the acetate-CoA ligase enzyme (EC 6.2.1.1 or 6.2.1.13) combined with overexpression of acetate kinase (EC 2.7.2.1) and overexpression of pyruvate decarboxylase (EC 4.1.1.1); or is modified to overexpress the acetate-CoA ligase enzyme (EC 6.2.1.1 or 6.2.1.13) combined with overexpression of acetate kinase (EC 2.7.2.1) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpression of acetate kinase (EC 2.7.2.1) and overexpression of acetyl phosphate-producing pyruvate oxidase (EC 1.2.3.3) and overexpression of pyruvate decarboxylase (EC 4.1.1.1); or is modified to overexpress the acetate-CoA ligase enzyme (EC 6.2.1.1 or 6.2.1.13) combined with overexpression of acetate kinase (EC 2.7.2.1) and overexpression of acetyl phosphate-producing pyruvate oxidase (EC 1.2.3.3) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpression of acetate kinase (EC 2.7.2.1) and overexpression of acetyl phosphate-producing pyruvate oxidase (EC 1.2.3.3) and overexpression of pyruvate decarboxylase (EC 4.1.1.1) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpression of acetate kinase (EC 2.7.2.1) and overexpression of pyruvate decarboxylase (EC 4.1.1.1) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpression of acetyl phosphate-producing pyruvate oxidase (EC 1.2.3.3) and overexpression of pyruvate decarboxylase (EC 4.1.1.1); or is modified to overexpress the acetate-CoA ligase enzyme (EC 6.2.1.1 or 6.2.1.13) combined overexpression of acetyl phosphate-producing pyruvate oxidase (EC 1.2.3.3) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpression of acetyl phosphate-producing pyruvate oxidase (EC 1.2.3.3) and overexpression of pyruvate decarboxylase (EC 4.1.1.1) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpression of pyruvate decarboxylase (EC 4.1.1.1) and overexpression of acetaldehyde dehydrogenase (EC 1.2.1.3, EC 1.2.1.4, or EC 1.2.1.5).

Further exemplary embodiments of the method or cell of the present invention provide for a cell which is modified to overexpress the acetate-CoA ligase enzyme (EC 6.2.1.1 or 6.2.1.13) combined with overexpressing Pantothenate kinase (EC 2.7.1.33) and overexpression of pyruvate oxidase (EC 1.2.5.1); or is modified to overexpress the acetate-CoA ligase enzyme (EC 6.2.1.1 or 6.2.1.13) combined with overexpressing Pantothenate kinase (EC 2.7.1.33) and overexpression of phosphate acetyltransferase (EC 2.3.1.8); or is modified to overexpress the acetate-CoA ligase enzyme (EC 6.2.1.1 or 6.2.1.13) combined with overexpressing Pantothenate kinase (EC 2.7.1.33) and overexpression of acetate kinase (EC 2.7.2.1); or is modified to overexpress the acetate-CoA ligase enzyme (EC 6.2.1.1 or 6.2.1.13) combined with overexpressing Pantothenate kinase (EC 2.7.1.33) and overexpression of acetyl phosphate-producing pyruvate oxidase (EC 1.2.3.3) or is modified to overexpress the acetate-CoA ligase enzyme (EC 6.2.1.1 or 6.2.1.13) combined with overexpressing Pantothenate kinase (EC 2.7.1.33) and overexpression of pyruvate decarboxylase (EC 4.1.1.1); or is modified to overexpress the acetate-CoA ligase enzyme (EC 6.2.1.1 or 6.2.1.13) combined with overexpressing Pantothenate kinase (EC 2.7.1.33) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpressing Pantothenate kinase (EC 2.7.1.33) and overexpression of pyruvate oxidase (EC 1.2.5.1) and overexpression of phosphate acetyltransferase (EC 2.3.1.8); or is modified to overexpress the acetate-CoA ligase enzyme (EC 6.2.1.1 or 6.2.1.13) combined with overexpressing Pantothenate kinase (EC 2.7.1.33) and overexpression of pyruvate oxidase (EC 1.2.5.1) and overexpression of acetate kinase (EC 2.7.2.1); or is modified to overexpress the acetate-CoA ligase enzyme (EC 6.2.1.1 or 6.2.1.13) combined with overexpressing Pantothenate kinase (EC 2.7.1.33) and overexpression of pyruvate oxidase (EC 1.2.5.1) and overexpression of acetyl phosphate-producing pyruvate oxidase (EC 1.2.3.3); or is modified to overexpress the acetate-CoA ligase enzyme (EC 6.2.1.1 or 6.2.1.13) combined with overexpressing Pantothenate kinase (EC 2.7.1.33) and overexpression of pyruvate oxidase (EC 1.2.5.1) and overexpression of pyruvate decarboxylase (EC 4.1.1.1); or is modified to overexpress the acetate-CoA ligase enzyme (EC 6.2.1.1 or 6.2.1.13) combined with overexpressing Pantothenate kinase (EC 2.7.1.33) and overexpression of pyruvate oxidase (EC 1.2.5.1) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpressing Pantothenate kinase (EC 2.7.1.33) and overexpression of pyruvate oxidase (EC 1.2.5.1) and overexpression of phosphate acetyltransferase (EC 2.3.1.8) and overexpression of acetate kinase (EC 2.7.2.1); or is modified to overexpress the acetate-CoA ligase enzyme (EC 6.2.1.1 or 6.2.1.13) combined with overexpressing Pantothenate kinase (EC 2.7.1.33) and overexpression of pyruvate oxidase (EC 1.2.5.1) and overexpression of phosphate acetyltransferase (EC 2.3.1.8) and overexpression of acetyl phosphate-producing pyruvate oxidase (EC 1.2.3.3); or is modified to overexpress the acetate-CoA ligase enzyme (EC 6.2.1.1 or 6.2.1.13) combined with overexpressing Pantothenate kinase (EC 2.7.1.33) and overexpression of pyruvate oxidase (EC 1.2.5.1) and overexpression of phosphate acetyltransferase (EC 2.3.1.8) and overexpression of pyruvate decarboxylase (EC 4.1.1.1); or is modified to overexpress the acetate-CoA ligase enzyme (EC 6.2.1.1 or 6.2.1.13) combined with overexpressing Pantothenate kinase (EC 2.7.1.33) and overexpression of pyruvate oxidase (EC 1.2.5.1) and overexpression of phosphate acetyltransferase (EC 2.3.1.8) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpressing Pantothenate kinase (EC 2.7.1.33) and overexpression of pyruvate oxidase (EC 1.2.5.1) and overexpression of phosphate acetyltransferase (EC 2.3.1.8) and overexpression of acetate kinase (EC 2.7.2.1) and overexpression of acetyl phosphate-producing pyruvate oxidase (EC 1.2.3.3); or is modified to overexpress the acetate-CoA ligase enzyme (EC 6.2.1.1 or 6.2.1.13) combined with overexpressing Pantothenate kinase (EC 2.7.1.33) and overexpression of pyruvate oxidase (EC 1.2.5.1) and overexpression of phosphate acetyltransferase (EC 2.3.1.8) and overexpression of acetate kinase (EC 2.7.2.1) and overexpression of pyruvate decarboxylase (EC 4.1.1.1); or is modified to overexpress the acetate-CoA ligase enzyme (EC 6.2.1.1 or 6.2.1.13) combined with overexpressing Pantothenate kinase (EC 2.7.1.33) and overexpression of pyruvate oxidase (EC 1.2.5.1) and overexpression of phosphate acetyltransferase (EC 2.3.1.8) and overexpression of acetate kinase (EC 2.7.2.1) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpressing Pantothenate kinase (EC 2.7.1.33) and overexpression of pyruvate oxidase (EC 1.2.5.1) and overexpression of phosphate acetyltransferase (EC 2.3.1.8) and overexpression of acetate kinase (EC 2.7.2.1) and overexpression of acetyl phosphate-producing pyruvate oxidase (EC 1.2.3.3) and overexpression of pyruvate decarboxylase (EC 4.1.1.1); or is modified to overexpress the acetate-CoA ligase enzyme (EC 6.2.1.1 or 6.2.1.13) combined with overexpressing Pantothenate kinase (EC 2.7.1.33) and overexpression of pyruvate oxidase (EC 1.2.5.1) and overexpression of phosphate acetyltransferase (EC 2.3.1.8) and overexpression of acetate kinase (EC 2.7.2.1) and overexpression of acetyl phosphate-producing pyruvate oxidase (EC 1.2.3.3) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpressing Pantothenate kinase (EC 2.7.1.33) and overexpression of pyruvate oxidase (EC 1.2.5.1) and overexpression of phosphate acetyltransferase (EC 2.3.1.8) and overexpression of acetate kinase (EC 2.7.2.1) and overexpression of acetyl phosphate-producing pyruvate oxidase (EC 1.2.3.3) and overexpression of pyruvate decarboxylase (EC 4.1.1.1) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpressing Pantothenate kinase (EC 2.7.1.33) and overexpression of pyruvate oxidase (EC 1.2.5.1) and overexpression of phosphate acetyltransferase (EC 2.3.1.8) and overexpression of acetate kinase (EC 2.7.2.1) and overexpression of pyruvate decarboxylase (EC 4.1.1.1) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpressing Pantothenate kinase (EC 2.7.1.33) and overexpression of pyruvate oxidase (EC 1.2.5.1) and overexpression of acetate kinase (EC 2.7.2.1) and overexpression of acetyl phosphate-producing pyruvate oxidase (EC 1.2.3.3); or is modified to overexpress the acetate-CoA ligase enzyme (EC 6.2.1.1 or 6.2.1.13) combined with overexpressing Pantothenate kinase (EC 2.7.1.33) and overexpression of pyruvate oxidase (EC 1.2.5.1) and overexpression of acetate kinase (EC 2.7.2.1) and overexpression of pyruvate decarboxylase (EC 4.1.1.1); or is modified to overexpress the acetate-CoA ligase enzyme (EC 6.2.1.1 or 6.2.1.13) combined with overexpressing Pantothenate kinase (EC 2.7.1.33) and overexpression of pyruvate oxidase (EC 1.2.5.1) and overexpression of acetate kinase (EC 2.7.2.1) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpressing Pantothenate kinase (EC 2.7.1.33) and overexpression of pyruvate oxidase (EC 1.2.5.1) and overexpression of acetate kinase (EC 2.7.2.1) and overexpression of acetyl phosphate-producing pyruvate oxidase (EC 1.2.3.3) and overexpression of pyruvate decarboxylase (EC 4.1.1.1); or is modified to overexpress the acetate-CoA ligase enzyme (EC 6.2.1.1 or 6.2.1.13) combined with overexpressing Pantothenate kinase (EC 2.7.1.33) and overexpression of pyruvate oxidase (EC 1.2.5.1) and overexpression of acetate kinase (EC 2.7.2.1) and overexpression of acetyl phosphate-producing pyruvate oxidase (EC 1.2.3.3) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpressing Pantothenate kinase (EC 2.7.1.33) and overexpression of pyruvate oxidase (EC 1.2.5.1) and overexpression of acetate kinase (EC 2.7.2.1) and overexpression of acetyl phosphate-producing pyruvate oxidase (EC 1.2.3.3) and overexpression of pyruvate decarboxylase (EC 4.1.1.1) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpressing Pantothenate kinase (EC 2.7.1.33) and overexpression of pyruvate oxidase (EC 1.2.5.1) and overexpression of phosphate acetyltransferase (EC 2.3.1.8) and overexpression of acetate kinase (EC 2.7.2.1) and overexpression of pyruvate decarboxylase (EC 4.1.1.1) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpressing Pantothenate kinase (EC 2.7.1.33) and overexpression of pyruvate oxidase (EC 1.2.5.1) and overexpression of phosphate acetyltransferase (EC 2.3.1.8) and overexpression of acetate kinase (EC 2.7.2.1) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpressing Pantothenate kinase (EC 2.7.1.33) and overexpression of pyruvate oxidase (EC 1.2.5.1) and overexpression of acetyl phosphate-producing pyruvate oxidase (EC 1.2.3.3) and overexpression of pyruvate decarboxylase (EC 4.1.1.1); or is modified to overexpress the acetate-CoA ligase enzyme (EC 6.2.1.1 or 6.2.1.13) combined with overexpressing Pantothenate kinase (EC 2.7.1.33) and overexpression of pyruvate oxidase (EC 1.2.5.1) and overexpression of acetyl phosphate-producing pyruvate oxidase (EC 1.2.3.3) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpressing Pantothenate kinase (EC 2.7.1.33) and overexpression of pyruvate oxidase (EC 1.2.5.1) and overexpression of acetyl phosphate-producing pyruvate oxidase (EC 1.2.3.3) and overexpression of pyruvate decarboxylase (EC 4.1.1.1) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpressing Pantothenate kinase (EC 2.7.1.33) and overexpression of pyruvate oxidase (EC 1.2.5.1) overexpression of pyruvate decarboxylase (EC 4.1.1.1) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpressing Pantothenate kinase (EC 2.7.1.33) and overexpression of pyruvate oxidase (EC 1.2.5.1) and overexpression of phosphate acetyltransferase (EC 2.3.1.8) and overexpression of acetyl phosphate-producing pyruvate oxidase (EC 1.2.3.3) and overexpression of pyruvate decarboxylase (EC 4.1.1.1); or is modified to overexpress acetate-CoA ligase enzyme (EC 6.2.1.1 or 6.2.1.13) combined with overexpressing Pantothenate kinase (EC 2.7.1.33) and overexpression of pyruvate oxidase (EC 1.2.5.1) and overexpression of phosphate acetyltransferase (EC 2.3.1.8) and overexpression of acetyl phosphate-producing pyruvate oxidase (EC 1.2.3.3) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpressing Pantothenate kinase (EC 2.7.1.33) and overexpression of pyruvate oxidase (EC 1.2.5.1) and overexpression of phosphate acetyltransferase (EC 2.3.1.8) and overexpression of acetyl phosphate-producing pyruvate oxidase (EC 1.2.3.3) and overexpression of pyruvate decarboxylase (EC 4.1.1.1) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpressing Pantothenate kinase (EC 2.7.1.33) and overexpression of pyruvate oxidase (EC 1.2.5.1) and overexpression of phosphate acetyltransferase (EC 2.3.1.8) and overexpression of pyruvate decarboxylase (EC 4.1.1.1) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpressing Pantothenate kinase (EC 2.7.1.33) and overexpression of phosphate acetyltransferase (EC 2.3.1.8) and overexpression of acetate kinase (EC 2.7.2.1); or is modified to overexpress the acetate-CoA ligase enzyme (EC 6.2.1.1 or 6.2.1.13) combined with overexpressing Pantothenate kinase (EC 2.7.1.33) and overexpression of phosphate acetyltransferase (EC 2.3.1.8) and overexpression of acetyl phosphate-producing pyruvate oxidase (EC 1.2.3.3); or is modified to overexpress the acetate-CoA ligase enzyme (EC 6.2.1.1 or 6.2.1.13) combined with overexpressing Pantothenate kinase (EC 2.7.1.33) and overexpression of phosphate acetyltransferase (EC 2.3.1.8) and overexpression of pyruvate decarboxylase (EC 4.1.1.1); or is modified to overexpress the acetate-CoA ligase enzyme (EC 6.2.1.1 or 6.2.1.13) combined with overexpressing Pantothenate kinase (EC 2.7.1.33) and overexpression of phosphate acetyltransferase (EC 2.3.1.8) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpressing Pantothenate kinase (EC 2.7.1.33) and overexpression of phosphate acetyltransferase (EC 2.3.1.8) and overexpression of acetate kinase (EC 2.7.2.1) and overexpression of acetyl phosphate-producing pyruvate oxidase (EC 1.2.3.3); or is modified to overexpress the acetate-CoA ligase enzyme (EC 6.2.1.1 or 6.2.1.13) combined with overexpressing Pantothenate kinase (EC 2.7.1.33) and overexpression of phosphate acetyltransferase (EC 2.3.1.8) and overexpression of acetate kinase (EC 2.7.2.1) and overexpression of pyruvate decarboxylase (EC 4.1.1.1); or is modified to overexpress the acetate-CoA ligase enzyme (EC 6.2.1.1 or 6.2.1.13) combined with overexpressing Pantothenate kinase (EC 2.7.1.33) and overexpression of phosphate acetyltransferase (EC 2.3.1.8) and overexpression of acetate kinase (EC 2.7.2.1) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpressing Pantothenate kinase (EC 2.7.1.33) and overexpression of phosphate acetyltransferase (EC 2.3.1.8) and overexpression of acetate kinase (EC 2.7.2.1) and overexpression of acetyl phosphate-producing pyruvate oxidase (EC 1.2.3.3) and overexpression of pyruvate decarboxylase (EC 4.1.1.1); or is modified to overexpress the acetate-CoA ligase enzyme (EC 6.2.1.1 or 6.2.1.13) combined with overexpressing Pantothenate kinase (EC 2.7.1.33) and overexpression of phosphate acetyltransferase (EC 2.3.1.8) and overexpression of acetate kinase (EC 2.7.2.1) and overexpression of acetyl phosphate-producing pyruvate oxidase (EC 1.2.3.3) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpressing Pantothenate kinase (EC 2.7.1.33) and overexpression of phosphate acetyltransferase (EC 2.3.1.8) and overexpression of acetate kinase (EC 2.7.2.1) and overexpression of acetyl phosphate-producing pyruvate oxidase (EC 1.2.3.3) and overexpression of pyruvate decarboxylase (EC 4.1.1.1) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpressing Pantothenate kinase (EC 2.7.1.33) and overexpression of phosphate acetyltransferase (EC 2.3.1.8) and overexpression of acetate kinase (EC 2.7.2.1) and overexpression of pyruvate decarboxylase (EC 4.1.1.1) and overexpression of acetaldehyde dehydrogenase (EC 1.2.1.3, EC 1.2.1.4, or EC 1.2.1.5; or is modified to overexpress the acetate-CoA ligase enzyme (EC 6.2.1.1 or 6.2.1.13) combined with overexpressing Pantothenate kinase (EC 2.7.1.33) and overexpression of phosphate acetyltransferase (EC 2.3.1.8) and overexpression of acetate kinase (EC 2.7.2.1) and overexpression of pyruvate decarboxylase (EC 4.1.1.1) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpressing Pantothenate kinase (EC 2.7.1.33) and overexpression of phosphate acetyltransferase (EC 2.3.1.8) and overexpression of acetate kinase (EC 2.7.2.1) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpressing Pantothenate kinase (EC 2.7.1.33) and overexpression of phosphate acetyltransferase (EC 2.3.1.8) and overexpression of acetyl phosphate-producing pyruvate oxidase (EC 1.2.3.3) and overexpression of pyruvate decarboxylase (EC 4.1.1.1); or is modified to overexpress the acetate-CoA ligase enzyme (EC 6.2.1.1 or 6.2.1.13) combined with overexpressing Pantothenate kinase (EC 2.7.1.33) and overexpression of phosphate acetyltransferase (EC 2.3.1.8) and overexpression of acetyl phosphate-producing pyruvate oxidase (EC 1.2.3.3) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpressing Pantothenate kinase (EC 2.7.1.33) and overexpression of phosphate acetyltransferase (EC 2.3.1.8) and overexpression of acetyl phosphate-producing pyruvate oxidase (EC 1.2.3.3) and overexpression of pyruvate decarboxylase (EC 4.1.1.1) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpressing Pantothenate kinase (EC 2.7.1.33) and overexpression of phosphate acetyltransferase (EC 2.3.1.8) and overexpression of pyruvate decarboxylase (EC 4.1.1.1) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpressing Pantothenate kinase (EC 2.7.1.33) and overexpression of acetate kinase (EC 2.7.2.1) and overexpression of acetyl phosphate-producing pyruvate oxidase (EC 1.2.3.3); or is modified to overexpress the acetate-CoA ligase enzyme (EC 6.2.1.1 or 6.2.1.13) combined with overexpressing Pantothenate kinase (EC 2.7.1.33) and overexpression of acetate kinase (EC 2.7.2.1) and overexpression of pyruvate decarboxylase (EC 4.1.1.1); or is modified to overexpress the acetate-CoA ligase enzyme (EC 6.2.1.1 or 6.2.1.13) combined with overexpressing Pantothenate kinase (EC 2.7.1.33) and overexpression of acetate kinase (EC 2.7.2.1) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpressing Pantothenate kinase (EC 2.7.1.33) and overexpression of acetate kinase (EC 2.7.2.1) and overexpression of acetyl phosphate-producing pyruvate oxidase (EC 1.2.3.3) and overexpression of pyruvate decarboxylase (EC 4.1.1.1); or is modified to overexpress the acetate-CoA ligase enzyme (EC 6.2.1.1 or 6.2.1.13) combined with overexpressing Pantothenate kinase (EC 2.7.1.33) and overexpression of acetate kinase (EC 2.7.2.1) and overexpression of acetyl phosphate-producing pyruvate oxidase (EC 1.2.3.3) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpressing Pantothenate kinase (EC 2.7.1.33) and overexpression of acetate kinase (EC 2.7.2.1) and overexpression of acetyl phosphate-producing pyruvate oxidase (EC 1.2.3.3) and overexpression of pyruvate decarboxylase (EC 4.1.1.1) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpressing Pantothenate kinase (EC 2.7.1.33) and overexpression of acetate kinase (EC 2.7.2.1) and overexpression of pyruvate decarboxylase (EC 4.1.1.1) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpressing Pantothenate kinase (EC 2.7.1.33) and overexpression of acetyl phosphate-producing pyruvate oxidase (EC 1.2.3.3) and overexpression of pyruvate decarboxylase (EC 4.1.1.1); or is modified to overexpress the acetate-CoA ligase enzyme (EC 6.2.1.1 or 6.2.1.13) combined with overexpressing Pantothenate kinase (EC 2.7.1.33) and overexpression of acetyl phosphate-producing pyruvate oxidase (EC 1.2.3.3) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpressing Pantothenate kinase (EC 2.7.1.33) and overexpression of acetyl phosphate-producing pyruvate oxidase (EC 1.2.3.3) and overexpression of pyruvate decarboxylase (EC 4.1.1.1) and overexpression of acetaldehyde dehydrogenase (EC 1.2.1.3, EC 1.2.1.4, or EC 1.2.1.5); or is modified to overexpress the acetate-CoA ligase enzyme (EC 6.2.1.1 or 6.2.1.13) combined with overexpressing Pantothenate kinase (EC 2.7.1.33) and overexpression of pyruvate decarboxylase (EC 4.1.1.1) and overexpression of acetaldehyde dehydrogenase (EC 1.2.1.3, EC 1.2.1.4, or EC 1.2.1.5).

Another exemplary embodiment of the method and/or cell of the present invention provides for a cell which is modified to overexpress the acetate-CoA ligase enzyme (EC 6.2.1.1 or 6.2.1.13) combined with overexpression of pyruvate formate lyase (EC 2.3.1.54) directly forming acetyl-CoA from pyruvate; or is modified to overexpress the acetate-CoA ligase enzyme (EC 6.2.1.1 or 6.2.1.13) combined with overexpression of CoA-acetylating pyruvate oxidase (EC 1.2.3.6); or is modified to overexpress the acetate-CoA ligase enzyme (EC 6.2.1.1 or 6.2.1.13) combined with overexpression of pyruvate dehydrogenase enzyme complex as defined herein; or is modified to overexpress the acetate-CoA ligase enzyme (EC 6.2.1.1 or 6.2.1.13) combined with overexpression of pyruvate synthase (EC 1.2.7.1); or is modified to overexpress the acetate-CoA ligase enzyme (EC 6.2.1.1 or 6.2.1.13) combined with overexpression of pyruvate formate lyase (EC 2.3.1.54) and overexpression of CoA-acetylating pyruvate oxidase (EC 1.2.3.6); or is modified to overexpress the acetate-CoA ligase enzyme (EC 6.2.1.1 or 6.2.1.13) combined with overexpression of pyruvate formate lyase (EC 2.3.1.54) and overexpression of CoA-acetylating pyruvate oxidase (EC 1.2.3.6); or is modified to overexpress the acetate-CoA ligase enzyme (EC 6.2.1.1 or 6.2.1.13) combined with overexpression of pyruvate formate lyase (EC 2.3.1.54) and overexpression of pyruvate dehydrogenase enzyme complex as defined herein; or is modified to overexpress the acetate-CoA ligase enzyme (EC 6.2.1.1 or 6.2.1.13) combined with overexpression of pyruvate formate lyase (EC 2.3.1.54) and overexpression of pyruvate synthase (EC 1.2.7.1); or is modified to overexpress the acetate-CoA ligase enzyme (EC 6.2.1.1 or 6.2.1.13) combined with overexpression of pyruvate formate lyase (EC 2.3.1.54) and overexpression of CoA-acetylating pyruvate oxidase (EC 1.2.3.6) and overexpression of pyruvate dehydrogenase enzyme complex as defined herein; or is modified to overexpress the acetate-CoA ligase enzyme (EC 6.2.1.1 or 6.2.1.13) combined with overexpression of pyruvate formate lyase (EC 2.3.1.54) and overexpression of CoA-acetylating pyruvate oxidase (EC 1.2.3.6) and overexpression of pyruvate synthase (EC 1.2.7.1); or is modified to overexpress the acetate-CoA ligase enzyme (EC 6.2.1.1 or 6.2.1.13) combined with overexpression of pyruvate formate lyase (EC 2.3.1.54) and overexpression of CoA-acetylating pyruvate oxidase (EC 1.2.3.6) and overexpression of pyruvate dehydrogenase enzyme complex as defined herein and overexpression of pyruvate synthase (EC 1.2.7.1); or is modified to overexpress the acetate-CoA ligase enzyme (EC 6.2.1.1 or 6.2.1.13) combined with overexpression of pyruvate formate lyase (EC 2.3.1.54) and overexpression of pyruvate dehydrogenase enzyme complex as defined herein; or is modified to overexpress the acetate-CoA ligase enzyme (EC 6.2.1.1 or 6.2.1.13) combined with overexpression of pyruvate formate lyase (EC 2.3.1.54) and overexpression of pyruvate synthase (EC 1.2.7.1); or is modified to overexpress the acetate-CoA ligase enzyme (EC 6.2.1.1 or 6.2.1.13) combined with overexpression of pyruvate formate lyase (EC 2.3.1.54) and overexpression of pyruvate dehydrogenase enzyme complex as defined herein and overexpression of pyruvate synthase (EC 1.2.7.1); or is modified to overexpress the acetate-CoA ligase enzyme (EC 6.2.1.1 or 6.2.1.13) combined with overexpression of CoA-acetylating pyruvate oxidase (EC 1.2.3.6) and overexpression of pyruvate dehydrogenase enzyme complex as defined herein; or is modified to overexpress the acetate-CoA ligase enzyme (EC 6.2.1.1 or 6.2.1.13) combined with overexpression of CoA-acetylating pyruvate oxidase (EC 1.2.3.6) and overexpression of pyruvate synthase (EC 1.2.7.1); or is modified to overexpress the acetate-CoA ligase enzyme (EC 6.2.1.1 or 6.2.1.13) combined with overexpression of CoA-acetylating pyruvate oxidase (EC 1.2.3.6) and overexpression of pyruvate dehydrogenase enzyme complex as defined herein and overexpression of pyruvate synthase (EC 1.2.7.1); or is modified to overexpress the acetate-CoA ligase enzyme (EC 6.2.1.1 or 6.2.1.13) combined with overexpression of pyruvate dehydrogenase enzyme complex as defined herein and overexpression of pyruvate synthase (EC 1.2.7.1); or is modified to overexpress pyruvate formate lyase (EC 2.3.1.54) and to overexpress CoA-acetylating pyruvate oxidase (EC 1.2.3.6); or is modified to overexpress pyruvate formate lyase (EC 2.3.1.54) and to overexpress pyruvate dehydrogenase enzyme complex as defined herein; or is modified to overexpress pyruvate formate lyase (EC 2.3.1.54) and to overexpress pyruvate synthase (EC 1.2.7.1); or is modified to overexpress pyruvate formate lyase (EC 2.3.1.54) and to overexpress CoA-acetylating pyruvate oxidase (EC 1.2.3.6) and to overexpress pyruvate dehydrogenase enzyme complex as defined herein; or is modified to overexpress pyruvate formate lyase (EC 2.3.1.54) and to overexpress CoA-acetylating pyruvate oxidase (EC 1.2.3.6) and to overexpress pyruvate synthase (EC 1.2.7.1); or is modified to overexpress pyruvate formate lyase (EC 2.3.1.54) and to overexpress CoA-acetylating pyruvate oxidase (EC 1.2.3.6) and to overexpress pyruvate dehydrogenase enzyme complex as defined herein and to overexpress pyruvate synthase (EC 1.2.7.1); or is modified to overexpress pyruvate formate lyase (EC 2.3.1.54) and to overexpress pyruvate dehydrogenase enzyme as defined herein; or is modified to overexpress pyruvate formate lyase (EC 2.3.1.54) and to overexpress pyruvate synthase (EC 1.2.7.1); or is modified to overexpress pyruvate formate lyase (EC 2.3.1.54) and to overexpress pyruvate dehydrogenase enzyme complex as defined herein and to overexpress pyruvate synthase (EC 1.2.7.1); or is modified to overexpress CoA-acetylating pyruvate oxidase (EC 1.2.3.6) and to overexpress pyruvate dehydrogenase enzyme complex as defined herein; or is modified to overexpress CoA-acetylating pyruvate oxidase (EC 1.2.3.6) and to overexpress pyruvate synthase (EC 1.2.7.1); or is modified to overexpress CoA-acetylating pyruvate oxidase (EC 1.2.3.6) and to overexpress pyruvate dehydrogenase enzyme complex as defined herein and to overexpress pyruvate synthase (EC 1.2.7.1); or is modified to overexpress pyruvate dehydrogenase enzyme complex as defined herein and to overexpress pyruvate synthase (EC 1.2.7.1); or is modified to overexpress CoA-acetylating pyruvate oxidase (EC 1.2.3.6) and to overexpress pyruvate dehydrogenase enzyme complex as defined herein; or is modified to overexpress CoA-acetylating pyruvate oxidase (EC 1.2.3.6) and to overexpress pyruvate synthase (EC 1.2.7.1); or is modified to overexpress CoA-acetylating pyruvate oxidase (EC 1.2.3.6) and to overexpress pyruvate dehydrogenase enzyme complex as defined herein and to overexpress pyruvate synthase (EC 1.2.7.1); or is modified to overexpress pyruvate dehydrogenase enzyme complex as defined herein and to overexpress pyruvate synthase (EC 1.2.7.1)

Alternative exemplary embodiments of the method and/or cell of the present invention provides for a cell which is modified to overexpress the acetate-CoA ligase enzyme (EC 6.2.1.1 or 6.2.1.13) combined with overexpressing Pantothenate kinase (EC 2.7.1.33) and overexpressing pyruvate formate lyase (EC 2.3.1.54); or is modified to overexpress the acetate-CoA ligase enzyme (EC 6.2.1.1 or 6.2.1.13) combined with overexpressing Pantothenate kinase (EC 2.7.1.33) and overexpressing CoA-acetylating pyruvate oxidase (EC 1.2.3.6); or is modified to overexpress the acetate-CoA ligase enzyme (EC 6.2.1.1 or 6.2.1.13) combined with overexpressing Pantothenate kinase (EC 2.7.1.33) and overexpressing the pyruvate dehydrogenase enzyme complex as defined herein; or is modified to overexpress the acetate-CoA ligase enzyme (EC 6.2.1.1 or 6.2.1.13) combined with overexpressing Pantothenate kinase (EC 2.7.1.33) and overexpressing pyruvate synthase (EC 1.2.7.1); or is modified to overexpress the acetate-CoA ligase enzyme (EC 6.2.1.1 or 6.2.1.13) combined with overexpressing Pantothenate kinase (EC 2.7.1.33) and overexpressing pyruvate formate lyase (EC 2.3.1.54) and overexpressing CoA-acetylating pyruvate oxidase (EC 1.2.3.6); or is modified to overexpress the acetate-CoA ligase enzyme (EC 6.2.1.1 or 6.2.1.13) combined with overexpressing Pantothenate kinase (EC 2.7.1.33) and overexpressing pyruvate formate lyase (EC 2.3.1.54) and overexpressing CoA-acetylating pyruvate oxidase (EC 1.2.3.6); or is modified to overexpress the acetate-CoA ligase enzyme (EC 6.2.1.1 or 6.2.1.13) combined with overexpressing Pantothenate kinase (EC 2.7.1.33) and overexpressing pyruvate formate lyase (EC 2.3.1.54) and overexpressing the pyruvate dehydrogenase enzyme complex as defined herein; or is modified to overexpress the acetate-CoA ligase enzyme (EC 6.2.1.1 or 6.2.1.13) combined with overexpressing Pantothenate kinase (EC 2.7.1.33) and overexpressing pyruvate formate lyase (EC 2.3.1.54) and overexpressing pyruvate synthase (EC 1.2.7.1); or is modified to overexpress the acetate-CoA ligase enzyme (EC 6.2.1.1 or 6.2.1.13) combined with overexpressing Pantothenate kinase (EC 2.7.1.33) and overexpressing pyruvate formate lyase (EC 2.3.1.54) and overexpressing CoA-acetylating pyruvate oxidase (EC 1.2.3.6) and overexpressing the pyruvate dehydrogenase enzyme complex as defined herein; or is modified to overexpress the acetate-CoA ligase enzyme (EC 6.2.1.1 or 6.2.1.13) combined with overexpressing Pantothenate kinase (EC 2.7.1.33) and overexpressing pyruvate formate lyase (EC 2.3.1.54) and overexpressing CoA-acetylating pyruvate oxidase (EC 1.2.3.6) and overexpressing pyruvate synthase (EC 1.2.7.1); or is modified to overexpress the acetate-CoA ligase enzyme (EC 6.2.1.1 or 6.2.1.13) combined with overexpressing Pantothenate kinase (EC 2.7.1.33) and overexpressing pyruvate formate lyase (EC 2.3.1.54) and CoA-acetylating pyruvate oxidase (EC 1.2.3.6) and overexpressing the pyruvate dehydrogenase enzyme complex as defined herein and overexpressing pyruvate synthase (EC 1.2.7.1); or is modified to overexpress the acetate-CoA ligase enzyme (EC 6.2.1.1 or 6.2.1.13) combined with overexpressing Pantothenate kinase (EC 2.7.1.33) and overexpressing pyruvate formate lyase (EC 2.3.1.54) and overexpressing the pyruvate dehydrogenase enzyme complex as defined herein; or is modified to overexpress the acetate-CoA ligase enzyme (EC 6.2.1.1 or 6.2.1.13) combined with overexpressing Pantothenate kinase (EC 2.7.1.33) and overexpressing pyruvate formate lyase (EC 2.3.1.54) and overexpressing pyruvate synthase (EC 1.2.7.1); or is modified to overexpress the acetate-CoA ligase enzyme (EC 6.2.1.1 or 6.2.1.13) combined with overexpressing Pantothenate kinase (EC 2.7.1.33) and overexpressing pyruvate formate lyase (EC 2.3.1.54) and overexpressing the pyruvate dehydrogenase enzyme complex as defined herein, and overexpressing pyruvate synthase (EC 1.2.7.1); or is modified to overexpress the acetate-CoA ligase enzyme (EC 6.2.1.1 or 6.2.1.13) combined with overexpressing Pantothenate kinase (EC 2.7.1.33) and overexpressing CoA-acetylating pyruvate oxidase (EC 1.2.3.6) and overexpressing the pyruvate dehydrogenase enzyme complex as defined herein; or is modified to overexpress the acetate-CoA ligase enzyme (EC 6.2.1.1 or 6.2.1.13) combined with overexpressing Pantothenate kinase (EC 2.7.1.33) and overexpressing CoA-acetylating pyruvate oxidase (EC 1.2.3.6) and overexpressing pyruvate synthase (EC 1.2.7.1); or is modified to overexpress the acetate-CoA ligase enzyme (EC 6.2.1.1 or 6.2.1.13) combined with overexpressing Pantothenate kinase (EC 2.7.1.33) and overexpressing CoA-acetylating pyruvate oxidase (EC 1.2.3.6) and overexpressing the pyruvate dehydrogenase enzyme complex as defined herein and overexpressing pyruvate synthase (EC 1.2.7.1); or is modified to overexpress the acetate-CoA ligase enzyme (EC 6.2.1.1 or 6.2.1.13) combined with overexpressing Pantothenate kinase (EC 2.7.1.33) and overexpressing the pyruvate dehydrogenase enzyme complex as defined herein and overexpressing pyruvate synthase (EC 1.2.7.1); or is modified to overexpress pyruvate formate lyase (EC 2.3.1.54) and overexpressing CoA-acetylating pyruvate oxidase (EC 1.2.3.6); or is modified to overexpress pyruvate formate lyase (EC 2.3.1.54) and overexpressing the pyruvate dehydrogenase enzyme complex as defined herein; or is modified to overexpress pyruvate formate lyase (EC 2.3.1.54) and overexpressing pyruvate synthase (EC 1.2.7.1); or is modified to overexpress pyruvate formate lyase (EC 2.3.1.54) and overexpressing CoA-acetylating pyruvate oxidase (EC 1.2.3.6) and overexpressing the pyruvate dehydrogenase enzyme complex as defined herein; or is modified to overexpress pyruvate formate lyase (EC 2.3.1.54) and overexpressing CoA-acetylating pyruvate oxidase (EC 1.2.3.6) and overexpressing pyruvate synthase (EC 1.2.7.1); or is modified to overexpress pyruvate formate lyase (EC 2.3.1.54) and overexpressing CoA-acetylating pyruvate oxidase (EC 1.2.3.6) and overexpressing the pyruvate dehydrogenase enzyme complex as defined herein and overexpressing pyruvate synthase (EC 1.2.7.1); or is modified to overexpress pyruvate formate lyase (EC 2.3.1.54) and overexpressing the pyruvate dehydrogenase enzyme complex as defined herein; or is modified to overexpress pyruvate formate lyase (EC 2.3.1.54) and overexpressing pyruvate synthase (EC 1.2.7.1); or is modified to overexpress pyruvate formate lyase (EC 2.3.1.54) and overexpressing the pyruvate dehydrogenase enzyme complex as defined herein and overexpressing pyruvate synthase (EC 1.2.7.1); or is modified to overexpress CoA-acetylating pyruvate oxidase (EC 1.2.3.6) and overexpressing the pyruvate dehydrogenase enzyme complex as defined herein; or is modified to overexpress CoA-acetylating pyruvate oxidase (EC 1.2.3.6) and overexpressing pyruvate synthase (EC 1.2.7.1); or is modified to overexpress CoA-acetylating pyruvate oxidase (EC 1.2.3.6) and overexpressing the pyruvate dehydrogenase enzyme complex as defined herein and overexpressing pyruvate synthase (EC 1.2.7.1); or is modified to overexpress the pyruvate dehydrogenase enzyme complex as defined herein and overexpressing pyruvate synthase (EC 1.2.7.1); or is modified to overexpress CoA-acetylating pyruvate oxidase (EC 1.2.3.6) and overexpressing the pyruvate dehydrogenase enzyme complex as defined herein; or is modified to overexpress CoA-acetylating pyruvate oxidase (EC 1.2.3.6) and overexpressing pyruvate synthase (EC 1.2.7.1); or is modified to overexpress CoA-acetylating pyruvate oxidase (EC 1.2.3.6) and overexpressing the pyruvate dehydrogenase enzyme complex as defined herein and overexpressing pyruvate synthase (EC 1.2.7.1); or is modified to overexpress the pyruvate dehydrogenase enzyme complex as defined herein and overexpressing pyruvate synthase (EC 1.2.7.1)

In another preferred embodiment, the cell comprises a pathway for production of a compound of present invention and further comprises a pathway for production of phosphoenolpyruvate (PEP).

In another preferred embodiment of the cell and/or the method of the invention, the cell is modified for enhanced synthesis and/or supply of PEP.

In a preferred embodiment and as a means for enhanced production and/or supply of PEP, one or more PEP-dependent, sugar-transporting phosphotransferase system(s) is/are disrupted such as but not limited to: 1) the N-acetyl-D-glucosamine Npi-phosphotransferase (EC 2.7.1.193), which is for instance encoded by the nagE gene (or the cluster nagABCD) in E. coli or Bacillus species, 2) ManXYZ which encodes the Enzyme II Man complex (mannose PTS permease, protein-Npi-phosphohistidine-D-mannose phosphotransferase) that imports exogenous hexoses (mannose, glucose, glucosamine, fructose, 2-deoxyglucose, mannosamine, N-acetylglucosamine, etc.) and releases the phosphate esters into the cell cytoplasm, 3) the glucose-specific PTS transporter (for instance encoded by PtsG/Crr) which takes up glucose and forms glucose-6-phosphate in the cytoplasm, 4) the sucrose-specific PTS transporter which takes up sucrose and forms sucrose-6-phosphate in the cytoplasm, 5) the fructose-specific PTS transporter (for instance encoded by the genes fruA and fruB and the kinase fruK which takes up fructose and forms in a first step fructose-1-phosphate and in a second step fructose1,6 bisphosphate, 6) the lactose PTS transporter (for instance encoded by lacE in Lactococcus casei) which takes up lactose and forms lactose-6-phosphate, 7) the galactitol-specific PTS enzyme which takes up galactitol and/or sorbitol and forms galactitol-1-phosphate or sorbitol-6-phosphate respectively, 8) the mannitol-specific PTS enzyme which takes up mannitol and/or sorbitol and forms mannitol-1-phosphate or sorbitol-6-phosphate respectively, and 9) the trehalose-specific PTS enzyme which takes up trehalose and forms trehalose-6-phosphate.

In another and/or additional preferred embodiment and as a means for enhanced production and/or supply of PEP, the full PTS system is disrupted by disrupting the PtsIH/Crr gene cluster. PtsI (Enzyme I) is a cytoplasmic protein that serves as the gateway for the phosphoenolpyruvate:sugar phosphotransferase system (PTS^{sugar}) of E. coli K-12. PtsI is one of two (PtsI and PtsH) sugar non-specific protein constituents of the PTS^{sugar} which along with a sugar-specific inner membrane permease effects a phosphotransfer cascade that results in the coupled phosphorylation and transport of a variety of carbohydrate substrates. HPr (histidine containing protein) is one of two sugar-non-specific protein constituents of the PTS^{sugar}. It accepts a phosphoryl group from phosphorylated Enzyme I (PtsI-P) and then transfers it to the EIIA domain of any one of the many sugar-specific enzymes (collectively known as Enzymes II) of the PTS^{sugar}. Crr or EIIA^{Glc} is phosphorylated by PEP in a reaction requiring PtsH and PtsI.

In another and/or additional preferred embodiment, the cell is further modified to compensate for the deletion of a PTS system of a carbon source by the introduction and/or overexpression of the corresponding permease. These are e.g. permeases or ABC transporters that comprise but are not limited to transporters that specifically import lactose such as e.g. the transporter encoded by the LacY gene from E. coli, sucrose such as e.g. the transporter encoded by the cscB gene from E. coli, glucose such as e.g. the transporter encoded by the galP gene from E. coli, fructose such as e.g. the transporter encoded by the fruI gene from Streptococcus mutans, or the Sorbitol/mannitol ABC transporter such as the transporter encoded by the cluster SmoEFGK of Rhodobacter sphaeroides, the trehalose/sucrose/maltose transporter such as the transporter encoded by the gene cluster ThuEFGK of Sinorhizobium meliloti and the N-acetylglucosamine/galactose/glucose transporter such as the transporter encoded by NagP of Shewanella oneidensis. Examples of combinations of PTS deletions with overexpression of alternative transporters are: 1) the deletion of the glucose PTS system, e.g. ptsG gene, combined with the introduction and/or overexpression of a glucose permease (e.g. galP of glcP), 2) the deletion of the fructose PTS system, e.g. one or more of the fruB, fruA, fruK genes, combined with the introduction and/or overexpression of fructose permease, e.g. fruI, 3) the deletion of the lactose PTS system, combined with the introduction and/or overexpression of lactose permease, e.g. LacY, and/or 4) the deletion of the sucrose PTS system, combined with the introduction and/or overexpression of a sucrose permease, e.g. cscB.

In a further preferred embodiment, the cell is modified to compensate for the deletion of a PTS system of a carbon source by the introduction of carbohydrate kinases, such as glucokinase (EC 2.7.1.1, EC 2.7.1.2, EC 2.7.1.63), galactokinase (EC 2.7.1.6), and/or fructokinase (EC 2.7.1.3, EC 2.7.1.4). Examples of combinations of PTS deletions with overexpression of alternative transporters and a kinase are: 1) the deletion of the glucose PTS system, e.g. ptsG gene, combined with the introduction and/or overexpression of a glucose permease (e.g. galP of glcP), combined with the introduction and/or overexpression of a glucokinase (e.g. glk), and/or 2) the deletion of the fructose PTS system, e.g. one or more of the fruB, fruA, fruK genes, combined with the introduction and/or overexpression of fructose permease, e.g. fruI, combined with the introduction and/or overexpression of a fructokinase (e.g. frk or mak).

In another and/or additional preferred embodiment and as a means for enhanced production and/or supply of PEP, the cell is modified by the introduction of or modification in any one or more of the list comprising phosphoenolpyruvate synthase activity (EC: 2.7.9.2 encoded for instance in E. coli by ppsA), phosphoenolpyruvate carboxykinase activity (EC 4.1.1.32 or EC 4.1.1.49 encoded for instance in Corynebacterium glutamicum by PCK or in E. coli by pckA, resp.), phosphoenolpyruvate carboxylase activity (EC 4.1.1.31 encoded for instance in E. coli by ppc), oxaloacetate decarboxylase activity (EC 4.1.1.112 encoded for instance in E. coli by eda), pyruvate kinase activity (EC 2.7.1.40 encoded for instance in E. coli by pykA and pykF), pyruvate carboxylase activity (EC 6.4.1.1 encoded for instance in B. subtilis by pyc) and malate dehydrogenase activity (EC 1.1.1.38 or EC 1.1.1.40 encoded for instance in E. coli by maeA or maeB, resp.).

In another and/or additional preferred embodiment and as a means for enhanced production and/or supply of PEP, the cell is modified by a reduced activity of phosphoenolpyruvate carboxylase activity, and/or pyruvate kinase activity, preferably a deletion of the genes encoding for phosphoenolpyruvate carboxylase, the pyruvate carboxylase activity and/or pyruvate kinase.

In an exemplary embodiment, the cell is metabolically engineered by different adaptations such as overexpression of phosphoenolpyruvate synthase combined with the deletion of a pyruvate kinase gene, overexpression of phosphoenolpyruvate synthase combined with the deletion of a phosphoenolpyruvate carboxylase gene, overexpression of phosphoenolpyruvate synthase combined with the deletion of a pyruvate carboxylase gene, overexpression of phosphoenolpyruvate carboxykinase combined with the deletion of a pyruvate kinase gene, overexpression of phosphoenolpyruvate carboxykinase combined with the deletion of a phosphoenolpyruvate carboxylase gene, overexpression of phosphoenolpyruvate carboxykinase combined with the deletion of a pyruvate carboxylase gene, overexpression of oxaloacetate decarboxylase combined with the deletion of a pyruvate kinase gene, overexpression of oxaloacetate decarboxylase combined with the deletion of a phosphoenolpyruvate carboxylase gene, overexpression of oxaloacetate decarboxylase combined with the deletion of a pyruvate carboxylase gene, overexpression of malate dehydrogenase combined with the deletion of a pyruvate kinase gene, overexpression of malate dehydrogenase combined with the deletion of a phosphoenolpyruvate carboxylase gene and/or overexpression of malate dehydrogenase combined with the deletion of a pyruvate carboxylase gene.

In another exemplary embodiment, the cell is metabolically engineered by different adaptations such as overexpression of phosphoenolpyruvate synthase combined with overexpression of a phosphoenolpyruvate carboxykinase, overexpression of phosphoenolpyruvate synthase combined with overexpression of an oxaloacetate decarboxylase, overexpression of phosphoenolpyruvate synthase combined with overexpression of a malate dehydrogenase, overexpression of phosphoenolpyruvate carboxykinase combined with overexpression of an oxaloacetate decarboxylase, overexpression of phosphoenolpyruvate carboxykinase combined with overexpression of a malate dehydrogenase, overexpression of oxaloacetate decarboxylase combined with overexpression of a malate dehydrogenase, overexpression of phosphoenolpyruvate synthase combined with overexpression of a phosphoenolpyruvate carboxykinase and overexpression of an oxaloacetate decarboxylase, overexpression of phosphoenolpyruvate synthase combined with overexpression of a phosphoenolpyruvate carboxykinase and overexpression of a malate dehydrogenase, overexpression of phosphoenolpyruvate synthase combined with overexpression of a phosphoenolpyruvate carboxykinase and overexpression of an oxaloacetate decarboxylase and overexpression of a malate dehydrogenase, overexpression of phosphoenolpyruvate carboxykinase combined with overexpression of an oxaloacetate decarboxylase and overexpression of a malate dehydrogenase and/or overexpression of phosphoenolpyruvate synthase combined with overexpression of an oxaloacetate decarboxylase and overexpression of a malate dehydrogenase.

In another exemplary embodiment, the cell is metabolically engineered by different adaptations such as overexpression of phosphoenolpyruvate synthase combined with overexpression of a phosphoenolpyruvate carboxykinase combined with the deletion of a pyruvate kinase gene, overexpression of phosphoenolpyruvate synthase combined with overexpression of an oxaloacetate decarboxylase combined with the deletion of a pyruvate kinase gene, overexpression of phosphoenolpyruvate synthase combined with overexpression of a malate dehydrogenase combined with the deletion of a pyruvate kinase gene, overexpression of phosphoenolpyruvate carboxykinase combined with overexpression of an oxaloacetate decarboxylase combined with the deletion of a pyruvate kinase gene, overexpression of phosphoenolpyruvate carboxykinase combined with overexpression of a malate dehydrogenase combined with the deletion of a pyruvate kinase gene, overexpression of an oxaloacetate decarboxylase combined with overexpression of a malate dehydrogenase combined with the deletion of a pyruvate kinase gene, overexpression of phosphoenolpyruvate synthase combined with overexpression of a phosphoenolpyruvate carboxykinase and overexpression of an oxaloacetate decarboxylase combined with the deletion of a pyruvate kinase gene, overexpression of phosphoenolpyruvate synthase combined with overexpression of a phosphoenolpyruvate carboxykinase and overexpression of a malate dehydrogenase combined with the deletion of a pyruvate kinase gene, overexpression of phosphoenolpyruvate synthase combined with overexpression of a phosphoenolpyruvate carboxykinase and overexpression of an oxaloacetate decarboxylase and overexpression of a malate dehydrogenase combined with the deletion of a pyruvate kinase gene, overexpression of a phosphoenolpyruvate carboxykinase combined with overexpression of an oxaloacetate decarboxylase and overexpression of a malate dehydrogenase combined with the deletion of a pyruvate kinase gene, overexpression of phosphoenolpyruvate synthase combined overexpression of oxaloacetate decarboxylase and overexpression of a malate dehydrogenase combined with the deletion of a pyruvate kinase gene.

In another exemplary embodiment, the cell is metabolically engineered by different adaptations such as overexpression of phosphoenolpyruvate synthase combined with overexpression of a phosphoenolpyruvate carboxykinase combined with the deletion of a phosphoenolpyruvate carboxylase gene, overexpression of phosphoenolpyruvate synthase combined with overexpression of an oxaloacetate decarboxylase combined with the deletion of a phosphoenolpyruvate carboxylase gene, overexpression of phosphoenolpyruvate synthase combined with overexpression of a malate dehydrogenase combined with the deletion of a phosphoenolpyruvate carboxylase gene, overexpression of a phosphoenolpyruvate carboxykinase combined with overexpression of an oxaloacetate decarboxylase combined with the deletion of a phosphoenolpyruvate carboxylase gene, overexpression of a phosphoenolpyruvate carboxykinase combined with overexpression of a malate dehydrogenase combined with the deletion of a phosphoenolpyruvate carboxylase gene, overexpression of an oxaloacetate decarboxylase combined with overexpression of a malate dehydrogenase combined with the deletion of a phosphoenolpyruvate carboxylase gene, overexpression of phosphoenolpyruvate synthase combined with overexpression of a phosphoenolpyruvate carboxykinase and overexpression of an oxaloacetate decarboxylase combined with the deletion of a phosphoenolpyruvate carboxylase gene, overexpression of phosphoenolpyruvate synthase combined with overexpression of a phosphoenolpyruvate carboxykinase and overexpression of a malate dehydrogenase combined with the deletion of a phosphoenolpyruvate carboxylase gene, overexpression of phosphoenolpyruvate synthase combined with overexpression of a phosphoenolpyruvate carboxykinase and overexpression of an oxaloacetate decarboxylase and overexpression of a malate dehydrogenase combined with the deletion of a phosphoenolpyruvate carboxylase gene, overexpression of a phosphoenolpyruvate carboxykinase combined with overexpression of an oxaloacetate decarboxylase and overexpression of a malate dehydrogenase combined with the deletion of a phosphoenolpyruvate carboxylase gene, overexpression of phosphoenolpyruvate synthase combined overexpression of an oxaloacetate decarboxylase and overexpression of a malate dehydrogenase combined with the deletion of a phosphoenolpyruvate carboxylase gene.

In another exemplary embodiment, the cell is metabolically engineered by different adaptations such as overexpression of phosphoenolpyruvate synthase combined with overexpression of a phosphoenolpyruvate carboxykinase combined with the deletion of a pyruvate carboxylase gene, overexpression of phosphoenolpyruvate synthase combined with overexpression of an oxaloacetate decarboxylase combined with the deletion of a pyruvate carboxylase gene, overexpression of phosphoenolpyruvate synthase combined with overexpression of a malate dehydrogenase combined with the deletion of a pyruvate carboxylase gene, overexpression of a phosphoenolpyruvate carboxykinase combined with overexpression of an oxaloacetate decarboxylase combined with the deletion of a pyruvate carboxylase gene, overexpression of a phosphoenolpyruvate carboxykinase combined with overexpression of a malate dehydrogenase combined with the deletion of a pyruvate carboxylase gene, overexpression of an oxaloacetate decarboxylase combined with overexpression of a malate dehydrogenase combined with the deletion of a pyruvate carboxylase gene, overexpression of phosphoenolpyruvate synthase combined with overexpression of a phosphoenolpyruvate carboxykinase and overexpression of an oxaloacetate decarboxylase combined with the deletion of a pyruvate carboxylase gene, overexpression of phosphoenolpyruvate synthase combined with overexpression of a phosphoenolpyruvate carboxykinase and overexpression of a malate dehydrogenase combined with the deletion of a pyruvate carboxylase gene, overexpression of phosphoenolpyruvate synthase combined with overexpression of a phosphoenolpyruvate carboxykinase and overexpression of an oxaloacetate decarboxylase and overexpression of a malate dehydrogenase combined with the deletion of a pyruvate carboxylase gene, overexpression of a phosphoenolpyruvate carboxykinase combined with overexpression of an oxaloacetate decarboxylase and overexpression of a malate dehydrogenase combined with the deletion of a pyruvate carboxylase gene, overexpression of phosphoenolpyruvate synthase combined with overexpression of an oxaloacetate decarboxylase and overexpression of a malate dehydrogenase combined with the deletion of a pyruvate carboxylase gene.

In another exemplary embodiment, the cell is metabolically engineered by different adaptations such as overexpression of phosphoenolpyruvate synthase combined with overexpression of a phosphoenolpyruvate carboxykinase combined with the deletion of a pyruvate kinase gene and a phosphoenolpyruvate carboxylase gene, overexpression of phosphoenolpyruvate synthase combined with overexpression of an oxaloacetate decarboxylase combined with the deletion of a pyruvate kinase gene and a phosphoenolpyruvate carboxylase gene, overexpression of phosphoenolpyruvate synthase combined with overexpression of a malate dehydrogenase combined with the deletion of a pyruvate kinase gene and a phosphoenolpyruvate carboxylase gene, overexpression of a phosphoenolpyruvate carboxykinase combined with overexpression of an oxaloacetate decarboxylase combined with the deletion of a pyruvate kinase gene and a phosphoenolpyruvate carboxylase gene, overexpression of a phosphoenolpyruvate carboxykinase combined with overexpression of a malate dehydrogenase combined with the deletion of a pyruvate kinase gene and a phosphoenolpyruvate carboxylase gene, overexpression of an oxaloacetate decarboxylase combined with overexpression of a malate dehydrogenase combined with the deletion of a pyruvate kinase gene and a phosphoenolpyruvate carboxylase gene, overexpression of a phosphoenolpyruvate synthase combined with overexpression of a phosphoenolpyruvate carboxykinase and overexpression of an oxaloacetate decarboxylase combined with the deletion of a pyruvate kinase gene and a phosphoenolpyruvate carboxylase gene, overexpression of a phosphoenolpyruvate synthase combined with overexpression of a phosphoenolpyruvate carboxykinase and overexpression of a malate dehydrogenase combined with the deletion of a pyruvate kinase gene and a phosphoenolpyruvate carboxylase gene, overexpression of a phosphoenolpyruvate synthase combined with overexpression of a phosphoenolpyruvate carboxykinase and overexpression of an oxaloacetate decarboxylase and overexpression of a malate dehydrogenase combined with the deletion of a pyruvate kinase gene and a phosphoenolpyruvate carboxylase gene, overexpression of a phosphoenolpyruvate carboxykinase combined with overexpression of an oxaloacetate decarboxylase and overexpression of a malate dehydrogenase combined with the deletion of a pyruvate kinase gene and a phosphoenolpyruvate carboxylase gene, overexpression of a phosphoenolpyruvate synthase combined with overexpression of an oxaloacetate decarboxylase and overexpression of a malate dehydrogenase combined with the deletion of a pyruvate kinase gene and a phosphoenolpyruvate carboxylase gene.

In another exemplary embodiment, the cell is metabolically engineered by different adaptations such as overexpression of phosphoenolpyruvate synthase combined with overexpression of a phosphoenolpyruvate carboxykinase combined with the deletion of a pyruvate kinase gene and a pyruvate carboxylase gene and a phosphoenolpyruvate carboxylase gene, overexpression of phosphoenolpyruvate synthase combined with overexpression of an oxaloacetate decarboxylase combined with the deletion of a pyruvate kinase gene and a pyruvate carboxylase gene and a phosphoenolpyruvate carboxylase gene, overexpression of phosphoenolpyruvate synthase combined with overexpression of a malate dehydrogenase combined with the deletion of a pyruvate kinase gene and a pyruvate carboxylase gene and a phosphoenolpyruvate carboxylase gene, overexpression of a phosphoenolpyruvate carboxykinase combined with overexpression of an oxaloacetate decarboxylase combined with the deletion of a pyruvate kinase gene and a pyruvate carboxylase gene and a phosphoenolpyruvate carboxylase gene, overexpression of a phosphoenolpyruvate carboxykinase combined with overexpression of a malate dehydrogenase combined with the deletion of a pyruvate kinase gene and a pyruvate carboxylase gene and a phosphoenolpyruvate carboxylase gene, overexpression of an oxaloacetate decarboxylase combined with overexpression of a malate dehydrogenase combined with the deletion of a pyruvate kinase gene and a pyruvate carboxylase gene and a phosphoenolpyruvate carboxylase gene, overexpression of phosphoenolpyruvate synthase combined with overexpression of a phosphoenolpyruvate carboxykinase and overexpression of an oxaloacetate decarboxylase combined with the deletion of a pyruvate kinase gene and a pyruvate carboxylase gene and a phosphoenolpyruvate carboxylase gene, overexpression of phosphoenolpyruvate synthase combined with overexpression of a phosphoenolpyruvate carboxykinase and overexpression of a malate dehydrogenase combined with the deletion of a pyruvate kinase gene and a pyruvate carboxylase gene and a phosphoenolpyruvate carboxylase gene, overexpression of phosphoenolpyruvate synthase combined with overexpression of a phosphoenolpyruvate carboxykinase and overexpression of an oxaloacetate decarboxylase and overexpression of a malate dehydrogenase combined with the deletion of a pyruvate kinase gene and pyruvate carboxylase gene and a phosphoenolpyruvate carboxylase gene, overexpression of a phosphoenolpyruvate carboxykinase combined with overexpression of an oxaloacetate decarboxylase and overexpression of a malate dehydrogenase combined with the deletion of a pyruvate kinase gene and a pyruvate carboxylase gene and a phosphoenolpyruvate carboxylase gene, overexpression of phosphoenolpyruvate synthase combined with overexpression of an oxaloacetate decarboxylase and overexpression of a malate dehydrogenase combined with the deletion of a pyruvate kinase gene and a pyruvate carboxylase gene and a phosphoenolpyruvate carboxylase gene.

A further preferred embodiment of the method and/or cell of the present invention provides for a cell which further is modified to reduce degradation of acetyl-CoA and/or its main precursor pyruvate. This can be achieved by deleting the genes encoding for lactate dehydrogenase (EC 1.1.2.3, EC 1.1.2.4, EC 1.1.2.5, EC 1.1.1.27, or EC 1.1.1.28), and/or pyruvate carboxylase (EC 6.4.1.1) and/or the genes encoding for the glyoxylate pathway (isocitrate lyase EC 4.1.3.1 and/or malate synthase EC 2.3.3.9).

In a preferred embodiment, the cell is modified by reduced expression or activity of any one or more of a) lactate dehydrogenase (EC 1.1.2.3, EC 1.1.2.4, EC 1.1.2.5, EC 1.1.1.27, EC 1.1.1.28), b) pyruvate carboxylase (EC 6.4.1.1), c) isocitrate lyase (EC 4.1.3.1); d) malate synthase (EC 2.3.3.9).

In an alternative and/or additional further aspect of the method and/or cell of the invention, the cell is modified in the expression or activity of at least one lactate dehydrogenase like e.g., from E. coli, S. cerevisiae, H. sapiens and R. norvegicus. As such, the cell has been modified to have at least one partially or fully knocked out or mutated lactate dehydrogenase encoding gene by means generally known by the person skilled in the art resulting in at least one protein with less functional or being disabled for lactate dehydrogenase activity. In a more preferred embodiment, the cell has a full knock-out in the IdhA encoding gene resulting in a cell lacking lactate dehydrogenase activity.

Preferably or alternatively, the Krebs cycle genes can be rendered less functional by either reduced expression or point mutations such as but not limited to A258T, A162V and/or A124T in the citrate synthase enzyme coded by gltA in E. coli (as described by Quandt et al. eLife 2015; 4:e09696. DOI: 10.7554/eLife.09696).

In a preferred embodiment of the method and/or cell of present invention, the cell comprises one or more pathway(s) for monosaccharide synthesis. Said pathways for monosaccharide synthesis comprise enzymes like e.g. carboxylases, decarboxylases, isomerases, epimerases, reductases, enolases, phosphorylases, carboxykinases, kinases, phosphatases, aldolases, hydrolases, dehydrogenases, enzymes involved in the synthesis of one or more nucleoside triphosphate(s) like UTP, GTP, ATP and CTP, enzymes involved in the synthesis of any one or more nucleoside mono- or diphosphates like e.g. UMP and UDP, respectively, and enzymes involved in the synthesis of phosphoenolpyruvate (PEP).

In another and/or additional preferred embodiment of the method and/or cell of present invention, the cell comprises one or more pathway(s) for phosphorylated monosaccharide synthesis. Said pathways for phosphorylated monosaccharide synthesis comprise enzymes involved in the synthesis of one or more monosaccharide(s), one or more nucleoside mono-, di- and/or triphosphate(s) and enzymes involved in the synthesis of phosphoenolpyruvate (PEP) like e.g., but not limited to PEP synthase, carboxylases, decarboxylases, isomerases, epimerases, reductases, enolases, phosphorylases, carboxykinases, kinases, phosphatases, aldolases, hydrolases and dehydrogenases. In another and/or additional preferred embodiment of the method and/or cell of present invention, the cell comprises one or more pathways for the synthesis of one or more nucleotide-activated sugars. Said pathways for nucleotide-activated sugar synthesis comprise enzymes like e.g. PEP synthase, carboxylases, decarboxylases, isomerases, epimerases, reductases, enolases, phosphorylases, carboxykinases, kinases, phosphatases, aldolases, hydrolases, dehydrogenases, mannose-6-phosphate isomerase, phosphomannomutase, mannose-1-phosphate guanylyltransferase, GDP-mannose 4,6-dehydratase, GDP-L-fucose synthase, fucose permease, fucose kinase, fucose-1-phosphate guanylyltransferase, L-fucokinase/GDP-fucose pyrophosphorylase, L-glutamine-D-fructose-6-phosphate aminotransferase, glucosamine-6-phosphate deaminase, phosphoglucosamine mutase, N-acetylglucosamine-6-phosphate deacetylase, N-acylglucosamine 2-epimerase, UDP-N-acetylglucosamine 2-epimerase, UDP-N-acetylglucosamine 4-epimerase, N-acetylglucosamine-6P 2-epimerase, glucosamine 6-phosphate N-acetyltransferase, N-acetylglucosamine-6-phosphate phosphatase, N-acetylmannosamine-6-phosphate phosphatase, N-acetylmannosamine kinase, phosphoacetylglucosamine mutase, N-acetylglucosamine-1-phosphate uridylyltransferase, glucosamine-1-phosphate acetyltransferase, sialic acid synthase, N-acetylneuraminate lyase, N-acylneuraminate-9-phosphate synthase, N-acylneuraminate-9-phosphatase, CMP-sialic acid synthase, N-acetylgalactosamine kinase, UDP-N-acetylgalactosamine pyrophosphorylase, galactose-1-epimerase, galactokinase, glucokinase, galactose-1-phosphate uridylyltransferase, UDP-glucose 4-epimerase, glucose-1-phosphate uridylyltransferase, phosphoglucomutase and/or N-acetylglucosamine-1-phosphate uridylyltransferase.

In another and/or additional preferred embodiment, the cell comprises at least one glycosyltransferase. Preferably at least one of said glycosyltransferase is involved in the production of said compound. Such glycosyltransferase can be chosen from the list comprising fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases and fucosaminyltransferases.

Preferably, the cell is modified in the expression or activity of at least one of said glycosyltransferases, wherein preferably said modification is obtained by overexpressing an endogenous glycosyltransferase and/or introducing and expressing a homologous or heterologous glycosyltransferase.

In a preferred embodiment, one of said glycosyltransferases is a fucosyltransferase that transfers a fucose from a GDP-fucose donor to lactose in an alpha1,2 and/or alpha-1,3 linkage, thereby producing fucosyllactose and/or difucosyllactose. In an alternative preferred embodiment of the method and/or cell of the invention, the fucosyltransferase is chosen from the list comprising alpha-1,2-fucosyltransferase, alpha-1,3-fucosyltransferase, alpha-1,4-fucosyltransferase, alpha-1,3/4-fucosyltransferase and alpha-1,6-fucosyltransferase.

Alternatively or preferably, the cell comprises (i) a GDP-fucose biosynthesis pathway comprising at least one enzyme chosen from the list comprising mannose-6-phosphate isomerase, phosphomannomutase, mannose-1-phosphate guanylyltransferase, GDP-mannose 4,6-dehydratase, GDP-L-fucose synthase, fucose permease, fucose kinase, fucose-1-phosphate guanylyltransferase, L-fucokinase/GDP-fucose pyrophosphorylase; and (ii) a fucosyltransferase.

In an alternative and/or additional embodiment of the method and/or cell of the invention, one of said glycosyltransferases is a sialyltransferase that transfers a N-acetyl Neuraminic acid (sia) from a CMP-Sia donor to lactose in an alpha-2,3-, alpha-2,6- and/or alpha-2,8-linkage, thereby producing sialyllactose and/or disialyllactose. Alternatively, the sialyltransferase is chosen from the list comprising alpha-2,3-sialyltransferase, alpha-2,6-sialyltransferase, and alpha-2,8-sialyltransferase.

Alternatively or preferably, the cell comprises any one or more of (i) a sialic acid biosynthesis pathway comprising at least one enzyme chosen from the list comprising UDP-GlcNAc 2-epimerase, N-acylglucosamine 2-epimerase and sialic acid synthase; (ii) an N-acylneuraminate cytidylyltransferase; and (iii) a sialyltransferase.

In an alternative and/or additional embodiment of the method and/or cell of the invention, one of said glycosyltransferases is galactosyltransferase, preferably chosen from the list comprising beta-1,3-galactosyltransferase, N-acetylglucosamine beta-1,3-galactosyltransferase, beta-1,4-galactosyltransferase, N-acetylglucosamine beta-1,4-galactosyltransferase, alpha-1,3-galactosyltransferase and alpha-1,4-galactosyltransferase.

Alternatively or preferably, said cell comprises a galactosylation pathway comprising (i) a UDP-galactose biosynthesis pathway comprising at least one enzyme chosen from the list comprising galactose-1-epimerase, galactokinase, glucokinase, galactose-1-phosphate uridylyltransferase, UDP-glucose 4-epimerase, glucose-1-phosphate uridylyltransferase, phosphoglucomutase; and (ii) a galactosyltransferase.

In an alternative and/or additional embodiment of the method and/or cell of the invention, one of said glycosyltransferases is glucosyltransferase, preferably chosen from the list comprising alpha-glucosyltransferase, beta-1,2-glucosyltransferase, beta-1,3-glucosyltransferase and beta-1,4-glucosyltransferase.

In an alternative and/or additional embodiment of the method and/or cell of the invention, one of said glycosyltransferases is mannosyltransferase, preferably chosen from the list comprising alpha-1,2-mannosyltransferase, alpha-1,3-mannosyltransferase and alpha-1,6-mannosyltransferase.

In an alternative and/or additional embodiment of the method and/or cell of the invention, one of said glycosyltransferases is N-acetylglucosaminyltransferase, preferably chosen from the list comprising galactoside beta-1,3-N-acetylglucosaminyltransferase and beta-1,6-N-acetylglucosaminyltransferase. Alternatively or preferably, said cell comprises an N-acetylglucosaminylation pathway comprising (i) at least one enzyme chosen from the list comprising L-glutamine-D-fructose-6-phosphate aminotransferase, N-acetylglucosamine-6-phosphate deacetylase, phosphoglucosamine mutase, N-acetylglucosamine-1-phosphate uridylyltransferase/glucosamine-1-phosphate acetyltransferase, N-acetylglucosaminyltransferase; and (ii) a N-acetylglucosaminyltransferase. More preferably, the cell comprises a pathway to synthesize lacto-N-tetraose (LNT) comprising a galactoside beta-1,3-N-acetylglucosaminyltransferase and an N-acetylglucosamine beta-1,3-galactosyltransferase. Alternatively, the cell comprises a pathway to synthesize lacto-N-neotetraose (LNnT) comprising a galactoside beta-1,3-N-acetylglucosaminyltransferase and an N-acetylglucosamine beta-1,4-galactosyltransferase.

In an alternative and/or additional embodiment of the method and/or cell of the invention, one of said glycosyltransferases is N-acetylgalactosaminyltransferase, preferably alpha-1,3-N-acetylgalactosaminyltransferase.

In a further aspect of the method and/or cell of the invention, the cell is modified in the expression or activity of at least one of said glycosyltransferases. In a preferred embodiment, said glycosyltransferase is an endogenous protein of the cell with a modified expression or activity, preferably said endogenous glycosyltransferase is overexpressed; alternatively said glycosyltransferase is a heterologous protein that is heterogeneously introduced and expressed in said cell, preferably overexpressed. Said endogenous glycosyltransferase can have a modified expression in the cell which also expresses a heterologous glycosyltransferase.

According to another preferred aspect of the method and/or cell of the invention, the cell expresses at least one alpha-2,3-sialyltransferase which has alpha-2,3-sialyltransferase activity.

In a preferred embodiment of the method and/or cell of the invention, the alpha-2,3-sialyltransferase is originating from Pasteurella multocida, full length or truncated version as described in the art.

In another aspect of the method and/or cell of the invention, the alpha-2,3-sialyltransferase is involved in the production of a compound of present invention comprising a disaccharide, an oligosaccharide, Neu(n)Ac-containing glycolipid, and/or a Neu(n)Ac-containing glycoprotein. In a preferred aspect of the method and/or cell of the invention, the alpha-2,3-sialyltransferase is involved in the production of a Neu(n)Ac-containing bioproduct, wherein (n) is 4, 5, 7, 8 or 9 or a combination thereof.

In a further aspect of the method and/or cell of the invention, the cell is modified in the expression or activity of at least one of said alpha-2,3-sialyltransferases. In a preferred embodiment, said alpha-2,3-sialyltransferase is an endogenous protein of the cell with a modified expression or activity, preferably said endogenous alpha-2,3-sialyltransferase is overexpressed; alternatively said alpha-2,3-sialyltransferase is a heterologous protein that is heterogeneously introduced and expressed in said cell, preferably overexpressed. Said endogenous alpha-2,3-sialyltransferase can have a modified expression in the cell which also expresses a heterologous alpha-2,3-sialyltransferase.

According to another and/or alternative preferred aspect of the method and/or cell of the invention, the cell comprises a fucosylation pathway comprising at least one enzyme chosen from the list comprising mannose-6-phosphate isomerase, phosphomannomutase, mannose-1-phosphate guanylyltransferase, GDP-mannose 4,6-dehydratase, GDP-L-fucose synthase, fucose permease, fucose kinase, fucose-1-phosphate guanylyltransferase, L-fucokinase/GDP-fucose pyrophosphorylase, fucosyltransferase.

According to another and/or alternative preferred aspect of the method and/or cell of the invention, the cell comprises a galactosylation pathway comprising at least one enzyme chosen from the list comprising galactose-1-epimerase, galactokinase, glucokinase, galactose-1-phosphate uridylyltransferase, UDP-glucose 4-epimerase, glucose-1-phosphate uridylyltransferase, phosphoglucomutase, galactosyltransferase.

According to another and/or alternative preferred aspect of the method and/or cell of the invention, the cell comprises an N-acetylglucosaminylation pathway comprising at least one enzyme chosen from the list comprising L-glutamine-D-fructose-6-phosphate aminotransferase, N-acetylglucosamine-6-phosphate deacetylase, phosphoglucosamine mutase, N-acetylglucosamine-1-phosphate uridylyltransferase/glucosamine-1-phosphate acetyltransferase, N-acetylglucosaminyltransferase.

The present invention provides different types of cells for the production of a compound with a metabolically engineered cell.

According to one preferred aspect of the method and/or cell of the invention, the cell endogenously comprises a pathway for the production of a compound as defined herein and is metabolically engineered for enhanced synthesis of acetyl-Coenzyme A.

According to another preferred aspect of the method and/or cell of the invention, the cell is metabolically engineered i) to comprise a pathway for the production of a compound as defined herein, and ii) for enhanced synthesis of acetyl-Coenzyme A.

According to a preferred aspect of the method and/or cell of the invention, the cell comprises a pathway for production of a Neu(n)Ac-containing bioproduct. According to another preferred aspect of the method and/or cell of the invention, said pathway for production of a Neu(n)Ac-containing bioproduct comprises at least one enzyme chosen from the list comprising Neu(n)Ac synthase, N-acylglucosamine 2-epimerase, UDP-N-acetylglucosamine 2-epimerase, N-acetylmannosamine-6-phosphate 2-epimerase, bifunctional UDP-GlcNAc 2-epimerase/kinase, N-acylneuraminate-9-phosphate synthetase, phosphatase, CMP-sialic acid synthase and sialyltransferase.

In a preferred embodiment, the cell comprises a pathway for production of a Neu(n)Ac-containing bioproduct wherein said cell expresses at least one enzyme chosen from the list comprising an N-acylglucosamine 2-epimerase like is known e.g. from several species including Bacteroides ovatus, E. coli, Homo sapiens, Rattus norvegicus, a Neu(n)Ac synthase as is disclosed in present invention, a CMP sialic acid synthase like is known e.g. from Neisseria meningitidis, and a sialyltransferase including an alpha-2,3-sialyltransferase, an alpha-2,6-sialyltransferase and/or an alpha-2,8-sialyltransferase, wherein the enzymes are as defined herein. N-acyl-D-glucosamine (GlcNAc) can be added to the cell and/or can be provided by an enzyme expressed in the cell or by the mechanism of the cell. Such cell producing GlcNAc can express a phosphatase converting GlcNAc-6-phosphate into GlcNAc, like any one or more of e.g. the E. coli HAD-like phosphatase genes comprising aphA, Cof, HisB, OtsB, SurE, Yaed, YcjU, YedP, YfbT, YidA, YigB, YihX, YniC, YqaB, YrbL, AppA, Gph, SerB, YbhA, YbiV, YbjL, Yfb, YieH, YjgL, YjjG, YrfG and YbiU, PsMupP from Pseudomonas putida, ScDOG1 from S. cerevisiae and BsAraL from Bacillus subtilis as described in WO18122225. Preferably, the cell is modified to produce GlcNAc. More preferably, the cell is modified for enhanced GlcNAc production. Said modification can be any one or more chosen from the group comprising knockout of a glucosamine-6-phosphate deaminase, an N-acetylglucosamine-6-phosphate deacetylase and/or an N-acetyl-D-glucosamine kinase and over-expression of an L-glutamine-D-fructose-6-phosphate aminotransferase and/or a glucosamine 6-phosphate N-acetyltransferase.

In an alternative and/or additional preferred embodiment, the cell comprises a pathway for production of a Neu(n)Ac-containing bioproduct wherein said cell expresses at least one enzyme chosen from the list comprising a UDP-N-acetylglucosamine 2-epimerase like is known e.g. from several species including Campylobacter jejuni, E. coli, Neisseria meningitidis, Bacillus subtilis, Citrobacter rodentium, a Neu(n)Ac synthase as is disclosed in present invention, a CMP sialic acid synthase like is known e.g from Neisseria meningitidis, and a sialyltransferase including an alpha-2,3-sialyltransferase, an alpha-2,6-sialyltransferase and/or an alpha-2,8-sialyltransferase, wherein the enzymes are as defined herein. UDP-N-acetylglucosamine (UDP-GlcNAc) can be added to the cell and/or can be provided by an enzyme expressed in the cell or by the metabolism of the cell. Such cell producing a UDP-GlcNAc can express enzymes converting, e.g. GlcNAc, which is to be added to the cell, to UDP-GlcNAc. These enzymes may be any one or more enzymes chosen from the list comprising an N-acetyl-D-glucosamine kinase, an N-acetylglucosamine-6-phosphate deacetylase, a phosphoglucosamine mutase, and an N-acetylglucosamine-1-phosphate uridylyltransferase/glucosamine-1-phosphate acetyltransferase from several species including Homo sapiens, Escherichia coli. Preferably, the cell is modified to produce UDP-GlcNAc. More preferably, the cell is modified for enhanced UDP-GlcNAc production. Said modification can be any one or more chosen from the group comprising knock-out of an N-acetylglucosamine-6-phosphate deacetylase, over-expression of an L-glutamine-D-fructose-6-phosphate aminotransferase, over-expression of a phosphoglucosamine mutase, and over-expression of an N-acetylglucosamine-1-phosphate uridylyltransferase/glucosamine-1-phosphate acetyltransferase.

In an alternative and/or additional preferred embodiment, the cell comprises a pathway for production of a Neu(n)Ac-containing bioproduct wherein said cell expresses at least one enzyme chosen from the list comprising an N-acetylmannosamine-6-phosphate 2-epimerase like is known e.g. from several species including E. coli, Haemophilus influenzae, Enterobacter sp., Streptomyces sp., an N-acylneuraminate-9-phosphate synthetase, an N-acylneuraminate-9-phosphatase like is known e.g. from Candidatus magnetomorum sp. HK-1 or Bacteroides thetaiotaomicron, a Neu(n)Ac synthase as is disclosed in present invention, a CMP sialic acid synthase like is known e.g. from Neisseria meningitidis, and a sialyltransferase including an alpha-2,3-sialyltransferase, an alpha-2,6-sialyltransferase and/or an alpha-2,8-sialyltransferase, wherein the enzymes are as defined herein. N-acetyl-D-glucosamine 6-phosphate (GlcNAc-6P) can be added to the cell and/or can be provided by an enzyme expressed in the cell or by the metabolism of the cell. Such cell producing GlcNAc-6P can express an enzyme converting, e.g., GlcN6P, which is to be added to the cell, to GlcNAc-6P. This enzyme may be a glucosamine 6-phosphate N-acetyltransferase from several species including Saccharomyces cerevisiae, Kluyveromyces lactis, Homo sapiens. Preferably, the cell is modified to produce GlcNAc-6P. More preferably, the cell is modified for enhanced GlcNAc-6P production. Said modification can be any one or more chosen from the group comprising knockout of a glucosamine-6-phosphate deaminase, an N-acetylglucosamine-6-phosphate deacetylase and over-expression of an L-glutamine-D-fructose-6-phosphate aminotransferase and/or a glucosamine 6-phosphate N-acetyltransferase.

In an alternative and/or additional preferred embodiment, the cell comprises a pathway for production of a Neu(n)Ac-containing bioproduct wherein said cell expresses at least one enzyme chosen from the list comprising a bifunctional UDP-GlcNAc 2-epimerase/kinase like is known e.g. from several species including Homo sapiens, Rattus norvegicus and Mus musculus, an N-acylneuraminate-9-phosphate synthetase, an N-acylneuraminate-9-phosphatase like is known e.g. from Candidatus magnetomorum sp. HK-1 or Bacteroides thetaiotaomicron, a Neu(n)Ac synthase as is disclosed in present invention, a CMP sialic acid synthase like is known e.g. from Neisseria meningitidis, and a sialyltransferase including an alpha-2,3-sialyltransferase, an alpha-2,6-sialyltransferase and/or an alpha-2,8-sialyltransferase, wherein the enzymes are as defined herein. UDP-N-acetylglucosamine can be added to the cell and/or can be provided by an enzyme expressed in the cell or by the metabolism of the cell. Such cell producing a UDP-GlcNAc can express enzymes converting, e.g. GlcNAc, which is to be added to the cell, to UDP-GlcNAc. These enzymes may be an N-acetyl-D-glucosamine kinase, an N-acetylglucosamine-6-phosphate deacetylase, a phosphoglucosamine mutase, and an N-acetylglucosamine-1-phosphate uridyltransferase/glucosamine-1-phosphate acetyltransferase from several species including Homo sapiens, Escherichia coli. Preferably, the cell is modified to produce UDP-GlcNAc. More preferably, the cell is modified for enhanced UDP-GlcNAc production. Said modification can be any one or more chosen from the group comprising knock-out of an N-acetylglucosamine-6-phosphate deacetylase, over-expression of an L-glutamine-D-fructose-6-phosphate aminotransferase, over-expression of a phosphoglucosamine mutase, and over-expression of an N-acetylglucosamine-1-phosphate uridyltransferase/glucosamine-1-phosphate acetyltransferase.

According to another preferred aspect of the method and/or cell of the invention, the cell is using a precursor for the synthesis of the compound of present invention. Herein, the precursor is fed to the cell from the cultivation medium or the culture medium. In another preferred embodiment, the cell is producing a precursor for the synthesis of said compound of present invention.

Additionally, or alternatively, the cell used herein is optionally engineered to import a precursor or an acceptor in the cell, by the introduction and/or overexpression of a transporter able to import the respective precursor or acceptor in the cell. Such transporter is for example a membrane protein belonging to the major facilitator superfamily (MFS), the ATP-binding cassette (ABC) transporter family or the PTS system involved in the uptake of e.g., mono-, di- and/or oligosaccharides.

Additionally, or alternatively, the cell used herein is optionally metabolically engineered to produce polyisoprenoid alcohols like e.g., phosphorylated dolichol that can act as lipid carrier.

Additionally, or alternatively, the cell used herein is optionally engineered to import lactose in the cell, by the introduction and/or overexpression of a lactose permease. Said lactose permease is for example encoded by the lacY gene or the lac12 gene.

Additionally, or alternatively, the cell expresses a membrane protein that is a transporter protein involved in transport of precursors, acceptors and/or compounds as defined herein across the outer membrane of the cell wall. Preferably the cell expresses at least one nucleic acid sequence encoding a protein selected from the group comprising a lactose transporter like e.g., the LacY or lac12 permease, a glucose transporter, a galactose transporter, a transporter for a nucleotide-activated sugar like for example a transporter for UDP-GlcNAc, a transporter protein involved in transport of a compound as defined herein, like e.g., Neu(n)Ac-containing bioproduct across the outer membrane of the cell wall. Preferably the cell is transformed to comprise at least one nucleic acid sequence encoding a membrane transporter protein, preferably selected from the group comprising a siderophore exporter, a major facilitator superfamily (MFS) transporter, an ATP-binding cassette (ABC) transporter or a sugar efflux transporter.

According to another preferred aspect of the method and/or cell of the present invention, the cell is capable to synthesize N-acetylmannosamine (ManNAc), N-acetylmannosamine-6-phosphate (ManNAc-6-phosphate) and/or phosphoenolpyruvate (PEP) as described herein.

In a preferred embodiment, the cell comprises a pathway for production of a compound of present invention comprising a pathway for production of ManNAc. ManNAc can be provided by an enzyme expressed in the cell or by the mechanism of the cell. Such cell producing ManNAc can express an N-acylglucosamine 2-epimerase like is known e.g., from several species including Bacteroides ovatus, E. coli, Homo sapiens, Rattus norvegicus that converts GlcNAc into ManNAc. Alternatively, and/or additionally, the cell producing ManNAc can express a UDP-N-acetylglucosamine 2-epimerase like is known e.g. from several species including Campylobacter jejuni, E. coli, Neisseria meningitidis, Bacillus subtilis, Citrobacter rodentium that converts UDP-GlcNAc into ManNAc. GlcNAc and/or UDP-GlcNAc can be added to the cell and/or provided by an enzyme expressed in the cell or by the mechanism of the cell as described herein. In a more preferred embodiment, the cell is modified for enhanced ManNAc production. Said modification can be any one or more chosen from the group comprising knock-out of N-acetylmannosamine kinase, over-expression of N-acetylneuraminate lyase.

In another preferred embodiment, the cell comprises a pathway for production of a compound of present invention comprising a pathway for production of ManNAc-6-phosphate. ManNAc-6-phosphate can be provided by an enzyme expressed in the cell or by the mechanism of the cell. Such cell producing ManNAc-6-phosphate can express a bifunctional UDP-GlcNAc 2-epimerase/kinase like is known e.g. from several species including Homo sapiens, Rattus norvegicus and Mus musculus that converts UDP-GlcNAc into ManNAc-6-phosphate. Alternatively, and/or additionally, the cell producing ManNAc-6-phosphate can express an N-acetylmannosamine-6-phosphate 2-epimerase that converts GlcNAc-6-phosphate into ManNAc-6-phosphate. UDP-GlcNAc and/or GlcNAc-6-phosphate can be added to the cell and/or provided by an enzyme expressed in the cell or by the mechanism of the cell as described herein. In a more preferred embodiment, the cell is modified for enhanced ManNAc-6-phosphate production. Said modification can be any one or more chosen from the group comprising over-expression of N-acetylglucosamine-6-phosphate deacetylase, over-expression of N-acetyl-D-glucosamine kinase, over-expression of phosphoglucosamine mutase, over-expression of N-acetylglucosamine-1-phosphate uridyltransferase/glucosamine-1-phosphate acetyltransferase.

According to another aspect of the method and/or cell of the invention, the cell is further capable to synthesize a nucleotide-activated sugar. In a preferred embodiment of the method and/or cell of the invention, the cell is capable to synthesize one or more nucleotide-activated sugars chosen from the list comprising UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), UDP-glucuronate, UDP-galacturonate, UDP-2-acetamido-2,6-dideoxy-L-arabino-4-hexulose, UDP-2-acetamido-2,6-dideoxy-L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid (CMP-Neu5Ac), CMP-N-glycolylneuraminic acid (CMP-Neu5Gc), GDP-fucose (GDP-Fuc), GDP-rhamnose and UDP-xylose. In a more preferred embodiment of the method and/or cell of the invention, the cell is capable to synthesize at least nucleotide-activated sugar that is derived from Neu(n)Ac comprising CMP-Neu4Ac, CMP-Neu5Ac, CMP-Neu5Ac9N₃, CMP-Neu4,5Ac₂, CMP-Neu5,7Ac₂, CMP-Neu5,9Ac₂, CMP-Neu5,7(8,9)Ac₂ and CMP-Neu5Gc. In an even more preferred embodiment of the method and/or cell of the invention, the cell uses at least one of the synthesized nucleotide-activated sugars in the production of a compound of present invention like e.g. a Neu(n)Ac-containing bioproduct.

The cell used herein is optionally metabolically engineered to express the de novo synthesis of UDP-GlcNAc. UDP-GlcNAc can be provided by an enzyme expressed in the cell or by the metabolism of the cell. Such cell producing a UDP-GlcNAc can express enzymes converting, e.g. GlcNAc, which is to be added to the cell, to UDP-GlcNAc. These enzymes may be any one or more of the list comprising an N-acetyl-D-glucosamine kinase, an N-acetylglucosamine-6-phosphate deacetylase, a phosphoglucosamine mutase, and an N-acetylglucosamine-1-phosphate uridyltransferase/glucosamine-1-phosphate acetyltransferase from several species including Homo sapiens, Escherichia coli. Preferably, the cell is modified to produce UDP-GlcNAc. More preferably, the cell is modified for enhanced UDP-GlcNAc production. Said modification can be any one or more chosen from the group comprising knock-out of an N-acetylglucosamine-6-phosphate deacetylase, over-expression of an L-glutamine-D-fructose-6-phosphate aminotransferase, over-expression of a phosphoglucosamine mutase, and over-expression of an N-acetylglucosamine-1-phosphate uridylyltransferase/glucosamine-1-phosphate acetyltransferase. Additionally, or alternatively, the cell used herein is optionally metabolically engineered to express the de novo synthesis of CMP-Neu5Ac. CMP-Neu5Ac can be provided by an enzyme expressed in the cell or by the metabolism of the cell. Such cell producing CMP-Neu5Ac can express an enzyme converting, e.g., sialic acid to CMP-Neu5Ac. This enzyme may be a CMP-sialic acid synthetase, like the N-acylneuraminate cytidylyltransferase from several species including Homo sapiens, Neisseria meningitidis, and Pasteurella multocida. Preferably, the cell is modified to produce CMP-Neu5Ac. More preferably, the cell is modified for enhanced CMP-Neu5Ac production. Said modification can be any one or more chosen from the group comprising knock-out of an N-acetylglucosamine-6-phosphate deacetylase, knock-out of a glucosamine-6-phosphate deaminase, over-expression of a CMP-sialic acid synthetase, and over-expression of an N-acylglucosamine 2-epimerase encoding gene.

Additionally, or alternatively, the cell used herein is optionally metabolically engineered to express the de novo synthesis of CMP-Neu5Gc. CMP-Neu5Gc can be synthesized directly from CMP-Neu5Ac via a hydroxylation reaction performed by a vertebrate CMP-Neu5Ac hydroxylase (CMAH) enzyme. Preferably, the cell is modified to produce CMP-Neu5Gc. More preferably, the cell is modified for enhanced CMP-Neu5Gc production.

Additionally, or alternatively, the cell used herein is optionally metabolically engineered to express the de novo synthesis of GDP-fucose. GDP-fucose can be provided by an enzyme expressed in the cell or by the metabolism of the cell. Such cell producing GDP-fucose can express an enzyme converting, e.g., fucose, which is to be added to the cell, to GDP-fucose. This enzyme may be, e.g., a bifunctional fucose kinase/fucose-1-phosphate guanylyltransferase, like Fkp from Bacteroides fragilis, or the combination of one separate fucose kinase together with one separate fucose-1-phosphate guanylyltransferase like they are known from several species including Homo sapiens, Sus scrofa and Rattus norvegicus. Preferably, the cell is modified to produce GDP-fucose. More preferably, the cell is modified for enhanced GDP-fucose production. Said modification can be any one or more chosen from the group comprising knock-out of a UDP-glucose:undecaprenyl-phosphate glucose-1-phosphate transferase encoding gene, over-expression of a GDP-L-fucose synthase encoding gene, over-expression of a GDP-mannose 4,6-dehydratase encoding gene, over-expression of a mannose-1-phosphate guanylyltransferase encoding gene, over-expression of a phosphomannomutase encoding gene and over-expression of a mannose-6-phosphate isomerase encoding gene.

Additionally, or alternatively, the cell used herein is optionally metabolically engineered to express the de novo synthesis of UDP-Gal. UDP-Gal can be provided by an enzyme expressed in the cell or by the metabolism of the cell. Such cell producing UDP-Gal can express an enzyme converting, e.g. UDP-glucose, to UDP-Gal. This enzyme may be, e.g., the UDP-glucose-4-epimerase GalE like as known from several species including Homo sapiens, Escherichia coli, and Rattus norvegicus. Preferably, the cell is modified to produce UDP-Gal. More preferably, the cell is modified for enhanced UDP-Gal production. Said modification can be any one or more chosen from the group comprising knock-out of a bifunctional 5′-nucleotidase/UDP-sugar hydrolase encoding gene, knock-out of a galactose-1-phosphate uridylyltransferase encoding gene and over-expression of a UDP-glucose-4-epimerase encoding gene. Additionally, or alternatively, the cell used herein is optionally metabolically engineered to express the de novo synthesis of UDP-GalNAc. UDP-GalNAc can be synthesized from UDP-GlcNAc by the action of a single-step reaction using a UDP-N-acetylglucosamine 4-epimerase like e.g. wbgU from Plesiomonas shigelloides, gne from Yersinia enterocolitica or wbpP from Pseudomonas aeruginosa serotype O6. Preferably, the cell is modified to produce UDP-GalNAc. More preferably, the cell is modified for enhanced UDP-GalNAc production.

Additionally, or alternatively, the cell used herein is optionally metabolically engineered to express the de novo synthesis of UDP-ManNAc. UDP-ManNAc can be synthesized directly from UDP-GlcNAc via an epimerization reaction performed by a UDP-GlcNAc 2-epimerase (like e.g. cap5P from Staphylococcus aureus, RffE from E. coli, Cps19fK from S. pneumoniae, and RfbC from S. enterica). Preferably, the cell is modified to produce UDP-ManNAc. More preferably, the cell is modified for enhanced UDP-ManNAc production.

According to another preferred aspect of the method and/or cell of the invention, the cell comprises a lower or reduced expression and/or abolished, impaired, reduced or delayed activity of any one or more of the proteins comprising beta-galactosidase, galactoside O-acetyltransferase, N-acetylglucosamine-6-phosphate deacetylase, glucosamine-6-phosphate deaminase, N-acetylglucosamine repressor, ribonucleotide monophosphatase, EIICBA-Nag, UDP-glucose:undecaprenyl-phosphate glucose-1-phosphate transferase, L-fuculokinase, L-fucose isomerase, N-acetylneuraminate lyase, N-acetylmannosamine kinase, N-acetylmannosamine-6-phosphate 2-epimerase, EIIAB-Man, EIIC-Man, EIID-Man, ushA, galactose-1-phosphate uridylyltransferase, glucose-1-phosphate adenylyltransferase, glucose-1-phosphatase, ATP-dependent 6-phosphofructokinase isozyme 1, ATP-dependent 6-phosphofructokinase isozyme 2, glucose-6-phosphate isomerase, aerobic respiration control protein, transcriptional repressor lclR, lon protease, glucose-specific translocating phosphotransferase enzyme IIBC component ptsG, glucose-specific translocating phosphotransferase (PTS) enzyme IIBC component malX, enzyme IIA^{Glc}, beta-glucoside specific PTS enzyme II, fructose-specific PTS multiphosphoryl transfer protein FruA and FruB, ethanol dehydrogenase aldehyde dehydrogenase, pyruvate-formate lyase, acetate kinase, phosphoacyltransferase, phosphate acetyltransferase, pyruvate decarboxylase.

According to another preferred aspect of the method and/or cell of the invention, the cell comprises a catabolic pathway for selected mono-, di- or oligosaccharides which is at least partially inactivated, the mono-, di-, or oligosaccharides being involved in and/or required for the synthesis of the compound as defined herein.

According to another preferred aspect of the method and/or cell of the invention, the cell produces 30 g/L or more of a compound as defined herein in the whole broth and/or supernatant. In a more preferred embodiment, the compound produced in the whole broth and/or supernatant has a purity of at least 80% measured on the total amount of the compound of present invention and its precursor produced by the cell in the whole broth and/or supernatant, respectively.

According to another aspect of the method and/or cell of the invention, the compound of present invention is chosen from the list comprising disaccharide and oligosaccharide, both as defined herein. In a preferred embodiment, the oligosaccharide is chosen from the list comprising a milk oligosaccharide, O-antigen, enterobacterial common antigen (ECA), the oligosaccharide repeats present in capsular polysaccharides, peptidoglycan, amino-sugars, Lewis-type antigen oligosaccharide and antigens of the human ABO blood group system. In a more preferred embodiment, the milk oligosaccharide is a mammalian milk oligosaccharide. In an even more preferred embodiment, the milk oligosaccharide is a human milk oligosaccharide.

According to another aspect of the method and/or cell of the invention, the Neu(n)Ac-containing bioproduct is chosen from the list comprising sialic acid, a disaccharide, an oligosaccharide or a sialylated compound comprising Neu5Ac, a Neu(n)Ac-containing glycolipid and a Neu(n)Ac-containing glycoprotein. In a preferred embodiment, the oligosaccharide is chosen from the list comprising a milk oligosaccharide, O-antigen, the oligosaccharide repeats present in capsular polysaccharides, peptidoglycan, amino-sugars and Lewis-type antigen. In another preferred embodiment, the oligosaccharide is a non-charged (neutral) oligosaccharide, a fucosylated oligosaccharide and/or acidic oligosaccharide.

According to another aspect of the method and/or cell of the invention, the cell is capable to synthesize a mixture of compounds as defined herein, preferably a mixture of oligosaccharides. In an alternative and/or additional aspect, the cell is capable to synthesize a mixture of di- and oligosaccharides, alternatively, the cell is capable to synthesize a mixture of sialic acid, di- and/or oligosaccharides.

In a specific exemplary embodiment, the method of the invention provides the production of a compound in high yield. The method comprises the step of culturing or fermenting, in an aqueous culture or fermentation medium containing lactose, a metabolically engineered cell, preferably an E. coli, more preferably an E. coli cell modified by knocking-out the genes LacZ and nagB genes. Even more preferably, additionally the E. coli lacY gene, a fructose kinase gene (frk) originating from Zymomonas mobilis and a sucrose phosphorylase (SP) originating from Bifidobacterium adolescentis and a sucrose permease (cscB) from Escherichia coli can be knocked in into the genome and expressed constitutively. Preferably, the constitutive promoters originate from the promoter library described by De Mey et al. (BMC Biotechnology, 2007) and Mutalik et al. (Nat. Methods 2013, No. 10, 354-360). The cell furthermore comprises an overexpression or heterologous expression of any one of the enzymes as described herein to enhance acetyl-Coenzyme A synthesis.

In a further preferred embodiment, the cell described herein is using a split metabolism having a production pathway and a biomass pathway as described in WO2012/007481, which is herein incorporated by reference. Said organism can, for example, be metabolically engineered to accumulate fructose-6-phosphate by altering the genes selected from the phosphoglucoisomerase gene, phosphofructokinase gene, fructose-6-phosphate aldolase gene, fructose isomerase gene, and/or fructose:PEP phosphotransferase gene.

Another preferred aspect of the invention provides for a method and a cell wherein a compound of present invention is produced in and/or by a microorganism chosen from the list consisting of a bacterium, fungus or yeast. The latter bacterium preferably belongs to the phylum of the Proteobacteria or the phylum of the Firmicutes or the phylum of the Cyanobacteria or the phylum Deinococcus-Thermus. The latter bacterium belonging to the phylum Proteobacteria belongs preferably to the family Enterobacteriaceae, preferably to the species Escherichia coli. The latter bacterium preferably relates to any strain belonging to the species Escherichia coli such as but not limited to Escherichia coli B, Escherichia coli C, Escherichia coli W, Escherichia coli K12, Escherichia coli Nissle. More specifically, the latter term relates to cultivated Escherichia coli strains—designated as E. coli K12 strains—which are well-adapted to the laboratory environment, and, unlike wild type strains, have lost their ability to thrive in the intestine. Well-known examples of the E. coli K12 strains are K12 Wild type, W3110, MG1655, M182, MC1000, MC1060, MC1061, MC4100, JM101, NZN111 and AA200. Hence, preferably the present invention specifically relates to a mutated and/or transformed Escherichia coli strain as indicated above wherein said E. coli strain is a K12 strain. More specifically, the present invention relates to a mutated and/or transformed Escherichia coli strain as indicated above wherein said K12 strain is E. coli MG1655. The latter bacterium belonging to the phylum Firmicutes belongs preferably to the Bacilli, preferably from the species Bacillus. The latter fungus belongs preferably to the genus Rhizopus, Dictyostelium, Penicillium, Mucor or Aspergillus. The latter yeast preferably belongs to the phylum of the Ascomycota or the phylum of the Basidiomycota or the phylum of the Deuteromycota or the phylum of the Zygomycetes. The latter yeast belongs preferably to the genus Saccharomyces, Zygosaccharomyces, Pichia, Komagataella, Hansenula, Yarrowia, Starmerella, Kluyveromyces or Debaromyces.

In an alternative aspect, the cell is chosen from the list comprising a plant, animal, or protozoan cell. Plant cells include cells of flowering and non-flowering plants, as well as algal cells, for example Chlamydomonas, Chlorella, etc. Preferably, said plant is a tobacco, alfalfa, rice, tomato, cotton, rapeseed, soy, maize, or corn plant. The latter animal cell is preferably derived from non-human mammals (e.g. cattle, buffalo, pig, sheep, mouse, rat), birds (e.g. chicken, duck, ostrich, turkey, pheasant), fish (e.g. swordfish, salmon, tuna, sea bass, trout, catfish), invertebrates (e.g. lobster, crab, shrimp, clams, oyster, mussel, sea urchin), reptiles (e.g. snake, alligator, turtle), amphibians (e.g. frogs) or insects (e.g. fly, nematode) or is a metabolically engineered cell line derived from human cells excluding embryonic stem cells. Both human and non-human mammalian cells are preferably chosen from the list comprising an epithelial cell like e.g., a mammary stem cell, mammary epithelial cell, an embryonic kidney cell, a fibroblast cell, a COS cell, a Chinese hamster ovary (CHO) cell, a murine myeloma cell like e.g., an N20, SP2/0 or YB2/0 cell, an NIH-3T3 cell, a non-mammary adult stem cell or derivatives thereof such as described in WO21067641. The latter insect cell is preferably derived from Spodoptera frugiperda like e.g., Sf9 or Sf21 cells, Bombyx mori, Mamestra brassicae, Trichoplusia ni like e.g., BTI-TN-5B1-4 cells or Drosophila melanogaster like e.g., Drosophila S2 cells. The latter protozoan cell preferably is a Leishmania tarentolae cell.

Another aspect provides for a cell to be stably cultured in a medium, wherein said medium can be any type of growth medium comprising minimal medium, complex medium or growth medium enriched in certain compounds like for example but not limited to vitamins, trace elements, amino acids.

The cell as used herein is capable to grow on a monosaccharide, disaccharide, oligosaccharide, polysaccharide, polyol, glycerol, a complex medium or a mixture thereof like e.g., a mixed feedstock, preferably a mixed monosaccharide feedstock like e.g., hydrolysed sucrose as the main carbon source. With the term main is meant the most important carbon source for the cell for the production of the compound of interest, biomass formation, carbon dioxide and/or by-products formation (such as acids and/or alcohols, such as acetate, lactate, and/or ethanol), i.e. 20, 30, 40, 50, 60, 70, 75, 80, 85, 90, 95, 98, 99% of all the required carbon is derived from the above-indicated carbon source. In one embodiment of the invention, said carbon source is the sole carbon source for said organism, i.e. 100% of all the required carbon is derived from the above-indicated carbon source. Common main carbon sources comprise but are not limited to glucose, glycerol, fructose, sucrose, maltose, lactose, arabinose, malto-oligosaccharides, maltotriose, sorbitol, xylose, rhamnose, galactose, mannose, methanol, ethanol, trehalose, starch, cellulose, hemi-cellulose, molasses, corn-steep liquor, high-fructose syrup, acetate, citrate, lactate and pyruvate. With the term complex medium is meant a medium for which the exact constitution is not determined. Examples are molasses, corn steep liquor, peptone, tryptone or yeast extract. As used herein, a precursor as defined herein cannot be used as a carbon source for the production of the compound of present invention.

In a further preferred aspect, the method for the production of a compound as defined herein comprises at least one of the following steps:
i) Use of a culture medium comprising at least one precursor and/or acceptor for the production of said compound, and/or
ii) Adding to the culture medium at least one precursor and/or acceptor feed for the production of said compound.

In another and/or additional further preferred aspect, the method for the production of a compound as defined herein comprises at least one of the following steps:
i) Use of a culture medium comprising at least one precursor and/or acceptor;
ii) Adding to the culture medium in a reactor or incubator at least one precursor and/or acceptor feed wherein the total reactor or incubator volume ranges from 250 mL (millilitre) to 10,000 m³ (cubic meter), preferably in a continuous manner, and preferably so that the final volume of the culture medium is not more than three-fold, preferably not more than two-fold, more preferably less than 2-fold of the volume of the culture medium before the addition of said precursor and/or acceptor feed;
iii) Adding to the culture medium in a reactor or incubator at least one precursor and/or acceptor feed wherein the total reactor or incubator volume ranges from 250 mL (millilitre) to 10,000 m³ (cubic meter), preferably in a continuous manner, and preferably so that the final volume of the culture medium is not more than three-fold, preferably not more than two-fold, more preferably less than 2-fold of the volume of the culture medium before the addition of said precursor and/or acceptor feed and wherein preferably, the pH of said precursor and/or acceptor feed is set between 2.0 and 10.0, preferably between 3 and 7, and wherein preferably, the temperature of said precursor and/or acceptor feed is kept between 20° C. and 80° C.
iv) Adding at least one precursor and/or acceptor feed in a continuous manner to the culture medium over the course of 1 day, 2 days, 3 days, 4 days, 5 days by means of a feeding solution;
v) Adding at least one precursor and/or acceptor feed in a continuous manner to the culture medium over the course of 1 day, 2 days, 3 days, 4 days, 5 days by means of a feeding solution and wherein preferably, the pH of said feeding solution is set between 2.0 and 10.0, preferably between 3 and 7, and wherein preferably, the temperature of said feeding solution is kept between 20° C. and 80° C.;
   said method resulting in a compound with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of said culture medium.

In another and/or additional further preferred aspect, the method for the production of a compound as described herein comprises at least one of the following steps:
i) Use of a culture medium comprising at least one precursor and/or acceptor;
ii) Adding to the culture medium in a reactor or incubator at least one precursor and/or acceptor in one pulse or in a discontinuous (pulsed) manner wherein the total reactor or incubator volume ranges from 250 mL (millilitre) to 10,000 m³ (cubic meter), preferably so that the final volume of the culture medium is not more than three-fold, preferably not more than two-fold, more preferably less than 2-fold of the volume of the culture medium before the addition of said precursor and/or acceptor feed pulse(s);
iii) Adding to the culture medium in a reactor or incubator at least one precursor and/or acceptor in one pulse or in a discontinuous (pulsed) manner wherein the total reactor or incubator volume ranges from 250 mL (millilitre) to 10,000 m³ (cubic meter), preferably so that the final volume of the culture medium is not more than three-fold, preferably not more than two-fold, more preferably less than 2-fold of the volume of the culture medium before the addition of said precursor and/or acceptor feed pulse(s) and wherein preferably, the pH of said feed pulse(s) is set between 2.0 and 10.0, preferably between 3 and 7, and wherein preferably, the temperature of said feed pulse(s) is kept between 20° C. and 80° C.;
iv) Adding at least one precursor and/or acceptor feed in a discontinuous (pulsed) manner to the culture medium over the course of 5 minutes, 10 minutes, 30 minutes, 1 hour, 2 hours, 4 hours, 10 hours, 12 hours, 1 day, 2 days, 3 days, 4 days, 5 days by means of a feeding solution;
v) Adding at least one precursor and/or acceptor feed in a discontinuous (pulsed) manner to the culture medium over the course of 5 minutes, 10 minutes, 30 minutes, 1 hour, 2 hours, 4 hours, 10 hours, 12 hours, 1 day, 2 days, 3 days, 4 days, 5 days by means of a feeding solution and wherein preferably, the pH of said feeding solution is set between 2.0 and 10.0, preferably between 3 and 7, and wherein preferably, the temperature of said feeding solution is kept between 20° C. and 80° C.;
   said method resulting in a compound with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of said culture medium.

In another and/or additional further preferred aspect, the method for the production of a compound as described herein comprises at least one of the following steps:
i) Use of a culture medium comprising at least 50, more preferably at least 75, more preferably at least 100, more preferably at least 120, more preferably at least 150 grams of precursor per litre of initial reactor or incubator volume wherein the total reactor or incubator volume ranges from 250 mL to 10,000 m³ (cubic meter);
ii) Use of a culture medium comprising at least 50, more preferably at least 75, more preferably at least 100, more preferably at least 120, more preferably at least 150 grams of acceptor per litre of initial reactor or incubator volume wherein the total reactor or incubator volume ranges from 250 mL to 10,000 m³ (cubic meter);
iii) Adding to the culture medium in a reactor or incubator a precursor feed comprising at least 50, more preferably at least 75, more preferably at least 100, more preferably at least 120, more preferably at least 150 grams of precursor per litre of initial reactor or incubator volume wherein the total reactor or incubator volume ranges from 250 mL to 10,000 m³ (cubic meter), preferably in a continuous manner, and preferably so that the final volume of the culture medium is not more than three-fold, preferably not more than two-fold, more preferably less than 2-fold of the volume of the culture medium before the addition of said precursor feed;
iv) Adding to the culture medium in a reactor or incubator an acceptor feed comprising at least 50, more preferably at least 75, more preferably at least 100, more preferably at least 120, more preferably at least 150 grams of acceptor per litre of initial reactor or incubator volume wherein the total reactor or incubator volume ranges from 250 mL to 10,000 m³ (cubic meter), preferably in a continuous manner, and preferably so that the final volume of the culture medium is not more than three-fold, preferably not more than two-fold, more preferably less than 2-fold of the volume of the culture medium before the addition of said acceptor feed;
v) Adding to the culture medium in a reactor or incubator a precursor feed comprising at least 50, more preferably at least 75, more preferably at least 100, more preferably at least 120, more preferably at least 150 grams of precursor per litre of initial reactor or incubator volume wherein the total reactor or incubator volume ranges from 250 mL to 10,000 m³ (cubic meter), preferably in a continuous manner, and preferably so that the final volume of the culture medium is not more than three-fold, preferably not more than two-fold, more preferably less than 2-fold of the volume of the culture medium before the addition of said precursor feed and wherein preferably, the pH of said precursor feed is set between 2.0 and 10.0 and wherein preferably, the temperature of said precursor feed is kept between 20° C. and 80° C.;
vi) Adding to the culture medium in a reactor or incubator an acceptor feed comprising at least 50, more preferably at least 75, more preferably at least 100, more preferably at least 120, more preferably at least 150 grams of acceptor per litre of initial reactor or incubator volume wherein the total reactor or incubator volume ranges from 250 mL to 10,000 m³ (cubic meter), preferably in a continuous manner, and preferably so that the final volume of the culture medium is not more than three-fold, preferably not more than two-fold, more preferably less than 2-fold of the volume of the culture medium before the addition of said acceptor feed and wherein preferably, the pH of said acceptor feed is set between 2.0 and 10.0 and wherein preferably, the temperature of said acceptor feed is kept between 20° C. and 80° C.;
vii) Adding a precursor and/or acceptor feed in a continuous manner to the culture medium over the course of 1 day, 2 days, 3 days, 4 days, 5 days by means of a feeding solution;
viii) Adding a precursor feed in a continuous manner to the culture medium over the course of 1 day, 2 days, 3 days, 4 days, 5 days by means of a precursor feeding solution and wherein the concentration of said precursor feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L and wherein preferably, the pH of said precursor feeding solution is set between 2.0 and 10.0 and wherein preferably, the temperature of said precursor feeding solution is kept between 20° C. and 80° C.;
ix) Adding an acceptor feed in a continuous manner to the culture medium over the course of 1 day, 2 days, 3 days, 4 days, 5 days by means of an acceptor feeding solution and wherein the concentration of said acceptor feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L and wherein preferably, the pH of said acceptor feeding solution is set between 2.0 and 10.0 and wherein preferably, the temperature of said acceptor feeding solution is kept between 20° C. and 80° C.;
said method resulting in a compound with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of said culture medium.

In another and/or additional further preferred aspect, the method for the production of a compound as described herein comprises at least one of the following steps:
i) Use of a culture medium comprising at least 50, more preferably at least 75, more preferably at least 100, more preferably at least 120, more preferably at least 150 grams of precursor per litre of initial reactor or incubator volume wherein the total reactor or incubator volume ranges from 250 mL to 10,000 m³ (cubic meter);
ii) Use of a culture medium comprising at least 50, more preferably at least 75, more preferably at least 100, more preferably at least 120, more preferably at least 150 grams of acceptor per litre of initial reactor or incubator volume wherein the total reactor or incubator volume ranges from 250 mL to 10,000 m³ (cubic meter);
iii) Adding to the culture medium in a reactor or incubator at least one precursor in one pulse or in a discontinuous (pulsed) manner comprising at least 50, more preferably at least 75, more preferably at least 100, more preferably at least 120, more preferably at least 150 grams of precursor per litre of initial reactor or incubator volume wherein the total reactor or incubator volume ranges from 250 mL to 10,000 m³ (cubic meter), and preferably so that the final volume of the culture medium is not more than three-fold, preferably not more than two-fold, more preferably less than 2-fold of the volume of the culture medium before the addition of said precursor feed pulse(s);
iv) Adding to the culture medium in a reactor or incubator at least one acceptor in one pulse or in a discontinuous (pulsed) manner comprising at least 50, more preferably at least 75, more preferably at least 100, more preferably at least 120, more preferably at least 150 grams of acceptor per litre of initial reactor or incubator volume wherein the total reactor or incubator volume ranges from 250 mL to 10,000 m³ (cubic meter), and preferably so that the final volume of the culture medium is not more than three-fold, preferably not more than two-fold, more preferably less than 2-fold of the volume of the culture medium before the addition of said acceptor feed pulse(s);
v) Adding to the culture medium in a reactor or incubator at least one precursor in one pulse or in a discontinuous (pulsed) manner comprising at least 50, more preferably at least 75, more preferably at least 100, more preferably at least 120, more preferably at least 150 grams of precursor per litre of initial reactor or incubator volume wherein the total reactor or incubator volume ranges from 250 mL to 10,000 m³ (cubic meter), and preferably so that the final volume of the culture medium is not more than three-fold, preferably not more than two-fold, more preferably less than 2-fold of the volume of the culture medium before the addition of said precursor feed pulse(s) and wherein preferably, the pH of said precursor feed pulse(s) is set between 2.0 and 10.0 and wherein preferably, the temperature of said precursor feed pulse(s) is kept between 20° C. and 80° C.;
vi) Adding to the culture medium in a reactor or incubator at least one acceptor in one pulse or in a discontinuous (pulsed) manner comprising at least 50, more preferably at least 75, more preferably at least 100, more preferably at least 120, more preferably at least 150 grams of acceptor per litre of initial reactor or incubator volume wherein the total reactor or incubator volume ranges from 250 mL to 10,000 m³ (cubic meter), and preferably so that the final volume of the culture medium is not more than three-fold, preferably not more than two-fold, more preferably less than 2-fold of the volume of the culture medium before the addition of said acceptor feed pulse(s) and wherein preferably, the pH of said acceptor feed pulse(s) is set between 2.0 and 10.0 and wherein preferably, the temperature of said acceptor feed pulse(s) is kept between 20° C. and 80° C.;
vii) Adding at least one precursor and/or acceptor feed in a discontinuous (pulsed) manner to the culture medium over the course of 5 minutes, 10 minutes, 30 minutes, 1 hour, 2 hours, 4 hours, 10 hours, 12 hours, 1 day, 2 days, 3 days, 4 days, 5 days by means of a discontinuous (pulsed) feeding solution;
viii) Adding a precursor feed in a discontinuous (pulsed) manner to the culture medium over the course of 5 minutes, 10 minutes, 30 minutes, 1 hour, 2 hours, 4 hours, 10 hours, 12 hours, 1 day, 2 days, 3 days, 4 days, 5 days by means of a discontinuous (pulsed) precursor feeding solution and wherein the concentration of said discontinuous (pulsed) precursor feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L and wherein preferably, the pH of said discontinuous (pulsed) precursor feeding solution is set between 2.0 and 10.0 and wherein preferably, the temperature of said discontinuous (pulsed) precursor feeding solution is kept between 20° C. and 80° C.;
ix) Adding an acceptor feed in a discontinuous (pulsed) manner to the culture medium over the course of 5 minutes, 10 minutes, 30 minutes, 1 hour, 2 hours, 4 hours, 10 hours, 12 hours, 1 day, 2 days, 3 days, 4 days, 5 days by means of a discontinuous (pulsed) acceptor feeding solution and wherein the concentration of said discontinuous (pulsed) acceptor feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L and wherein preferably, the pH of said discontinuous (pulsed) acceptor feeding solution is set between 2.0 and 10.0 and wherein preferably, the temperature of said discontinuous (pulsed) acceptor feeding solution is kept between 20° C. and 80° C.;
said method resulting in a compound with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of said culture medium.

In a further, more preferred aspect, the method for the production of a compound as described herein comprises at least one of the following steps:
i) Use of a culture medium comprising at least 50, more preferably at least 75, more preferably at least 100, more preferably at least 120, more preferably at least 150 grams of lactose per litre of initial reactor or incubator volume wherein the total reactor or incubator volume ranges from 250 mL to 10,000 m³ (cubic meter);
ii) Adding to the culture medium in a reactor or incubator a lactose feed comprising at least 50, more preferably at least 75, more preferably at least 100, more preferably at least 120, more preferably at least 150 gram of lactose per litre of initial reactor or incubator volume wherein the total reactor or incubator volume ranges from 250 mL (millilitre) to 10,000 m³ (cubic meter), preferably in a continuous manner, and preferably so that the final volume of the culture medium is not more than three-fold, preferably not more than two-fold, more preferably less than 2-fold of the volume of the culture medium before the addition of said lactose feed;
iii) Adding to the culture medium in a reactor or incubator a lactose feed comprising at least 50, more preferably at least 75, more preferably at least 100, more preferably at least 120, more preferably at least 150 gram of lactose per litre of initial reactor or incubator volume wherein the total reactor or incubator volume ranges from 250 mL (millilitre) to 10,000 m³ (cubic meter), preferably in a continuous manner, and preferably so that the final volume of the culture medium is not more than three-fold, preferably not more than two-fold, more preferably less than 2-fold of the volume of the culture medium before the addition of said lactose feed and wherein preferably, the pH of said lactose feed is set between 2.0 and 10.0 and wherein preferably, the temperature of said lactose feed is kept between 20° C. and 80° C.;
iv) Adding a lactose feed in a continuous manner to the culture medium over the course of 1 day, 2 days, 3 days, 4 days, 5 days by means of a feeding solution;
v) Adding a lactose feed in a continuous manner to the culture medium over the course of 1 day, 2 days, 3 days, 4 days, 5 days by means of a lactose feeding solution and wherein the concentration of said lactose feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably the pH of said solution is set between 2.0 and 10.0, preferably between 3 and 7, and wherein preferably the temperature of said lactose feeding solution is kept between 20° C. and 80° C.;
   said method resulting in a compound of present invention, e.g. a Neu(n)Ac-modified lactose or Neu(n)Ac-modified lactose containing oligosaccharide, with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of said culture medium.

Preferably the lactose feed is accomplished by adding lactose from the beginning of the cultivation in a concentration of at least 5 mM, preferably in a concentration of 30, 40, 50, 60, 70, 80, 90, 100, 150 mM, more preferably in a concentration >300 mM.

In another aspect the lactose feed is accomplished by adding lactose to the culture medium in a concentration, such that throughout the production phase of the cultivation a lactose concentration of at least 5 mM, preferably 10 mM or 30 mM is obtained.

In a further embodiment of the methods described herein the cells are cultivated for at least about 60, 80, 100, or about 120 hours or in a continuous manner.

In a preferred embodiment, a carbon and energy source, preferably glucose, glycerol, fructose, maltose, arabinose, maltodextrines, malto-oligosaccharides, maltotriose, sorbitol, xylose, rhamnose, sucrose, galactose, mannose, methanol, ethanol, trehalose, starch, cellulose, hemi-cellulose, polyols, corn-steep liquor, high-fructose syrup, succinate, malate, acetate, citrate, lactate and pyruvate, is also added, preferably continuously to the culture medium, preferably with the precursor and/or acceptor.

In another and/or additional preferred embodiment, a carbon source is provided, preferably sucrose, in the culture medium for 3 or more days, preferably up to 7 days; and/or provided, in the culture medium, at least 100, advantageously at least 105, more advantageously at least 110, even more advantageously at least 120 grams of sucrose per litre of initial culture volume in a continuous manner, so that the final volume of the culture medium is not more than three-fold, advantageously not more than two-fold, more advantageously less than two-fold of the volume of the culturing medium before the culturing.

In another preferred embodiment of the method of present invention, the cell uses at least one precursor for the synthesis of a compound of present invention. In a more preferred embodiment, the cell uses two or more precursors for the synthesis of a compound of present invention.

In another preferred embodiment of the method of present invention, the culture medium contains at least one molecule selected from the group comprising lactose, galactose, sialic acid, fucose, GlcNAc, GalNAc, lacto-N-biose (LNB), N-acetyllactosamine (LacNAc).

Preferably, when performing the method as described herein, a first phase of exponential cell growth is provided by adding a carbon source, preferably glucose or sucrose, to the culture medium before the precursor and/or acceptor, preferably lactose, is added to the culture medium in a second phase.

In an alternative preferable embodiment, in the method as described herein, the precursor and/or acceptor, preferably the lactose, is added already in the first phase of exponential growth together with the carbon-based substrate.

According to the present invention, the methods as described herein preferably comprise a step of separating the compound of present invention from said cultivation.

The terms “separating from said cultivation” means harvesting, collecting, or retrieving said compound from the cell and/or the medium of its growth.

The compound can be separated in a conventional manner from the aqueous culture medium, in which the cell was grown. In case said compound is still present in the cells producing the compound, conventional manners to free or to extract said compound out of the cells can be used, such as cell destruction using high pH, heat shock, sonication, French press, homogenization, enzymatic hydrolysis, chemical hydrolysis, solvent hydrolysis, detergent, hydrolysis, . . . . The culture medium and/or cell extract together and separately can then be further used for separating said compound.

This preferably involves clarifying said compound to remove suspended particulates and contaminants, particularly cells, cell components, insoluble metabolites and debris produced by culturing the metabolically engineered cell. In this step, said compound can be clarified in a conventional manner. Preferably, said compound is clarified by centrifugation, flocculation, decantation and/or filtration. Another step of separating compound preferably involves removing substantially all the eventually remaining proteins, peptides, amino acids, RNA, DNA, endotoxins and glycolipids that could interfere with the subsequent separation step, from said compound, preferably after it has been clarified. In this step, remaining proteins and related impurities can be removed from said compound in a conventional manner. Preferably, remaining proteins, salts, by-products, colour, endotoxins and other related impurities are removed from said compound by ultrafiltration, nanofiltration, two-phase partitioning, reverse osmosis, microfiltration, activated charcoal or carbon treatment, treatment with non-ionic surfactants, enzymatic digestion, tangential flow high-performance filtration, tangential flow ultrafiltration, electrophoresis (e.g. using slab-polyacrylamide or sodium dodecyl sulphate-polyacrylamide gel electrophoresis (PAGE)), affinity chromatography (using affinity ligands including e.g. DEAE-Sepharose, poly-L-lysine and polymyxin-B, endotoxin-selective adsorber matrices), ion exchange chromatography (such as but not limited to cation exchange, anion exchange, mixed bed ion exchange, inside-out ligand attachment), hydrophobic interaction chromatography and/or gel filtration (i.e., size exclusion chromatography), particularly by chromatography, more particularly by ion exchange chromatography or hydrophobic interaction chromatography or ligand exchange chromatography or electrodialysis. With the exception of size exclusion chromatography, remaining proteins and related impurities are retained by a chromatography medium or a selected membrane.

In a further preferred embodiment, the methods as described herein also provide for a further purification of the compound of present invention. A further purification of said compound may be accomplished, for example, by use of (activated) charcoal or carbon, nanofiltration, ultrafiltration, electrophoresis, enzymatic treatment or ion exchange, temperature adjustment, pH adjustment or pH adjustment with an alkaline or acidic solution to remove any remaining DNA, protein, LPS, endotoxins, or other impurity. Alcohols, such as ethanol, and aqueous alcohol mixtures can also be used. Another purification step is accomplished by crystallization, evaporation or precipitation of said compound. Another purification step is to dry, e.g. spray dry, lyophilize, spray freeze dry, freeze spray dry, band dry, belt dry, vacuum band dry, vacuum belt dry, drum dry, roller dry, vacuum drum dry or vacuum roller dry the produced compound.

In an exemplary embodiment, the separation and purification of the compound is made in a process, comprising the following steps in any order:
a) contacting the cultivation or a clarified version thereof with a nanofiltration membrane with a molecular weight cut-off (MWCO) of 600-3500 Da ensuring the retention of the produced Neu(n)Ac-containing compound and allowing at least a part of the proteins, salts, by-products, colour and other related impurities to pass,
b) conducting a diafiltration process on the retentate from step a), using said membrane, with an aqueous solution of an inorganic electrolyte, followed by optional diafiltration with pure water to remove excess of the electrolyte,
c) and collecting the retentate enriched in said compound in the form of a salt from the cation of said electrolyte.

In an alternative exemplary embodiment, the separation and purification of said compound is made in a process, comprising the following steps in any order: subjecting the cultivation or a clarified version thereof to two membrane filtration steps using different membranes, wherein
one membrane has a molecular weight cut-off of between about 300 to about 500 Dalton, and
the other membrane as a molecular weight cut-off of between about 600 to about 800 Dalton.

In an alternative exemplary embodiment, the separation and purification of said compound is made in a process, comprising treating the cultivation or a clarified version thereof with a strong cation exchange resin in H+-form in a step and with a weak anion exchange resin in free base form, in another step, wherein said steps can be performed in any order.

In an alternative exemplary embodiment, the separation and purification of said compound is made in the following way. The cultivation comprising the produced compound, biomass, medium components and contaminants is applied to the following purification steps:
i) separation of biomass from the cultivation,
ii) cationic ion exchanger treatment for the removal of positively charged material,
iii) anionic ion exchanger treatment for the removal of negatively charged material,
iv) nanofiltration step and/or electrodialysis step,
wherein a purified solution comprising the produced compound at a purity of greater than or equal to 80 percent is provided. Optionally the purified solution is dried by any one or more drying steps chosen from the list comprising spray drying, lyophilization, spray freeze drying, freeze spray drying, band drying, belt drying, vacuum band drying, vacuum belt drying, drum drying, roller drying, vacuum drum drying and vacuum roller drying.

In an alternative exemplary embodiment, the separation and purification of the compound is made in a process, comprising the following steps in any order: enzymatic treatment of the cultivation; removal of the biomass from the cultivation; ultrafiltration; nanofiltration; and a column chromatography step. Preferably such column chromatography is a single column or a multiple column. Further preferably the column chromatography step is simulated moving bed chromatography. Such simulated moving bed chromatography preferably comprises i) at least 4 columns, wherein at least one column comprises a weak or strong cation exchange resin; and/or ii) four zones I, II, III and IV with different flow rates; and/or iii) an eluent comprising water; and/or iv) an operating temperature of 15 degrees to 60 degrees centigrade.

In a specific embodiment, the present invention provides the produced compound which is dried to powder by any one or more drying steps chosen from the list comprising spray drying, lyophilization, spray freeze drying, freeze spray drying, band drying, belt drying, vacuum band drying, vacuum belt drying, drum drying, roller drying, vacuum drum drying and vacuum roller drying, wherein the dried powder contains <15 percent-wt. of water, preferably <10 percent-wt. of water, more preferably <7 percent-wt. of water, most preferably <5 percent-wt. of water.

Another aspect of the present invention provides the use of a cell as defined herein, in a method for the production of a compound as defined herein. A further aspect of the present invention provides the use of a method as defined herein for the production of a compound as defined herein.

Furthermore, the invention also relates to the compound as defined herein obtained by the methods according to the invention, as well as to the use of a polynucleotide, the vector, cells, microorganisms or the polypeptide as described above for the production of said compound. Said compound may be used for the manufacture of a preparation, as food additive, prebiotic, symbiotic, for the supplementation of baby food, adult food, infant animal feed, adult animal feed, or as either therapeutically or pharmaceutically active compound or in cosmetic applications. With the novel methods, the compound can easily and effectively be provided, without the need for complicated, time and cost consuming synthetic processes.

For identification of the compound of present invention produced in the cell as described herein, the monosaccharide or the monomeric building blocks (e.g. the monosaccharide or glycan unit composition), the anomeric configuration of side chains, the presence and location of substituent groups, degree of polymerization/molecular weight and the linkage pattern can be identified by standard methods known in the art, such as, e.g. methylation analysis, reductive cleavage, hydrolysis, GC-MS (gas chromatography-mass spectrometry), MALDI-MS (Matrix-assisted laser desorption/ionization-mass spectrometry), ESI-MS (Electrospray ionization-mass spectrometry), HPLC (High-Performance Liquid chromatography with ultraviolet or refractive index detection), HPAEC-PAD (High-Performance Anion-Exchange chromatography with Pulsed Amperometric Detection), CE (capillary electrophoresis), IR (infrared)/Raman spectroscopy, and NMR (Nuclear magnetic resonance) spectroscopy techniques. The crystal structure can be solved using, e.g., solid-state NMR, FT-IR (Fourier transform infrared spectroscopy), and WAXS (wide-angle X-ray scattering). The degree of polymerization (DP), the DP distribution, and polydispersity can be determined by, e.g., viscosimetry and SEC (SEC-HPLC, high performance size-exclusion chromatography). To identify the monomeric components of the compound methods such as e.g. acid-catalysed hydrolysis, HPLC (high performance liquid chromatography) or GLC (gas-liquid chromatography) (after conversion to alditol acetates) may be used. To determine the glycosidic linkages, the compound is methylated with methyl iodide and strong base in DMSO, hydrolysis is performed, a reduction to partially methylated alditols is achieved, an acetylation to methylated alditol acetates is performed, and the analysis is carried out by GLC/MS (gas-liquid chromatography coupled with mass spectrometry). To determine the glycan sequence, a partial depolymerization is carried out using an acid or enzymes to determine the structures. To identify the anomeric configuration, the compound is subjected to enzymatic analysis, e.g. it is contacted with an enzyme that is specific for a particular type of linkage, e.g., beta-galactosidase, or alpha-glucosidase, etc., and NMR may be used to analyse the products.

The separated and preferably also purified compound as described herein is incorporated into a food (e.g., human food or feed), dietary supplement, pharmaceutical ingredient, cosmetic ingredient or medicine. In some embodiments, the compound is mixed with one or more ingredients suitable for food, feed, dietary supplement, pharmaceutical ingredient, cosmetic ingredient or medicine.

In some embodiments, the dietary supplement comprises at least one prebiotic ingredient and/or at least one probiotic ingredient.

A “prebiotic” is a substance that promotes growth of microorganisms beneficial to the host, particularly microorganisms in the gastrointestinal tract. In some embodiments, a dietary supplement provides multiple prebiotics, including the compound being a prebiotic produced and/or purified by a process disclosed in this specification, to promote growth of one or more beneficial microorganisms. Examples of prebiotic ingredients for dietary supplements include other prebiotic molecules (such as HMOs) and plant polysaccharides (such as inulin, pectin, b-glucan and xylooligosaccharide). A “probiotic” product typically contains live microorganisms that replace or add to gastrointestinal microflora, to the benefit of the recipient. Examples of such microorganisms include Lactobacillus species (for example, L. acidophilus and L. bulgaricus), Bifidobacterium species (for example, B. animalis, B. longum and B. infantis (e.g., Bi-26)), and Saccharomyces boulardii. In some embodiments, a compound produced and/or purified by a process of this specification is orally administered in combination with such microorganism.

Examples of further ingredients for dietary supplements include oligosaccharides (such as 2′-fucosyllactose, 3-fucosyllactose, 3′-sialyllactose, 6′-sialyllactose), disaccharides (such as lactose), monosaccharides (such as glucose, galactose, L-fucose, sialic acid, glucosamine and N-acetylglucosamine), thickeners (such as gum arabic), acidity regulators (such as trisodium citrate), water, skimmed milk, and flavourings.

In some embodiments, the compound, such as an oligosaccharide is incorporated into a human baby food (e.g., infant formula). Infant formula is generally a manufactured food for feeding to infants as a complete or partial substitute for human breast milk. In some embodiments, infant formula is sold as a powder and prepared for bottle- or cup-feeding to an infant by mixing with water. The composition of infant formula is typically designed to be roughly mimic human breast milk. In some embodiments, a compound like e.g. a (Neu(n)Ac-containing) oligosaccharide produced and/or purified by a process in this specification is included in infant formula to provide nutritional benefits similar to those provided by the oligosaccharides in human breast milk. In some embodiments, the compound like a (Neu(n)Ac-containing) oligosaccharide is mixed with one or more ingredients of the infant formula. Examples of infant formula ingredients include non-fat milk, carbohydrate sources (e.g., lactose), protein sources (e.g., whey protein concentrate and casein), fat sources (e.g., vegetable oils—such as palm, high oleic safflower oil, rapeseed, coconut and/or sunflower oil; and fish oils), vitamins (such as vitamins A, Bb, Bi2, C and D), minerals (such as potassium citrate, calcium citrate, magnesium chloride, sodium chloride, sodium citrate and calcium phosphate) and possibly human milk oligosaccharides (HMOs). Such HMOs may include, for example, DiFL, lacto-N-triose II, LNT, LNnT, lacto-N-fucopentaose I, lacto-N-neofucopentaose, lacto-N-fucopentaose II, lacto-N-fucopentaose III, lacto-N-fucopentaose V, lacto-N-neofucopentaose V, lacto-N-difucohexaose I, lacto-N-difucohexaose II, 6′-galactosyllactose, 3′-galactosyllactose, lacto-N-hexaose and lacto-N-neohexaose.

In some embodiments, the one or more infant formula ingredients comprise non-fat milk, a carbohydrate source, a protein source, a fat source, and/or a vitamin and mineral.

In some embodiments, the one or more infant formula ingredients comprise lactose, whey protein concentrate and/or high oleic safflower oil.

In some embodiments, the concentration of the compound, like a (Neu(n)Ac-containing) oligosaccharide in the infant formula is approximately the same concentration as the concentration of the compound generally present in human breast milk.

In some embodiments, the compound is incorporated into a feed preparation, wherein said feed is chosen from the list comprising pet food, animal milk replacer, veterinary product, veterinary feed supplement, nutrition supplement, post weaning feed, or creep feed.

As will be shown in the examples herein, the newly identified method and cell of the present invention have proven to be useful in the fermentative production of a compound, being a disaccharide, oligosaccharide and/or Neu(n)Ac-containing bioproduct. The method and the cell of the invention preferably provide at least one of the following further surprising advantages:
High and/or better titres of the compound (g/L),
High and/or better production rate r (g compound/L/h),
High and/or better cell performance index CPI (g compound/g X),
High and/or better specific productivity Qp (g compound/g X/h),
High and/or better yield on carbon source Ys (g compound/g carbon source),
High and/or better carbon source uptake/conversion rate Qs (g carbon source/g X/h),
High and/or better acceptor conversion/consumption rate rs (g acceptor/h),
High and/or enhanced excretion or secretion of the compound, and/or
High and/or enhanced growth speed of the production host,
   when compared to a production host for a compound with an identical genetic background but lacking the enhanced acetyl-CoenzymeA synthesis. The carbon source being glucose, glycerol, fructose, sucrose, maltose, lactose, arabinose, malto-oligosaccharides, maltotriose, sorbitol, xylose, rhamnose, galactose, mannose, methanol, ethanol, trehalose, starch, cellulose, hemi-cellulose, molasses, corn-steep liquor, high-fructose syrup, acetate, citrate, lactate and pyruvate or any one as defined herein; the “acceptor” as defined above.

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization described above and below are those well-known and commonly employed in the art. Standard techniques are used for nucleic acid and peptide synthesis. Generally, purification steps are performed according to the manufacturer's specifications.

Further advantages follow from the specific embodiments and the examples. It goes without saying that the abovementioned features and the features which are still to be explained below can be used not only in the respectively specified combinations, but also in other combinations or on their own, without departing from the scope of the present invention.

Moreover, the present invention relates to the following specific embodiments:
1. Cell for the production of a compound, said cell comprising a pathway for the production of said compound, wherein said compound is a disaccharide, oligosaccharide and/or a Neu(n)Ac-containing bioproduct, wherein (n) is 4, 5, 7, 8 or 9 or a combination thereof, characterised in that said cell is metabolically engineered for enhanced synthesis of acetyl-Coenzyme A.
2. Cell according to any one of embodiment 1, wherein said enhanced acetyl-Coenzyme A synthesis is obtained by enhanced expression or activity of any one or more of the enzymes: i) acetyl-Coenzyme A ligase (EC 6.2.1.1 or 6.2.1.13); ii) pyruvate dehydrogenase (EC 1.2.5.1); iii) pantothenate kinase (EC 2.7.1.33); iv) acetyl phosphate-producing pyruvate oxidase (EC 1.2.3.3); v) acetate kinase (EC 2.7.2.1); vi) phosphate acetyltransferase (EC 2.3.1.8); vii) pyruvate decarboxylase (EC 4.1.1.1); viii) acetaldehyde dehydrogenase (EC 1.2.1.3, EC 1.2.1.4, or EC 1.2.1.5); ix) pyruvate formate lyase (EC 2.3.1.54); x) CoA-acetylating pyruvate oxidase (EC 1.2.3.6), xi) pyruvate synthase (EC 1.2.7.1), or xii) pyruvate dehydrogenase enzyme complex (EC 1.2.4.1 (pyruvate dehydrogenase E1 component), EC 2.3.1.12 (pyruvate dehydrogenase, E2 subunit), EC 1.8.1.4 (lipoamide dehydrogenase, E3 subunit)).
3. Cell according to embodiment 2, wherein said enhanced acetyl-CoA synthesis is obtained by a method selected from the group consisting of: a) increasing the copy number of any one or more of the genes encoding enzyme i) to x) or the enzyme complex of xi), b) modifying an expression regulatory sequence of any one or more of said gene, and c) combinations thereof.
4. Cell according to any one of embodiment 2 to 3, wherein said enhanced synthesis is obtained by overexpressing any one or more of the genes encoding an endogenous enzyme i) to xi) or the enzyme complex of xii); and/or introducing and expressing any one or more of a homologous or heterologous gene encoding an enzyme i) to xi) or the enzyme complex of xii).
5. Cell according to any one of embodiment 2 to 4, wherein any one or more of said enzyme i) to xi) or the enzyme complex of xii) is presented to the cell in one or more gene expression modules wherein expression is regulated by one or more regulatory sequences.
6. Cell according to embodiment 5, wherein said expression modules are integrated in the cell's genome and/or presented to the cell on a vector comprising plasmid, cosmid, phage, liposome or virus, which is to be stably transformed into said cell.
7. Cell according to any one of previous embodiments, wherein said cell is modified for enhanced synthesis and/or supply of PEP.
8. Cell according to any one of the previous embodiment 1 to 7, wherein said cell is further modified for reduced degradation of acetyl-CoA and/or its main precursor pyruvate.
9. Cell according to any one of the previous embodiments 1 to 8, wherein said cell is modified for reduced expression of or deleting the genes encoding for any one or more of a) lactate dehydrogenase (EC 1.1.2.3, EC 1.1.2.4, EC 1.1.2.5, EC 1.1.1.27, EC 1.1.1.28), b) pyruvate carboxylase (EC 6.4.1.1), c) isocitrate lyase (EC 4.1.3.1); d) malate synthase (EC 2.3.3.9).
10. Cell according to any one of the previous embodiments 1 to 9, wherein said cell is further modified for rendering less functional the Krebs cycle genes by either reduced expression or point mutations preferably A258T, A162V and/or A124T in the citrate synthase enzyme coded by gltA in E coli.
11. Cell according to any one of the previous embodiments 1 to 10, wherein said disaccharide is chosen from the list of comprising lactose (Gal-b1,4-Glc), lacto-N-biose (Gal-b1,3-GlcNAc), N-acetyllactosamine (Gal-b1,4-GlcNAc), LacDiNAc (GalNAc-b1,4-GlcNAc), N-acetylgalactosaminylglucose (GalNAc-b1,4-Glc), Neu5Ac-a2,3-Gal, Neu5Ac-a2,6-Gal and fucopyranosyl-(1-4)-N-glycolylneuraminic acid (Fuc-(1-4)-Neu5Gc).
12. Cell according to any one of the previous embodiments 1 to 10, wherein said oligosaccharide is a milk oligosaccharide, preferably a mammalian milk oligosaccharide, more preferably a human milk oligosaccharide; or is a Lewis-type antigen oligosaccharide, O-antigen, enterobacterial common antigen (ECA), the oligosaccharide repeats present in capsular polysaccharides, peptidoglycan, amino-sugar or antigen of the human ABO blood group system, preferably said milk oligosaccharide is a human milk oligosaccharide.
13. Cell according to any one of the embodiments 1 to 10, wherein said Neu(n)Ac-containing bioproduct is chosen from the list comprising sialic acid, a disaccharide, an oligosaccharide, sialylated compound comprising Neu5Ac, a Neu(n)Ac-containing glycolipid, a Neu(n)Ac-containing glycoprotein.
14. Cell according to any one of the embodiments 1 to 10, 12, 13, wherein said oligosaccharide is a neutral oligosaccharide, a fucosylated oligosaccharide and/or acidic oligosaccharide.
15. Cell according to any one of embodiments 1 to 10, 12 to 14, wherein said oligosaccharide is chosen from the list comprising 3-fucosyllactose, 2′-fucosyllactose, 6-fucosyllactose, 2′,3-difucosyllactose, 2′,2-difucosyllactose, 3,4-difucosyllactose, 6′-sialyllactose, 3′-sialyllactose, 3,6-disialyllactose, 6,6′-disialyllactose, 8,3-disialyllactose, 3,6-disialyllacto-N-tetraose, lactodifucotetraose, lacto-N-tetraose, lacto-N-neotetraose, lacto-N-fucopentaose II, lacto-N-fucopentaose I, lacto-N-fucopentaose III, lacto-N-fucopentaose V, lacto-N-fucopentaose VI, sialyllacto-N-tetraose c, sialyllacto-N-tetraose b, sialyllacto-N-tetraose a, lacto-N-difucohexaose I, lacto-N-difucohexaose II, lacto-N-hexaose, lacto-N-neohexaose, para-lacto-N-hexaose, monofucosylmonosialyllacto-N-tetraose c, monofucosyl para-lacto-N-hexaose, monofucosyllacto-N-hexaose III, isomeric fucosylated lacto-N-hexaose III, isomeric fucosylated lacto-N-hexaose I, sialyllacto-N-hexaose, sialyllacto-N-neohexaose II, difucosyl-para-lacto-N-hexaose, difucosyllacto-N-hexaose, difucosyllacto-N-hexaose a, difucosyllacto-N-hexaose c.
16. Cell according to any one of the embodiments 2 to 15, wherein said acetyl-Coenzyme A ligase is originating from Escherichia coli species comprising but not limited to E. coli B, E. coli BL21, E. coli BL21(DE3), E. coli C, E. coli DH5alpha, E. coli K-12, E. coli Nissle, E. coli Top10, E. coli W, or wherein said acetyl-Coenzyme A synthetase is originating from Salmonella typhi, Vibrio cholera, Saccharomyces cerevisiae, Bacillus subtilis, Mycobacterium tuberculosis, Campylobacter jejuni, Yersinia pestis, Corynebacteriales, preferably said acetyl-Coenzyme A ligase is the E. coli UniProtKB-P27550 enzyme, or is the S. cerevisiae UniProt KB Q01574 enzyme, or is the S. cerevisiae UniProt KB P52910 enzyme, or is the B. subtilis UniProt KB P39062 enzyme, or is the H. sapiens UniProt KB Q9NR19 enzyme.
17. Cell according to any one of embodiment 1 to 16, wherein the cell comprises and expresses at least one glycosyltransferase.
18. Cell according to embodiment 17, wherein said glycosyltransferase is selected from the list comprising fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases and fucosaminyltransferases.
19. Cell according to embodiment 17 or 18, wherein said cell is modified in the expression or activity of at least one of said glycosyltransferases, wherein preferably said modification is obtained by overexpressing an endogenous glycosyltransferase and/or introducing and expressing a homologous or heterologous glycosyltransferase.
20. Cell according to any one of embodiment 17 to 19, wherein one of said glycosyltransferases is a fucosyltransferase that transfers a fucose from a GDP-fucose donor to lactose in an alpha1,2 and/or alpha-1,3 linkage, thereby producing fucosyllactose and/or difucosyllactose.
21. Cell according to any one of embodiment 17 to 20, said cell comprising (i) a GDP-fucose biosynthesis pathway comprising at least one enzyme chosen from the list comprising mannose-6-phosphate isomerase, phosphomannomutase, mannose-1-phosphate guanylyltransferase, GDP-mannose 4,6-dehydratase, GDP-L-fucose synthase, fucose permease, fucose kinase, fucose-1-phosphate guanylyltransferase; and preferably (ii) a fucosyltransferase.
22. Cell according to any one of embodiment 17 to 21, wherein one of said glycosyltransferases is a sialyltransferase that transfers a N-acetyl Neuraminic acid (sia) from a CMP-Sia donor to lactose in an alpha-2,3-, alpha-2,6- and/or alpha-2,8-linkage, thereby producing sialyllactose and/or disialyllactose.
23. Cell according to any one of embodiment 17 to 22, said cell comprising any one or more of (i) a sialic acid biosynthesis pathway comprising at least one enzyme chosen from the list comprising UDP-GlcNAc 2-epimerase, N-acylglucosamine 2-epimerase and sialic acid synthase; (ii) an N-acylneuraminate cytidylyltransferase; and (iii) a sialyltransferase.
24. Cell according to any one of embodiment 17 to 23, wherein one of said glycosyltransferases is a galactosyltransferase chosen from the list comprising beta-1,3-galactosyltransferase, N-acetylglucosamine beta-1,3-galactosyltransferase, beta-1,4-galactosyltransferase, N-acetylglucosamine beta-1,4-galactosyltransferase, alpha-1,3-galactosyltransferase and alpha-1,4-galactosyltransferase.
25. Cell according to any one of embodiment 1 to 24, said cell comprising a galactosylation pathway comprising (i) a UDP-galactose biosynthesis pathway comprising at least one enzyme chosen from the list comprising galactose-1-epimerase, galactokinase, glucokinase, galactose-1-phosphate uridylyltransferase, UDP-glucose 4-epimerase, glucose-1-phosphate uridylyltransferase, glucophosphomutase; and (ii) a galactosyltransferase.
26. Cell according to any one of embodiment 1 to 25, wherein said cell comprises an N-acetylglucosaminylation pathway comprising (i) at least one enzyme chosen from the list comprising L-glutamine-D-fructose-6-phosphate aminotransferase, N-acetylglucosamine-6-phosphate deacetylase, phosphoglucosamine mutase, N-acetylglucosamine-1-phosphate uridylyltransferase/glucosamine-1-phosphate acetyltransferase, N-acetylglucosaminyltransferase; and (ii) a N-acetylglucosaminyltransferase.
27. Cell according to any one of embodiment 1 to 26, wherein said cell comprises a pathway to synthesize lacto-N-tetraose (LNT) comprising a galactoside beta-1,3-N-acetylglucosaminyltransferase and an N-acetylglucosamine beta-1,3-galactosyltransferase.
28. Cell according to any one of embodiment 1 to 26, wherein said cell comprises a pathway to synthesize lacto-N-neotetraose (LNnT) comprising a galactoside beta-1,3-N-acetylglucosaminyltransferase and an N-acetylglucosamine beta-1,4-galactosyltransferase.
29. Cell according to any one of the previous embodiments 1 to 28, wherein said cell is further capable to synthesize a nucleotide-activated sugar to be used in the production of said compound.
30. Cell according to embodiment 29, wherein said nucleotide-activated sugar is chosen from the list comprising UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), UDP-glucuronate, UDP-galacturonate, UDP-2-acetamido-2,6-dideoxy-L-arabino-4-hexulose, UDP-2-acetamido-2,6-dideoxy-L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid (CMP-Neu5Ac), CMP-N-glycolylneuraminic acid (CMP-Neu5Gc), GDP-fucose (GDP-Fuc), GDP-rhamnose and UDP-xylose.
31. Cell according to any one of the previous embodiments 1 to 30, wherein the cell comprises a catabolic pathway for selected mono-, di- or oligosaccharides which is at least partially inactivated, the mono-, di-, or oligosaccharides being involved in and/or required for the synthesis of said compound.
32. Cell according to any one of the previous embodiments 1 to 31, wherein said cell is using a precursor for the synthesis of said compound said precursor being fed to the cell from the cultivation medium.
33. Cell according to any one of the previous embodiments 1 to 32, wherein said cell is producing a precursor for the synthesis of said compound.
34. Cell according to any one of the previous embodiments 1 to 33, wherein said cell produces 30 g/L or more of compound in the whole broth and/or supernatant and/or wherein said compound in the whole broth and/or supernatant has a purity of at least 80% measured on the total amount of compound and its precursor produced by said cell in the whole broth and/or supernatant, respectively.
35. Cell according to any one of the previous embodiments 1 to 34, wherein said cell is a bacterium, fungus, yeast, a plant cell, an animal cell, or a protozoan cell,
   preferably said bacterium is of an Escherichia coli strain, more preferably of an Escherichia coli strain which is a K-12 strain, even more preferably the Escherichia coli K-12 strain is E. coli MG1655,
   preferably said fungus belongs to a genus chosen from the group comprising Rhizopus, Dictyostelium, Penicillium, Mucor or Aspergillus,
   preferably said yeast belongs to a genus chosen from the group comprising Saccharomyces, Zygosaccharomyces, Pichia, Komagataella, Hansenula, Yarrowia, Starmerella, Kluyveromyces or Debaromyces,
   preferably said plant cell is an algal cell or is derived from tobacco, alfalfa, rice, tomato, cotton, rapeseed, soy, maize, or corn plant,
   preferably said animal cell is derived from non-human mammals, birds, fish, invertebrates, reptiles, amphibians or insects or is a genetically modified cell line derived from human cells excluding embryonic stem cells, more preferably said human and non-human mammalian cell is an epithelial cell, an embryonic kidney cell, a fibroblast cell, a COS cell, a Chinese hamster ovary (CHO) cell, a murine myeloma cell, an NIH-3T3 cell, a non-mammary adult stem cell or derivatives thereof, more preferably said insect cell is derived from Spodoptera frugiperda, Bombyx mori, Mamestra brassicae, Trichoplusia ni or Drosophila melanogaster,
   preferably said protozoan cell is a Leishmania tarentolae cell.
36. Cell according to any one of the previous embodiments 1 to 35, wherein said cell is stably cultured in a medium.
37. Cell according to any one of the previous embodiments 1 to 36, wherein the cell is capable to synthesize a mixture of compounds.
38. Method for the production of a compound wherein said compound is a disaccharide, oligosaccharide and/or a Neu(n)Ac-containing bioproduct, wherein (n) is 4, 5, 7, 8 or 9 or a combination thereof, the method comprising the steps of:
   a. providing a cell capable to produce said compound, wherein said cell is metabolically engineered for enhanced synthesis of acetyl-Coenzyme A,
   b. cultivating the cell under conditions permissive for producing said compound,
   c. and preferably separating the desired compound from said cultivation.
39. Method for the production of a wherein said compound is a disaccharide, oligosaccharide and/or a Neu(n)Ac-containing bioproduct, wherein (n) is 4, 5, 7, 8 or 9 or a combination thereof by a genetically modified cell, comprising the steps of:
   a) providing a cell according to any one of the embodiments 1 to 36, and
   b) culturing the cell in a medium under conditions permissive for the production of said compound,
   c) preferably separating said compound from the cultivation.
40. Method for the production of a mixture of compounds by a genetically modified cell, comprising the steps of:
   a) providing a cell according to embodiment 37, and
   b) culturing the cell in a medium under conditions permissive for the production of said compounds,
   c) preferably separating said mixture of compounds from the cultivation.
41. Method according to any one of embodiment 38 to 40, the method further comprising at least one of the following steps:
   i) Adding to the culture medium in a reactor at least one precursor and/or acceptor feed wherein the total reactor volume ranges from 250 mL (millilitre) to 10,000 m³ (cubic meter), preferably in a continuous manner, and preferably so that the final volume of the culture medium is not more than three-fold, preferably not more than two-fold, more preferably less than 2-fold of the volume of the culture medium before the addition of said precursor and/or acceptor feed;
   ii) Adding at least one precursor and/or acceptor feed in a continuous manner to the culture medium over the course of 1 day, 2 days, 3 days, 4 days, 5 days by means of a feeding solution;
   iii) Adding at least one precursor and/or acceptor feed in a continuous manner to the culture medium over the course of 1 day, 2 days, 3 days, 4 days, 5 days by means of a feeding solution and wherein preferably, the pH of said feeding solution is set between 3 and 7 and
      wherein preferably, the temperature of said feeding solution is kept between 20° C. and 80° C.; said method resulting in a Neu(n)Ac-containing compound with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of said culture medium.
42. Method according to any one of embodiment 38 to 40, the method further comprising at least one of the following steps:
   i) adding to the culture medium a lactose feed comprising at least 50, more preferably at least 75, more preferably at least 100, more preferably at least 120, more preferably at least 150 gram of lactose per litre of initial reactor volume wherein the reactor volume ranges from 250 mL to 10,000 m³ (cubic meter), preferably in a continuous manner, and preferably so that the final volume of the culture medium is not more than three-fold, preferably not more than two-fold, more preferably less than 2-fold of the volume of the culture medium before the addition of said lactose feed;
   ii) adding a lactose feed in a continuous manner to the culture medium over the course of 1 day, 2 days, 3 days, 4 days, 5 days by means of a feeding solution;
   iii) adding a lactose feed in a continuous manner to the culture medium over the course of 1 day, 2 days, 3 days, 4 days, 5 days by means of a feeding solution and wherein the concentration of said lactose feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably the pH of said solution is set between 3 and 7 and wherein preferably the temperature of said feed solution is kept between 20° C. and 80° C.;
said method resulting in an oligosaccharide with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of said culture medium.
43. Method according to embodiment 42, wherein the lactose feed is accomplished by adding lactose from the beginning of the cultivating in a concentration of at least 5 mM, preferably in a concentration of 30, 40, 50, 60, 70, 80, 90, 100, 150 mM, more preferably in a concentration >300 mM.
44. Method according to any one of embodiment 42 or 43, wherein said lactose feed is accomplished by adding lactose to the cultivation medium in a concentration, such, that throughout the production phase of the cultivation a lactose concentration of at least 5 mM, preferably 10 mM or 30 mM is obtained.
45. Method according to any one of embodiment 42 to 44, wherein the cells are cultivated for at least about 60, 80, 100, or about 120 hours or in a continuous manner.
46. Method according to any one of embodiment 42 to 45, wherein a carbon and energy source, preferably glucose, glycerol, fructose, maltose, arabinose, maltodextrines, malto-oligosaccharides, maltotriose, sorbitol, xylose, rhamnose, sucrose, galactose, mannose, methanol, ethanol, trehalose, starch, cellulose, hemi-cellulose, polyols, corn-steep liquor, high-fructose syrup, succinate, malate, acetate, citrate, lactate and pyruvate, is also added, preferably continuously to the culture medium, preferably with the lactose.
47. Method according to any one of embodiment 38 to 46, wherein said cell uses at least one precursor for the synthesis of said oligosaccharide, preferably said cell uses two or more precursors for the synthesis of said oligosaccharide.
48. Method according to any one of embodiment 38 to 47, wherein the culture medium contains at least one compound selected from the group comprising lactose, galactose, sialic acid, fucose, GlcNAc, GalNAc, lacto-N-biose (LNB), N-acetyllactosamine (LacNAc).
49. Method according to any one of embodiment 38 to 48, wherein a first phase of exponential cell growth is provided by adding a carbon-based substrate, preferably glucose or sucrose, to the culture medium before the lactose is added to the culture medium in a second phase.
50. Method according to any one of embodiment 38 to 49, wherein said cell is producing at least one precursor for the synthesis of said compound.
51. Method according to any one of embodiment 47 to 50, wherein said precursor for the synthesis of said oligosaccharide is completely converted into said compound.
52. Method according to any one of embodiment 38 to 51, wherein the compound is separated from the culture medium and/or the bacterial cell.
53. Method according to any one of embodiment 38 to 52, wherein said separation comprises at least one of the following steps: clarification, ultrafiltration, nanofiltration, reverse osmosis, microfiltration, activated charcoal or carbon treatment, tangential flow high-performance filtration, tangential flow ultrafiltration, affinity chromatography, ion exchange chromatography, hydrophobic interaction chromatography and/or gel filtration, ligand exchange chromatography.
54. Method according to any one of embodiment 38 to 53, wherein said method further comprises purification of said compound.
55. Method according to embodiment 54, wherein said purification comprises at least one of the following steps: use of activated charcoal or carbon, use of charcoal, nanofiltration, ultrafiltration or ion exchange, use of alcohols, use of aqueous alcohol mixtures, crystallization, evaporation, precipitation, drying, spray drying or lyophilization.
56. Use of a cell as defined in any one of embodiment 1 to 36 in the fermentative production of a compound, preferably oligosaccharide.
57. Use of a cell according to embodiment 37 in the fermentative production of a mixture of compounds, preferably comprising at least two oligosaccharides.
58. Use of a method according to any one of embodiment 38 to 55 for the production of a compound, preferably an oligosaccharide.

Moreover, the present invention relates to the following preferred specific embodiments:
1. Cell for the production of a compound, said cell comprising a pathway for the production of said compound, wherein said compound is a disaccharide, oligosaccharide and/or a Neu(n)Ac-containing bioproduct, wherein (n) is 4, 5, 7, 8 or 9 or a combination thereof, characterised in that said cell is metabolically engineered for enhanced synthesis of acetyl-Coenzyme A.
2. Cell according to preferred embodiment 1, wherein said enhanced acetyl-Coenzyme A synthesis is obtained by enhanced expression or activity of any one or more of the enzymes: i) acetyl-Coenzyme A ligase (EC 6.2.1.1 or 6.2.1.13); ii) pyruvate dehydrogenase (EC 1.2.5.1); iii) pantothenate kinase (EC 2.7.1.33); iv) acetyl phosphate-producing pyruvate oxidase (EC 1.2.3.3); v) acetate kinase (EC 2.7.2.1); vi) phosphate acetyltransferase (EC 2.3.1.8); vii) pyruvate decarboxylase (EC 4.1.1.1); viii) acetaldehyde dehydrogenase (EC 1.2.1.3, EC 1.2.1.4, or EC 1.2.1.5); ix) pyruvate formate lyase (EC 2.3.1.54); x) CoA-acetylating pyruvate oxidase (EC 1.2.3.6), xi) pyruvate synthase (EC 1.2.7.1), or xii) pyruvate dehydrogenase enzyme complex (EC 1.2.4.1 (pyruvate dehydrogenase E1 component), EC 2.3.1.12 (pyruvate dehydrogenase, E2 subunit), EC 1.8.1.4 (lipoamide dehydrogenase, E3 subunit)).
3. Cell according to preferred embodiment 2, wherein said enhanced acetyl-CoA synthesis is obtained by a method selected from the group consisting of: a) increasing the copy number of any one or more of the genes encoding enzyme i) to x) or the enzyme complex of xi), b) modifying an expression regulatory sequence of any one or more of said genes, and c) combinations thereof.
4. Cell according to any one of preferred embodiment 2 or 3, wherein said enhanced synthesis is obtained by overexpressing any one or more of the genes encoding an endogenous enzyme i) to xi) or the enzyme complex of xii); and/or introducing and expressing any one or more of a homologous or heterologous gene encoding an enzyme i) to xi) or the enzyme complex of xii).
5. Cell according to any one of preferred embodiments 2 to 4, wherein any one or more of said enzyme i) to xi) or the enzyme complex of xii) is presented to the cell in one or more gene expression modules wherein expression is regulated by one or more regulatory sequences.
6. Cell according to preferred embodiment 5, wherein said expression modules are integrated in the cell's genome and/or presented to the cell on a vector comprising plasmid, cosmid, phage, liposome or virus, which is to be stably transformed into said cell.
7. Cell according to any one of previous preferred embodiments, wherein said cell is modified for enhanced synthesis and/or supply of phosphoenolpyruvate (PEP).
8. Cell according to any one of previous preferred embodiments, wherein said cell is further modified for reduced degradation of acetyl-CoA and/or its main precursor pyruvate.
9. Cell according to any one of previous preferred embodiments, wherein said cell is modified for reduced expression of or deleting the genes encoding for any one or more of a) lactate dehydrogenase (EC 1.1.2.3, EC 1.1.2.4, EC 1.1.2.5, EC 1.1.1.27, EC 1.1.1.28), b) pyruvate carboxylase (EC 6.4.1.1), c) isocitrate lyase (EC 4.1.3.1); d) malate synthase (EC 2.3.3.9).
10. Cell according to any one of previous preferred embodiments, wherein said cell is further modified for rendering less functional the Krebs cycle genes by either reduced expression or point mutations preferably A258T, A162V and/or A124T in the citrate synthase enzyme coded by gltA in E coli.
11. Cell according to any one of previous preferred embodiments, wherein said disaccharide is chosen from the list comprising lactose (Gal-b1,4-Glc), lacto-N-biose (Gal-b1,3-GlcNAc), N-acetyllactosamine (Gal-b1,4-GlcNAc), LacDiNAc (GalNAc-b1,4-GlcNAc), N-acetylgalactosaminylglucose (GalNAc-b1,4-Glc), Neu5Ac-a2,3-Gal, Neu5Ac-a2,6-Gal and fucopyranosyl-(1-4)-N-glycolylneuraminic acid (Fuc-(1-4)-Neu5Gc).
12. Cell according to any one of preferred embodiments 1 to 10, wherein said oligosaccharide is a milk oligosaccharide, preferably a mammalian milk oligosaccharide, more preferably a human milk oligosaccharide, a Lewis-type antigen oligosaccharide, O-antigen, enterobacterial common antigen (ECA), the oligosaccharide repeats present in capsular polysaccharides, peptidoglycan, amino-sugar or antigen of the human ABO blood group system.
13. Cell according to any one of preferred embodiments 1 to 10, wherein said Neu(n)Ac-containing bioproduct is chosen from the list comprising sialic acid, a disaccharide, an oligosaccharide, sialylated compound comprising Neu5Ac, a Neu(n)Ac-containing glycolipid, a Neu(n)Ac-containing glycoprotein.
14. Cell according to any one of preferred embodiments 1 to 10, 12, 13, wherein said oligosaccharide is a non-charged (neutral) oligosaccharide, a fucosylated oligosaccharide and/or acidic oligosaccharide.
15. Cell according to any one of preferred embodiments 1 to 10, 12 to 14, wherein said oligosaccharide is chosen from the list comprising 3-fucosyllactose, 2′-fucosyllactose, 6-fucosyllactose, 2′,3-difucosyllactose, 2′,2-difucosyllactose, 3,4-difucosyllactose, 6′-sialyllactose, 3′-sialyllactose, 3,6-disialyllactose, 6,6′-disialyllactose, 8,3-disialyllactose, 3,6-disialyllacto-N-tetraose, lactodifucotetraose, lacto-N-tetraose, lacto-N-neotetraose, lacto-N-fucopentaose II, lacto-N-fucopentaose I, lacto-N-fucopentaose III, lacto-N-fucopentaose V, lacto-N-fucopentaose VI, sialyllacto-N-tetraose c, sialyllacto-N-tetraose b, sialyllacto-N-tetraose a, lacto-N-difucohexaose I, lacto-N-difucohexaose II, lacto-N-hexaose, lacto-N-neohexaose, para-lacto-N-hexaose, monofucosylmonosialyllacto-N-tetraose c, monofucosyl para-lacto-N-hexaose, monofucosyllacto-N-hexaose III, isomeric fucosylated lacto-N-hexaose III, isomeric fucosylated lacto-N-hexaose I, sialyllacto-N-hexaose, sialyllacto-N-neohexaose II, difucosyl-para-lacto-N-hexaose, difucosyllacto-N-hexaose, difucosyllacto-N-hexaose a, difucosyllacto-N-hexaose c.
16. Cell according to any one of preferred embodiments 2 to 15, wherein said acetyl-Coenzyme A ligase is originating from Escherichia coli species comprising but not limited to E. coli B, E. coli BL21, E. coli BL21(DE3), E. coli C, E. coli DH5alpha, E. coli K-12, E. coli Nissle, E. coli Top10, E. coli W, or wherein said acetyl-Coenzyme A synthetase is originating from Salmonella typhi, Vibrio cholera, Saccharomyces cerevisiae, Bacillus subtilis, Mycobacterium tuberculosis, Campylobacter jejuni, Yersinia pestis, Corynebacteriales, preferably said acetyl-Coenzyme A ligase is the E. coli UniProtKB—P27550 enzyme, or is the S. cerevisiae UniProt KB Q01574 enzyme, or is the S. cerevisiae UniProt KB P52910 enzyme, or is the B. subtilis UniProt KB P39062 enzyme, or is the H. sapiens UniProt KB Q9NR19 enzyme.
17. Cell according to any one of previous preferred embodiments, wherein the cell comprises and expresses at least one glycosyltransferase.
18. Cell according to preferred embodiment 17, wherein said glycosyltransferase is selected from the list comprising fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases and fucosaminyltransferases.
19. Cell according to preferred embodiment 17 or 18, wherein said cell is modified in the expression or activity of at least one of said glycosyltransferases, wherein preferably said modification is obtained by overexpressing an endogenous glycosyltransferase and/or introducing and expressing a homologous or heterologous glycosyltransferase.
20. Cell according to any one of preferred embodiments 17 to 19, wherein one of said glycosyltransferases is a fucosyltransferase that transfers a fucose from a GDP-fucose donor to lactose in an alpha-1,2- and/or alpha-1,3 linkage, thereby producing fucosyllactose and/or difucosyllactose.
21. Cell according to any one of preferred embodiments 17 to 20, said cell comprising (i) a GDP-fucose biosynthesis pathway comprising at least one enzyme chosen from the list comprising mannose-6-phosphate isomerase, phosphomannomutase, mannose-1-phosphate guanylyltransferase, GDP-mannose 4,6-dehydratase, GDP-L-fucose synthase, fucose permease, fucose kinase, fucose-1-phosphate guanylyltransferase, L-fucokinase/GDP-fucose pyrophosphorylase; and (ii) a fucosyltransferase.
22. Cell according to any one of preferred embodiments 17 to 21, wherein one of said glycosyltransferases is a sialyltransferase that transfers an N-acetyl-neuraminic acid (sia) from a CMP-Sia donor to lactose in an alpha-2,3-, alpha-2,6- and/or alpha-2,8-linkage, thereby producing sialyllactose and/or disialyllactose.
23. Cell according to any one of preferred embodiments 17 to 22, said cell comprising any one or more of (i) a sialic acid biosynthesis pathway comprising at least one enzyme chosen from the list comprising UDP-GlcNAc 2-epimerase, N-acylglucosamine 2-epimerase and sialic acid synthase; (ii) an N-acylneuraminate cytidylyltransferase; and (iii) a sialyltransferase.
24. Cell according to any one of preferred embodiments 17 to 23, wherein one of said glycosyltransferases is a galactosyltransferase chosen from the list comprising beta-1,3-galactosyltransferase, N-acetylglucosamine beta-1,3-galactosyltransferase, beta-1,4-galactosyltransferase, N-acetylglucosamine beta-1,4-galactosyltransferase, alpha-1,3-galactosyltransferase and alpha-1,4-galactosyltransferase.
25. Cell according to any one of previous preferred embodiments, said cell comprising a galactosylation pathway comprising (i) a UDP-galactose biosynthesis pathway comprising at least one enzyme chosen from the list comprising galactose-1-epimerase, galactokinase, glucokinase, galactose-1-phosphate uridylyltransferase, UDP-glucose 4-epimerase, glucose-1-phosphate uridylyltransferase, phosphoglucomutase; and (ii) a galactosyltransferase.
26. Cell according to any one of previous preferred embodiments, wherein said cell comprises an N-acetylglucosaminylation pathway comprising (i) at least one enzyme chosen from the list comprising L-glutamine-D-fructose-6-phosphate aminotransferase, N-acetylglucosamine-6-phosphate deacetylase, phosphoglucosamine mutase, N-acetylglucosamine-1-phosphate uridylyltransferase/glucosamine-1-phosphate acetyltransferase; and (ii) a N-acetylglucosaminyltransferase.
27. Cell according to any one of previous preferred embodiments, wherein said cell comprises a pathway to synthesize lacto-N-tetraose (LNT) comprising a galactoside beta-1,3-N-acetylglucosaminyltransferase and an N-acetylglucosamine beta-1,3-galactosyltransferase.
28. Cell according to any one of preferred embodiments 1 to 27, wherein said cell comprises a pathway to synthesize lacto-N-neotetraose (LNnT) comprising a galactoside beta-1,3-N-acetylglucosaminyltransferase and an N-acetylglucosamine beta-1,4-galactosyltransferase.
29. Cell according to any one of previous preferred embodiments, wherein said cell is further capable to synthesize a nucleotide-activated sugar to be used in the production of said compound.
30. Cell according to preferred embodiment 29, wherein said nucleotide-activated sugar is chosen from the list comprising UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), UDP-glucuronate, UDP-galacturonate, UDP-2-acetamido-2,6-dideoxy-L-arabino-4-hexulose, UDP-2-acetamido-2,6-dideoxy-L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid (CMP-Neu5Ac), CMP-N-glycolylneuraminic acid (CMP-Neu5Gc), GDP-fucose (GDP-Fuc), GDP-rhamnose and UDP-xylose.
31. Cell according to any one of previous preferred embodiments, wherein the cell comprises a catabolic pathway for selected mono-, di- or oligosaccharides which is at least partially inactivated, the mono-, di-, or oligosaccharides being involved in and/or required for the synthesis of said compound.
32. Cell according to any one of previous preferred embodiments, wherein said cell is using a precursor for the synthesis of said compound said precursor being fed to the cell from the culture medium.
33. Cell according to any one of previous preferred embodiments, wherein said cell is producing a precursor for the synthesis of said compound.
34. Cell according to any one of previous preferred embodiments, wherein said cell produces 30 g/L or more of compound in the whole broth and/or supernatant and/or wherein said compound in the whole broth and/or supernatant has a purity of at least 80% measured on the total amount of compound and its precursor produced by said cell in the whole broth and/or supernatant, respectively.
35. Cell according to any one of previous preferred embodiments, wherein said cell is a bacterium, fungus, yeast, a plant cell, an animal cell, or a protozoan cell,
   preferably said bacterium is of an Escherichia coli strain, more preferably of an Escherichia coli strain which is a K-12 strain, even more preferably the Escherichia coli K-12 strain is E. coli MG1655,
   preferably said fungus belongs to a genus chosen from the group comprising Rhizopus, Dictyostelium, Penicillium, Mucor or Aspergillus,
   preferably said yeast belongs to a genus chosen from the group comprising Saccharomyces, Zygosaccharomyces, Pichia, Komagataella, Hansenula, Yarrowia, Starmerella, Kluyveromyces or Debaromyces,
   preferably said plant cell is an algal cell or is derived from tobacco, alfalfa, rice, tomato, cotton, rapeseed, soy, maize, or corn plant,
   preferably said animal cell is derived from non-human mammals, birds, fish, invertebrates, reptiles, amphibians or insects or is a metabolically engineered cell line derived from human cells excluding embryonic stem cells, more preferably said human and non-human mammalian cell is an epithelial cell, an embryonic kidney cell, a fibroblast cell, a COS cell, a Chinese hamster ovary (CHO) cell, a murine myeloma cell, an NIH-3T3 cell, a non-mammary adult stem cell or derivatives thereof, more preferably said insect cell is derived from Spodoptera frugiperda, Bombyx mori, Mamestra brassicae, Trichoplusia ni or Drosophila melanogaster,
   preferably said protozoan cell is a Leishmania tarentolae cell.
36. Cell according to any one of previous preferred embodiments, wherein said cell is stably cultured in a medium.
37. Cell according to any one of previous preferred embodiments, wherein the cell is capable to synthesize a mixture of compounds.
38. Method for the production of a compound wherein said compound is a disaccharide, oligosaccharide and/or a Neu(n)Ac-containing bioproduct, wherein (n) is 4, 5, 7, 8 or 9 or a combination thereof, the method comprising the steps of:
   a. providing a cell capable to produce said compound, wherein said cell is metabolically engineered for enhanced synthesis of acetyl-Coenzyme A,
   b. cultivating the cell under conditions permissive for producing said compound,
   c. and preferably separating the desired compound from said cultivation.
39. Method for the production of a compound, wherein said compound is a disaccharide, oligosaccharide and/or a Neu(n)Ac-containing bioproduct, wherein (n) is 4, 5, 7, 8 or 9 or a combination thereof by a metabolically engineered cell, comprising the steps of:
   a) providing a cell according to any one of the preferred embodiments 1 to 36, and
   b) culturing the cell in a culture medium under conditions permissive for the production of said compound,
   c) preferably separating said compound from the cultivation.
40. Method according to any one of preferred embodiments 38 or 39, the method further comprising:
   i) Use of a culture medium comprising at least one precursor and/or acceptor for the production of said compound, and/or
   ii) Adding to the culture medium at least one precursor and/or acceptor feed for the production of said compound.
41. Method according to any one of preferred embodiments 38 to 40, the method further comprising at least one of the following steps:
   i) Use of a culture medium comprising at least one precursor and/or acceptor;
   ii) Adding to the culture medium in a reactor or incubator at least one precursor and/or acceptor feed wherein the total reactor or incubator volume ranges from 250 mL (millilitre) to 10,000 m³ (cubic meter), preferably in a continuous manner, and preferably so that the final volume of the culture medium is not more than three-fold, preferably not more than two-fold, more preferably less than 2-fold of the volume of the culture medium before the addition of said precursor and/or acceptor feed;
   iii) Adding to the culture medium in a reactor or incubator at least one precursor and/or acceptor feed wherein the total reactor or incubator volume ranges from 250 mL (millilitre) to 10,000 m³ (cubic meter), preferably in a continuous manner, and preferably so that the final volume of the culture medium is not more than three-fold, preferably not more than two-fold, more preferably less than 2-fold of the volume of the culture medium before the addition of said precursor and/or acceptor feed and wherein preferably, the pH of said precursor and/or acceptor feed is set between 2.0 and 10.0 and wherein preferably, the temperature of said precursor and/or acceptor feed is kept between 20° C. and 80° C.;
   iv) Adding at least one precursor and/or acceptor feed in a continuous manner to the culture medium over the course of 1 day, 2 days, 3 days, 4 days, 5 days by means of a feeding solution;
   v) Adding at least one precursor and/or acceptor feed in a continuous manner to the culture medium over the course of 1 day, 2 days, 3 days, 4 days, 5 days by means of a feeding solution and wherein preferably, the pH of said feeding solution is set between 2.0 and 10.0 and wherein preferably, the temperature of said feeding solution is kept between 20° C. and 80° C.;
   said method resulting in a compound with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of said culture medium.
42. Method according to any one of preferred embodiments 38 to 40, the method further comprising at least one of the following steps:
   i) Use of a culture medium comprising at least 50, more preferably at least 75, more preferably at least 100, more preferably at least 120, more preferably at least 150 grams of precursor per litre of initial reactor or incubator volume wherein the total reactor or incubator volume ranges from 250 mL to 10,000 m³ (cubic meter);
   ii) Use of a culture medium comprising at least 50, more preferably at least 75, more preferably at least 100, more preferably at least 120, more preferably at least 150 grams of acceptor per litre of initial reactor or incubator volume wherein the total reactor or incubator volume ranges from 250 mL to 10,000 m³ (cubic meter);
   iii) Adding to the culture medium in a reactor or incubator a precursor feed comprising at least 50, more preferably at least 75, more preferably at least 100, more preferably at least 120, more preferably at least 150 grams of precursor per litre of initial reactor or incubator volume wherein the total reactor or incubator volume ranges from 250 mL to 10,000 m³ (cubic meter), preferably in a continuous manner, and preferably so that the final volume of the culture medium is not more than three-fold, preferably not more than two-fold, more preferably less than 2-fold of the volume of the culture medium before the addition of said precursor feed;
   iv) Adding to the culture medium in a reactor or incubator an acceptor feed comprising at least 50, more preferably at least 75, more preferably at least 100, more preferably at least 120, more preferably at least 150 grams of acceptor per litre of initial reactor or incubator volume wherein the total reactor or incubator volume ranges from 250 mL to 10,000 m³ (cubic meter), preferably in a continuous manner, and preferably so that the final volume of the culture medium is not more than three-fold, preferably not more than two-fold, more preferably less than 2-fold of the volume of the culture medium before the addition of said acceptor feed;
   v) Adding to the culture medium in a reactor or incubator a precursor feed comprising at least 50, more preferably at least 75, more preferably at least 100, more preferably at least 120, more preferably at least 150 grams of precursor per litre of initial reactor or incubator volume wherein the total reactor or incubator volume ranges from 250 mL to 10,000 m³ (cubic meter), preferably in a continuous manner, and preferably so that the final volume of the culture medium is not more than three-fold, preferably not more than two-fold, more preferably less than 2-fold of the volume of the culture medium before the addition of said precursor feed and wherein preferably, the pH of said precursor feed is set between 2.0 and 10.0 and wherein preferably, the temperature of said precursor feed is kept between 20° C. and 80° C.;
   vi) Adding to the culture medium in a reactor or incubator an acceptor feed comprising at least 50, more preferably at least 75, more preferably at least 100, more preferably at least 120, more preferably at least 150 grams of acceptor per litre of initial reactor or incubator volume wherein the total reactor or incubator volume ranges from 250 mL to 10,000 m³ (cubic meter), preferably in a continuous manner, and preferably so that the final volume of the culture medium is not more than three-fold, preferably not more than two-fold, more preferably less than 2-fold of the volume of the culture medium before the addition of said acceptor feed and wherein preferably, the pH of said acceptor feed is set between 2.0 and 10.0 and wherein preferably, the temperature of said acceptor feed is kept between 20° C. and 80° C.;
   vii) Adding a precursor and/or acceptor feed in a continuous manner to the culture medium over the course of 1 day, 2 days, 3 days, 4 days, 5 days by means of a feeding solution;
   viii) Adding a precursor feed in a continuous manner to the culture medium over the course of 1 day, 2 days, 3 days, 4 days, 5 days by means of a precursor feeding solution and wherein the concentration of said precursor feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L and wherein preferably, the pH of said precursor feeding solution is set between 2.0 and 10.0 and wherein preferably, the temperature of said precursor feeding solution is kept between 20° C. and 80° C.;
   ix) Adding an acceptor feed in a continuous manner to the culture medium over the course of 1 day, 2 days, 3 days, 4 days, 5 days by means of an acceptor feeding solution and wherein the concentration of said acceptor feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L and wherein preferably, the pH of said acceptor feeding solution is set between 2.0 and 10.0 and wherein preferably, the temperature of said acceptor feeding solution is kept between 20° C. and 80° C.;
said method resulting in a compound with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of said culture medium.
43. Method according to any one of preferred embodiments 38 to 40, the method further comprising at least one of the following steps:
   i) Use of a culture medium comprising at least 50, more preferably at least 75, more preferably at least 100, more preferably at least 120, more preferably at least 150 grams of lactose per litre of initial reactor or incubator volume wherein the total reactor or incubator volume ranges from 250 mL to 10,000 m³ (cubic meter);
   ii) Adding to the culture medium in a reactor or incubator a lactose feed comprising at least 50, more preferably at least 75, more preferably at least 100, more preferably at least 120, more preferably at least 150 gram of lactose per litre of initial reactor or incubator volume wherein the total reactor or incubator volume ranges from 250 mL to 10,000 m³ (cubic meter), preferably in a continuous manner, and preferably so that the final volume of the culture medium is not more than three-fold, preferably not more than two-fold, more preferably less than 2-fold of the volume of the culture medium before the addition of said lactose feed;
   iii) Adding to the culture medium in a reactor or incubator a lactose feed comprising at least 50, more preferably at least 75, more preferably at least 100, more preferably at least 120, more preferably at least 150 grams of lactose per litre of initial reactor or incubator volume wherein the total reactor or incubator volume ranges from 250 mL to 10,000 m³ (cubic meter), preferably in a continuous manner, and preferably so that the final volume of the culture medium is not more than three-fold, preferably not more than two-fold, more preferably less than 2-fold of the volume of the culture medium before the addition of said lactose feed and wherein preferably, the pH of said lactose feed is set between 2.0 and 10.0 and wherein preferably, the temperature of said lactose feed is kept between 20° C. and 80° C.;
   iv) Adding a lactose feed in a continuous manner to the culture medium over the course of 1 day, 2 days, 3 days, 4 days, 5 days by means of a feeding solution;
   v) Adding a lactose feed in a continuous manner to the culture medium over the course of 1 day, 2 days, 3 days, 4 days, 5 days by means of a lactose feeding solution and wherein the concentration of said lactose feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably the pH of said lactose feeding solution is set between 2.0 and 10.0 and wherein preferably the temperature of said lactose feeding solution is kept between 20° C. and 80° C.;
said method resulting in a compound with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of said culture medium.
44. Method according to preferred embodiment 43, wherein the lactose feed is accomplished by adding lactose from the beginning of the cultivation in a concentration of at least 5 mM, preferably in a concentration of 30, 40, 50, 60, 70, 80, 90, 100, 150 mM, more preferably in a concentration >300 mM.
45. Method according to any one of preferred embodiment 43 or 44, wherein said lactose feed is accomplished by adding lactose to the culture medium in a concentration, such, that throughout the production phase of the cultivation a lactose concentration of at least 5 mM, preferably 10 mM or 30 mM is obtained.
46. Method for the production of a mixture of compounds by a metabolically engineered cell, comprising the steps of:
   a) providing a cell according to preferred embodiment 37, and
   b) culturing the cell in a culture medium under conditions permissive for the production of said compounds,
   c) preferably separating said mixture of compounds from the cultivation.
47. Method according to any one of preferred embodiments 38 to 46, wherein the cells are cultivated for at least about 60, 80, 100, or about 120 hours or in a continuous manner.
48. Method according to any one of preferred embodiments 38 to 47, wherein a carbon and energy source, preferably glucose, glycerol, fructose, maltose, arabinose, maltodextrines, malto-oligosaccharides, maltotriose, sorbitol, xylose, rhamnose, sucrose, galactose, mannose, methanol, ethanol, trehalose, starch, cellulose, hemi-cellulose, polyols, corn-steep liquor, high-fructose syrup, succinate, malate, acetate, citrate, lactate and pyruvate, is also added, preferably continuously to the culture medium, preferably with the precursor and/or acceptor.
49. Method according to any one of preferred embodiments 38 to 48, wherein said cell uses at least one precursor for the synthesis of said compound, preferably said cell uses two or more precursors for the synthesis of said compound.
50. Method according to any one of preferred embodiments 38 to 49, wherein the culture medium contains at least one molecule selected from the group comprising lactose, galactose, sialic acid, fucose, GlcNAc, GalNAc, lacto-N-biose (LNB), N-acetyllactosamine (LacNAc).
51. Method according to any one of preferred embodiments 38 to 50, wherein a first phase of exponential cell growth is provided by adding a carbon-based substrate, preferably glucose or sucrose, to the culture medium before the precursor and/or acceptor is added to the culture medium in a second phase.
52. Method according to any one of preferred embodiments 38 to 51, wherein said cell is producing at least one precursor for the synthesis of said compound.
53. Method according to any one of preferred embodiments 40 to 42, 46 to 52, wherein said precursor for the synthesis of said compound is completely converted into said compound.
54. Method according to any one of preferred embodiments 38 to 53, wherein the compound is separated from the culture medium and/or the cell.
55. Method according to preferred embodiment 54, wherein said separation comprises at least one of the following steps: clarification, ultrafiltration, nanofiltration, two-phase partitioning, reverse osmosis, microfiltration, activated charcoal or carbon treatment, treatment with non-ionic surfactants, enzymatic digestion, tangential flow high-performance filtration, tangential flow ultrafiltration, affinity chromatography, ion exchange chromatography, hydrophobic interaction chromatography and/or gel filtration, ligand exchange chromatography, electrodialysis.
56. Method according to any one of preferred embodiments 38 to 55, wherein said method further comprises purification of said compound.
57. Method according to preferred embodiment 56, wherein said purification comprises at least one of the following steps: use of activated charcoal or carbon, use of charcoal, nanofiltration, ultrafiltration, electrophoresis, enzymatic treatment or ion exchange, temperature adjustment, pH adjustment, pH adjustment with an alkaline or acidic solution, use of alcohols, use of aqueous alcohol mixtures, crystallization, evaporation, precipitation, drying, spray drying, lyophilization, spray freeze drying, freeze spray drying, band drying, belt drying, vacuum band drying, vacuum belt drying, drum drying, roller drying, vacuum drum drying or vacuum roller drying.
58. Use of a cell as defined in any one of preferred embodiments 1 to 36 in the fermentative production of a compound, preferably an oligosaccharide.
59. Use of a cell according to preferred embodiment 37 in the fermentative production of a mixture of compounds, preferably comprising at least two oligosaccharides.
60. Use of a method according to any one of preferred embodiments 38 to 57 for the production of a compound, preferably an oligosaccharide.

The invention will be described in more detail in the examples. The following examples will serve as further illustration and clarification of the present invention and are not intended to be limiting. As such acetyl-Coenzyme A ligase was chosen to enhance the synthesis of acetyl-Coenzyme A and learn what the effect of such enhanced acetyl-Coenzyme A synthesis is on disaccharide, oligosaccharide and or Neu(n)Ac-containing bioproduct production.

The skilled person will understand that acetyl-CoA ligase is a model enzyme for enhanced synthesis of acetyl-Coenzyme A and that the other proposed methods to enhance the synthesis of acetyl-Coenzyme A will produce the same effect on the production of disaccharide, oligosaccharide and or Neu(n)Ac-containing bioproduct.

### EXAMPLES

### Example 1. Materials and Methods

A. Escherichia coli

### Media

The Luria Broth (LB) medium consisted of 1% tryptone peptone (Difco, Erembodegem, Belgium), 0.5% yeast extract (Difco) and 0.5% sodium chloride (VWR. Leuven, Belgium). The minimal medium used in the cultivation experiments in 96-well plates or in shake flasks contained 2.00 g/L NH4Cl, 5.00 g/L (NH4)2SO4, 2.993 g/L KH2PO4, 7.315 g/L K2HPO4, 8.372 g/L MOPS, 0.5 g/L NaCl, 0.5 g/L MgSO4·7H2O, 30 g/L sucrose or another carbon source when specified in the examples, 1 ml/L vitamin solution, 100 μL/L molybdate solution, and 1 mL/L selenium solution. As specified in the respective examples, 20 g/L lactose was additionally added to the medium as precursor. The minimal medium was set to a pH of 7 with 1M KOH. Vitamin solution consisted of 3.6 g/L FeCl2·4H2O, 5 g/L CaCl2·2H2O, 1.3 g/L MnCl2·2H2O, 0.38 g/L CuCl2·2H2O, 0.5 g/L CoCl2·6H2O, 0.94 g/L ZnCl2, 0.0311 g/L H3BO4, 0.4 g/L Na2EDTA·2H2O and 1.01 g/L thiamine·HCl. The molybdate solution contained 0.967 g/L NaMoO4·2H2O. The selenium solution contained 42 g/L Seo2.

The minimal medium for fermentations contained 6.75 g/L NH4Cl, 1.25 g/L (NH4)2SO4, 2.93 g/L KH2PO4 and 7.31 g/L KH2PO4, 0.5 g/L NaCl, 0.5 g/L MgSO4·7H2O, 30 g/L sucrose, 1 mL/L vitamin solution, 100 μL/L molybdate solution, and 1 mL/L selenium solution with the same composition as described above. As specified in the respective examples, 100 g/L lactose was additionally added to the medium as precursor.

Complex medium was sterilized by autoclaving (121° C., 21′) and minimal medium by filtration (0.22 μm Sartorius). When necessary, the medium was made selective by adding an antibiotic (e.g. chloramphenicol (20 mg/L), carbenicillin (100 mg/L), spectinomycin (40 mg/L) and/or kanamycin (50 mg/L)).

### Plasmids

pKD46 (Red helper plasmid, Ampicillin resistance), pKD3 (contains an FRT-flanked chloramphenicol resistance (cat) gene), pKD4 (contains an FRT-flanked kanamycin resistance (kan) gene), and pCP20 (expresses FLP recombinase activity) plasmids were obtained from Prof. R. Cunin (Vrije Universiteit Brussel, Belgium in 2007). Plasmids were maintained in the host E. coli DH5alpha (F⁻, phi80dlacZdeltaM15, delta(lacZYAargF) U169, deoR, recA1, endA1, hsdR17(rk⁻, mk⁺), phoA, supE44, lambda⁻, thi-1, gyrA96, relA1) bought from Invitrogen.

### Strains and Mutations

Escherichia coli K12 MG1655 [lambda⁻, F⁻, rph-1] was obtained from the Coli Genetic Stock Center (US), CGSC Strain #: 7740, in March 2007. Gene disruptions as well as gene introductions were performed using the technique published by Datsenko and Wanner (PNAS 97 (2000), 6640-6645). This technique is based on antibiotic selection after homologous recombination performed by lambda Red recombinase. Subsequent catalysis of a flippase recombinase ensures removal of the antibiotic selection cassette in the final production strain.

Transformants carrying a Red helper plasmid pKD46 were grown in 10 ml LB media with ampicillin, (100 mg/L) and L-arabinose (10 mM) at 30° C. to an OD₆ₒₒₙₘ of 0.6. The cells were made electrocompetent by washing them with 50 mL of ice-cold water, a first time, and with 1 mL ice cold water, a second time. Then, the cells were resuspended in 50 μL of ice-cold water. Electroporation was done with 50 μL of cells and 10-100 ng of linear double-stranded-DNA product by using a Gene Pulser™ (BioRad) (600Ω, 25 μFD, and 250 volts). After electroporation, cells were added to 1 mL LB media incubated 1 h at 37° C., and finally spread onto LB-agar containing 25 mg/L of chloramphenicol or 50 mg/L of kanamycin to select antibiotic resistant transformants. The selected mutants were verified by PCR with primers upstream and downstream of the modified region and were grown in LB-agar at 42° C. for the loss of the helper plasmid. The mutants were tested for ampicillin sensitivity. The linear ds-DNA amplicons were obtained by PCR using pKD3, pKD4 and their derivates as template. The primers used had a part of the sequence complementary to the template and another part complementary to the side on the chromosomal DNA where the recombination must take place. For the genomic knock-out, the region of homology was designed 50-nt upstream and 50-nt downstream of the start and stop codon of the gene of interest. For the genomic knock-in, the transcriptional starting point (+1) had to be respected. PCR products were PCR-purified, digested with DpnI, repurified from an agarose gel, and suspended in elution buffer (5 mM Tris, pH 8.0). The selected mutants (chloramphenicol or kanamycin resistant) were transformed with pCP20 plasmid, which is an ampicillin and chloramphenicol resistant plasmid that shows temperature-sensitive replication and thermal induction of FLP synthesis. The ampicillin-resistant transformants were selected at 30° C., after which a few were colony purified in LB at 42° C. and then tested for loss of all antibiotic resistance and of the FLP helper plasmid. The gene knockouts and knock-ins are checked with control primers.

In one example a sialic acid producing base strain derived from E. coli K12 MG1655 was created by knocking out the genes asl, IdhA, poxB, atpI-gidB and ackA-pta, and knocking out the operons lacZYA, nagAB and the genes nanA, nanE and nanK. Additionally, the E. coli lacY gene was introduced at the location of lacZYA. A fructose kinase gene (frk) originating from Zymomonas mobilis, an E. coli W sucrose transporter (cscB), a sucrose phosphorylase (SP) originating from Bifidobacterium adolescentis, an E. coli mutant fructose-6-P-aminotransferase (EcglmS*54, as described by Deng et al. (Biochimie 88, 419-29 (2006)), glucosamine-6-P-aminotransferase from Saccharomyces cerevisiae (ScGNA1), an N-acetylglucosamine-2-epimerase from Bacteroides ovatus (BoAGE) and a sialic acid synthase from Campylobacter jejuni (CjneuB) or a sialic acid synthase from Neisseria meningitidis (NmNeuB) were knocked in into the genome.

Alternatively and/or additionally, sialic acid production can be obtained by genomic knock-ins of constitutive transcriptional units containing a UDP-N-acetylglucosamine 2-epimerase like e.g. NeuC from Campylobacter jejuni and any one or more copies of an N-acetylneuraminate synthase like e.g. NeuB from Neisseria meningitidis or from Campylobacter jejuni.

Alternatively and/or additionally, sialic acid production can be obtained by genomic knock-ins of constitutive transcriptional units containing a phosphoglucosamine mutase like e.g. glmM from E. coli, an N-acetylglucosamine-1-phosphate uridyltransferase/glucosamine-1-phosphate acetyltransferase like e.g. glmU from E. coli, a UDP-N-acetylglucosamine 2-epimerase like e.g. NeuC from Campylobacter jejuni and any one or more copies of an N-acetylneuraminate synthase like e.g. NeuB from Neisseria meningitidis or from Campylobacter jejuni.

Alternatively and/or additionally, sialic acid production can be obtained by genomic knock-ins of constitutive transcriptional units containing a bifunctional UDP-GlcNAc 2-epimerase/N-acetylmannosamine kinase like e.g. from Mus musculus (strain C57BL/6J), an N-acylneuraminate-9-phosphate synthetase, and an N-acylneuraminate-9-phosphatase like e.g. from Candidatus magnetomorum sp. HK-1 or from Bacteroides thetaiotaomicron (strain ATCC 29148).

Alternatively and/or additionally, sialic acid production can be obtained by genomic knock-ins of constitutive transcriptional units containing a phosphoglucosamine mutase like e.g. glmM from E. coli, an N-acetylglucosamine-1-phosphate uridyltransferase/glucosamine-1-phosphate acetyltransferase like e.g. glmU from E. coli, a bifunctional UDP-GlcNAc 2-epimerase/N-acetylmannosamine kinase like e.g. from M. musculus (strain C57BL/6J), an N-acylneuraminate-9-phosphate synthetase, and an N-acylneuraminate-9-phosphatase like e.g. from Candidatus magnetomorum sp. HK-1 or from Bacteroides thetaiotaomicron (strain ATCC 29148).

Sialic acid production can further be optimized in the mutant E. coli strain with genomic knock-outs of the E. coli genes comprising any one or more of nagA, nagB, nagC, nagD, nagE, nanA, nanE, nanK, manX, manY and manZ as described in WO18122225, and/or genomic knock-outs of the E. coli genes comprising any one or more of poxB, ldhA, adhE, aldB, pflA, pflC, ybiY, ackA and/or pta, and with genomic knock-in of constitutive transcriptional units comprising any one or more of an L-glutamine-D-fructose-6-phosphate aminotransferase like e.g. the mutant glmS*54 from E. coli (differing from the wild-type E. coli glmS by an A39T, an R250C and an G472S mutation).

For sialylated oligosaccharide production, said sialic acid production strains further need to express one or more copies of an N-acylneuraminate cytidylyltransferases like e.g. NeuA from Pasteurella multocida, NeuA from C. jejuni or NeuA from Haemophilus influenzae, and one or more copies of a beta-galactoside alpha-2,3-sialyltransferase, e. g. chosen from the list comprising PmultST2 from P. multocida subsp. multocida str. Pm70, NmeniST3 from N. meningitidis and PmultST3 from P. multocida, a beta-galactoside alpha-2,6-sialyltransferase, such as the one chosen from the list comprising PdST6 from Photobacterium damselae and P-JT-ISH-224-ST6 from Photobacterium sp. JT-ISH-224, and/or an alpha-2,8-sialyltransferase, such as e.g. from Mus musculus. Constitutive transcriptional units of the N-acylneuraminate cytidylyltransferases and the sialyltransferases can be delivered to the mutant strain either via genomic knock-in or via expression plasmids. If the mutant strains producing sialic acid and CMP-sialic acid were intended to make sialylated lactose structures, the strains were additionally modified with genomic knock-outs of the E. coli LacZ, LacY and LacA genes and with a genomic knock-in of a constitutive transcriptional unit for a lactose permease like e.g. the E. coli LacY. All mutant strains producing sialic acid, CMP-sialic acid and/or sialylated oligosaccharides could optionally be adapted for growth on sucrose via genomic knock-ins of constitutive transcriptional units containing a sucrose transporter (CscB), e.g. from E. coli W, a fructose kinase (Frk) e.g. originating from Z. mobilis and a sucrose phosphorylase e.g. originating from B. adolescentis.

In an example to allow production of 6′-SL, the sialic acid base strain was further modified by introducing two constructs both expressing a CMP-sialic acid synthetase from Neisseria meningitidis (NmneuA) and an α-2,6-sialyltransferase from Photobacterium damselae (PdbST) into the genome.

In an example to allow production of 3′-SL, the sialic acid base strain was further modified by introducing a construct expressing a CMP-sialic acid synthetase from Neisseria meningitidis (NmneuA) and an α-2,3-sialyltransferase from Neisseria meningitidis (NmST) which were knocked in into the genome.

In an example to allow production of sialylated LacNAc (sLacNAc), the sialic acid base strain was further modified by a CMP-sialic acid synthetase from Neisseria meningitidis (NmneuA) and a sialyltransferase which were knocked in into the genome. For 6′-sLacNAc, a sialyltransferase from Photobacterium damselae (PdbST) was used and for 3′-sLacNAc, a sialyltransferase from Neisseria meningitidis (NmST) was used.

In an example to allow production of sialylated LNB (sLNB), the sialic acid base strain was further modified by introducing a CMP-sialic acid synthetase from Neisseria meningitidis (NmneuA) and a sialyltransferase which were knocked in into the genome. For 6′-sLNB, a sialyltransferase from Photobacterium damselae (PdbST) was used and for 3′-sLNB, a sialyltransferase from Neisseria meningitidis (NmST) was used.

In an example to allow production of LSTa and LSTb, the sialic acid base strain was further modified by introducing a beta-1,3-GlcNAc transferase from Neisseria meningitidis (NmlgtA), a beta-1,3-galactosyltransferase from E. coli O55:H7 (EcwbgO), a CMP-sialic acid synthetase and an alpha-2,3-sialyltransferase or an alpha-2,6-sialyltransferase for production of LSTa or LSTb, respectively. Alternatively, sialic acid can be fed to an optimized lacto-N-tetraose producing strain with expression of a beta-1,3-GlcNAc transferase from Neisseria meningitidis (NmlgtA) and a beta-1,3-galactosyltransferase from E. coli O55:H7 (EcwbgO) (as described and demonstrated in Example 8 of WO18122225), and additional expression of a CMP-sialic acid synthetase and an α-2,3-sialyltransferase or an α-2,6-sialyltransferase to allow LSTa or LSTb production, respectively.

In an example to allow production of LSTc and LSTd, the sialic acid base strain was further modified by introducing a beta-1,3-GlcNAc transferase from Neisseria meningitidis (NmlgtA), a beta-1,4-galactosyltransferase from Neisseria meningitidis (NmlgtB), a CMP-sialic acid synthetase and an alpha-2,3-sialyltransferase or an alpha-2,6-sialyltransferase for production of LSTc or LSTd, respectively. Alternatively, sialic acid can be fed to an optimized lacto-N-neotetraose producing strain with expression of a beta-1,3-GlcNAc transferase from Neisseria meningitidis (NmlgtA) and a beta-1,4-galactosyltransferase from Neisseria meningitidis (NmlgtB) (as described and demonstrated in Example 8 of WO18122225), and additional expression of a CMP-sialic acid synthetase and an alpha-2,3-sialyltransferase or an alpha-2,6-sialyltransferase to allow LSTc or LSTd production, respectively.

All these genes were constitutively expressed with promoters originating from the promoter library described by De Mey et al. (BMC Biotechnology, 2007) or by Mutalik et al. (Nat. Methods 2013, No. 10, 354-360). UTRs originated from Mutalik et al. (Nat. Methods 2013, No. 10, 354-360) and terminators originated from Dunn et al. (Nucleic Acids Res. 1980, 8(10), 2119-32) and Orosz et al. (Eur. J. Biochem. 1991, 201, 653-59). These genetic modifications are also described in WO18122225.

In an example to produce lacto-N-triose (LN3, LNT-II, GlcNAc-b1,3-Gal-b1,4-Glc) and oligosaccharides originating thereof comprising lacto-N-tetraose (LNT) and lacto-N-neotetraose (LNnT), the mutant strain was derived from E. coli K12 MG1655 and modified with a knock-out of the E. coli LacZ and nagB genes and with a genomic knock-in of a constitutive transcriptional unit for the galactoside beta-1,3-N-acetylglucosaminyltransferase (LgtA) from N. meningitidis. In an example for LNT or LNnT production, the mutant strain producing LN3 is further modified with constitutive transcriptional units for the N-acetylglucosamine beta-1,3-galactosyltransferase (WbgO) from E. coli O55:H7 or the N-acetylglucosamine beta-1,4-galactosyltransferase (LgtB) from N. meningitidis, respectively, that can be delivered to the strain either via genomic knock-in or from an expression plasmid. Optionally, multiple copies of the LgtA, wbgO and/or LgtB genes could be added to the mutant E. coli strains. In these strains, LNT and/or LNnT production can be enhanced by improved UDP-GlcNAc production by modification of the strains with one or more genomic knock-ins of a constitutive transcriptional unit for the mutant L-glutamine-D-fructose-6-phosphate aminotransferase glmS*54 from E. coli as described above. In addition, the strains can optionally be modified for enhanced UDP-galactose production with genomic knockouts of the E. coli ushA and galT genes. The mutant E. coli strains can also optionally be adapted with a genomic knock-in of a constitutive transcriptional unit for the UDP-glucose-4-epimerase (galE) from E. coli, the phosphoglucosamine mutase (glmM) from E. coli and the N-acetylglucosamine-1-phosphate uridyltransferase/glucosamine-1-phosphate acetyltransferase (glmU) from E. coli.

In an example for production of fucosylated oligosaccharides with LN3 as a core trisaccharide, the E. coli strains modified for production of LN3, LNT and/or LNnT were further modified with knockouts of the E. coli wcaJ and thyA genes and with expression plasmids comprising constitutive transcriptional units for the H. pylori alpha-1,2-fucosyltransferase (HpFutC) and/or the H. pylori alpha-1,3-fucosyltransferase (HpFucT) and with a constitutive transcriptional unit for the E. coli thyA as selective marker. The constitutive transcriptional units of the fucosyltransferase genes could also be present in the mutant E. coli strain via genomic knock-ins. GDP-fucose production can further be optimized by genomic knockouts of the E. coli genes comprising glgC, agp, pfkA, pfkB, pgi, arcA, iclR, pgi and Ion as described in WO2016075243 and WO2012007481. GDP-fucose production can additionally be optimized comprising genomic knock-ins of constitutive transcriptional units for the E. coli manA, manB, manC, gmd and fcl. GDP-fucose production can also be obtained by genomic knockouts of the E. coli fucK and fucI genes and genomic knock-ins of constitutive transcriptional units containing the fucose permease (fucP) from E. coli and the bifunctional fucose kinase/fucose-1-phosphate guanylyltransferase (fkp) from Bacteroides fragilis.

All mutant strains could also optionally be adapted for growth on sucrose via genomic knock-ins of constitutive transcriptional units containing a sucrose transporter (CscB) from E. coli W, a fructose kinase (Frk) originating from Z. mobilis and a sucrose phosphorylase originating from B. adolescentis. Furthermore, the mutant strains could be modified for enhanced lactose uptake via genomic knock-in of a constitutive transcriptional unit for the lactose permease lacY from E. coli.

Furthermore, all mutant strains could be optionally adapted for intracellular lactose synthesis by genomic knock-outs of lacZ, glk and the galETKM operon, together with genomic knock-ins of constitutive transcriptional units for lgtB from N. meningitidis and the UDP-glucose 4-eprimerase (galE) from E. coli. Preferably but not necessarily, the glycosyltransferases were N-terminally fused to an MBP-tag to enhance their solubility (Fox et al., Protein Sci. 2001, 10(3), 622-630).

All constitutive promoters, UTRs and terminator sequences originated from the libraries described by Cambray et al. (Nucleic Acids Res. 2013, 41(9), 5139-5148), Chen et al. (Nat. Methods 2013, 10(7), 659-664), De Mey et al. (BMC Biotechnol. 2007, 4(34), 1-14), Dunn et al. (Nucleic Acids Res. 1980, 8(10), 2119-2132), Kim and Lee (FEBS Letters 1997, 407(3), 353-356) and Mutalik et al. (Nat. Methods 2013, No. 10, 354-360). All genes were ordered synthetically at Twist Bioscience (twistbioscience.com) or IDT (eu.idtdna.com) and the codon usage was adapted using the tools of the supplier.

In an example for fucosyllactose production, a mutant strain derived from E. coli K12 MG1655 was created by knocking out the genes lacZ, lacY lacA, glgC, agp, pfkA, pfkB, pgi, arcA, iclR, wcaJ, lon and thyA. Additionally, the E. coli lacY gene, a fructose kinase gene (frk) originating from Zymomonas mobilis, an E. coli W sucrose transporter (cscB) and a sucrose phosphorylase (SP) originating from Bifidobacterium adolescentis were knocked in into the genome and expressed constitutively. The constitutive promoters originate from the promoter library described by De Mey et al. (BMC Biotechnology, 2007). These genetic modifications are also described in WO2016075243 and WO2012007481. The α1,3- or α1,2-fucosyltransferase genes were presented to the mutant strain from a plasmid as described herein resulting in the production of 2′fucosyllactose, 3-fucosyllactose or 2′,3-difucosyllactose.

An alternative mutant strain can be derived from E. coli K12 JM109 wherein the genes lacZ, rcsA and wcaJ are knocked out. α1,3- or α1,2-fucosyltransferase genes are presented to said mutant strain from a plasmid as described herein resulting in the production of 2′fucosyllactose, 3-fucosyllactose or 2′,3-difucosyllactose. Said strain is enabled to internalize lactose by means of allo-lactose or IPTG, inducing the lactose permease gene lacY.

Another alternative mutant strain can be derived from E coli BL21. The genes lacZ, fucI, fucK and wzxC-wcaJ are knocked out in said strain. In order to improve the synthesis of GDP-fucose in said mutant strain the genes encoding for phosphomannomutase (manB), mannose-1-phosphate guanosyltransferase (manC), GDP-mannose-4,6-dehydratase (gmd), and GDP-L-fucose synthase (wcaG) from E. coli K12 were overexpressed in a similar way as described above. Intracellular lactose synthesis is accomplished by overexpression of the gene encoding for beta-1,4-galactosyltransferase encoded by the gene IgtB. To enhance the synthesis of UDP-galactose the operon encoding for galETKM is knocked out and the gene encoding for UDP-glucose epimerase is overexpressed. α1,3- or α1,2-fucosyltransferase genes are presented to said mutant strain from a plasmid as described herein resulting in the production of 2′fucosyllactose, 3-fucosyllactose or 2′,3-difucosyllactose.

Another alternative mutant strain can be derived from E. coli K12. The genes lacZ, fucI, fucK and wzxC-wcaJ are knocked out in said strain. In order to improve the synthesis of GDP-fucose in said mutant strain the genes encoding for phosphomannomutase (manB), mannose-1-phosphate guanosyltransferase (manC), GDP-mannose-4,6-dehydratase (gmd), and GDP-L-fucose synthase (wcaG) from E. coli K12 were overexpressed in a similar way as described above. In addition, said strain is modified with genomic knock-ins of the fucose permease (fucP) gene from E. coli and the bifunctional fucose kinase/fucose-1-phosphate guanylyltransferase (fkp) gene from Bacteroides fragilis. α1,3- or α1,2-fucosyltransferase genes are presented to said mutant strain from a plasmid as described herein resulting in the production of 2′fucosyllactose, 3-fucosyllactose or 2′,3-difucosyllactose. Said strain is enabled to internalize lactose by means of allo-lactose or IPTG, inducing the lactose permease gene lacY.

Another alternative mutant strain can be derived from E. coli K12. The genes lacZ, and wzxC-wcaJ are knocked out in said strain. In order to improve the synthesis of GDP-fucose in said mutant strain the genes encoding for phosphomannomutase (manB), mannose-1-phosphate guanosyltransferase (manC), GDP-mannose-4,6-dehydratase (gmd), and GDP-L-fucose synthase (wcaG) from E. coli K12 were overexpressed in a similar way as described above. To improve the formation of fructose-6-phosphate from gluconeogenic substrates such as glycerol, acetate, lactate, ethanol, succinate, pyruvate, the genes encoding for phosphofructokinase (pfkA and pfkB) are knocked out and the genes encoding for fructose-1,6-bisphosphate aldolase (fbaB) and a heterologous fructose-1,6-bisphosphate phosphatase (fbpase) from Pisum sativum were overexpressed. α1,3- or α1,2-fucosyltransferase genes are presented to said mutant strain from a plasmid as described herein resulting in the production of 2′fucosyllactose, 3-fucosyllactose or 2′,3-difucosyllactose.

All constitutive promoters and UTRs originate from the libraries described by De Mey et al. (BMC Biotechnology, 2007) and Mutalik et al. (Nat. Methods 2013, No. 10, 354-360). All genes were ordered synthetically at Twist Bioscience (twistbioscience.com) or IDT (eu.idtdna.com) and the codon usage was adapted using the tools of the supplier.

Preferably but not necessarily, the glycosyltransferases and/or proteins involved in nucleotide-activated sugar synthesis were N- and/or C-terminally fused to a solubility enhancer tag like e.g. a SUMO-tag, an MBP-tag, His, FLAG, Strep-II, Halo-tag, NusA, thioredoxin, GST and/or the Fh8-tag to enhance their solubility (Costa et al., Front. Microbiol. 2014, https://doi.org/10.3389/fmicb.2014.00063; Fox et al., Protein Sci. 2001, 10(3), 622-630; Jia and Jeaon, Open Biol. 2016, 6: 160196).

Optionally, the mutant E. coli strains are modified to create a glycominimized E. coli strain comprising genomic knock-out of any one or more of non-essential glycosyltransferase genes comprising pgaC, pgaD, rfe, rffT, rffM, bcsA, bcsB, bcsC, wcaA, wcaC, wcaE, wcaI, wcaJ, wcaL, waaH, waaF, waaC, waaU, waaZ, waaJ, waaO, waaB, waaS, waaG, waaQ, wbbI, arnC, arnT, yfdH, wbbK, opgG, opgH, ycjM, glgA, glgB, malQ, otsA and yaiP. All strains are stored in cryovials at −80° C. (overnight LB culture mixed in a 1:1 ratio with 70% glycerol).

### Cultivation Conditions

A preculture of 96-well microtiter plate experiments was started from a cryovial, in 150 μL LB and was incubated overnight at 37° C. on an orbital shaker at 800 rpm. This culture was used as inoculum for a 96 well square microtiter plate, with 400 μL minimal medium by diluting 400×. These final 96-well culture plates were then incubated at 37° C. on an orbital shaker at 800 rpm for 72 h, or shorter, or longer. To measure sugar concentrations at the end of the cultivation experiment whole broth samples were taken from each well by boiling the culture broth for 15 min at 60° C. before spinning down the cells (=average of intra- and extracellular sugar concentrations). Also, a dilution of the cultures was made to measure the optical density at 600 nm. The cell performance index or CPI was determined by dividing the concentrations of the oligosaccharide measured in the whole broth by the biomass, in relative percentages compared to the reference strain. The biomass is empirically determined to be approximately ⅓^{rd} of the optical density measured at 600 nm. The export ratio of the oligosaccharide was determined by dividing the concentrations of the oligosaccharide measured in the supernatant by the concentrations of the oligosaccharide measured in the whole broth, in relative percentages compared to the reference strain.

A preculture for the bioreactor was started from an entire 1 mL cryovial of a certain strain, inoculated in 250 mL or 500 mL minimal medium in a 1 L or 2.5 L shake flask and incubated for 24 h at 37° C. on an orbital shaker at 200 rpm. A 5 L bioreactor was then inoculated (250 mL inoculum in 2 L batch medium); the process was controlled by MFCS control software (Sartorius Stedim Biotech, Melsungen, Germany). Culturing condition were set to 37° C., and maximal stirring; pressure gas flow rates were dependent on the strain and bioreactor. The pH was controlled at 6.8 using 0.5 M H2S04 and 20% NH4OH. The exhaust gas was cooled. 10% solution of silicone antifoaming agent was added when foaming raised during the fermentation.

### Optical Density

Cell density of the cultures was frequently monitored by measuring optical density at 600 nm (Implen Nanophotometer NP80, Westburg, Belgium or with a Spark 10M microplate reader, Tecan, Switzerland).

B. Saccharomyces cerevisiae

### Media

Strains were grown on Synthetic Defined yeast medium with Complete Supplement Mixture (SD CSM) or CSM drop-out (SD CSM-Ura, SD CSM-Trp, SD CSM-His) containing 6.7 g/L Yeast Nitrogen Base without amino acids (YNB w/o AA, Difco), 20 g/L agar (Difco) (solid cultures), 22 g/L glucose monohydrate or 20 g/L lactose and 0.79 g/L CSM or 0.77 g/L CSM-Ura, 0.77 g/L CSM-Trp, or 0.77 g/L CSM-His (MP Biomedicals).

### Strains

S. cerevisiae BY4742 created by Brachmann et al. (Yeast (1998) 14:115-32) was used, available in the Euroscarf culture collection. All mutant strains were created by homologous recombination or plasmid transformation using the method of Gietz (Yeast 11:355-360, 1995).

Kluyveromyces marxianus lactis is available at the LMG culture collection (Ghent, Belgium).

### Plasmids

In an example to produce sialic acid and CMP-sialic acid, a yeast expression plasmid can be derived from the pRS420-plasmid series (Christianson et al., 1992, Gene 110: 119-122) containing the TRP1 selection marker and constitutive transcriptional units for one or more copies of an L-glutamine-D-fructose-6-phosphate aminotransferase like e.g. the mutant glmS*54 from E. coli, a phosphatase like e.g. the E. coli SurE, an N-acylglucosamine 2-epimerase like e.g. AGE from B. ovatus, one or more copies of an N-acetylneuraminate synthase like e.g. NeuB from N. meningitidis or from C. jejuni, and one or more copies of an N-acylneuraminate cytidylyltransferase like e.g. NeuA from C. jejuni, NeuA from H. influenzae and/or NeuA from P. multocida. Optionally, a constitutive transcriptional unit comprising one or more copies for a glucosamine 6-phosphate N-acetyltransferase like e.g. GNA1 from S. cerevisiae was added as well.

In an example to produce sialylated oligosaccharides, the plasmid further comprised constitutive transcriptional units for a lactose permease like e.g. LAC12 from Kluyveromyces lactis, and one or more copies of a beta-galactoside alpha-2,3-sialyltransferase like e.g. PmultST3 from P. multocida, NmeniST3 from N. meningitidis or PmultST2 from P. multocida subsp. multocida str. Pm70, a beta-galactoside alpha-2,6-sialyltransferase like e.g. PdST6 from P. damselae and/or P-JT-ISH-224-ST6 from Photobacterium sp. JT-ISH-224, and/or an alpha-2,8-sialyltransferase like e.g. from M. musculus.

In an example to produce GDP-fucose, a yeast expression plasmid like p2a_2μ_Fuc (Chan 2013, Plasmid 70, 2-17) can be used for expression of foreign genes in S. cerevisiae. This plasmid contains an ampicillin resistance gene and a bacterial origin of replication to allow for selection and maintenance in E. coli and the 2μ yeast ori and the Ura3 selection marker for selection and maintenance in yeast. This plasmid is further modified with constitutive transcriptional units for a lactose permease like e.g. LAC12 from K. lactis, a GDP-mannose 4,6-dehydratase like e.g. gmd from E. coli and a GDP-L-fucose synthase like e.g. fcl from E. coli. The yeast expression plasmid p2a_2μ_Fuc2 can be used as an alternative expression plasmid of the p2a_2μ_Fuc plasmid comprising next to the ampicillin resistance gene, the bacterial ori, the 2p yeast ori and the Ura3 selection marker constitutive transcriptional units for a lactose permease like e.g. LAC12 from K. lactis, a fucose permease like e.g. fucP from E. coli and a bifunctional fucose kinase/fucose-1-phosphate guanylyltransferase like e.g. fkp from B. fragilis. To further produce fucosylated oligosaccharides, the p2a_2μ_Fuc and its variant the p2a_2μ_Fuc2, additionally contained (a) constitutive transcriptional unit(s) for one or more fucosyltransferases like Helicobacter pylori UA1234 HpFutC or HpFucT.

In an example to produce UDP-galactose, a yeast expression plasmid can be derived from the pRS420-plasmid series (Christianson et al., 1992, Gene 110: 119-122) containing the HIS3 selection marker and a constitutive transcriptional unit for a UDP-glucose-4-epimerase like e.g. galE from E. coli. This plasmid was further modified with constitutive transcriptional units for a lactose permease like e.g. LAC12 from K. lactis, a galactoside beta-1,3-N-acetylglucosaminyltransferase like e.g. IgtA from N. meningitidis to produce LN3. To further produce LN3-derived oligosaccharides like LNT or LNnT, an N-acetylglucosamine beta-1,3-galactosyltransferase like e.g. WbgO from E. coli O55:H7 or an N-acetylglucosamine beta-1,4-galactosyltransferase like e.g. IgtB from N. meningitidis, respectively, was also added on the plasmid. Preferably but not necessarily, the glycosyltransferases and/or the proteins involved in nucleotide-activated sugar synthesis were N- and/or C-terminally fused to a SUMOstar tag (e.g. obtained from pYSUMOstar, Life Sensors, Malvern, PA) to enhance their solubility.

Optionally, the mutant yeast strains were modified with a genomic knock-in of a constitutive transcriptional unit encoding a chaperone protein like e.g. Hsp31, Hsp32, Hsp33, Sno4, Kar2, Ssb1, Sse1, Sse2, Ssa1, Ssa2, Ssa3, Ssa4, Ssb2, Ecm10, Ssc1, Ssq1, Ssz1, Lhs1, Hsp82, Hsc82, Hsp78, Hsp104, Tcp1, Cct4, Cct8, Cct2, Cct3, Cct5, Cct6 or Cct7 (Gong et al., 2009, Mol. Syst. Biol. 5: 275).

Plasmids were maintained in the host E. coli DH5alpha (F⁻, phi80dlacZdeltaM15, delta(lacZYA-argF)U169, deoR, recA1, endA1, hsdR17(rk⁻, mk⁺), phoA, supE44, lambda⁻, thi-1, gyrA96, relA1) bought from Invitrogen.

### Gene Expression Promoters

Genes are expressed using synthetic constitutive promoters, as described by Blazeck (Biotechnology and Bioengineering, Vol. 109, No. 11, 2012).

### Heterologous and Homologous Expression

Genes that needed to be expressed, be it from a plasmid or from the genome were synthetically synthetized with one of the following companies: DNA2.0, Gen9, IDT or Twist Bioscience. Expression could be further facilitated by optimizing the codon usage to the codon usage of the expression host. Genes were optimized using the tools of the supplier.

### Cultivations Conditions

In general, yeast strains were initially grown on SD CSM plates to obtain single colonies. These plates were grown for 2-3 days at 30° C. Starting from a single colony, a preculture was grown over night in 5 mL at 30° C., shaking at 200 rpm. Subsequent 125 mL shake flask experiments were inoculated with 2% of this preculture, in 25 mL media. These shake flasks were incubated at 30° C. with an orbital shaking of 200 rpm.

C. Bacillus subtilis

### Media

Two different media are used, namely a rich Luria Broth (LB) and a minimal medium for shake flask (MMsf). The minimal medium uses a trace element mix.

Trace element mix consisted of 0.735 g/L CaCl2·2H2O, 0.1 g/L MnCl2·2H2O, 0.033 g/L CuCl2·2H2O, 0.06 g/L CoCl2·6H2O, 0.17 g/L ZnCl2, 0.0311 g/L H3BO4, 0.4 g/L Na2EDTA·2H2O and 0.06 g/L Na2MoO4. The Fe-citrate solution contained 0.135 g/L FeCl3·6H2O, 1 g/L Na-citrate (Hoch 1973 PMC1212887).

The Luria Broth (LB) medium consisted of 1% tryptone peptone (Difco, Erembodegem, Belgium), 0.5% yeast extract (Difco) and 0.5% sodium chloride (VWR, Leuven, Belgium). Luria Broth agar (LBA) plates consisted of the LB media with 12 g/L agar (Difco, Erembodegem, Belgium) added.

The minimal medium for the shake flasks (MMfs) experiments contained 2.00 g/L (NH₄)2SO₄, 7.5 g/L KH₂PO₄, 17.5 g/L K₂HPO₄, 1.25 g/L Na-citrate, 0.25 g/L MgSO₄.7H₂O, 0.05 g/L tryptophan, from 10 up to 30 g/L glucose or another carbon source including but not limited to fructose, maltose, sucrose, glycerol and maltotriose when specified in the examples, 10 ml/L trace element mix and 10 ml/L Fe-citrate solution. The medium was set to a pH of 7 with 1M KOH. Depending on the experiment lactose, LNB or LacNAc could be added as a precursor.

Complex medium, e.g. LB, was sterilized by autoclaving (121° C., 21′) and minimal medium by filtration (0.22 μm Sartorius). When necessary, the medium was made selective by adding an antibiotic (e.g. zeocin (20 mg/L)).

### Strains, Plasmids and Mutations

Bacillus subtilis 168, available at Bacillus Genetic Stock Center (Ohio, USA).

Plasmids for gene deletion via Cre/lox are constructed as described by Yan et al. (Appl. & Environm. Microbial., September 2008, p 5556-5562). Gene disruption is done via homologous recombination with linear DNA and transformation via electroporation as described by Xue et al. (J. Microb. Meth. 34 (1999) 183-191). The method of gene knockouts is described by Liu et al. (Metab. Engine. 24 (2014) 61-69). This method uses 1000 bp homologies up- and downstream of the target gene.

Integrative vectors as described by Popp et al. (Sci. Rep., 2017, 7, 15158) are used as expression vector and could be further used for genomic integrations if necessary. A suitable promoter for expression can be derived from the part repository (iGem): sequence id: Bba_K143012, Bba_K823000, Bba_K823002 or Bba_K823003. Cloning can be performed using Gibson Assembly, Golden Gate assembly, Cliva assembly, LCR or restriction ligation.

In an example for the production of lactose-based oligosaccharides, Bacillus subtilis mutant strains are created to contain a gene coding for a lactose importer (such as the E. coli lacY gene). For 2′FL, 3FL and diFL production, an alpha-1,2- and/or alpha-1,3-fucosyltransferase expression construct is additionally added to the strains. For LNT and LNnT production, expression constructs are added that code for a galactoside beta-1,3-N-acetylglucosaminyltransferase (IgtA) from Neisseria meningitidis and either an N-acetylglucosamine beta-1,3-galactosyltransferase (wbgO) from Escherichia coli O55:H7 for LNT production or an N-acetylglucosamine beta-1,4-galactosyltransferase (IgtB) from Neisseria meningitidis for LNnT production. For 3′-SL and 6′-SL production, the strains are described in WO18122225. A sialic acid producing B. subtilis strain is obtained by overexpressing the native fructose-6-P-aminotransferase (BsglmS) to enhance the intracellular glucosamine-6-phosphate pool. Further on, the enzymatic activities of the genes nagA, nagB and gamA were disrupted by genetic knockouts and a glucosamine-6-P-aminotransferase from S. cerevisiae (ScGNA1), an N-acetylglucosamine-2-epimerase from Bacteroides ovatus (BoAGE) and a sialic acid synthase from Campylobacter jejuni (CjneuB) were overexpressed on the genome. To allow production of 6′-SL, a CMP-sialic acid synthetase from Neisseria meningitidis (NmneuA) and a sialyltransferase from Photobacterium damselae (PdbST) were overexpressed. To allow production of 3′-SL, a CMP-sialic acid synthetase from Neisseria meningitidis (NmneuA) and a sialyltransferase from Neisseria meningitidis (NmST) were overexpressed.

### Heterologous and Homologous Expression

Genes that needed to be expressed be it from a plasmid or from the genome, were synthetically synthetized with one of the following companies: DNA2.0, Gen9, Twist Biosciences or IDT.

Expression could be further facilitated by optimizing the codon usage to the codon usage of the expression host. Genes were optimized using the tools of the supplier.

### Cultivation Conditions

A preculture of 96-well microtiter plate experiments was started from a cryovial or a single colony from an LB plate, in 150 μL LB and was incubated overnight at 37° C. on an orbital shaker at 800 rpm. This culture was used as inoculum for a 96-well square microtiter plate, with 400 μL MMsf medium by diluting 400×. Each strain was grown in multiple wells of the 96-well plate as biological replicates. These final 96-well culture plates were then incubated at 37° C. on an orbital shaker at 800 rpm for 72 h, or shorter, or longer. At the end of the cultivation experiment samples were taken from each well to measure the supernatant concentration (extracellular sugar concentrations, after 5 min. spinning down the cells), or by boiling the culture broth for 15 min at 60° C. before spinning down the cells (=whole broth concentration, intra- and extracellular sugar concentrations, as defined herein).

Also, a dilution of the cultures was made to measure the optical density at 600 nm. The cell performance index or CPI was determined by dividing the compound's concentrations, e.g. sialyllactose concentrations, measured in the whole broth by the biomass, in relative percentages compared to the reference strain. The biomass is empirically determined to be approximately ⅓^{rd} of the optical density measured at 600 nm. The compound export ratio was determined by dividing the compound concentrations measured in the supernatant by the compound concentrations measured in the whole broth, in relative percentages compared to the reference strain.

D. Corynebacterium glutamicum

### Media

Two different media are used to cultivate C. glutamicum: i.e. a rich tryptone-yeast extract (TY) medium and a minimal medium. The TY medium consisted of 1.6% tryptone (Difco), 1% yeast extract (Difco) and 0.5% sodium chloride (VWR). TY agar (TYA) plates consisted of the TY media, with 12 g/L agar (Difco) added. The minimal medium for the shake flask experiments contained 20 g/L (NH₄)₂SO₄, 5 g/L urea, 1 g/L KH₂PO₄, 1 g/L K₂HPO₄, 0.25 g/L MgSO₄·7H₂O, 42 g/L MOPS, from 10 up to 30 g/L glucose (or another carbon source including but not limited to fructose, maltose, sucrose, glycerol and maltotriose) and 1 mL/L trace element mix. Depending on the experiment lactose is added as a precursor. The trace element mix consisted of 10 g/L CaCl₂, 10 g/L FeSO₄·7H₂O, 10 g/L MnSO₄·H₂O, 1 g/L ZnSO₄·7H₂O, 0.2 g/L CuSO₄, 0.02 g/L NiCl₂·6H₂O, 0.2 g/L biotin (pH 7.0) and 0.03 g/L protocatechuic acid.

Complex medium, e.g. TY, was sterilized by autoclaving (121° C., 21 min) and minimal medium by filtration (0.22 am Sartorius). When necessary, the medium was made selective by adding an antibiotic (e.g. kanamycin, ampicillin).

### Strains and Mutations

Corynebacterium glutamicum ATCC 13032 was used as available at the American Type Culture Collection. Integrative plasmid vectors based on the Cre/loxP technique as described by Suzuki et al. (Appl. Microbiol. Biotechnol., 2005 April, 67(2):225-33) and temperature-sensitive shuttle vectors as described by Okibe et al. (J. Microbiol. Meth. 85, 2011, 155-163) are constructed for gene deletions, mutations and insertions. Suitable promoters for (heterologous) gene expression can be derived from Yim et al. (Biotechnol. Bioeng., 2013 November, 110(11):2959-69). Cloning can be performed using Gibson Assembly, Golden Gate assembly, Cliva assembly, LCR or restriction ligation.

In an example for sialic acid production, the engineered strain was derived from C. glutamicum comprising knockouts of the C. glutamicum Idh, cgl2645 and nagB genes and genomic knock-ins of constitutive transcriptional units containing a phosphoglucosamine mutase like e.g. glmM from E. coli, an N-acetylglucosamine-1-phosphate uridyltransferase/glucosamine-1-phosphate acetyltransferase like e.g. glmU from E. coli, a UDP-N-acetylglucosamine 2-epimerase like e.g. neuC from C. jejuni and an N-acetylneuraminate synthase like e.g. neuB from N. meningitidis. To enhance the intracellular glucosamine-6-phosphate pool, the modified strain can further be modified with a genomic knock-in of one or more constitutive transcriptional units containing a glutamine-fructose-6-P-aminotransferase like e.g. the native glutamine-fructose-6-P-aminotransferase glmS. In an example for sialylated oligosaccharide production, the sialic acid production strains further need to express an N-acylneuraminate cytidylyltransferase like e.g. neuA from P. multocida, and a beta-galactoside alpha-2,6-sialyltransferase like e.g. PdST6 from Photobacterium damselae. Constitutive transcriptional units of the N-acylneuraminate cytidylyltransferase and the sialyltransferases can be delivered to the engineered strain either via genomic knock-in or via expression plasmids. If the engineered strains producing sialic acid and CMP-sialic acid were intended to make sialylated lactose structures, the strains were additionally modified with a genomic knock-in of a constitutive transcriptional unit for a lactose permease like e.g. the E. coli LacY.

In an example for LN3 production, the engineered strain was derived from C. glutamicum comprising knockouts of the C. glutamicumIdh, cgI2645 and nagB genes and genomic knock-ins of constitutive transcriptional units containing a galactoside beta-1,3-N-acetylglucosaminyltransferase like e.g. IgtA from N. meningitidis and a lactose permease like e.g. LacY from E. coli. In an example for LNnT production, the LN3 producing strain was further transformed with a constitutive transcriptional unit for an N-acetylglucosamine beta-1,4-galactosyltransferase like e.g. IgtB from N. meningitidis.

In an example for the production of lactose-based oligosaccharides, C. glutamicum mutant strains are created to contain a gene coding for a lactose importer such as e.g. the E. coli LacY.

In an example for fucosylated oligosaccharide synthesis, the engineered strain was derived from C. glutamicum comprising knockouts of the C. glutamicumIdh, cgI2645 and nagB genes and genomic knock-ins of constitutive transcriptional units containing an alpha-1,2-fucosyltransferase like e.g. HpFutC from H. pylori and/or an alpha-1,3-fucosyltransferase like e.g. HpFucT from H. pylori.

Genes that needed to be expressed, be it from a plasmid or from the genome were synthetically synthetized with one of the following companies: DNA2.0, Gen9, Twist Biosciences or IDT.

Expression could be further facilitated by optimizing the codon usage to the codon usage of the expression host. Genes were optimized using the tools of the supplier.

### Cultivation Conditions

A preculture of 96-well microtiter plate experiments was started from a cryovial or a single colony from a TY plate, in 150 μL TY and was incubated overnight at 37° C. on an orbital shaker at 800 rpm. This culture was used as inoculum for a 96-well square microtiter plate, with 400 μL minimal medium by diluting 400×. Each strain was grown in multiple wells of the 96-well plate as biological replicates. These final 96-well culture plates were then incubated at 37° C. on an orbital shaker at 800 rpm for 72 h, or shorter, or longer. At the end of the cultivation experiment samples were taken from each well to measure the supernatant concentration (extracellular sugar concentrations, after 5 min. spinning down the cells), or by boiling the culture broth for 15 min at 60° C. before spinning down the cells (=whole broth concentration, intra- and extracellular sugar concentrations, as defined herein).

Also, a dilution of the cultures was made to measure the optical density at 600 nm. The cell performance index or CPI was determined by dividing the oligosaccharide concentrations, e.g. sialyllactose concentrations, measured in the whole broth by the biomass, in relative percentages compared to the reference strain. The biomass is empirically determined to be approximately ⅓rd of the optical density measured at 600 nm.

E. Chlamydomonas reinhardtii

### Media

C. reinhardtii cells were cultured in Tris-acetate-phosphate (TAP) medium (pH 7.0). The TAP medium uses a 1000× stock Hutner's trace element mix. Hutner's trace element mix consisted of 50 g/L Na₂EDTA·H₂O (Titriplex III), 22 g/L ZnSO₄·7H₂O, 11.4 g/L H₃BO₃, 5 g/L MnCl₂·4H₂O, 5 g/L FeSO₄·7H₂O, 1.6 g/L CoCl₂·6H₂O, 1.6 g/L CuSO₄·5H₂O and 1.1 g/L (NH₄)₆MoO₃.

The TAP medium contained 2.42 g/L Tris (tris(hydroxymethyl)aminomethane), 25 mg/L salt stock solution, 0.108 g/L K₂HPO₄, 0.054 g/L KH₂PO₄ and 1.0 mL/L glacial acetic acid. The salt stock solution consisted of 15 g/L NH₄Cl, 4 g/L MgSO₄·7H₂O and 2 g/L CaCl₂·2H₂O. As precursor for saccharide synthesis, precursors like e.g. galactose, glucose, fructose, fucose, GlcNAc could be added. Medium was sterilized by autoclaving (121° C., 21 min). For stock cultures on agar slants TAP medium was used containing 1% agar (of purified high strength, 1000 g/cm2).

### Strains, Plasmids and Mutations

C. reinhardtii wild-type strains 21 gr (CC-1690, wild-type, mt+), 6145C (CC-1691, wild-type, mt−), CC-125 (137c, wild-type, mt+), CC-124 (137c, wild-type, mt−) as available from Chlamydomonas Resource Center (https://www.chlamycollection.org), University of Minnesota, U.S.A.

Expression plasmids originated from pSI103, as available from Chlamydomonas Resource Center. Cloning can be performed using Gibson Assembly, Golden Gate assembly, Cliva assembly, LCR or restriction ligation. Suitable promoters for (heterologous) gene expression can be derived from e.g. Scranton et al. (Algal Res. 2016, 15: 135-142). Targeted gene modification (like gene knock-out or gene replacement) can be carried using the Crispr-Cas technology as described e.g. by Jiang et al. (Eukaryotic Cell 2014, 13(11): 1465-1469).

Transformation via electroporation was performed as described by Wang et al. (Biosci. Rep. 2019, 39: BSR2018210). Cells were grown in liquid TAP medium under constant aeration and continuous light with a light intensity of 8000 Lx until the cell density reached 1.0-2.0×10⁷ cells/mL. Then, the cells were inoculated into fresh liquid TAP medium in a concentration of 1.0×10⁶ cells/mL and grown under continuous light for 18-20 h until the cell density reached 4.0×10⁶ cells/mL. Next, cells were collected by centrifugation at 1250 g for 5 min at room temperature, washed and resuspended with pre-chilled liquid TAP medium containing 60 mM sorbitol (Sigma, U.S.A.), and iced for 10 min. Then, 250 μL of cell suspension (corresponding to 5.0×10⁷ cells) were placed into a pre-chilled 0.4 cm electroporation cuvette with 100 ng plasmid DNA (400 ng/mL). Electroporation was performed with 6 pulses of 500 V each having a pulse length of 4 ms and pulse interval time of 100 ms using a BTX ECM830 electroporation apparatus (1575Ω, 50 μFD). After electroporation, the cuvette was immediately placed on ice for 10 min. Finally, the cell suspension was transferred into a 50 mL conical centrifuge tube containing 10 mL of fresh liquid TAP medium with 60 mM sorbitol for overnight recovery at dim light by slowly shaking. After overnight recovery, cells were recollected and plated with starch embedding method onto selective 1.5% (w/v) agar-TAP plates containing ampicillin (100 mg/L) or chloramphenicol (100 mg/L). Plates were then incubated at 23+−0.5° C. under continuous illumination with a light intensity of 8000 Lx. Cells were analysed 5-7 days later.

In an example for CMP-sialic acid synthesis, C. reinhardtii cells were modified with constitutive transcriptional units for a UDP-N-acetylglucosamine-2-epimerase/N-acetylmannosamine kinase like e.g. GNE from Homo sapiens or a mutant form of the human GNE polypeptide comprising the R263L mutation, an N-acylneuraminate-9-phosphate synthetase like e.g. NANS from Homo sapiens and an N-acylneuraminate cytidylyltransferase like e.g. CMAS from Homo sapiens. In an example for production of sialylated oligosaccharides, C. reinhardtii cells are modified with a CMP-sialic acid transporter like e.g. CST from Mus musculus, and a beta-galactoside alpha-2,6-sialyltransferase like e.g. PdST6 from Photobacterium damselae.

In an example for production of UDP-galactose, C. reinhardtii cells were modified with transcriptional units comprising the gene encoding the galactokinase from Arabidopsis thaliana and the gene encoding the UDP-sugar pyrophosphorylase (USP) from A. thaliana.

In an example for LN3 production, C. reinhardtii cells were modified with a constitutive transcriptional unit comprising a galactoside beta-1,3-N-acetylglucosaminyltransferase like e.g. IgtA from N. meningitidis. In an example for LNnT production, the LN3 producing strain was further modified with a constitutive transcriptional unit comprising an N-acetylglucosamine beta-1,4-galactosyltransferase like e.g. IgtB from N. meningitidis.

In an example for production of GDP-fucose, C. reinhardtii cells are modified with a transcriptional unit for a GDP-fucose synthase like e.g. from Arabidopsis thaliana.

In an example for fucosylation, C. reinhardtii cells can be modified with an expression plasmid comprising a constitutive transcriptional unit for an alpha-1,2-fucosyltransferase like e.g. HpFutC from H. pylori and/or an alpha-1,3-fucosyltransferase like e.g. HpFucT from H. pylori.

### Heterologous and Homologous Expression

Genes that needed to be expressed, be it from a plasmid or from the genome were synthetically synthetized with one of the following companies: DNA2.0, Gen9, Twist Biosciences or IDT.

Expression could be further facilitated by optimizing the codon usage to the codon usage of the expression host. Genes were optimized using the tools of the supplier.

### Cultivation Conditions

Cells of C. reinhardtii were cultured in selective TAP-agar plates at 23+/−0.5° C. under 14/10 h light/dark cycles with a light intensity of 8000 Lx. Cells were analysed after 5 to 7 days of cultivation.

For high-density cultures, cells could be cultivated in closed systems like e.g. vertical or horizontal tube photobioreactors, stirred tank photobioreactors or flat panel photobioreactors as described by Chen et al. (Bioresour. Technol. 2011, 102: 71-81) and Johnson et al. (Biotechnol. Prog. 2018, 34: 811-827).

### F. Animal Cells

Isolation of Mesenchymal Stem Cells from Adipose Tissue of Different Animals

Fresh adipose tissue is obtained from slaughterhouses (e.g. cattle, pigs, sheep, chicken, ducks, catfish, snake, frogs) or liposuction (e.g., in case of humans, after informed consent) and kept in phosphate buffer saline supplemented with antibiotics. Enzymatic digestion of the adipose tissue is performed followed by centrifugation to isolate mesenchymal stem cells. The isolated mesenchymal stem cells are transferred to cell culture flasks and grown under standard growth conditions, e.g., 37° C., 5% CO2. The initial culture medium includes DMEM-F12, RPMI, and Alpha-MEM medium (supplemented with 15% foetal bovine serum), and 1% antibiotics. The culture medium is subsequently replaced with 10% FBS (foetal bovine serum)-supplemented media after the first passage. For example, Ahmad and Shakoori (2013, Stem Cell Regen. Med. 9(2): 29-36), which is incorporated herein by reference in its entirety for all purposes, describes certain variation(s) of the method(s) described herein in this example.

Isolation of Mesenchymal Stem Cells from Milk

This example illustrates isolation of mesenchymal stem cells from milk collected under aseptic conditions from human or any other mammal(s) such as described herein. An equal volume of phosphate buffer saline is added to diluted milk, followed by centrifugation for 20 min. The cell pellet is washed thrice with phosphate buffer saline and cells are seeded in cell culture flasks in DMEM-F12, RPMI, and Alpha-MEM medium supplemented with 10% foetal bovine serum and 1% antibiotics under standard culture conditions. For example, Hassiotou et al. (2012, Stem Cells. 30(10): 2164-2174), which is incorporated herein by reference in its entirety for all purposes, describes certain variation(s) of the method(s) described herein in this example.

### Differentiation of Stem Cells Using 2D and 3D Culture Systems

The mesenchymal cells isolated from adipose tissue of different animals or from milk as described above can be differentiated into mammary-like epithelial and luminal cells in 2D and 3D culture systems. See, for example, Huynh et al. 1991. Exp Cell Res. 197(2): 191-199; Gibson et al. 1991, In Vitro Cell Dev Biol Anim. 27(7): 585-594; Blatchford et al. 1999; Animal Cell Technology’: Basic & Applied Aspects, Springer, Dordrecht. 141-145; Williams et al. 2009, Breast Cancer Res 11(3): 26-43; and Arevalo et al. 2015, Am J Physiol Cell Physiol. 310(5): C348-C356; each of which is incorporated herein by reference in their entireties for all purposes.

For 2D culture, the isolated cells were initially seeded in culture plates in growth media supplemented with 10 ng/mL epithelial growth factor and 5 pg/mL insulin. At confluence, cells were fed with growth medium supplemented with 2% fetal bovine serum, 1% penicillin-streptomycin (100 U/mL penicillin, 100 ug/mL streptomycin), and 5 pg/mL insulin for 48 h. To induce differentiation, the cells were fed with complete growth medium containing 5 pg/mL insulin, 1 pg/mL hydrocortisone, 0.65 ng/mL triiodothyronine, 100 nM dexamethasone, and 1 pg/mL prolactin. After 24 h, serum is removed from the complete induction medium.

For 3D culture, the isolated cells were trypsinized and cultured in Matrigel, hyaluronic acid, or ultra-low attachment surface culture plates for six days and induced to differentiate and lactate by adding growth media supplemented with 10 ng/mL epithelial growth factor and 5 pg/mL insulin. At confluence, cells were fed with growth medium supplemented with 2% foetal bovine serum, 1% penicillin-streptomycin (100 U/mL penicillin, 100 ug/mL streptomycin), and 5 pg/mL insulin for 48 h. To induce differentiation, the cells were fed with complete growth medium containing 5 pg/mL insulin, 1 pg/mL hydrocortisone, 0.65 ng/mL triiodothyronine, 100 nM dexamethasone, and 1 pg/mL prolactin. After 24 h, serum is removed from the complete induction medium.

### Method of Making Mammary-Like Cells

In a next step, the cells are brought to induced pluripotency by reprogramming with viral vectors encoding for Oct4, Sox2, Klf4, and c-Myc. The resultant reprogrammed cells are then cultured in Mammocult media (available from Stem Cell Technologies), or mammary cell enrichment media (DMEM, 3% FBS, estrogen, progesterone, heparin, hydrocortisone, insulin, EGF) to make them mammary-like, from which expression of select milk components can be induced. Alternatively, epigenetic remodelling is performed using remodelling systems such as CRISPR/Cas9, to activate select genes of interest, such as casein, a-lactalbumin to be constitutively on, to allow for the expression of their respective proteins, and/or to down-regulate and/or knock-out select endogenous genes as described e.g. in WO21067641, which is incorporated herein by reference in its entirety for all purposes.

### Cultivation

Completed growth media includes high glucose DMEM/F12, 10% FBS, 1% NEAA, 1% pen/strep, 1% ITS-X, 1% F-Glu, 10 ng/mL EGF, and 5 pg/mL hydrocortisone. Completed lactation media includes high glucose DMEM/F12, 1% NEAA, 1% pen/strep, 1% ITS-X, 1% F-Glu, 10 ng/mL EGF, 5 pg/mL hydrocortisone, and 1 pg/mL prolactin (5 ug/mL in Hyunh 1991). Cells are seeded at a density of 20,000 cells/cm2 onto collagen coated flasks in completed growth media and left to adhere and expand for 48 hours in completed growth media, after which the media is switched out for completed lactation media. Upon exposure to the lactation media, the cells start to differentiate and stop growing. Within about a week, the cells start secreting lactation product(s) such as milk lipids, lactose, casein and whey into the media. A desired concentration of the lactation media can be achieved by concentration or dilution by ultrafiltration. A desired salt balance of the lactation media can be achieved by dialysis, for example, to remove unwanted metabolic products from the media. Hormones and other growth factors used can be selectively extracted by resin purification, for example the use of nickel resins to remove His-tagged growth factors, to further reduce the levels of contaminants in the lactated product.

### G. Analysis

### Productivity

The specific productivity Qp is the specific production rate of the product (which is the oligosaccharide), typically expressed in mass units of product per mass unit of biomass per time unit (=g oligosaccharide/g biomass/h). The Qp value has been determined for each phase of the fermentation runs, i.e. Batch and Fed-Batch phase, by measuring both the amount of product and biomass formed at the end of each phase and the time frame each phase lasted.

The specific productivity Qs is the specific consumption rate of the substrate, e.g. sucrose, typically expressed in mass units of substrate per mass unit of biomass per time unit (=g sucrose/g biomass/h). The Qs value has been determined for each phase of the fermentation runs, i.e. Batch and Fed-Batch phase, by measuring both the total amount of sucrose consumed and biomass formed at the end of each phase and the time frame each phase lasted.

The yield on sucrose Ys is the fraction of product that is made from substrate and is typically expressed in mass unit of product per mass unit of substrate (=g oligosaccharide/g sucrose). The Ys has been determined for each phase of the fermentation runs, i.e. Batch and Fed-Batch phase, by measuring both the total amount of oligosaccharide produced and total amount of sucrose consumed at the end of each phase.

The yield on biomass Yx is the fraction of biomass that is made from substrate and is typically expressed in mass unit of biomass per mass unit of substrate (=g biomass/g sucrose). The Yp has been determined for each phase of the fermentation runs, i.e. Batch and Fed-Batch phase, by measuring both the total amount of biomass produced and total amount of sucrose consumed at the end of each phase.

The rate is the speed by which the product is made in a fermentation run, typically expressed in concentration of product made per time unit (=g oligosaccharide/L/h). The rate is determined by measuring the concentration of the oligosaccharide with LN3 as a core trisaccharide that has been made at the end of the Fed-Batch phase and dividing this concentration by the total fermentation time.

The lactose conversion rate is the speed by which lactose is consumed in a fermentation run, typically expressed in mass units of lactose per time unit (=g lactose consumed/h). The lactose conversion rate is determined by measurement of the total lactose that is consumed during a fermentation run, divided by the total fermentation time.

### Growth Rate/Speed Measurement

The maximal growth rate (μMax) was calculated based on the observed optical densities at 600 nm using the R package grofit.

### Analytical Analysis

Standards such as but not limited to sucrose, lactose, lacto-N-triose II (LN3), lacto-N-tetraose (LNT), lacto-N-neo-tetraose (LNnT), LNFP-I, LNFP-II, LNFP-III, LNFP-V, LNFP-VI, 2′FL, 3FL, 6′SL, 3′SL, LSTa, LSTc and LSTd were purchased from Carbosynth (UK), Elicityl (France) and IsoSep (Sweden). Other compounds were analysed with in-house made standards.

Neutral (non-charged) oligosaccharides were analysed on a Waters Acquity H-class UPLC with Evaporative Light Scattering Detector (ELSD) or a Refractive Index (RI) detection. A volume of 0.7 μL sample was injected on a Waters Acquity UPLC BEH Amide column (2.1×100 mm; 130 Å; 1.7 μm) column with an Acquity UPLC BEH Amide VanGuard column, 130 Å, 2.1×5 mm. The column temperature was 50° C. The mobile phase consisted of a ¼ water and ¾ acetonitrile solution to which 0.2% triethylamine was added. The method was isocratic with a flow of 0.130 mL/min. The ELS detector had a drift tube temperature of 50° C. and the N2 gas pressure was 50 psi, the gain 200 and the data rate 10 pps. The temperature of the RI detector was set at 35° C.

Sialylated oligosaccharides were analysed on a Waters Acquity H-class UPLC with Refractive Index (RI) detection. A volume of 0.5 μL sample was injected on a Waters Acquity UPLC BEH Amide column (2.1×100 mm; 130 Å; 1.7 μm). The column temperature was 50° C. The mobile phase consisted of a mixture of 70% acetonitrile, 26% ammonium acetate buffer (150 mM) and 4% methanol to which 0.05% pyrrolidine was added. The method was isocratic with a flow of 0.150 mL/min. The temperature of the RI detector was set at 35° C.

Both neutral and sialylated sugars were analysed on a Waters Acquity H-class UPLC with Refractive Index (RI) detection. A volume of 0.5 μL sample was injected on a Waters Acquity UPLC BEH Amide column (2.1×100 mm; 130 Å; 1.7 μm). The column temperature was 50° C. The mobile phase consisted of a mixture of 72% acetonitrile and 28% ammonium acetate buffer (100 mM) to which 0.1% triethylamine was added. The method was isocratic with a flow of 0.260 mL/min. The temperature of the RI detector was set at 35° C.

For analysis on a mass spectrometer, a Waters Xevo TQ-MS with Electron Spray Ionisation (ESI) was used with a desolvation temperature of 450° C., a nitrogen desolvation gas flow of 650 L/h and a cone voltage of 20 V. The MS was operated in selected ion monitoring (SIM) in negative mode for all oligosaccharides. Separation was performed on a Waters Acquity UPLC with a Thermo Hypercarb column (2.1×100 mm; 3 μm) on 35° C. A gradient was used wherein eluent A was ultrapure water with 0.1% formic acid and wherein eluent B was acetonitrile with 0.1% formic acid. The oligosaccharides were separated in 55 min using the following gradient: an initial increase from 2 to 12% of eluent B over 21 min, a second increase from 12 to 40% of eluent B over 11 min and a third increase from 40 to 100% of eluent B over 5 min. As a washing step 100% of eluent B was used for 5 min. For column equilibration, the initial condition of 2% of eluent B was restored in 1 min and maintained for 12 min.

Both neutral and sialylated sugars at low concentrations (below 50 mg/L) were analysed on a Dionex HPAEC system with pulsed amperometric detection (PAD). A volume of 5 μL of sample was injected on a Dionex CarboPac PA200 column 4×250 mm with a Dionex CarboPac PA200 guard column 4×50 mm. The column temperature was set to 30° C. A gradient was used wherein eluent A was deionized water, wherein eluent B was 200 mM Sodium hydroxide and wherein eluent C was 500 mM Sodium acetate. The oligosaccharides were separated in 60 min while maintaining a constant ratio of 25% of eluent B using the following gradient: an initial isocratic step maintained for 10 min of 75% of eluent A, an initial increase from 0 to 4% of eluent C over 8 min, a second isocratic step maintained for 6 min of 71% of eluent A and 4% of eluent C, a second increase from 4 to 12% of eluent C over 2.6 min, a third isocratic step maintained for 3.4 min of 63% of eluent A and 12% of eluent C and a third increase from 12 to 48% of eluent C over 5 min. As a washing step 48% of eluent C was used for 3 min. For column equilibration, the initial condition of 75% of eluent A and 0% of eluent C was restored in 1 min and maintained for 11 min. The applied flow was 0.5 mL/min.

### Normalization of the Data

For all types of cultivation conditions, data obtained from the mutant strains was normalized against data obtained in identical cultivation conditions with reference strains having an identical genetic background as the mutant strains but lacking the extra modification for enhancing the synthesis acetyl-Coenzyme A. The dashed horizontal line on each plot that is shown in the examples, indicates the setpoint to which all adaptations were normalized. All data is given in relative percentages to that setpoint.

TABLE A
Overview of enzymes used

| | | | | | Country of origin |
|---|---|---|---|---|---|
| | alternative | | | | of digital |
| Name/ | name/ | | | | sequence |
| Identifier | identifier | Organism | function | Origin | information |
| acs | EcAcs/ | Escherichia | acetyl-coenzyme A ligase | Synthetic | USA |
| | UniProtKB ID | coli K-12 | | | |
| | P27550 | MG1655 | | | |
| acs | ScAcs/ | Saccharomyces | acetyl-coenzyme A ligase | Synthetic | USA |
| | UniprotKB ID | cerevisiae | | | |
| | Q01574 or | | | | |
| | P52910 | | | | |
| acs | BsAcs/ | Bacillus | acetyl-coenzyme A ligase | Synthetic | USA |
| | UniprotKB ID | subtilis | | | |
| | P39062 | | | | |
| AGE | BoAGE | Bacteroides | N-acylglucosamine-2- | Synthetic | USA |
| | | ovatus | epimerase | | |
| ST | PmultST2 | Pasteurella | beta-galactoside alpha- | Synthetic | Unknown |
| | | multocida | 2,3-sialyltransferase | | |
| | | subsp. | | | |
| | | multocida str. | | | |
| | | Pm70 | | | |
| ST | NmeniST3 | Neisseria | beta-galactoside alpha- | Synthetic | United Kingdom |
| | | meningitidis | 2,3-sialyltransferase | | |
| ST | NmST | Neisseria | a-2,3-sialyltransferase | Synthetic | United Kingdom |
| | | meningitidis | | | |
| ST | PmultST3 | Pasteurella | beta-galactoside alpha- | Synthetic | USA |
| | | multocida | 2,3-sialyltransferase | | |
| ST | PdST6 | Photobacterium | alpha-2,6- | Synthetic | Japan |
| | | damselae | sialyltransferase | | |
| ST | P-JT-ISH-224- | Photobacterium | alpha-2,6- | Synthetic | Japan |
| | ST6 | sp. JT-ISH- | sialyltransferase | | |
| | | 224 | | | |
| ST | | Mus | alpha-2,8- | Synthetic | USA |
| | | musculus | sialyltransferase | | |
| CscB | EcCscB | Escherichia | sucrose transporter | Synthetic | USA |
| | | coli W | | | |
| eda | | Escherichia | oxaloacetate | Synthetic | USA |
| | | coli K-12 | decarboxylase activity | | |
| | | MG1655 | | | |
| fbaB | | Pisum | fructose-1,6-bisphosphate | Synthetic | Unknown |
| | | sativum | aldolase | | |
| fbpase | | Pisum | fructose-1,6-bisphosphate | Synthetic | Unknown |
| | | sativum | phosphatase | | |
| fcl | | Escherichia | GDP-L-fucose synthase | Synthetic | USA |
| | | coli K12 | | | |
| | | MG1655 | | | |
| fkp | | Bacteroides | bifunctional fucose | Synthetic | United Kingdom |
| | | fragilis NCTC | kinase/fucose-1- | | |
| | | 9343 | phosphate | | |
| | | | guanylyltransferase | | |
| Frk | Zmfrk | Zymomonas | fructose kinase | Synthetic | United Kingdom |
| | | mobilis | | | |
| fucP | EcfucP | Escherichia | fucose permease | Synthetic | USA |
| | | coli K12 | | | |
| | | MG1655 | | | |
| FucT | HpFucT | Helicobacter | alpha-1,3- | Synthetic | United Kingdom |
| | | pylori UA1234 | fucosyltransferase | | |
| FutC | HpFutC | Helicobacter | alpha-1,2- | Synthetic | United Kingdom |
| | | pylori UA1234 | fucosyltransferase | | |
| galE | | Escherichia | UDP-glucose-4- | Synthetic | USA |
| | | coli K-12 | epimerase | | |
| | | MG1655 | | | |
| glmM | EcglmM | Escherichia | phosphoglucosamine | Synthetic | USA |
| | | coli K12 | mutase | | |
| | | MG1655 | | | |
| glmS | BsglmS | Bacillus | fructose-6-P- | Synthetic | USA |
| | | subtilis | aminotransferase | | |
| glmS | CgglmS | Corynebacterium | fructose-6-P- | Synthetic | USA |
| | | glutamicum | aminotransferase | | |
| glmS*54 | EcglmS*54 | Escherichia | mutant fructose-6-P- | Synthetic | USA |
| | | coli K12 | aminotransferase/ L- | | |
| | | MG1655 | glutamine-D-fructose-6- | | |
| | | | phosphate | | |
| | | | aminotransferase | | |
| glmU | EcglmU | Escherichia | N-acetylglucosamine-1- | Synthetic | USA |
| | | coli K12 | phosphate | | |
| | | MG1655 | uridyltransferase/glucosa | | |
| | | | mine-1-phosphate | | |
| | | | acetyltransferase | | |
| gmd | Ecgmd | Escherichia | GDP-mannose-4,6- | Synthetic | USA |
| | | coli K-12 | dehydratase | | |
| | | MG1655 | | | |
| GNA1 | ScGNA1 | Saccharomyces | glucosamine-6-P- | Synthetic | USA |
| | | cerevisiae | aminotransferase/ a | | |
| | | | glucosamine 6-phosphate | | |
| | | | N-acetyltransferase | | |
| GNE | MmGNE | Mus | bifunctional UDP-GlcNAc | Synthetic | USA |
| | | musculus | 2-epimerase/N- | | |
| | | (strain | acetylmannosamine | | |
| | | C57BL/6J) | kinase | | |
| GNE | HsGNE | Homo | bifunctional UDP-GlcNAc | Synthetic | USA |
| | | sapiens | 2-epimerase/N- | | |
| | | | acetylmannosamine | | |
| | | | kinase | | |
| LAC12 | KILAC12 | Kluyveromyces | lactose permease | Synthetic | USA |
| | | lactis | | | |
| LacY | EcLacY | Escherichia | lactose permease | Synthetic | USA |
| | | coli K12 | | | |
| | | MG1655 | | | |
| IgtA | NmLgtA | Neisseria | beta-1,3-GlcNAc | Synthetic | United Kingdom |
| | | meningitidis | transferase | | |
| LgtB | NmLgtB | Neisseria | (N-acetylglucosamine) | Synthetic | United Kingdom |
| | | meningitidis | beta-1,4- | | |
| | | MC58 | galactosyltransferase | | |
| manA | EcManA | Escherichia | Mannose-6-phosphate | Synthetic | USA |
| | | coli K-12 | isomerase | | |
| | | MG1655 | | | |
| manB | EcManB | Escherichia | phosphomannomutase | Synthetic | USA |
| | | coli K12 | | | |
| | | MG1655 | | | |
| manC | EcManC | Escherichia | mannose-1-phosphate | Synthetic | USA |
| | | coli K-12 | guanosyltransferase | | |
| | | MG1655 | | | |
| NeuA | PmNeuA | Pasteurella | N-acylneuraminate | Synthetic | USA |
| | | multocida | cytidylyltransferases | | |
| NeuA | CjNeuA | Campylobacter | N-acylneuraminate | Synthetic | USA |
| | | jejuni | cytidylyltransferases/ | | |
| | | | CMP-sialic acid | | |
| | | | synthetase | | |
| NeuA | HiNeuA | Haemophilus | N-acylneuraminate | Synthetic | USA |
| | | influenzae | cytidylyltransferases | | |
| NeuA | NmNeuA | Neisseria | CMP-sialic acid | Synthetic | United Kingdom |
| | | meningitidis | synthetase | | |
| NeuB | CjNeuB | Campylobacter | sialic acid synthase | Synthetic | USA |
| | | jejuni | | | |
| NeuB | NmNeuB | Neisseria | sialic acid synthase | Synthetic | United Kingdom |
| | | meningitidis | | | |
| NeuC | CjNeuC | Campylobacter | UDP-N- | Synthetic | USA |
| | | jejuni | acetylglucosamine 2- | | |
| | | | epimerase | | |
| ppsA | EcppsA | Escherichia | phosphoenolpyruvate | Synthetic | USA |
| | | coli K-12 | synthase | | |
| | | MG1655 | | | |
| SP | BaSP | Bifidobacterium | sucrose phosphorylase | Synthetic | Germany |
| | | adolescentis | | | |
| wbgO | EcWbgO | Escherichia | N-acetylglucosamine | Synthetic | Germany |
| | | coli O55:H7 | beta-1,3- | | |
| | | | galactosyltransferase | | |
| wcaG | EcwcaG | Escherichia | GDP-L-fucose synthase | Synthetic | USA |
| | | coli K12 | | | |
| | | MG1655 | | | |
| NANS | HsNANS | Homo | N-acylneuraminate-9- | Synthetic | USA |
| | | sapiens | phosphate synthetase | | |
| 2-keto-3- | | Bacteroides | N-acylneuraminate-9- | Synthetic | Unknown |
| deoxy-D- | | thetaiotaomicron | phosphatase | | |
| glycero-D- | | (strain | | | |
| galacto-9- | | ATCC 29148) | | | |
| phosphono- | | | | | |
| nonic acid | | | | | |
| phosphatase | | | | | |
| KPA15328.1 | | Candidatus | N-acylneuraminate-9- | Synthetic | Germany |
| | | Magnetomorum | phosphatase | | |
| | | sp. HK-1 | | | |
| CMAS | HsCMAS | Homo | N-acylneuraminate | Synthetic | USA |
| | | sapiens | cytidylyltransferase | | |
| CST | MmCST | Mus | CMP-sialic acid | Synthetic | USA |
| | | musculus | transporter | | |
| | | (strain | | | |
| | | C57BL/6J) | | | |
| KIN | AtKIN | Arabidopsis | Galactokinase | Synthetic | USA |
| | | thaliana | | | |
| USP | AtUSP | Arabidopsis | UDP-sugar | Synthetic | USA |
| | | thaliana | pyrophosphorylase | | |
| GER1 | AtGER1 | Arabidopsis | GDP-fucose synthase | Synthetic | USA |
| | | thaliana | | | |

### Example 2. Production of Sialic Acid in an E. coli Host Overexpressing an E. coli Acetyl-Coenzyme A Synthetase (Ecacs, UniProtKB ID P27550)

An E. coli mutant strain producing sialic acid as described in Example 1 was used to additionally create strains overexpressing an E. coli acetyl-Coenzyme A synthetase (Ecacs, UniProtKB ID P27550) on plasmid. This enzyme is able to scavenge acetate to form acetyl-Coenzyme A with the usage of ATP. Different expression levels of the Ecacs gene were established by varying the gene's promoter and 5′UTR as enlisted in Table 1. The genes were expressed using promoters from Mutalik et al. (Nat. Methods 2013, No. 10, 354-360), as described herein as “PROM0025” and “PROM0034”. UTRs used as described herein as “UTR0012”, “UTR0051” and “UTR0053” were obtained from Mutalik et al. (Nat. Methods 2013, No. 10, 354-360). The terminators used in the examples is described as “TER0004” and is obtained from Cambray et al. (Nucleic Acids Res. 2013, 41(9), 5139-48).

These strains with different expression levels of the Ecacs gene (S_ACS1, S_ACS2 and S_ACS3) were evaluated in a growth experiment as described in Example 1 and compared to their parent strain (Reference) lacking overexpression of an E. coli acetyl-Coenzyme A synthetase (Ecacs, UniProtKB ID P27550). Each strain was grown in 3 multiple wells of a 96-well plate.

Table 2 shows the titer of sialic acid, the titer of acetate and the maximal growth speed (Mumax) of the different strains overexpressing an E. coli acetyl-Coenzyme A synthetase (Ecacs, UniProtKB ID P27550) on plasmid, both in relative % normalized to the reference strain (average value±standard deviation). The data indicates that, compared to a reference strain, an improved sialic acid titer is obtained, and an equal or better maximal growth speed is obtained in the strains overexpressing an extra E. coli acetyl-Coenzyme A synthetase. Furthermore, compared to a reference strain, the levels of acetate produced by the strains overexpressing an extra E. coli acetyl-Coenzyme A synthetase was strongly reduced.

TABLE 1

| Strain | Promoter part | UTR part | Terminator part |
|---|---|---|---|
| S_ACS1 | PROM0034 | UTR0053 | TER0004 |
| S_ACS2 | PROM0034 | UTR0051 | TER0004 |
| S_ACS3 | PROM0025 | UTR0012 | TER0004 |

TABLE 2

| | Normalized | Normalized | Normalized |
|---|---|---|---|
| | titer of Sialic | Mumax (avg | titer of acetate |
| Strain | acid (avg % ± sd) | % ± sd) | (avg % ± sd) |
| S_ACS1 | 659 ± 87 | 100 ± 12 | 0 ± 0 |
| S_ACS2 | 654 ± 169 | 106 ± 13 | 0 ± 0 |
| S_ACS3 | 686 ± 88 | 104 ± 12 | 28 ± 20 |
| Reference | 100 ± 8 | 100 ± 16 | 100 ± 2 |

### Example 3. Production of 6′-SL in an E. coli Host Overexpressing an E. coli Acetyl-Coenzyme A Synthetase (Ecacs, UniProtKB ID P27550)

An E. coli mutant strain producing 6′-SL as described in Example 1 was used to additionally create strains overexpressing an E. coli acetyl-Coenzyme A synthetase (Ecacs, UniProtKB ID P27550) on plasmid. This enzyme is able to scavenge acetate to form acetyl-Coenzyme A with the usage of ATP. Different expression levels of the Ecacs gene were established by varying the gene's promoter and 5′UTR as enlisted in Table 3. The genes were expressed using promoters from Mutalik et al. (Nat. Methods 2013, No. 10, 354-360), as described herein as “PROM0025” and “PROM0032”. UTRs used as described herein as “UTR0029” and “UTR0051” were obtained from Mutalik et al. (Nat. Methods 2013, No. 10, 354-360). The terminators used in the examples is described as “TER0004” and is obtained from Cambray et al. (Nucleic Acids Res. 2013, 41(9), 5139-48).

These strains with different expression levels of the Ecacs gene (S_ACS4 and S_ACS5) were evaluated in a growth experiment as described in Example 1 and compared to their parent strain (Reference) lacking overexpression of an E. coli acetyl-Coenzyme A synthetase (Ecacs, UniProtKB ID P27550). Each strain was grown in 3 multiple wells of a 96-well plate.

Table 4 shows the titer of 6′-SL, the titer of acetate and the maximal growth speed (Mumax) of the different strains overexpressing an E. coli acetyl-Coenzyme A synthetase (Ecacs, UniProtKB ID P27550) on plasmid, both in relative % normalized to the reference strain (average value±standard deviation). The data indicates that, compared to a reference strain, an improved 6′-SL titer is obtained, and an equal or better maximal growth speed is obtained in the strains overexpressing an extra E. coli acetyl-Coenzyme A synthetase. Furthermore, compared to a reference strain, the levels of acetate produced by the strains overexpressing an extra E. coli acetyl-Coenzyme A synthetase was strongly reduced.

TABLE 3

| Strain | Promoter part | UTR part | Terminator part |
|---|---|---|---|
| S_ACS4 | PROM0032 | UTR0051 | TER0004 |
| S_ACS5 | PROM0025 | UTR0029 | TER0004 |

TABLE 4

| | Normalized | Normalized | Normalized |
|---|---|---|---|
| | titer of 6′- | Mumax (avg | titer of acetate |
| Strain | SL (avg % ± sd) | % ± sd) | (avg % ± sd) |
| S_ACS4 | 122 ± 5 | 98 ± 11 | 40 ± 1 |
| S_ACS5 | 117 ± 3 | 105 ± 13 | 27 ± 19 |
| Reference | 100 ± 1 | 100 ± 16 | 100 ± 2 |

### Example 4. Production of 6′-SL in an E. coli Host Completely Lacking an Acetyl-Coenzyme A Synthetase

An E. coli mutant strain producing 6′-SL as described in Example 1 was used to additionally create a strain that completely lacks an acetyl-Coenzyme A synthetase in its cell. This E. coli Ecacs knock-out strain (S_ACS6) was evaluated and compared to its parent strain (Reference) still having the native Ecacs operon in a growth experiment as described in Example 1. Each strain was grown in 3 multiple wells of a 96-well plate.

Table 5 shows the titer of 6′-SL, the titer of acetate and the maximal growth speed (Mumax) of the strain lacking any E. coli acetyl-Coenzyme A ligase (Ecacs, UniProtKB ID P27550), both in relative % normalized to the reference strain (average value±standard deviation). The data indicates that, compared to a reference strain, a reduced 6′-SL and maximal growth speed is obtained, and a higher acetate titer is obtained in strains completely lacking the E. coli acetyl-Coenzyme A ligase (Ecacs, UniProtKB ID P27550). The surprising effect of further increasing the acetyl-CoA synthesis was established with further overexpression of the acetate-CoA ligase enzyme (EC 6.2.1.1 or 6.2.1.13, Ecacs, UniProtKB ID P27550) as shown in Example 3.

TABLE 5

| | Normalized | Normalized | Normalized |
|---|---|---|---|
| | titer of 6′- | Mumax (avg | titer of acetate |
| Strain | SL (avg % ± sd) | % ± sd) | (avg % ± sd) |
| S_ACS6 | 61 ± 6 | 90 ± 11 | 114 ± 4 |
| Reference | 100 ± 1 | 100 ± 16 | 100 ± 2 |

### Example 5. Overexpression of an Extra Acetyl-Coenzyme A Ligase Knocked-In at the Genome of E. coli Leads to Higher 6′-SL Production and a Strong Reduction in Acetate by-Product Formation in 5 L Fed-Batch Fermentations

An E. coli mutant strain producing 6′-SL as described in Example 1 was used to additionally create a strain overexpressing an extra E. coli acetyl-Coenzyme A ligase (Ecacs, UniProtKB ID P27550) knocked-in at its genome under control of “PROM0025”, “UTR0029” (Mutalik et al., Nat. Methods 2013, No. 10, 354-360) and “TER0004” (Cambray et al., Nucleic Acids Res. 2013, 41(9), 5139-48). This enzyme is able to scavenge acetate to form acetyl-Coenzyme A with the usage of ATP. In addition, this strain was modified with an extra 6′-SL production plasmid containing a neuA from Pasteurella multicoda (PmultneuA) and an ST from Photobacterium damselae (PdbST). This strain (S_ACS7) was evaluated and compared to its parent strain lacking an extra Ecacs overexpression knock-in (Reference) in two independent fed-batch fermentations performed as described in Example 1.

Table 6 shows the 6′-SL titers of the S_ACS7, its 6′-SL production rate and its acetate titers at the end of the fed-batch bioreactor runs, both in relative % normalized to the reference strain lacking an extra Ecacs overexpression knock-in (average value±standard deviation). The data indicates that, compared to a reference strain, higher 6′-SL titers and production rates are obtained in the strain overexpressing the Ecacs gene in the genome. Additionally, the acetate titers at the end of the fermentations are strongly reduced.

TABLE 6

| | Normalized | Normalized | Normalized |
|---|---|---|---|
| | titer of 6′- | Mumax (avg | titer of acetate |
| Strain | SL (avg % ± sd) | % ± sd) | (avg % ± sd) |
| S_ACS7 | 149 ± 3 | 191 ± 44 | 24 ± 15 |
| Reference | 100 ± 3 | 100 ± 32 | 100 ± 4 |

### Example 6. Production of Oligosaccharides in an E. coli Host Overexpressing an Acetyl-Coenzyme A Ligase

E. coli mutant strains for the production of oligosaccharides, and more specifically human milk oligosaccharides such as 2′FL, 3FL, diFL, 3′SL, 6′SL, LNT, LNnT, LSTa, LSTb, LSTc or LSTd are engineered as described in Example 1. Such strains are further modified to additionally enhance the synthesis of acetyl-Coenzyme A by the overexpression of an E. coli acetyl-Coenzyme A ligase (Ecacs, UniProtKB ID P27550). Any of these aforementioned strains are able to produce any of the listed HMOs, and in similar or potentially higher amounts than the respective reference strains lacking the extra overexpression of an E. coli acetyl-Coenzyme A ligase. Additionally, the strains grow similarly well or better than their respective reference strains.

These strains can also be evaluated in fed-batch fermentations at bioreactor scale, as described in Example 1. Sucrose can be used as a carbon source and lactose as the precursor for oligosaccharide formation. Examples of other carbon sources are glucose, glycerol, fructose, arabinose, maltotriose, sorbitol, xylose, rhamnose and mannose. The strain's performance in the bioreactor will be similar or better compared to their reference strains in any of the measured parameters listed in Example 1, materials and methods.

### Example 7. Production of Oligosaccharides in a Bacillus subtilis Host Overexpressing an Acetyl-Coenzyme A Ligase

In another embodiment, the production of oligosaccharides, and more specifically human milk oligosaccharides such as 2′FL, 3FL, diFL, 3′SL, 6′SL, LNT or LNnT can be established by engineering a Bacillus subtilis host strain as described in Example 1. These strains could be modified to additionally enhance the synthesis of acetyl-Coenzyme A by the overexpression of a codon-optimized E. coli acetyl-Coenzyme A ligase (Ecacs, UniProtKB ID P27550) or the native acetyl-Coenzyme A ligase from Bacillus subtilis (acsA, UniProtKB ID P39062).

Any of these aforementioned strains are able to produce any of the listed HMOs, and in similar or potentially higher amounts than the respective reference strains lacking the overexpression of an acetyl-Coenzyme A ligase. Additionally, the strains grow similarly well or better than their respective reference strains.

### Example 8. Production of Oligosaccharides in a Saccharomyces cerevisiae Host Overexpressing an Acetyl-Coenzyme A Ligase

In another embodiment, the production of oligosaccharides, and more specifically human milk oligosaccharides such as 2′FL, 3FL, diFL, 3′SL, 6′SL, LNT or LNnT can be established by engineering a Saccharomyces cerevisiae host strain as described in Example 1. These strains could be modified to additionally enhance the synthesis of acetyl-Coenzyme A by the overexpression of a codon-optimized E. coli acetyl-Coenzyme A ligase (Ecacs, UniProtKB ID P27550) or the native acetyl-Coenzyme A ligase 1 from Saccharomyces cerevisiae (ACS1, UniProtKB ID Q01574) or the native acetyl-Coenzyme A ligase 2 from Saccharomyces cerevisiae (ACS2, UniProtKB ID P52910).

Any of these aforementioned strains are able to produce any of the listed HMOs, and in similar or potentially higher amounts than the respective reference strains lacking the overexpression of an acetyl-Coenzyme A ligase. Additionally, the strains grow similarly well or better than their respective reference strains.

### Example 9. Production of Oligosaccharides in a Corynebacterium glutamicum Host Overexpressing an Acetyl-Coenzyme A Synthetase

In another example, the production of oligosaccharides, and more specifically human milk oligosaccharides such as 2′FL, 3FL, diFL, 3′SL, 6′SL, LNT or LNnT can be established by engineering a Corynebacterium glutamicum host strain as described in Example 1. These strains could be modified to additionally enhance the synthesis of acetyl-Coenzyme A by the overexpression of a codon-optimized E. coli Acetyl-coenzyme A synthetase (Ecacs, UniProtKB ID P27550) or an Acetyl-coenzyme A synthetase from Corynebacterium sepedonicum (UniProtKB ID B0RAD6). Any of these aforementioned strains are able to produce any of the listed HMOs, and in similar or potentially higher amounts than the respective reference strains lacking the overexpression of an Acetyl-coenzyme A synthetase. Additionally, the strains grow similarly well or better than their respective reference strains.

### Example 10. Production of Oligosaccharides in a Chlamydomonas reinhardtii Host Overexpressing an Acetyl-Coenzyme A Synthetase

In another example, the production of oligosaccharides, and more specifically human milk oligosaccharides such as 2′FL, 3FL, diFL, 3′SL, 6′SL, LNT or LNnT can be established by engineering a Chlamydomonas reinhardtii host strain as described in Example 1. These strains could be modified to additionally enhance the synthesis of acetyl-Coenzyme A by the overexpression of a codon-optimized E. coli Acetyl-coenzyme A synthetase (Ecacs, UniProtKB ID P27550) or a native Acetyl-coenzyme A synthetase (UniProtKB ID A8JFR9) or an Acetyl-coenzyme A synthetase from Arabidopsis thaliana (UniProtKB ID B9DGD6). Any of these aforementioned strains are able to produce any of the listed HMOs, and in similar or potentially higher amounts than the respective reference strains lacking the overexpression of an Acetyl-coenzyme A synthetase. Additionally, the strains grow similarly well or better than their respective reference strains.

### Example 11. Production of LSTc in an Animal Cell Overexpressing an Acetyl-Coenzyme A Synthetase

Isolated mesenchymal cells and re-programmed into mammary-like cells as described in Example 1 are modified via CRISPR-CAS to express the GlcN6P synthase from Homo sapiens (UniProtKB ID Q06210), the glucosamine 6-phosphate N-acetyltransferase from Homo sapiens (UniProtKB ID Q96EK6), the phosphoacetylglucosamine mutase from Homo sapiens (UniProtKB ID 095394), the UDP-N-acetylhexosamine pyrophosphorylase from Homo sapiens (UniProtKB ID Q16222), the galactoside beta-1,3-N-acetylglucosaminyltransferase LgtA from N. meningitidis, the N-acetylglucosamine beta-1,4-galactosyltransferase IgtB from N. meningitidis, the N-acylneuraminate cytidylyltransferases neuA from Mus musculus (UniProtKB ID Q99KK2) and the alpha-2,6-sialyltransferase PdST6 from P. damselae to produce LSTc. These cells can be further modified to modified to additionally enhance the synthesis of acetyl-Coenzyme A by the overexpression of acss2 from Mus musculus (UniProtKB ID Q9QXG4) and/or acss1 from Mus musculus (UniProtKB ID Q99NB1). Cells are seeded at a density of 20,000 cells/cm2 onto collagen coated flasks in completed growth media and left to adhere and expand for 48 hours in completed growth media, after which the media is switched out for completed lactation media for about 7 days. After cultivation as described in Example 17, cells are subjected to UPLC to analyse for production of LSTc.

### Example 12. Production of Oligosaccharides in an E. coli Host with a Knock-Out of the Isocitrate Lyase (aceA) and a Knock-Out of the Malate Synthase (aceB) Gene

E. coli mutant strains for the production of oligosaccharides, and more specifically human milk oligosaccharides such as 2′FL, 3FL, diFL, 3′SL, 6′SL, LNT, LNnT, LSTa, LSTb, LSTc or LSTd are engineered as described in Example 1. Such strains are further modified by a knock-out of the isocitrate lyase (aceA, UniProtKB ID P0A9G6) and a knock-out of the malate synthase (aceB, UniProtKB ID P08997). Any of these aforementioned strains are able to produce any of the listed HMOs, and in similar or potentially higher amounts than the respective reference strains which express the native E. coli isocitrate lyase and the native E. coli malate synthase. Additionally, the strains grow similarly well or better than their respective reference strains.

These strains can also be evaluated in fed-batch fermentations at bioreactor scale, as described in Example 1. Sucrose can be used as a carbon source and lactose as the precursor for oligosaccharide formation. Examples of other carbon sources are glucose, glycerol, fructose, arabinose, maltotriose, sorbitol, xylose, rhamnose and mannose. The strain's performance in the bioreactor will be similar or better compared to their reference strains in any of the measured parameters listed in Example 1, materials and methods.

### Example 13. Production of Oligosaccharides in an E. coli Host with Reduced Expression of the E. coli Citrate Synthase gltA Gene and/or with Expression of a Modified gltA Gene

E. coli mutant strains for the production of oligosaccharides, and more specifically human milk oligosaccharides such as 2′FL, 3FL, diFL, 3′SL, 6′SL, LNT, LNnT, LSTa, LSTb, LSTc or LSTd are engineered as described in Example 1. Such strains are further modified to have a reduced expression of the E. coli citrate synthase (gltA, UniProtKB ID P0ABH7) compared to the native expression levels of said E. coli gltA gene and/or by expression of a mutant E. coli citrate synthase gltA* differing from the wild-type gltA (UniProtKB ID P0ABH7) by a A258T, A162V and/or A124T mutation. Reduced expression of said gltA gene can be obtained by e.g. CrispR to alter the promoter sequence controlling the E. coli gltA expression. Any of these aforementioned strains are able to produce any of the listed HMOs, and in similar or potentially higher amounts than the respective reference strains with unmodified expression of the native E. coli gltA gene. Additionally, the strains grow similarly well or better than their respective reference strains.

These strains can also be evaluated in fed-batch fermentations at bioreactor scale, as described in Example 1. Sucrose can be used as a carbon source and lactose as the precursor for oligosaccharide formation. Examples of other carbon sources are glucose, glycerol, fructose, arabinose, maltotriose, sorbitol, xylose, rhamnose and mannose. The strain's performance in the bioreactor will be similar or better compared to their reference strains in any of the measured parameters listed in Example 1, materials and methods.

1.-60. (canceled)

61. A cell for producing a compound, the cell comprising:
a pathway for producing the compound,
wherein the compound is a disaccharide, oligosaccharide and/or a Neu(n)Ac-containing bioproduct, wherein (n) is 4, 5, 7, 8 or 9 or a combination thereof, and
wherein the cell is metabolically engineered for enhanced synthesis of acetyl-Coenzyme A.

62. The cell of claim 61, wherein the enhanced acetyl-Coenzyme A synthesis is obtained by enhanced expression and/or activity of at least one of the enzymes:
i) acetyl-Coenzyme A ligase (EC 6.2.1.1 or 6.2.1.13);
ii) pyruvate dehydrogenase (EC 1.2.5.1);
iii) pantothenate kinase (EC 2.7.1.33);
iv) acetyl phosphate-producing pyruvate oxidase (EC 1.2.3.3);
v) acetate kinase (EC 2.7.2.1);
vi) phosphate acetyltransferase (EC 2.3.1.8);
vii) pyruvate decarboxylase (EC 4.1.1.1);
viii) acetaldehyde dehydrogenase (EC 1.2.1.3, EC 1.2.1.4, or EC 1.2.1.5);
ix) pyruvate formate lyase (EC 2.3.1.54);
x) CoA-acetylating pyruvate oxidase (EC 1.2.3.6);
xi) pyruvate synthase (EC 1.2.7.1); or
xii) pyruvate dehydrogenase enzyme complex (EC 1.2.4.1 (pyruvate dehydrogenase E1 component), EC 2.3.1.12 (pyruvate dehydrogenase, E2 subunit), and EC 1.8.1.4 (lipoamide dehydrogenase, E3 subunit)).

63. The cell of claim 62, wherein the enhanced acetyl-CoA synthesis is obtained by a method selected from the group consisting of:
a) increasing the copy number of any one or more of the genes encoding enzyme i) to xi) or the enzyme complex of xii),
b) modifying an expression regulatory sequence of any one or more of the genes encoding enzyme i) to xi) or the enzyme complex of xii), and
c) a combination thereof.

64. The cell of claim 62, wherein the enhanced synthesis is obtained by overexpressing any one or more of the genes encoding an endogenous enzyme i) to xi) or the enzyme complex of xii); and/or introducing and expressing any one or more of a homologous or heterologous gene encoding an enzyme i) to xi) or the enzyme complex of xii).

65. The cell of claim 62, wherein any one or more of the enzymes i) to xi) or the enzyme complex of xii) is presented to the cell in at least one gene expression module wherein expression is regulated by at least one regulatory sequence.

66. The cell of claim 65, wherein the expression module(s) are integrated in the cell's genome and/or presented to the cell on a vector comprising plasmid, cosmid, phage, liposome or virus, which is to be stably transformed into the cell.

67. The cell of claim 61, wherein the cell is modified for enhanced synthesis and/or supply of phosphoenolpyruvate (PEP).

68. The cell of claim 61, wherein the cell is further modified for reduced degradation of acetyl-CoA and/or its main precursor pyruvate.

69. The cell of claim 61, wherein the cell is modified for reduced expression of or deleting genes encoding at least one of:
a) lactate dehydrogenase (EC 1.1.2.3, EC 1.1.2.4, EC 1.1.2.5, EC 1.1.1.27, EC 1.1.1.28),
b) pyruvate carboxylase (EC 6.4.1.1),
c) isocitrate lyase (EC 4.1.3.1); and
d) malate synthase (EC 2.3.3.9).

70. The cell of claim 61, wherein the cell is further modified for rendering less functional the Krebs cycle genes by either reduced expression or point mutations optionally A258T, A162V and/or A124T in the citrate synthase enzyme coded by gltA in E coli.

71. The cell of claim 61, wherein the disaccharide is selected from the group consisting of lactose (Gal-b1,4-Glc), lacto-N-biose (Gal-b1,3-GlcNAc), N-acetyllactosamine (Gal-b1,4-GlcNAc), LacDiNAc (GalNAc-b1,4-GlcNAc), N-acetylgalactosaminylglucose (GalNAc-b1,4-Glc), Neu5Ac-a2,3-Gal, Neu5Ac-a2,6-Gal, and fucopyranosyl-(1-4)-N-glycolylneuraminic acid (Fuc-(1-4)-Neu5Gc).

72. The cell of claim 61, wherein the oligosaccharide is a milk oligosaccharide, a mammalian milk oligosaccharide, a human milk oligosaccharide, a Lewis-type antigen oligosaccharide, O-antigen, enterobacterial common antigen (ECA), the oligosaccharide repeats present in capsular polysaccharides, peptidoglycan, amino-sugar or antigen of the human ABO blood group system.

73. The cell of claim 61, wherein the Neu(n)Ac-containing bioproduct is selected from the group consisting of sialic acid, a disaccharide, an oligosaccharide, sialylated compound comprising Neu5Ac, a Neu(n)Ac-containing glycolipid, a Neu(n)Ac-containing glycoprotein.

74. The cell of claim 61, wherein the oligosaccharide is a non-charged (neutral) oligosaccharide, a fucosylated oligosaccharide, and/or acidic oligosaccharide.

75. The cell of claim 61, wherein the oligosaccharide is selected from the group consisting of 3-fucosyllactose, 2′-fucosyllactose, 6-fucosyllactose, 2′,3-difucosyllactose, 2′,2-difucosyllactose, 3,4-difucosyllactose, 6′-sialyllactose, 3′-sialyllactose, 3,6-disialyllactose, 6,6′-disialyllactose, 8,3-disialyllactose, 3,6-disialyllacto-N-tetraose, lactodifucotetraose, lacto-N-tetraose, lacto-N-neotetraose, lacto-N-fucopentaose II, lacto-N-fucopentaose I, lacto-N-fucopentaose III, lacto-N-fucopentaose V, lacto-N-fucopentaose VI, sialyllacto-N-tetraose c, sialyllacto-N-tetraose b, sialyllacto-N-tetraose a, lacto-N-difucohexaose I, lacto-N-difucohexaose II, lacto-N-hexaose, lacto-N-neohexaose, para-lacto-N-hexaose, monofucosylmonosialyllacto-N-tetraose c, monofucosyl para-lacto-N-hexaose, monofucosyllacto-N-hexaose III, isomeric fucosylated lacto-N-hexaose III, isomeric fucosylated lacto-N-hexaose I, sialyllacto-N-hexaose, sialyllacto-N-neohexaose II, difucosyl-para-lacto-N-hexaose, difucosyllacto-N-hexaose, difucosyllacto-N-hexaose a, and difucosyllacto-N-hexaose c.

76. The cell of claim 62, wherein the acetyl-Coenzyme A ligase originates from an Escherichia coli species selected from the group optionally consisting of E. coli B, E. coli BL21, E. coli BL21(DE3), E. coli C, E. coli DH5alpha, E. coli K-12, E. coli Nissle, E. coli Top10, E. coli W, or originates from Salmonella typhi, Vibrio cholera, Saccharomyces cerevisiae, Bacillus subtilis, Mycobacterium tuberculosis, Campylobacter jejuni, Yersinia pestis, and Corynebacteriales, optionally the acetyl-Coenzyme A ligase is the E. coli UniProtKB-P27550 enzyme, or is the S. cerevisiae UniProt KB Q01574 enzyme, or is the S. cerevisiae UniProt KB P52910 enzyme, or is the B. subtilis UniProt KB P39062 enzyme, or is the H. sapiens UniProt KB Q9NR19 enzyme.

77. The cell of claim 61, wherein the cell comprises and expresses at least one glycosyltransferase.

78. The cell of claim 77, wherein the glycosyltransferase is selected from the group consisting of fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases, and fucosaminyltransferases.

79. The cell of claim 77, wherein the cell is modified in the expression or activity of at least one glycosyltransferase, optionally the modification is obtained by overexpressing an endogenous glycosyltransferase and/or introducing and expressing a homologous or heterologous glycosyltransferase.

80. The cell of claim 77, wherein one of the glycosyltransferases is a fucosyltransferase that transfers a fucose from a GDP-fucose donor to lactose in an alpha-1,2- and/or alpha-1,3 linkage, thereby producing fucosyllactose and/or difucosyllactose.

81. The cell of claim 77, the cell comprising:
(i) a GDP-fucose biosynthesis pathway comprising at least one enzyme selected from the group consisting of mannose-6-phosphate isomerase, phosphomannomutase, mannose-1-phosphate guanylyltransferase, GDP-mannose 4,6-dehydratase, GDP-L-fucose synthase, fucose permease, fucose kinase, fucose-1-phosphate guanylyltransferase, and L-fucokinase/GDP-fucose pyrophosphorylase; and
(ii) a fucosyltransferase.

82. The cell of claim 77, wherein one of the glycosyltransferases is a sialyltransferase that transfers an N-acetyl-neuraminic acid (sia) from a CMP-Sia donor to lactose in an alpha-2,3-, alpha-2,6- and/or alpha-2,8-linkage, thereby producing sialyllactose and/or disialyllactose.

83. The cell of claim 77, the cell comprising at least one of:
(i) a sialic acid biosynthesis pathway comprising at least one enzyme selected from the group consisting of UDP-GlcNAc 2-epimerase, N-acylglucosamine 2-epimerase and sialic acid synthase;
(ii) an N-acylneuraminate cytidylyltransferase; and
(iii) a sialyltransferase.

84. The cell of claim 77, wherein one of the glycosyltransferases is a galactosyltransferase selected from the group consisting of beta-1,3-galactosyltransferase, N-acetylglucosamine beta-1,3-galactosyltransferase, beta-1,4-galactosyltransferase, N-acetylglucosamine beta-1,4-galactosyltransferase, alpha-1,3-galactosyltransferase, and alpha-1,4-galactosyltransferase.

85. The cell of claim 61, the cell comprising a galactosylation pathway comprising:
(i) a UDP-galactose biosynthesis pathway comprising at least one enzyme selected from the group consisting of galactose-1-epimerase, galactokinase, glucokinase, galactose-1-phosphate uridylyltransferase, UDP-glucose 4-epimerase, glucose-1-phosphate uridylyltransferase, phosphoglucomutase; and
(ii) a galactosyltransferase.

86. The cell of claim 61, wherein the cell comprises an N-acetylglucosaminylation pathway comprising:
(i) at least one enzyme selected from the group consisting of L-glutamine-D-fructose-6-phosphate aminotransferase, N-acetylglucosamine-6-phosphate deacetylase, phosphoglucosamine mutase, N-acetylglucosamine-1-phosphate uridylyltransferase/glucosamine-1-phosphate acetyltransferase; and
(ii) a N-acetylglucosaminyltransferase.

87. The cell of claim 61, wherein the cell comprises a pathway to synthesize lacto-N-tetraose (LNT) comprising a galactoside beta-1,3-N-acetylglucosaminyltransferase and an N-acetylglucosamine beta-1,3-galactosyltransferase.

88. The cell of claim 61, wherein the cell comprises a pathway to synthesize lacto-N-neotetraose (LNnT) comprising a galactoside beta-1,3-N-acetylglucosaminyltransferase and an N-acetylglucosamine beta-1,4-galactosyltransferase.

89. The cell of claim 61, wherein the cell is further capable of synthesizing a nucleotide-activated sugar to be used in the production of the compound.

90. The cell of claim 89, wherein the nucleotide-activated sugar is selected from the group consisting of UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), UDP-glucuronate, UDP-galacturonate, UDP-2-acetamido-2,6-dideoxy-L-arabino-4-hexulose, UDP-2-acetamido-2,6-dideoxy-L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid (CMP-Neu5Ac), CMP-N-glycolylneuraminic acid (CMP-Neu5Gc), GDP-fucose (GDP-Fuc), GDP-rhamnose, and UDP-xylose.

91. The cell of claim 61, wherein the cell comprises a catabolic pathway for selected mono-, di- or oligosaccharides which is at least partially inactivated, the mono-, di-, or oligosaccharides being involved in and/or required for the synthesis of the compound.

92. The cell of claim 61, wherein the cell utilizes a precursor for the synthesis of the compound the precursor being fed to the cell from the culture medium.

93. The cell of claim 61, wherein the cell produces a precursor for the synthesis of the compound.

94. The cell of claim 61, wherein the cell produces 30 g/L or more of compound in the whole broth and/or supernatant and/or wherein the compound in the whole broth and/or supernatant has a purity of at least 80% measured on the total amount of compound and its precursor produced by the cell in the whole broth and/or supernatant, respectively.

95. The cell of claim 61, wherein the cell is a bacterium, fungus, yeast, a plant cell, an animal cell, or a protozoan cell.

96. The cell of claim 61, wherein the cell is stably cultured in a medium.

97. The cell of claim 61, wherein the cell is capable of synthesizing a mixture of compounds.

98. A method for producing a compound, wherein the compound is a disaccharide, oligosaccharide and/or a Neu(n)Ac-containing bioproduct, wherein (n) is 4, 5, 7, 8 or 9 or a combination thereof, the method comprising the steps of:
a. providing a cell capable of producing the compound, wherein the cell is metabolically engineered for enhanced synthesis of acetyl-Coenzyme A,
b. cultivating the cell under conditions permissive for producing the compound, and
c. optionally separating the desired compound from the cultivation.

99. A method for producing a compound, wherein the compound is a disaccharide, oligosaccharide, and/or a Neu(n)Ac-containing bioproduct, wherein (n) is 4, 5, 7, 8 or 9 or a combination thereof by a metabolically engineered cell, the method comprising the steps of:
a) providing the cell of claim 61, and
b) culturing the cell in a culture medium under conditions permissive for producing the compound, and
c) optionally separating the compound from the cultivation.

100. The method according to claim 98, the method further comprising:
i) utilizing a culture medium comprising at least one precursor and/or acceptor for producing the compound, and/or
ii) adding to the culture medium at least one precursor and/or acceptor feed for producing the compound.

101. The method according to claim 99, the method further comprising:
i) utilizing a culture medium comprising at least one precursor and/or acceptor for producing the compound, and/or
ii) adding to the culture medium at least one precursor and/or acceptor feed for producing the compound.

102. The method according to claim 99, the method further comprising at least one of the following steps:
i) utilizing a culture medium comprising at least one precursor and/or acceptor for producing the compound;
ii) adding to the culture medium in a reactor or incubator at least one precursor and/or acceptor feed wherein the total reactor or incubator volume ranges from 250 mL (milliliter) to 10,000 m³, optionally in a continuous manner, optionally so that the final volume of the culture medium is not more than three-fold of the volume of the culture medium before the addition of the precursor and/or acceptor feed;
iii) adding to the culture medium in a reactor or incubator at least one precursor and/or acceptor feed wherein the total reactor or incubator volume ranges from 250 mL (milliliter) to 10,000 m³, optionally in a continuous manner, optionally so that the final volume of the culture medium is not more than three-fold of the volume of the culture medium before the addition of the precursor and/or acceptor feed and wherein optionally, the pH of the precursor and/or acceptor feed is set between 2.0 and 10.0, and wherein optionally the temperature of the precursor and/or acceptor feed is kept between 20° C. and 80° C.;
iv) adding at least one precursor and/or acceptor feed in a continuous manner to the culture medium over the course of 1 day, 2 days, 3 days, 4 days, 5 days by means of a feeding solution;
v) adding at least one precursor and/or acceptor feed in a continuous manner to the culture medium over the course of 1 day, 2 days, 3 days, 4 days, 5 days by means of a feeding solution and wherein optionally the pH of the precursor and/or acceptor feed is set between 2.0 and 10.0, and wherein optionally the temperature of the precursor and/or acceptor feed is kept between 20° C. and 80° C.;
the method resulting in a compound with a concentration of at least 50 g/L in the final volume of the culture medium.

103. The method according to claim 99, the method further comprising at least one of the following steps:
i) using a culture medium comprising at least 50 grams of precursor per liter of initial reactor or incubator volume wherein the total reactor or incubator volume ranges from 250 mL to 10,000 m³;
ii) using a culture medium comprising at least 50 grams of acceptor per liter of initial reactor or incubator volume wherein the total reactor or incubator volume ranges from 250 mL to 10,000 m³;
iii) adding to the culture medium in a reactor or incubator a precursor feed comprising at least 50 grams of precursor per liter of initial reactor or incubator volume wherein the total reactor or incubator volume ranges from 250 mL to 10,000 m³ optionally in a continuous manner, optionally so that the final volume of the culture medium is not more than three-fold of the volume of the culture medium before the addition of the precursor feed;
iv) adding to the culture medium in a reactor or incubator an acceptor feed comprising at least 50 grams of acceptor per liter of initial reactor or incubator volume wherein the total reactor or incubator volume ranges from 250 mL to 10,000 m³ optionally in a continuous manner, optionally so that the final volume of the culture medium is not more than three-fold of the volume of the culture medium before the addition of the acceptor feed;
v) adding to the culture medium in a reactor or incubator a precursor feed comprising at least 50 grams of precursor per liter of initial reactor or incubator volume wherein the total reactor or incubator volume ranges from 250 mL to 10,000 m³ optionally in a continuous manner, optionally so that the final volume of the culture medium is not more than three-fold of the volume of the culture medium before the addition of the precursor feed and wherein optionally the pH of the precursor feed is set between 2.0 and 10.0, and wherein optionally the temperature of the precursor feed is kept between 20° C. and 80° C.;
vi) adding to the culture medium in a reactor or incubator an acceptor feed comprising at least 50 grams of acceptor per liter of initial reactor or incubator volume wherein the total reactor or incubator volume ranges from 250 mL to 10,000 m³ optionally in a continuous manner, optionally so that the final volume of the culture medium is not more than three-fold of the volume of the culture medium before the addition of the acceptor feed and wherein optionally the pH of the acceptor feed is set between 2.0 and 10.0, and wherein optionally the temperature of the acceptor feed is kept between 20° C. and 80° C.;
vii) adding a precursor and/or acceptor feed in a continuous manner to the culture medium over the course of 1 day, 2 days, 3 days, 4 days, 5 days by means of a feeding solution;
viii) adding a precursor feed in a continuous manner to the culture medium over the course of 1 day, 2 days, 3 days, 4 days, 5 days by means of a precursor feeding solution and wherein the concentration of the precursor feeding solution is 50 g/L and wherein optionally the pH of the precursor feed is set between 2.0 and 10.0, and wherein optionally the temperature of the precursor feed is kept between 20° C. and 80° C.;
ix) adding an acceptor feed in a continuous manner to the culture medium over the course of 1 day, 2 days, 3 days, 4 days, 5 days by means of an acceptor feeding solution and wherein the concentration of the acceptor feeding solution is 50 g/L and wherein optionally the pH of the acceptor feed is set between 2.0 and 10.0, and wherein optionally the temperature of the acceptor feed is kept between 20° C. and 80° C.;
the method resulting in a compound with a concentration of at least 50 g/L in the final volume of the culture medium.

104. The method according to claim 99, the method further comprising at least one of the following steps:
i) using a culture medium comprising at least 50 grams of lactose per liter of initial reactor or incubator volume wherein the total reactor or incubator volume ranges from 250 mL to 10,000 m³;
ii) adding to the culture medium in a reactor or incubator a lactose feed comprising at least 50 grams of lactose per liter of initial reactor or incubator volume wherein the total reactor or incubator volume ranges from 250 mL to 10,000 m³ optionally in a continuous manner, optionally so that the final volume of the culture medium is not more than three-fold of the volume of the culture medium before the addition of the lactose feed;
iii) adding to the culture medium in a reactor or incubator a lactose feed comprising at least 50 grams of lactose per liter of initial reactor or incubator volume wherein the total reactor or incubator volume ranges from 250 mL to 10,000 m³ optionally in a continuous manner, optionally so that the final volume of the culture medium is not more than three-fold of the volume of the culture medium before the addition of the lactose feed and wherein optionally the pH of the lactose feed is set between 2.0 and 10.0, and wherein optionally the temperature of the lactose feed is kept between 20° C. and 80° C.;
iv) adding a lactose feed in a continuous manner to the culture medium over the course of 1 day, 2 days, 3 days, 4 days, 5 days by means of a feeding solution;
v) adding a lactose feed in a continuous manner to the culture medium over the course of 1 day, 2 days, 3 days, 4 days, 5 days by means of a lactose feeding solution and wherein the concentration of the lactose feeding solution is 50 g/L and wherein optionally the pH of the lactose feed is set between 2.0 and 10.0, and wherein optionally the temperature of the lactose feed is kept between 20° C. and 80° C.;
the method resulting in a compound with a concentration of at least 50 g/L in the final volume of the culture medium.

105. The method according to claim 104, wherein the lactose feed is accomplished by adding lactose from the beginning of the cultivation in a concentration of at least 5 mM.

106. The method according to claim 104, wherein the lactose feed is accomplished by adding lactose to the culture medium in a concentration, such that throughout the production phase of the cultivation, a lactose concentration of at least 5 mM is obtained.

107. The method for producing a mixture of compounds by a metabolically engineered cell, comprising the steps of:
a) providing the cell of claim 97, and
b) culturing the cell in a culture medium under conditions permissive for producing the compounds, and
c) optionally separating the mixture of compounds from the cultivation.

108. The method according to claim 99, wherein the cells are cultivated for at least about 60, 80, 100, or about 120 hours or in a continuous manner.

109. The method according to claim 99, wherein a carbon and energy source is also added, optionally continuously to the culture medium, optionally with the precursor and/or acceptor.

110. The method according to claim 99, wherein the cell uses at least one precursor for the synthesis of the compound.

111. The method according to claim 99, wherein the culture medium contains at least one molecule selected from the group comprising lactose, galactose, sialic acid, fucose, GlcNAc, GalNAc, lacto-N-biose (LNB), N-acetyllactosamine (LacNAc).

112. The method according to claim 99, wherein a first phase of exponential cell growth is provided by adding a carbon-based substrate to the culture medium before the precursor and/or acceptor is added to the culture medium in a second phase.

113. The method according to claim 99, wherein the cell is producing at least one precursor for the synthesis of the compound.

114. The method according to claim 101, wherein the precursor for the synthesis of the compound is completely converted into the compound.

115. The method according to claim 99, wherein the compound is separated from the culture medium and/or the cell.

116. The method according to claim 115, wherein the separation comprises at least one of the following steps: clarification, ultrafiltration, nanofiltration, two-phase partitioning, reverse osmosis, microfiltration, activated charcoal or carbon treatment, treatment with non-ionic surfactants, enzymatic digestion, tangential flow high-performance filtration, tangential flow ultrafiltration, affinity chromatography, ion exchange chromatography, hydrophobic interaction chromatography and/or gel filtration, ligand exchange chromatography, electrodialysis.

117. The method according to claim 99, wherein the method further comprises purification of the compound.

118. The method according to claim 117, wherein the purification comprises at least one of the following steps: using activated charcoal or carbon, using charcoal, nanofiltration, ultrafiltration, electrophoresis, enzymatic treatment or ion exchange, temperature adjustment, pH adjustment, pH adjustment with an alkaline or acidic solution, using alcohols, using aqueous alcohol mixtures, crystallization, evaporation, precipitation, drying, spray drying, lyophilization, spray freeze drying, freeze spray drying, band drying, belt drying, vacuum band drying, vacuum belt drying, drum drying, roller drying, vacuum drum drying or vacuum roller drying.

119. A method of using the cell of claim 61 in a fermentative process, the method comprising:
cultivating the cell to produce a compound.

120. The method according to claim 119, wherein the compound is a mixture of compounds.

121. The method according to claim 119, wherein the compound is an oligosaccharide.
